(19) United States
(12) Patent Application Publication
(10) Pub. No.: US 2026/0035431 A1
(43) Pub. Date: Feb. 5, 2026

(54) IGF-1-RELATED COMPOSITIONS AND USES OF THE SAME

(71) Applicant: Cavalry Biosciences, Inc., San Francisco, CA (US)

(72) Inventors: Nathaniel Eames DAVID, San Francisco, CA (US); Robert Norman O’BRIEN, Burlington, VT (US); Timothy R. STOWE, South San Francisco, CA (US); Jan VAN DEURSEN, Novato, CA (US); James W. WEST, Bend, OR (US); Patrick Brit VENTURA, Richmond, CA (US); Janine VAN REE, Novato, CA (US)

(21) Appl. No.: 19/300,509

(22) Filed: Aug. 14, 2025

Related U.S. Application Data
(63) Continuation of application No. PCT/US2025/020947, filed on Mar. 21, 2025.
(60) Provisional application No. 63/569,529, filed on Mar. 25, 2024.
(60) Provisional application No. 63/569,531, filed on Mar. 25, 2024.
(60) Provisional application No. 63/663,861, filed on Jun. 25, 2024.
(60) Provisional application No. 63/663,866, filed on Jun. 25, 2024.

(51) Int. Cl. C07K 14/65; A61K 38/30; A61P 27/02

(52) U.S. Cl. CPC C07K 14/65; A61P 27/02; A61K 38/30

(57) ABSTRACT

The present disclosure provides composition for use in treating disorders, such as eye disorders (e.g., disorders affecting Meibomian glands). Composition(s) for use in such disorders may act on the IGF-1 pathway, such as by binding to (e.g., agonizing) IGF1R. Such compositions may evade IGF binding proteins. Related methods, kits, and pharmaceutical compositions are also disclosed.

### CROSS-REFERENCE

This application is a continuation of International Application PCT/US2025/020947, filed Mar. 21, 2025, which claims the benefit of U.S. Provisional Application No. 63/569,529, filed on Mar. 25, 2024, U.S. Provisional Application No. 63/569,531, filed on Mar. 25, 2024, U.S. Provisional Application No. 63/663,861, filed on Jun. 25, 2024, and U.S. Provisional Application No. 63/663,866, filed on Jun. 25, 2024, each of which is incorporated herein by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Aug. 14, 2025, is named 61766.723.601_SL.xml and is 700,000 bytes in size.

### BACKGROUND OF THE INVENTION

Insulin-like growth factor 1 (IGF-1) is a hormone similar in molecular structure to insulin which plays an important role in childhood growth, and has anabolic effects in adults. IGF-1 is a protein that in humans is encoded by the IGF1 gene. Processed and mature wild-type human IGF-1 consists of 70 amino acids in a single chain with three intramolecular disulfide bridges. Production of IGF-1 is stimulated by growth hormone (GH). IGF-1 is often bound to one or more of 6 binding proteins (IGF-BPs). IGF-1 is produced primarily in liver and serves as an endocrine (as well as paracrine and autocrine) hormone mediating the action of GH in peripheral tissues such as muscle, cartilage, bone, kidney, nerves, skin, lungs, and the liver itself. Improved versions of IGF-1 are needed that have one or more advantageous properties, such as properties that are useful in treating various diseases and disorders including, for example, Meibomian Gland Dysfunction (MGD).

### SUMMARY OF THE INVENTION

In an aspect, the present disclosure provides an engineered polypeptide comprising an IGF-1 variant, wherein said engineered polypeptide has reduced affinity to at least one IGF binding protein (IGFBP) as compared to the affinity between wild-type IGF-1 (SEQ ID NO: 1) and the IGFBP. In some embodiments, the engineered polypeptide comprises the amino acid sequence of any one of SEQ ID NOS: 2-771. In some embodiments, the IGF-1 variant is a truncation. In some embodiments, the IGF-1 variant comprises or consists of the amino sequence of SEQ ID NO: 3. In some embodiments, the IGF-1 variant comprises one or more amino acid substitutions relative to wild-type IGF-1 (SEQ ID NO: 1). In some embodiments, the IGF-1 variant comprises an amino acid deletion at position 37 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 6. In some embodiments, the IGF-1 variant comprises an amino acid substitution at position 60 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 2. In some embodiments, the IGF-1 variant comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 4. In some embodiments, the IGF-1 variant comprises the amino sequence of SEQ ID NO: 5. In some embodiments, the IGF-1 variant comprises the amino sequence of SEQ ID NO: 7. In some embodiments, the IGF-1 variant comprises the amino sequence of SEQ ID NO: 12. In some embodiments, the IGF-1 variant comprises an amino acid sequence selected from any one of SEQ ID NOS: 13-771. In some embodiments, the IGF-1 variant comprises an amino acid sequence selected from any one of SEQ ID NOS: 13-767. In some embodiments, the IGF-1 variant comprises the amino sequence of SEQ ID NO: 16. In some embodiments, the IGF-1 variant comprises the amino sequence of SEQ ID NO: 17. In some embodiments, the IGF-1 variant comprises the amino sequence of SEQ ID NO: 18. In some embodiments, the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 15 and 21-38. In some embodiments, the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 58-75. In some embodiments, the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 95-112. In some embodiments, the IGF-1 variant comprises (1) a truncation of the first three amino acid of the wild-type IGF-1 (SEQ ID NO: 1) and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 39-57. In some embodiments, the IGF-1 variant comprises (1) a truncation of the first three amino acid of the wild-type IGF-1 (SEQ ID NO: 1) and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 76-94. In some embodiments, the IGF-1 variant comprises (1) a truncation of the first three amino acid of the wild-type IGF-1 (SEQ ID NO: 1) and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 113-131. In some embodiments, the one or more amino acid substitutions relative to wild-type IGF-1 (SEQ ID NO: 1) are at positions 12, 49, 54, or a combination thereof. In some embodiments, the IGF-1 variant comprises an amino acid sequence selected from any one of SEQ ID NOS: 132-197. In some embodiments, the IGF-1 variant comprises an amino acid substitution at position 5 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 578-596. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 198-216. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 217-235. In some embodiments, the IGF-1 variant comprises an amino acid substitution at position 7 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 597-615. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 236-254. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 255-273. In some embodiments, the IGF-1 variant comprises an amino acid substitution at position 10 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 616-634. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 274-292. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 293-311. In some embodiments, the IGF-1 variant comprises an amino acid substitution at position 17 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 635-653. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 312-330. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 331-349. In some embodiments, the IGF-1 variant comprises an amino acid substitution at position 22 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 654-672. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 350-368. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 369-387. In some embodiments, the IGF-1 variant comprises an amino acid substitution at position 23 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 673-691. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 388-406. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 407-425. In some embodiments, the IGF-1 variant comprises an amino acid substitution at position 24 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 692-710. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 426-444. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 445-463. In some embodiments, the IGF-1 variant comprises an amino acid substitution at position 25 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 711-729. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 464-482. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 483-501. In some embodiments, the IGF-1 variant comprises an amino acid substitution at position 34 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 730-748. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 502-520. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 521-539. In some embodiments, the IGF-1 variant comprises an amino acid substitution at position 57 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 749-767. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 540-558. In some embodiments, the amino acid substitution at position 3 is a lysine. In some embodiments, the IGF-1 variant comprises the amino acid sequence of SEQ ID NO: 768 or 770. In some embodiments, the amino acid substitution at position 3 is a glycine. In some embodiments, the IGF-1 variant comprises an amino acid sequence selected from SEQ ID NO: 769 or 771. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 549-577. In some embodiments, the IGF-1 variant is coupled to a cell-penetrating peptide (CPP) or skin-penetrating peptide (SPP). In some embodiments, the IGF-1 variant is coupled to a cell-penetrating peptide selected from the group consisting of SEQ ID NO: 10 and SEQ ID NO: 11.

In some embodiments, the IGF-1 variant is coupled to the skin-penetrating peptide of SEQ ID NO: 9. In some embodiments, the IGF-1 variant comprises the amino acid sequence of SEQ ID NO: 8. In some embodiments, the at least one IGFBP comprises IGFBP2. In some embodiments, the at least one IGFBP comprises IGFBP3. In some embodiments, the at least one IGFBP comprises IGFBP1. In some embodiments, the at least one IGFBP comprises IGFBP4. In some embodiments, the at least one IGFBP comprises IGFBP5. In some embodiments, the at least one IGFBP comprises IGFBP6.

In another aspect, the present disclosure provides a pharmaceutical composition comprising a therapeutically effective amount of an IGF-1 variant that has reduced affinity to at least one IGF binding protein (IGFBP) as compared to the affinity for the interaction between wild-type IGF-1 (SEQ ID NO: 1) and the IGFBP, wherein the pharmaceutical composition is formulated for local administration. In some embodiments, the pharmaceutical composition is formulated for local administration to an eye or eyelid.

In another aspect, the present disclosure provides a pharmaceutical composition comprising a therapeutically effective amount of an IGF-1 variant, wherein a local administration of the pharmaceutical composition to an eye or eyelid results in one or more of an increase in a size of meibomian glands; a decrease in meibomian gland atrophy; a reversal of age-associated meibomian gland atrophy; an increase in a function of one or more meibocytes; an increase in corneal epithelial cell proliferation; an increase in meibocyte proliferation; an increase in corneal healing rate; an increase in IGF1 receptor (IGF1R) activation in the meibomian glands; an increase duration of IGF1R activation in the meibomian glands; and an increase of lipid content of the meibomian glands. In some embodiments, the IGF-1 variant has reduced affinity to at least one IGF binding protein relative to wild-type IGF-1 to the IGFBP.

In another aspect, the present disclosure provides a non-naturally occurring IGF-1 variant that has reduced affinity to at least one IGF binding protein (IGFP) as compared to the affinity for the interaction between wild-type IGF-1 (SEQ ID NO: 1) and the IGFBP. In another aspect, the present disclosure provides a pharmaceutical composition comprising a therapeutically effective amount of the non-naturally occurring IGF-1 variant described herein. In some embodiments, the pharmaceutical composition further comprises one or more pharmaceutically acceptable excipients. In some embodiments, the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone. In some embodiments, the pharmaceutical composition is formulated for delivery via an eyedrop. In some embodiments, the pharmaceutical composition comprises a cream for administration to one or both eyelids of a subject. In some embodiments, the IGF-1 variant has at least 80%, at least 85%, at least 90%, at least 95%, or at least 98% sequence identity to SEQ ID NO: 1. In some embodiments, the pharmaceutical composition or IGF-1 variant, when administered to subjects suffering from Meibomian gland dysfunction, results in a median increase in surface area or volume of meibomian glands within the inner eyelid surface of the subject as compared to subjects who do not receive the pharmaceutical composition or IGF-1 variant. In some embodiments, the pharmaceutical composition or IGF-1 variant, when administered to subjects suffering from Meibomian gland dysfunction, results in a median increase in lipid content within a Meibomian gland of the subject as compared to subjects who do not receive the pharmaceutical composition or IGF-1 variant. In some embodiments, the pharmaceutical composition or IGF-1 variant, when administered to subjects suffering from Meibomian gland dysfunction results, in a median increase in lipid quality within a Meibomian gland of the subject as compared to subjects who do not receive the pharmaceutical composition or IGF-1 variant. In some embodiments, the pharmaceutical composition or IGF-1 variant, when administered to subjects suffering from Meibomian gland dysfunction, results in a median increase in release of lipid from acini of a Meibomian gland of subjects as compared to a subjects who do not receive the pharmaceutical composition or IGF-1 variant. In some embodiments, the pharmaceutical composition or IGF-1 variant, when administered to subjects suffering from meibomian gland dysfunction results in a median increase in duration of phosphorylation of Akt in meibocytes relative to subjects who do not receive the pharmaceutical composition or IGF-1 variant. the IGF-1 variant is a truncation. In some embodiments, the IGF-1 variant comprises or consists of the amino sequence of SEQ ID NO: 3. In some embodiments, the IGF-1 variant comprises one or more amino acid substitutions relative to wild-type IGF-1 (SEQ ID NO: 1). In some embodiments, the IGF-1 variant comprises an amino acid deletion at position 37 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, wherein the IGF-1 variant comprises the amino sequence of SEQ ID NO: 6. In some embodiments, the IGF-1 variant comprises an amino acid substitution at position 60 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, wherein the IGF-1 variant comprises the amino sequence of SEQ ID NO: 2. In some embodiments, the IGF-1 variant comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 4. In some embodiments, the IGF-1 variant comprises the amino sequence of SEQ ID NO: 5. In some embodiments, the IGF-1 variant comprises the amino sequence of SEQ ID NO: 7. In some embodiments, the IGF-1 variant comprises the amino sequence of SEQ ID NO: 12. In some embodiments, the IGF-1 variant comprises an amino acid sequence selected from any one of SEQ ID NOS: 13-771. In some embodiments, the IGF-1 variant comprises an amino acid sequence selected from any one of SEQ ID NOS: 13-767. In some embodiments, the IGF-1 variant comprises the amino sequence of SEQ ID NO: 16. In some embodiments, the IGF-1 variant comprises the amino sequence of SEQ ID NO: 17. In some embodiments, the IGF-1 variant comprises the amino sequence of SEQ ID NO: 18. In some embodiments, the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 15 and 21-38. In some embodiments, the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 58-75.

In some embodiments, the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 95-112. In some embodiments, the IGF-1 variant comprises (1) a truncation of the first three amino acid of the wild-type IGF-1 (SEQ ID NO: 1) and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 39-57. In some embodiments, the IGF-1 variant comprises (1) a truncation of the first three amino acid of the wild-type IGF-1 (SEQ ID NO: 1) and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 76-94. In some embodiments, the IGF-1 variant comprises (1) a truncation of the first three amino acid of the wild-type IGF-1 (SEQ ID NO: 1) and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 113-131. In some embodiments, the one or more amino acid substitutions relative to wild-type IGF-1 (SEQ ID NO: 1) are at positions 12, 49, 54, or a combination thereof. In some embodiments, the IGF-1 variant comprises an amino acid sequence selected from any one of SEQ ID NOS: 132-197. In some embodiments, the IGF-1 variant comprises an amino acid substitution at position 5 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 578-596. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 198-216. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 217-235. In some embodiments, the IGF-1 variant comprises an amino acid substitution at position 7 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 597-615. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 236-254. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 255-273. In some embodiments, the IGF-1 variant comprises an amino acid substitution at position 10 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 616-634. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 274-292. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 293-311. In some embodiments, the IGF-1 variant comprises an amino acid substitution at position 17 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 635-653. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 312-330. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 331-349. In some embodiments, the IGF-1 variant comprises an amino acid substitution at position 22 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, he IGF-1 variant comprises an amino acid sequence selected from any one of SEQ ID NOS: 654-672. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 350-368. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 369-387. In some embodiments, the IGF-1 variant comprises an amino acid substitution at position 23 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 673-691. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 388-406. In some embodiments, the IGF-1 variant further comprises a truncation of the first three amino acid of the wild-type IGF-1 (SEQ ID NO: 1). In some embodiments, IGF-1 variant comprises an amino acid sequence selected from any one of SEQ ID NOS: 407-425. In some embodiments, the IGF-1 variant comprises an amino acid substitution at position 24 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, IGF-1 variant comprises an amino acid sequence selected from any one of SEQ ID NOS: 692-710. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 426-444. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 445-463. In some embodiments, the IGF-1 variant comprises an amino acid substitution at position 25 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 711-729. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 464-482. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 483-501. In some embodiments, the IGF-1 variant comprises an amino acid substitution at position 34 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 730-748. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, wherein the IGF-1 variant comprises an amino acid sequence selected from any one of SEQ ID NOS: 502-520. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 521-539. In some embodiments, the IGF-1 variant comprises an amino acid substitution at position 57 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises an amino acid sequence selected from any one of SEQ ID NOS: 749-767. In some embodiments, the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the IGF-1 variant comprises an amino acid sequence selected from any one of SEQ ID NOS: 540-558. In some embodiments, the IGF-1 variant further comprises a truncation of the first three amino acid of the wild-type IGF-1 (SEQ ID NO: 1). In some embodiments, the IGF-1 variant comprises an amino acid sequence selected from any one of SEQ ID NOS: 549-577. In some embodiments, the IGF-1 variant is coupled to a cell-penetrating peptide (CPP) or skin-penetrating peptide (SPP). In some embodiments, the IGF-1 variant is coupled to a cell-penetrating peptide selected from the group consisting of SEQ ID NO: 10 and SEQ ID NO: 11. In some embodiments, the IGF-1 variant is coupled to the skin-penetrating peptide of SEQ ID NO: 9. In some embodiments, the IGF-1 variant comprises the amino acid sequence of SEQ ID NO: 8. In some embodiments, the at least one IGFBP comprises IGFBP2. In some embodiments, at least one IGFBP comprises IGFBP3. In some embodiments, the at least one IGFBP comprises IGFBP1. In some embodiments, the at least one IGFBP comprises IGFBP4. In some embodiments, the at least one IGFBP comprises IGFBP5. In some embodiments, the at least one IGFBP comprises IGFBP6. In some embodiments, administration of the pharmaceutical composition or IGF-1 variant to an eye or eyelid of the subject results in an increase in a size of the meibomian glands. In some embodiments, administration of the pharmaceutical composition or IGF-1 variant to an eye or eyelid of the subject results in a decrease in meibomian gland atrophy. In some embodiments, administration of the pharmaceutical composition or IGF-1 variant to an eye or eyelid of the subject results in a reversal of age-associated meibomian gland atrophy. In some embodiments, administration of the pharmaceutical composition or IGF-1 variant to an eye or eyelid of the subject results in an increase in a function of one or more meibocytes. In some embodiments, administration of the pharmaceutical composition or IGF-1 variant to an eye or eyelid of the subject results in an increase in corneal epithelial cell proliferation. In some embodiments, administration of the pharmaceutical composition or IGF-1 variant to an eye or eyelid of the subject results in an increase in meibocyte proliferation. In some embodiments, administration of the pharmaceutical composition or IGF-1 variant to an eye or eyelid of the subject results in an increase in meibocyte differentiation. In some embodiments, administration of the pharmaceutical composition or IGF-1 variant to an eye or eyelid of the subject results in an increase in corneal healing. In some embodiments, administration of the pharmaceutical composition or IGF-1 variant to an eye or eyelid of the subject results in an increase in IGF1 receptor (IGF1R) activation in the meibomian glands. In some embodiments, administration of the pharmaceutical composition or IGF-1 variant to an eye or eyelid of the subject results in an increase duration of IGF1R activation in the meibomian glands. In some embodiments, administration of the pharmaceutical composition or IGF-1 variant to an eye or eyelid of the subject results in an increase of lipid content of the meibomian glands. In some embodiments, the pharmaceutical composition or IGF-1 variant, when administered to spheroids of IHGMGE cells, results in, on average, an increase in lipid content in the spheroids.

In another aspect, the present disclosure provides a pharmaceutical composition comprising: a therapeutically effective amount of an IGF-1 variant comprising a polypeptide comprising an amino acid sequence of any one of SEQ ID NOS: 2-8 and 12-767 (or alternatively any one of SEQ ID NOS: 2-8 and 12-771); and one or more pharmaceutically acceptable excipients. In some embodiments, the pharmaceutical composition is a solution for delivery as an eyedrop. In some embodiments, the pharmaceutical composition comprises a cream for administration to one or both eyelids. In some embodiments, the pharmaceutical composition is formulated for systemic delivery. In some embodiments, the polypeptide is a human IGF-1R agonist. In some embodiments, administration of the pharmaceutical composition to subjects suffering from meibomian gland dysfunction results in a median increase in surface area or volume of meibomian glands within the inner eyelid surface. In some embodiments, administration of the pharmaceutical composition to subjects suffering from meibomian gland dysfunction results in a median increase in lipid content within meibomian glands. In some embodiments, administration of the pharmaceutical composition to subjects suffering from meibomian gland dysfunction results in a median increase in release of lipid from acini of the meibomian glands. In some embodiments, administration of the pharmaceutical composition to subjects suffering from meibomian gland dysfunction results in a median increase in release of lipid from acini of the meibomian glands. In some embodiments, administration of the pharmaceutical composition to subjects suffering from meibomian gland dysfunction results in a median increase in phosphorylation of Akt in meibocytes. In some embodiments, the pharmaceutical composition does not comprise any additional phospholipidosis-inducing agent. In some embodiments, the pharmaceutical composition does not comprise any one of azithromycin or doxycycline. In some embodiments, the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone. In some embodiments, the polypeptide comprises an amino acid sequence selected from any one of SEQ ID NOS: 2-12. In some embodiments, the polypeptide comprises an amino acid sequence selected from any one of SEQ ID NOS: 13-771. In some embodiments, the polypeptide comprises an amino acid sequence selected from any one of SEQ ID NOS: 13-767. In some embodiments, the polypeptide comprises the amino acid sequence of SEQ ID NO: 16. In some embodiments, the polypeptide comprises the amino acid sequence of SEQ ID NO: 17. In some embodiments, the polypeptide comprises the amino acid sequence of SEQ ID NO: 18. In some embodiments, the polypeptide comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 15 and 21-38. In some embodiments, the polypeptide comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 58-75. In some embodiments, the polypeptide comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 95-112. In some embodiments, the polypeptide comprises (1) a truncation of the first three amino acid of the wild-type IGF-1 (SEQ ID NO: 1) and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 39-57. In some embodiments, the polypeptide comprises (1) a truncation of the first three amino acid of the wild-type IGF-1 (SEQ ID NO: 1) and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 76-94. In some embodiments, the polypeptide comprises (1) a truncation of the first three amino acid of the wild-type IGF-1 (SEQ ID NO: 1) and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 113-131. In some embodiments, the polypeptide comprises one or more amino acid substitutions relative to wild-type IGF-1 (SEQ ID NO: 1), wherein the one or more amino acid substitutions relative to wild-type IGF-1 (SEQ ID NO: 1) are at positions 12, 49, 54, or a combination thereof. In some embodiments, the polypeptide comprises an amino acid sequence selected from any one of SEQ ID NOS: 132-197. In some embodiments, the polypeptide comprises an amino acid substitution at position 5 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 578-596. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 198-216. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 217-235. In some embodiments, the polypeptide comprises an amino acid substitution at position 7 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 597-615. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 236-254. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 255-273. In some embodiments, the polypeptide comprises an amino acid substitution at position 10 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 616-634. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 274-292. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 293-311. In some embodiments, the polypeptide comprises an amino acid substitution at position 17 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 635-653. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 312-330. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 331-349. In some embodiments, the polypeptide comprises an amino acid substitution at position 22 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 654-672. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 350-368. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 369-387. In some embodiments, the polypeptide comprises an amino acid substitution at position 23 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 673-691. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 388-406. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 407-425. In some embodiments, the polypeptide comprises an amino acid substitution at position 24 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 692-710. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 426-444. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 445-463. In some embodiments, the polypeptide comprises an amino acid substitution at position 25 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 711-729. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 464-482. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 483-501. In some embodiments, the polypeptide comprises an amino acid substitution at position 34 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 730-748. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 502-520. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 521-539. In some embodiments, the polypeptide comprises an amino acid substitution at position 57 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the polypeptide comprises an amino acid sequence selected from any one of SEQ ID NOS: 749-767. In some embodiments, the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the amino acid substitution at position 3 is an arginine. In some embodiments, the polypeptide comprises an amino acid sequence selected from any one of SEQ ID NOS: 540-558. In some embodiments, the polypeptide further comprises a truncation of the first three amino acid of the wild-type IGF-1 (SEQ ID NO: 1). In some embodiments, the polypeptide comprises an amino acid sequence selected from any one of SEQ ID NOS: 549-577. In some embodiments, the polypeptide further comprises a cell-penetrating peptide (CPP) or skin-penetrating peptide (SPP). In some embodiments, the polypeptide comprises a cell-penetrating peptide selected from the group consisting of SEQ ID NO: 10 and SEQ ID NO: 11. In some embodiments, the polypeptide comprises the skin-penetrating peptide of SEQ ID NO: 9.

In another aspect, the present disclosure provides a kit comprising: the pharmaceutical composition or IGF-1 variant provided herein; and an eyedropper for delivery of the pharmaceutical composition or IGF-1 variant as an eyedrop. In another aspect, the present disclosure provides a method of treating a disease or disorder in a subject in need thereof, the method comprising administering to the subject a pharmaceutical composition comprising a non-naturally occurring IGF-1 variant that has reduced affinity to an IGF binding protein (IGFBP) relative to the affinity for the interaction between wild-type IGF-1 (SEQ ID NO: 1) and the IGFBP. In another aspect, the present disclosure provides a method for treating an eye disorder in a subject in need thereof, the method comprising administering a pharmaceutical composition to a subject having an eye disorder, wherein the pharmaceutical composition comprises a therapeutically effective amount of an IGF-1 variant that has reduced affinity to an IGF binding protein (IGFBP) relative to the affinity for the interaction between wild-type IGF-1 (SEQ ID NO: 1) and the IGFBP. In another aspect, the present disclosure provides a method for local administration of an IGF-1 variant to a subject, the method comprising administering an IGF-1 variant to a subject, wherein the IGF-1 variant has reduced affinity to an IGF binding protein (IGFBP) relative to the affinity of an interaction between the wild-type IGF-1 (SEQ ID NO: 1) and the IGFBP. In some embodiments, the pharmaceutical composition is the pharmaceutical composition or IGF-1 variant provided herein. In some embodiments, the pharmaceutical composition or IGF-1 variant is administered to an eye or eyelid of the subject. In some embodiments, the pharmaceutical composition or IGF-1 variant is administered to the eye of the subject via an eyedropper. In some embodiments, the pharmaceutical composition or IGF-1 variant is administered to an outer eyelid of the subject. In some embodiments, the pharmaceutical composition or IGF-1 variant is a cream. In some embodiments, the pharmaceutical composition or IGF-1 variant is administered to a subject suffering from Meibomian gland dysfunction. In some embodiments, administering the pharmaceutical composition or IGF-1 variant to the subject results in an increase in surface area or volume of meibomian glands within the inner eyelid surface of the subject. In some embodiments, administering the pharmaceutical composition to the subject results in an increase in lipid content within a Meibomian gland of the subject. In some embodiments, administering the pharmaceutical composition to the subject results in an increase in release of lipid from acini of a Meibomian gland of the subject. In some embodiments, administering the pharmaceutical composition to the subject results in an increase in duration of phosphorylation of Akt in meibocytes. In some embodiments, the method does not comprise administration of any additional phospholipidosis-inducing agent. In some embodiments, the method does not comprise administration of one or both of azithromycin and doxycycline. In some embodiments, the eye disorder comprises dry eye disease. In some embodiments, the eye disorder comprises meibomian gland dysfunction. In some embodiments, the eye disorder comprises Sjorgren's syndrome.

In another aspect, the present disclosure provides a pharmaceutical composition comprising the engineered polypeptide provided herein. In another aspect, the present disclosure provides a kit comprising the engineered polypeptide provided herein and instructions for use. In some embodiments, the instructions for use recite a treatment of an eye disease or disorder. In another aspect, the present disclosure provides a method of treating a disease or disorder in a subject in need thereof, wherein the method comprises administering the engineered polypeptide provided herein or the pharmaceutical composition provided herein. In some embodiments, the disease or disorder is an eye disorder. In some embodiments, the eye disorder comprises dry eye disease. In some embodiments, the eye disorder comprises meibomian gland dysfunction. In some embodiments, wherein the eye disorder comprises Sjorgren's syndrome. In some embodiments, wherein the method does not comprise administration of one or both of azithromycin and doxycycline.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the dose-dependent effects of IGF-1 (SEQ ID NO: 1), IGF-1 Ea (SEQ ID NO: 7), IGF-1 Des1-3 R37X (SEQ ID NO: 6), IGF-1 Des1-3 (SEQ ID NO: 8), and IGF-1 E3R (SEQ ID NO: 4) on AKT S473 phosphorylation in DU145 cells. Wild-type IGF-1 and IGF-1 variants have similar EC₅₀ values in DU145 cells.

FIG. 2 illustrates reduced IGFBP affinity for various IGF-1 mutants. In L6 rat myoblast conditioned media containing multiple IGF binding proteins, competition assays report reduction in BP affinity for IGF-1 LR3 (SEQ ID NO: 12), LG3 (long IGF-1 E3G), long IGF-1, and IGF-1 Des 1-3 (SEQ ID NO: 3) to be 690 times, 112 times, 5.5 times, and 38 times respectively (see Francis, G L et al., 8(3) J. Mol. Endocrinol. 213-223, 1992). In a separate study using bovine IGFBP2, IGF-1 E3R (SEQ ID NO: 4) and IGF E3G were reported to have reduction in binding affinity for IGFBP by 230 times and 59 times, respectively (see King, R. et al., 8 J. Mol. Endocrinol. 29-41, 1992).

FIGS. 3A and 3B provide that IGF-1 LR3 (SEQ ID NO: 12) and IGF-1 E3R (SEQ ID NO: 4) are less inhibited by IGFBP2 and IGFBP3 compared to wild-type IGF-1 (SEQ ID NO: 1). FIG. 3A depicts the effects of wild-type IGF-1, IGF-1 LR3, and IGF-1 E3R on AKT S473 phosphorylation in DU145 cells in the presence of IGFBP2 at an IGF-1 to IGFBP2 ratio of 1:1, 1:2, or 1:4, respectively. FIG. 3B depicts the effects of wild-type IGF-1, IGF-1 LR3, and IGF-1 E3R on AKT S473 phosphorylation in DU145 cells in the presence of IGFBP3 at an IGF-1 to IGFBP3 ratio of 1:1, 1:2, or 1:4, respectively.

FIG. 4 depicts the dose-dependent effects of IGF-1 on AKT S473 phosphorylation in immortalized human meibomian gland epithelial cells (IHMGECs). The EC₅₀ value for IGF-1 is about 0.07 nanomolar (nM).

FIG. 5 depicts the results of live cell imaging assays: IHMGECs spread out and proliferate at increased rates upon IGF-1 stimulation in a dose-dependent manner.

FIG. 6 depicts the results of human IGF-1 stimulation in IGHMECs following the cell viability assay: the cells proliferate in response to IGF-1 in a dose-dependent matter.

FIGS. 7A and 7B depict immortalized Human Meibomian Gland Epithelial (IHMGE) cells form spheroids resembling meibomian gland acini when grown in a 3D culture. FIG. 7A depicts images of 2D and 3D spheroid cultures; brightfield images were collected with transmitted light. FIG. 7B depicts immunofluorescence images of nuclei markers, spheroids expressing markers (Krt5) of the meibomian gland acini basal compartment, and the merged images of the two.

FIG. 8 provides that spheroids grown in the presence of IGF-1 E3R are significantly larger than those grown with wild-type IGF-1. The diameters of spheroids cultured in the presence of 0 nM, 0.1 nM, 1.6 nM, or 10 nM of IGF-1 or IGF-1 E3R were measured on day seven of differentiation.

FIG. 9 provides IGF-1 and IGF-1 E3R proportionally increase lipid content proportional to spheroid diameter. Lipidtox intensity of spheroids cultured in the presence of 0 nM, 0.1 nM, 1.6 nM, or 10 nM of IGF-1 or IGF-1 E3R were measured on day seven of differentiation.

FIG. 10 provides that IGFBP2 is expressed higher compared to IGFBP1, IGFBP3, IGFBP4 and IGFBP6 in IHMGE spheroid cultures.

FIG. 11A illustrates the study design of transcriptional analysis of the effects of wild-type IGF1 treatment on IHMGE spheroids. FIG. 11B depicts that four IGFBPs were significantly upregulated by treatment of IHMGE spheroids with wild-type IGF-1 in the transcriptional analysis. FIG. 11C depicts that IGF-1 treatment significantly upregulates genes involved in fatty acid transport, lipid synthesis, and meibogenesis.

FIG. 12 provides that IGFBP2 is highly expressed in the basal compartment of the meibomian gland in mice. FIG. 12 depicts representative immunofluorescent images of meibomian glands labeling both of nuclei (DAPI, green) and proliferating cells (Ki67, red), IGFBP2, and the merged image of the two.

FIGS. 13A and 13B depict increased proliferation in meibomian gland acini in young mice compared to older mice. Cell proliferation in the Meibomian gland is reduced with age.

FIG. 13A depicts that the number of proliferating cells (Ki67+) in acini of aged meibomian glands was lower than that observed in young ones. Statistical comparison made using student's t-test, n=10 per group. FIG. 13B depicts representative immunofluorescent images of young (above) and old (below) meibomian glands labeling nuclei (DAPI, green) and proliferating cells (Ki67, red). Cells positive for both markers (Double positive) in acini are highlighted in blue using image analysis in ImageJ.

FIGS. 14A and 14B provide a graph and images showing increased proliferation in meibomian gland acini in aged mice upon systemic IGF-1 LR3 treatment. FIG. 14A shows that IGF1 LR3 can induce proliferation in the meibomian gland of aged mice. Mice were administered 2 doses of IGF-1 LR3 at 10 mg/kg by intraperitoneal injection 12 hours apart and taken down at 24 or 48 hours after the first dose. The number of proliferating cells (Ki67+) in acini of vehicle-treated (left) or IGF-1 LR3-treated (right) meibomian glands was quantified by comparing the number of Ki67 positive cells in meibomian gland acini. Statistical comparison made using Two-way ANOVA followed by Tukey's multiple comparison test. N=10 per group. FIG. 14B shows representative immunofluorescent images of meibomian glands from 24 hour vehicle-treated (above) and 24 hour IGF-1 LR3-treated (below) labeling nuclei (DAPI, green) and proliferating cells (Ki67, red). Cells positive for both markers (Double positive) in acini are highlighted in blue using image analysis in ImageJ.

FIGS. 15A and 15B provide a graph and images showing atrophy reversal and increased size in meibomian gland area in aged mice upon systemic IGF-1 LR3 treatment. FIG. 15A depicts that mice were untreated (young) or administered a IGF-1 LR3 at 10 mg/kg by intraperitoneal injection on 5 consecutive days per week for 4 weeks and subjected to transillumination meibography before sacrifice. Quantitation of meibography data shows increased gland area in IGF-1 LR3-treated old mice compared to vehicle-treated control old mice. Pairwise comparisons were made using one-tailed Mann-Whitney test, n=7-10 mice/group. FIG. 15B depicts representative images from transillumination meibography experiments with one acinus area in each image.

FIGS. 16A and 16B provide a graph and images showing increased lipid synthesis in Meibomian glands regardless of age upon systemic IGF-1 LR3 treatment. FIG. 16A depicts that mice were untreated (young) or administered a IGF-1 LR3 at 10 mg/kg by intraperitoneal injection on 5 consecutive days per week for 4 weeks before sacrifice. Nuclei (DAPI, red) and meibomian gland lipids (Lipidtox, green) were labeled in eyelid sections and lipid droplet density was quantified in ImageJ. Statistical comparisons were made using One-way ANOVA followed by Tukey's multiple comparison test. N=7-10 per group. FIG. 16B depicts representative images from each group.

FIG. 17 provides a graph showing that IGF-1 LR3 can be delivered systemically or by ocular drops to activate IGF1 receptor in the eyelid. Mice were administered a single dose of IGF-1 LR3 at 10 mg/kg by intraperitoneal injection or treated with vehicle (PBS) or PBS containing 5 mg/ml IGF-1 LR3 via eyedrops administered to the ocular surface. Animals were sacrificed 30 minutes after dosing and eyelids were homogenized and assays for pAKT levels by pAKT ELISA as a reporter for activation of IGF1R. Statistical comparisons were made using One-way ANOVA followed by Tukey's multiple comparison test. n=3 per group.

FIG. 18 provides that IGF-1 LR3 prolongs duration of increased IGF1R signaling compared to wild-type IGF-1. The effects of wild-type IGF-1 and IGF-1 LR3 on AKT S473 phosphorylation was measured after 0.5 hour or 2 hours post dosing. Statistical analysis: One-way Anova. n=3 per group.

FIG. 19 provides that both IGF-1 LR3 and IGF-1 Des 1-3, which are reduced in their ability to bind binding proteins, prolong duration of IGF1R activation at 2 hours post dosing compared to wild-type IGF-1. n=5/group. The effects of IGF-1, IGF-1 LR3 and IGF-1 Des 1-3 on AKT S473 phosphorylation was measured at 2 hours post dosing in eyelids of tested animals. Statistical analysis: One-way Anova.

FIG. 20 provides that IGF-1 LR3 and IGF-1 E3R, which are reduced in their ability to bind binding proteins, are similarly potent in vivo compared to IGF-1 at 1 hour post dosing. The effects of IGF-1, IGF-1 LR3 and IGF-1 E3R on AKT S473 phosphorylation was measured at 1 hour post dosing in eyelids of tested animals. n=3/group. Statistical analysis: One-way Anova.

FIG. 21 provides that IGF-1 LR3 stimulates dose-responsive proliferation in the aged mouse meibomian glands. The number of Ki67-labeled proliferating cells per 100 μm perimeter was measured following the treatment of 0 mg/ml, 0.3 mg/ml, 1 mg/ml, or 3 mg/ml of IGF-1 LR3 eyedrop in young or aged mice. n=5/group. Statistical analysis: One-way Anova.

FIGS. 22A and 22B provide that IGF-1 LR3 regenerates atrophied meibomian glands in aged mice. Daily treatment with IGF-1 LR3 for 1 month increases area of meibomian glands comparing pre- and post-treatment. FIG. 22A depicts individual animal glands before and after treatment of IGF-1 LR3 or vehicle treatment. FIG. 22B depicts the quantification of changes in gland area with the treatment of IGF-1 LR3 or vehicle as assessed by transillumination meibography. n=10/group. Statistical analysis: One-way ANOVA.

FIG. 23 provides that IGF-1 LR3 and IGF-1 E3R induce similar levels of proliferation in meibomian glands in aged mice after two weeks of daily eyedrop dosing. The number of Ki67-labeled proliferating cells per 100 m perimeter was measured following the treatment of vehicle or IGF-1 ER3 eyedrop in young or aged mice. n=4-5/group. Statistical analysis: One-way ANOVA.

FIG. 24A depicts the dose-dependent effects of IGF-1 (SEQ ID NO: 1) and IGF-1 E3R (SEQ ID NO: 4) on AKT S473 phosphorylation in DU145 cells. FIG. 24B depicts the dose-dependent effects of IGF-1 (SEQ ID NO: 1) and linker IGF-1 (SEQ ID NO: 13) on AKT S473 phosphorylation in DU145 cells. FIG. 24C depicts the dose-dependent effects of IGF-1 (SEQ ID NO: 1) and linker IGF-1 E3R (SEQ ID NO: 14) on AKT S473 phosphorylation in DU145 cells. FIG. 24D depicts the dose-dependent effects of IGF-1 (SEQ ID NO: 1) and IGF-1 E3R D12A (SEQ ID NO: 15) on AKT S473 phosphorylation in DU145 cells. FIG. 24E depicts the dose-dependent effects of IGF-1 (SEQ ID NO: 1) and IGF-1 E3R D20A (SEQ ID NO: 16) on AKT S473 phosphorylation in DU145 cells. FIG. 24F depicts the dose-dependent effects of IGF-1 (SEQ ID NO: 1) and IGF-1 E3R F49L (SEQ ID NO: 17) on AKT S473 phosphorylation in DU145 cells. FIG. 24G depicts the dose-dependent effects of IGF-1 (SEQ ID NO: 1) and IGF-1 E3R L54A (SEQ ID NO: 18) on AKT S473 phosphorylation in DU145 cells. FIG. 24H depicts the dose-dependent effects of IGF-1 (SEQ ID NO: 1) and IGF-1 E3R A13C/L57C (SEQ ID NO: 19) on AKT S473 phosphorylation in DU145 cells. FIG. 24I depicts the dose-dependent effects of IGF-1 (SEQ ID NO: 1) and IGF-1 E3R L14C/L57C (SEQ ID NO: 20) on AKT S473 phosphorylation in DU145 cells. Data points reflect the mean of two biological measurements normalized to pAKT levels for IGF1 at equivalent concentrations with error represented as SD.

FIGS. 25A and 25B depict inhibition of IGF-1 activity for various IGF-1 mutants in the presence of IGFBP2 and IGFBP3, respectively. FIG. 25A depicts IGFBP2 inhibition of IGF-1 (SEQ ID NO: 1), IGF-1 E3R (SEQ ID NO: 4), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), IGF-1 E3R A13C/L57C (SEQ ID NO: 19), and IGF-1 E3R L14C/L57C (SEQ ID NO: 20). FIG. 25B depicts IGFBP3 inhibition of IGF-1 (SEQ ID NO: 1), IGF-1 E3R (SEQ ID NO: 4), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), IGF-1 E3R A13C/L57C (SEQ ID NO: 19), and IGF-1 E3R L14C/L57C (SEQ ID NO: 20). Individual biological data points are indicated, and means reflect the average of 2-3 biological replicates.

FIG. 26 shows the dose-dependent effects of IGF-1 (SEQ ID NO: 1), IGF-1 E3R (SEQ ID NO: 4), IGF-1 E3R F49L (SEQ ID NO: 17), and IGF-1 F49L (SEQ ID NO: 134) on AKT S473 phosphorylation in DU145 cells. Individual data points reflect the mean of 1 to 21 individual experiments, with two biological measurements per experiment. Each data point is normalized to the maximum pAKT level for each molecule with error represented as standard deviation (SD). The mean EC50 value is indicated.

FIG. 27 shows the effects of wild-type IGF-1, IGF-1 E3R (SEQ ID NO: 4), IGF-1 E3R F49L (SEQ ID NO: 17), and IGF-1 F49L (SEQ ID NO: 134) on AKT S473 phosphorylation in DU145 cells in the presence of IGFBP2 or IGFBP3. Individual biological data points are indicated and means (+/−SD) reflect the average of 5 to 28 biological replicates.

FIG. 28 shows that IGFBP2 and IGFBP3 inhibit spheroid growth in the presence of IGF-1, while they have no effect on spheroid growth in the presence of IGF-1 E3R F49L (SEQ ID NO: 17). Spheroid diameter was measured after 12 days culturing in differentiation medium in the presence or absence of 2 nM wild-type IGF-1, 2 nM IGF-1 E3R F49L (SEQ ID NO: 17), 4 nM IGFBP2, or 4 nM IGFBP3. Data depict the mean diameter of 3 wells, containing 45 to 78 spheroids per well. The error is represented as SD.

FIG. 29 shows meibocyte proliferation in young versus aged mice, and further shows meibocyte proliferation in aged mice dosed with IGF-1 E3R F49L (SEQ ID NO: 17) once-daily or twice-daily. Results showed that daily or twice-daily eyedrop dosing of IGF-1 E3R F49L (SEQ ID NO: 17) has the same effect on proliferation induction in the meibomian gland. Individual animal data indicated and means reflect the average of 5 animals per group. **** p<0.0001.

FIG. 30 shows effects of once-daily eyedrop dosing of vehicle control or IGF-1 E3R F49L (SEQ ID NO: 17) at 1 mg/ml for one month on meibomian gland density (left panel) and meibomian gland area (right panel) as measured by transillumination meibography in aged mice. *p<0.05

FIG. 31 shows effects of once-daily eyedrop dosing of vehicle control (left panel) or IGF-1 E3R F49L (SEQ ID NO: 17) (right panel) for one month on resistance to 10 days of desiccation stress-induced corneal damage after cessation of dosing calculated from an average of 13 animals/group. *p<0.05

### DETAILED DESCRIPTION OF THE INVENTION

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” means plus or minus 10% of the number that the term refers to.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable” refers to approved or approvable by a regulatory agency of the Federal or a state government or listed in the U.S. Pharmacopeia (U.S.P.) or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not. The term “sequence identity” or a percent (%) of sequence identity, as used herein is the percentage of residues in a candidate sequence that are identical with the residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term “effective amount” also applies to a dose that will provide an image for detection by an appropriate imaging method. The specific dose may vary depending on one or more of the particular agent chosen, the dosing regimen to be followed, whether it is administered in combination with other compounds, timing of administration, the tissue to be imaged, and the physical delivery system in which it is carried. An effective amount of an active agent may be administered in a single dose or in multiple doses.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

While certain embodiments of the present application have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

### Engineered Polypeptides

The present disclosure provides engineered polypeptides and methods of treatment for a disease and disorder. The disease or disorder may include, for example, Meibomian glands or dry eye disease. The engineered polypeptides may modulate (e.g., upregulate or otherwise activate) the activity of the IGF1R. The polypeptide may have reduced affinity to at least one IGF binding protein (IGFBP) relative to wild-type IGF-1 (SEQ ID NO: 1) to the IGFBP. In one aspect, the engineered polypeptides act locally. The level of one or both of (1) free endogenous IGF-1 or (2) the engineered polypeptides (IGF-1 variants with reduced affinity to one or more IGFBPs) that bind to IGF1R may be increased upon administration to a subject. The local activation of IGF1R by the engineered polypeptides may result in a prolonged or extended pharmacodynamic effect. In another aspect, the engineered polypeptides act systemically. The systemic half-life of the engineered polypeptides decreases due to their reduced affinity to IGFBPs.

In one aspect, the IGF-1 variants are non-naturally occurring IGF-1 variants. IGF-1 and IGF-1 variants can bind to one or more IGFBPs. IGFBPs can lengthen the half-life of circulating wild-type IGF-1. In one aspect, IGFBPs may act to enhance systemic IGF-1 signaling due to increased levels of circulating IGF-1 and IGF-1 variants. In another aspect, IGFBPs may act to attenuate local IGF-1 signaling due to decreased levels of locally free IGF-1 and IGF-1 variants. In one aspect, the one or more IGFBPs may include one or more of be IGFBP1, IGFBP2, IGFBP3, IGFBP4, IGFBP5, IGFBP6, or any combination thereof. In some embodiments, the one or more IGFBPs comprise IGFBP1. In some embodiments, the one or more IGFBPs comprise IGFBP2. In some embodiments, the one or more IGFBPs comprise IGFBP3. In some embodiments, the one or more IGFBPs comprise IGFBP4. In some embodiments, the one or more IGFBPs comprise IGFBP1 and IGFBP2. In some embodiments, the one or more IGFBPs comprise IGFBP1 and IGFBP3. In some embodiments, the one or more IGFBPs comprise IGFBP1 and IGFBP4. In some embodiments, the one or more IGFBPs comprise IGFBP2 and IGFBP3. In some embodiments, the one or more IGFBPs comprise IGFBP2 and IGFBP3. In some embodiments, the one or more IGFBPs comprise IGFBP3 and IGFBP4. In some embodiments, the one or more IGFBPs comprise IGFBP1, IGFBP2, and IGFBP3. In some embodiments, the one or more IGFBPs comprise IGFBP1, IGFBP2, and IGFBP4. In some embodiments, the one or more IGFBPs comprise IGFBP1, IGFBP3, and IGFBP4. In some embodiments, the one or more IGFBPs comprise IGFBP2, IGFBP3, and IGFBP4. In some embodiments, the one or more IGFBPs comprise IGFBP1, IGFBP2, IGFBP3, and IGFBP4.

In some embodiments, the engineered polypeptide is configured to evade one or more IGFBPs. In some embodiments, the evasion of the one or more IGFBPs increases availability of the engineered polypeptide for binding to an IGF receptor. In one aspect, the one or more IGFBPs may include one or more of IGFBP1, IGFBP2, IGFBP3, IGFBP4, IGFBP5, IGFBP6, or any combination thereof. In some embodiments, the one or more IGFBPs comprise IGFBP1. In some embodiments, the one or more IGFBPs comprise IGFBP2. In some embodiments, the one or more IGFBPs comprise IGFBP3. In some embodiments, the one or more IGFBPs comprise IGFBP4. In some embodiments, the one or more IGFBPs comprise IGFBP1 and IGFBP2. In some embodiments, the one or more IGFBPs comprise IGFBP1 and IGFBP3. In some embodiments, the one or more IGFBPs comprise IGFBP1 and IGFBP4. In some embodiments, the one or more IGFBPs comprise IGFBP2 and IGFBP3. In some embodiments, the one or more IGFBPs comprise IGFBP2 and IGFBP3. In some embodiments, the one or more IGFBPs comprise IGFBP3 and IGFBP4. In some embodiments, the one or more IGFBPs comprise IGFBP1, IGFBP2, and IGFBP3. In some embodiments, the one or more IGFBPs comprise IGFBP1, IGFBP2, and IGFBP4. In some embodiments, the one or more IGFBPs comprise IGFBP1, IGFBP3, and IGFBP4. In some embodiments, the one or more IGFBPs comprise IGFBP2, IGFBP3, and IGFBP4. In some embodiments, the one or more IGFBPs comprise IGFBP1, IGFBP2, IGFBP3, and IGFBP4.

In some embodiments, the increased availability of the engineered polypeptide for binding to an IGF receptor leads to a treatment of a disease or a disorder thereof. In some embodiments, the disease or disorder affects muscle, eye, cartilage, bone, kidney, nerves, skin, lungs, liver, or any combination thereof. In some embodiments, the disease or disorder is IGF-1-related. In some embodiments, a symptom of the disease or disorder comprises a lower activation of an IGF receptor in a subject as compared to the activation of an IGF receptor in a subject that does not have the disease or the disorder.

TABLE 1
Sequences of Engineered Polypeptides

| SEQ ID | | |
|---|---|---|
| NO: | Name | Sequence |
| 1 | Wild-type | GPETLCGAELVDALQFVCGDRGFYFNKPTGYGSSSR |
| | IGF-1 | RAPQTGIVDECCFRSCDLRRLEMYCAPLKPAKSA |
| 2 | IGF-1 Y60L | GPETLCGAELVDALQFVCGDRGFYFNKPTGYGSSSR |
| | | RAPQTGIVDECCFRSCDLRRLEMLCAPLKPAKSA |
| 3 | IGF1 Des1- | TLCGAELVDALQFVCGDRGFYFNKPTGYGSSSRRAP |
| | 3 | QTGIVDECCFRSCDLRRLEMYCAPLKPAKSA |
| 4 | IGF1 E3R | GPRTLCGAELVDALQFVCGDRGFYFNKPTGYGSSSR |
| | | RAPQTGIVDECCFRSCDLRRLEMYCAPLKPAKSA |
| 5 | IGF1 R37X | GPETLCGAELVDALQFVCGDRGFYFNKPTGYGSSSR |
| | | APQTGIVDECCFRSCDLRRLEMYCAPLKPAKSA |
| 6 | Des1-3 | TLCGAELVDALQFVCGDRGFYFNKPTGYGSSSRAPQ |
| | R37X | TGIVDECCFRSCDLRRLEMYCAPLKPAKSA |
| 7 | IGF1Ea | GPETLCGAELVDALQFVCGDRGFYFNKPTGYGSSSR |
| | | RAPQTGIVDECCFRSCDLRRLEMYCAPLKPAKSARSV |
| | | RAQRHTDMPKTQKEVHLKNASRGSAGNKNYRM |
| 8 | CPP-Des1-3 | MRAAAPAVAATLCGAELVDALQFVCGDRGFYFNKP |
| | | TGYGSSSRRAPQTGIVDECCFRSCDLRRLEMYCAPLK |
| | | PAKSA |
| 9 | skin- | MRAAAPAVAA |
| | penetrating | |
| | peptide | |
| 10 | CPP | CGRKKRRQRRRPPQC |
| 11 | CPP | RRRRRRRRR |
| 12 | IGF-1 LR3 | MFPAMPLSSLFVNGPRTLCGAELVDALQFVCGDRGF |
| | | YFNKPTGYGSSSRRAPQTGIVDECCFRSCDLRRLEMY |
| | | CAPLKPAKSA |
| 13 | Linker IGF1 | GGGGSGGGGSGGGGPETLCGAELVDALQFVCGDRG |
| | | FYFNKPTGYGSSSRRAPQTGIVDECCFRSCDLRRLEM |
| | | YCAPLKPAKSA |
| 14 | Linker IGF1 | GGGGSGGGGGGGGPRTLCGAELVDALQFVCGDRG |
| | E3R | FYFNKPTGYGSSSRRAPQTGIVDECCFRSCDLRRLEM |
| | | YCAPLKPAKSA |
| 15 | E3R D12A | GPRTLCGAELVAALQFVCGDRGFYFNKPTGYGSSSR |
| | | RAPQTGIVDECCFRSCDLRRLEMYCAPLKPAKSA |
| 16 | E3R D20A | GPRTLCGAELVDALQFVCGARGFYFNKPTGYGSSSR |
| | | RAPQTGIVDECCFRSCDLRRLEMYCAPLKPAKSA |
| 17 | E3R F49L | GPRTLCGAELVDALQFVCGDRGFYFNKPTGYGSSSR |
| | | RAPQTGIVDECCLRSCDLRRLEMYCAPLKPAKSA |
| 18 | E3R L54A | GPRTLCGAELVDALQFVCGDRGFYFNKPTGYGSSSR |
| | | RAPQTGIVDECCFRSCDARRLEMYCAPLKPAKSA |
| 19 | E3R A13C/ | GPRTLCGAELVDCLQFVCGDRGFYFNKPTGYGSSSR |
| | L57C | RAPQTGIVDECCFRSCDLRRCEMYCAPLKPAKSA |
| 20 | E3R L14C/ | GPRTLCGAELVDACQFVCGDRGFYFNKPTGYGSSSR |
| | L57C | RAPQTGIVDECCFRSCDLRRCEMYCAPLKPAKSA |
| 21 | E3R D12C | GPRTLCGAELVCALQFVCGDRGFYFNKPTGYGSSSR |
| | | RAPQTGIVDECCFRSCDLRRLEMYCAPLKPAKSA |
| 22 | E3R D12E | GPRTLCGAELVEALQFVCGDRGFYFNKPTGYGSSSR |
| | | RAPQTGIVDECCFRSCDLRRLEMYCAPLKPAKSA |
| 23 | E3R D12F | GPRTLCGAELVFALQFVCGDRGFYFNKPTGYGSSSRR |
| | | APQTGIVDECCFRSCDLRRLEMYCAPLKPAKSA |
| 24 | E3R D12G | GPRTLCGAELVGALQFVCGDRGFYFNKPTGYGSSSR |
| | | RAPQTGIVDECCFRSCDLRRLEMYCAPLKPAKSA |
| 25 | E3R D12H | GPRTLCGAELVHALQFVCGDRGFYFNKPTGYGSSSR |
| | | RAPQTGIVDECCFRSCDLRRLEMYCAPLKPAKSA |
| 26 | E3R D12I | GPRTLCGAELVIALQFVCGDRGFYFNKPTGYGSSSRR |
| | | APQTGIVDECCFRSCDLRRLEMYCAPLKPAKSA |
| 27 | E3R D12K | GPRTLCGAELVKALQFVCGDRGFYFNKPTGYGSSSR |
| | | RAPQTGIVDECCFRSCDLRRLEMYCAPLKPAKSA |
| 28 | E3R D12L | GPRTLCGAELVLALQFVCGDRGFYFNKPTGYGSSSR |
| | | RAPQTGIVDECCFRSCDLRRLEMYCAPLKPAKSA |
| 29 | E3R D12M | GPRTLCGAELVMALQFVCGDRGFYFNKPTGYGSSSR |
| | | RAPQTGIVDECCFRSCDLRRLEMYCAPLKPAKSA |
| 30 | E3R D12N | GPRTLCGAELVNALQFVCGDRGFYFNKPTGYGSSSR |
| | | RAPQTGIVDECCFRSCDLRRLEMYCAPLKPAKSA |
| 31 | E3R D12P | GPRTLCGAELVPALQFVCGDRGFYFNKPTGYGSSSRR |
| | | APQTGIVDECCFRSCDLRRLEMYCAPLKPAKSA |
| 32 | E3R D12Q | GPRTLCGAELVQALQFVCGDRGFYFNKPTGYGSSSR |
| | | RAPQTGIVDECCFRSCDLRRLEMYCAPLKPAKSA |
| 33 | E3R D12R | GPRTLCGAELVRALQFVCGDRGFYFNKPTGYGSSSR |
| | | RAPQTGIVDECCFRSCDLRRLEMYCAPLKPAKSA |
| 34 | E3R D12S | GPRTLCGAELVSALQFVCGDRGFYFNKPTGYGSSSRR |
| | | APQTGIVDECCFRSCDLRRLEMYCAPLKPAKSA |
| 35 | E3R D12T | GPRTLCGAELVTALQFVCGDRGFYFNKPTGYGSSSR |
| | | RAPQTGIVDECCFRSCDLRRLEMYCAPLKPAKSA |
| 36 | E3R D12V | GPRTLCGAELVVALQFVCGDRGFYFNKPTGYGSSSR |
| | | RAPQTGIVDECCFRSCDLRRLEMYCAPLKPAKSA |
| 37 | E3R D12W | GPRTLCGAELVWALQFVCGDRGFYFNKPTGYGSSSR |
| | | RAPQTGIVDECCFRSCDLRRLEMYCAPLKPAKSA |
| 38 | E3R D12Y | GPRTLCGAELVYALQFVCGDRGFYFNKPTGYGSSSR |
| | | RAPQTGIVDECCFRSCDLRRLEMYCAPLKPAKSA |
| 39 | Des1-3 | TLCGAELVAALQFVCGDRGFYFNKPTGYGSSSRRAP |
| | D12A | QTGIVDECCFRSCDLRRLEMYCAPLKPAKSA |
| 40 | Des1-3 | TLCGAELVCALQFVCGDRGFYFNKPTGYGSSSRRAP |
| | D12C | QTGIVDECCFRSCDLRRLEMYCAPLKPAKSA |
| 41 | Des 1-3 | TLCGAELVEALQFVCGDRGFYFNKPTGYGSSSRRAP |
| | D12E | QTGIVDECCFRSCDLRRLEMYCAPLKPAKSA |
| 42 | Des1-3 | TLCGAELVFALQFVCGDRGFYFNKPTGYGSSSRRAP |
| | D12F | QTGIVDECCFRSCDLRRLEMYCAPLKPAKSA |
| 43 | Des1-3 | TLCGAELVGALQFVCGDRGFYFNKPTGYGSSSRRAP |
| | D12G | QTGIVDECCFRSCDLRRLEMYCAPLKPAKSA |
| 44 | Des1-3 | TLCGAELVHALQFVCGDRGFYFNKPTGYGSSSRRAP |
| | D12H | QTGIVDECCFRSCDLRRLEMYCAPLKPAKSA |
| 45 | Des1-3 | TLCGAELVIALQFVCGDRGFYFNKPTGYGSSSRRAPQ |
| | D12I | TGIVDECCFRSCDLRRLEMYCAPLKPAKSA |
| 46 | Des1-3 | TLCGAELVKALQFVCGDRGFYFNKPTGYGSSSRRAP |
| | D12K | QTGIVDECCFRSCDLRRLEMYCAPLKPAKSA |
| 47 | Des1-3 | TLCGAELVLALQFVCGDRGFYFNKPTGYGSSSRRAP |
| | D12L | QTGIVDECCFRSCDLRRLEMYCAPLKPAKSA |
| 48 | Des1-3 | TLCGAELVMALQFVCGDRGFYFNKPTGYGSSSRRAP |
| | D12M | QTGIVDECCFRSCDLRRLEMYCAPLKPAKSA |
| 49 | Des1-3 | TLCGAELVNALQFVCGDRGFYFNKPTGYGSSSRRAP |
| | D12N | QTGIVDECCFRSCDLRRLEMYCAPLKPAKSA |
| 50 | Des1-3 | TLCGAELVPALQFVCGDRGFYFNKPTGYGSSSRRAP |
| | D12P | QTGIVDECCFRSCDLRRLEMYCAPLKPAKSA |
| 51 | Des1-3 | TLCGAELVQALQFVCGDRGFYFNKPTGYGSSSRRAP |
| | D12Q | QTGIVDECCFRSCDLRRLEMYCAPLKPAKSA |
| 52 | Des1-3 | TLCGAELVRALQFVCGDRGFYFNKPTGYGSSSRRAP |
| | D12R | QTGIVDECCFRSCDLRRLEMYCAPLKPAKSA |
| 53 | Des 1-3 | TLCGAELVSALQFVCGDRGFYFNKPTGYGSSSRRAP |
| | D12S | QTGIVDECCFRSCDLRRLEMYCAPLKPAKSA |
| 54 | Des1-3 | TLCGAELVTALQFVCGDRGFYFNKPTGYGSSSRRAP |
| | D12T | QTGIVDECCFRSCDLRRLEMYCAPLKPAKSA |
| 55 | Des1-3 | TLCGAELVVALQFVCGDRGFYFNKPTGYGSSSRRAP |
| | D12V | QTGIVDECCFRSCDLRRLEMYCAPLKPAKSA |
| 56 | Des1-3 | TLCGAELVWALQFVCGDRGFYFNKPTGYGSSSRRAP |
| | D12W | QTGIVDECCFRSCDLRRLEMYCAPLKPAKSA |
| 57 | Des1-3 | TLCGAELVYALQFVCGDRGFYFNKPTGYGSSSRRAP |
| | D12Y | QTGIVDECCFRSCDLRRLEMYCAPLKPAKSA |
| 58 | E3R F49A | GPRTLCGAELVDALQFVCGDRGFYFNKPTGYGSSSR |
| | | RAPQTGIVDECCARSCDLRRLEMYCAPLKPAKSA |
| 59 | E3R F49C | GPRTLCGAELVDALQFVCGDRGFYFNKPTGYGSSSR |
| | | RAPQTGIVDECCCRSCDLRRLEMYCAPLKPAKSA |
| 60 | E3R F49D | GPRTLCGAELVDALQFVCGDRGFYFNKPTGYGSSSR |
| | | RAPQTGIVDECCDRSCDLRRLEMYCAPLKPAKSA |
| 61 | E3R F49E | GPRTLCGAELVDALQFVCGDRGFYFNKPTGYGSSSR |
| | | RAPQTGIVDECCERSCDLRRLEMYCAPLKPAKSA |
| 62 | E3R F49G | GPRTLCGAELVDALQFVCGDRGFYFNKPTGYGSSSR |
| | | RAPQTGIVDECCGRSCDLRRLEMYCAPLKPAKSA |
| 63 | E3R F49H | GPRTLCGAELVDALQFVCGDRGFYFNKPTGYGSSSR |
| | | RAPQTGIVDECCHRSCDLRRLEMYCAPLKPAKSA |
| 64 | E3R F49I | GPRTLCGAELVDALQFVCGDRGFYFNKPTGYGSSSR |
| | | RAPQTGIVDECCIRSCDLRRLEMYCAPLKPAKSA |
| 65 | E3R F49K | GPRTLCGAELVDALQFVCGDRGFYFNKPTGYGSSSR |
| | | RAPQTGIVDECCKRSCDLRRLEMYCAPLKPAKSA |
| 66 | E3R F49M | GPRTLCGAELVDALQFVCGDRGFYFNKPTGYGSSSR |
| | | RAPQTGIVDECCMRSCDLRRLEMYCAPLKPAKSA |
| 67 | E3R F49N | GPRTLCGAELVDALQFVCGDRGFYFNKPTGYGSSSR |
| | | RAPQTGIVDECCNRSCDLRRLEMYCAPLKPAKSA |
| 68 | E3R F49P | GPRTLCGAELVDALQFVCGDRGFYFNKPTGYGSSSR |
| | | RAPQTGIVDECCPRSCDLRRLEMYCAPLKPAKSA |
| 69 | E3R F49Q | GPRTLCGAELVDALQFVCGDRGFYFNKPTGYGSSSR |
| | | RAPQTGIVDECCQRSCDLRRLEMYCAPLKPAKSA |
| 70 | E3R F49R | GPRTLCGAELVDALQFVCGDRGFYFNKPTGYGSSSR |
| | | RAPQTGIVDECCRRSCDLRRLEMYCAPLKPAKSA |
| 71 | E3R F49S | GPRTLCGAELVDALQFVCGDRGFYFNKPTGYGSSSR |
| | | RAPQTGIVDECCSRSCDLRRLEMYCAPLKPAKSA |
| 72 | E3R F49T | GPRTLCGAELVDALQFVCGDRGFYFNKPTGYGSSSR |
| | | RAPQTGIVDECCTRSCDLRRLEMYCAPLKPAKSA |
| 73 | E3R F49V | GPRTLCGAELVDALQFVCGDRGFYFNKPTGYGSSSR |
| | | RAPQTGIVDECCVRSCDLRRLEMYCAPLKPAKSA |
| 74 | E3R F49W | GPRTLCGAELVDALQFVCGDRGFYFNKPTGYGSSSR |
| | | RAPQTGIVDECCWRSCDLRRLEMYCAPLKPAKSA |
| 75 | E3R F49Y | GPRTLCGAELVDALQFVCGDRGFYFNKPTGYGSSSR |
| | | RAPQTGIVDECCYRSCDLRRLEMYCAPLKPAKSA |
| 76 | Des1-3 | TLCGAELVDALQFVCGDRGFYFNKPTGYGSSSRRAP |
| | F49A | QTGIVDECCARSCDLRRLEMYCAPLKPAKSA |
| 77 | Des1-3 | TLCGAELVDALQFVCGDRGFYFNKPTGYGSSSRRAP |
| | F49C | QTGIVDECCCRSCDLRRLEMYCAPLKPAKSA |
| 78 | Des 1-3 | TLCGAELVDALQFVCGDRGFYFNKPTGYGSSSRRAP |
| | F49D | QTGIVDECCDRSCDLRRLEMYCAPLKPAKSA |
| 79 | Des 1-3 | TLCGAELVDALQFVCGDRGFYFNKPTGYGSSSRRAP |
| | F49E | QTGIVDECCERSCDLRRLEMYCAPLKPAKSA |
| 80 | Des1-3 | TLCGAELVDALQFVCGDRGFYFNKPTGYGSSSRRAP |
| | F49G | QTGIVDECCGRSCDLRRLEMYCAPLKPAKSA |
| 81 | Des1-3 | TLCGAELVDALQFVCGDRGFYFNKPTGYGSSSRRAP |
| | F49H | QTGIVDECCHRSCDLRRLEMYCAPLKPAKSA |
| 82 | Des1-3 | TLCGAELVDALQFVCGDRGFYFNKPTGYGSSSRRAP |
| | F49I | QTGIVDECCIRSCDLRRLEMYCAPLKPAKSA |
| 83 | Des1-3 | TLCGAELVDALQFVCGDRGFYFNKPTGYGSSSRRAP |
| | F49K | QTGIVDECCKRSCDLRRLEMYCAPLKPAKSA |
| 84 | Des 1-3 | TLCGAELVDALQFVCGDRGFYFNKPTGYGSSSRRAP |
| | F49L | QTGIVDECCLRSCDLRRLEMYCAPLKPAKSA |
| 85 | Des1-3 | TLCGAELVDALQFVCGDRGFYFNKPTGYGSSSRRAP |
| | F49M | QTGIVDECCMRSCDLRRLEMYCAPLKPAKSA |
| 86 | Des1-3 | TLCGAELVDALQFVCGDRGFYFNKPTGYGSSSRRAP |
| | F49N | QTGIVDECCNRSCDLRRLEMYCAPLKPAKSA |
| 87 | Des 1-3 | TLCGAELVDALQFVCGDRGFYFNKPTGYGSSSRRAP |
| | F49P | QTGIVDECCPRSCDLRRLEMYCAPLKPAKSA |
| 88 | Des1-3 | TLCGAELVDALQFVCGDRGFYFNKPTGYGSSSRRAP |
| | F49Q | QTGIVDECCQRSCDLRRLEMYCAPLKPAKSA |
| 89 | Des1-3 | TLCGAELVDALQFVCGDRGFYFNKPTGYGSSSRRAP |
| | F49R | QTGIVDECCRRSCDLRRLEMYCAPLKPAKSA |
| 90 | Des 1-3 | TLCGAELVDALQFVCGDRGFYFNKPTGYGSSSRRAP |
| | F49S | QTGIVDECCSRSCDLRRLEMYCAPLKPAKSA |
| 91 | Des 1-3 | TLCGAELVDALQFVCGDRGFYFNKPTGYGSSSRRAP |
| | F49T | QTGIVDECCTRSCDLRRLEMYCAPLKPAKSA |
| 92 | Des1-3 | TLCGAELVDALQFVCGDRGFYFNKPTGYGSSSRRAP |
| | F49V | QTGIVDECCVRSCDLRRLEMYCAPLKPAKSA |
| 93 | Des1-3 | TLCGAELVDALQFVCGDRGFYFNKPTGYGSSSRRAP |
| | F49W | QTGIVDECCWRSCDLRRLEMYCAPLKPAKSA |
| 94 | Des1-3 | TLCGAELVDALQFVCGDRGFYFNKPTGYGSSSRRAP |
| | F49Y | QTGIVDECCYRSCDLRRLEMYCAPLKPAKSA |
| 95 | E3R L54C | GPRTLCGAELVDALQFVCGDRGFYFNKPTGYGSSSR |
| | | RAPQTGIVDECCFRSCDCRRLEMYCAPLKPAKSA |
| 96 | E3R L54D | GPRTLCGAELVDALQFVCGDRGFYFNKPTGYGSSSR |
| | | RAPQTGIVDECCFRSCDDRRLEMYCAPLKPAKSA |
| 97 | E3R L54E | GPRTLCGAELVDALQFVCGDRGFYFNKPTGYGSSSR |
| | | RAPQTGIVDECCFRSCDERRLEMYCAPLKPAKSA |
| 98 | E3R L54F | GPRTLCGAELVDALQFVCGDRGFYFNKPTGYGSSSR |
| | | RAPQTGIVDECCFRSCDFRRLEMYCAPLKPAKSA |
| 99 | E3R L54G | GPRTLCGAELVDALQFVCGDRGFYFNKPTGYGSSSR |
| | | RAPQTGIVDECCFRSCDGRRLEMYCAPLKPAKSA |
| 100 | E3R L54H | GPRTLCGAELVDALQFVCGDRGFYFNKPTGYGSSSR |
| | | RAPQTGIVDECCFRSCDHRRLEMYCAPLKPAKSA |
| 101 | E3R L54I | GPRTLCGAELVDALQFVCGDRGFYFNKPTGYGSSSR |
| | | RAPQTGIVDECCFRSCDIRRLEMYCAPLKPAKSA |
| 102 | E3R L54K | GPRTLCGAELVDALQFVCGDRGFYFNKPTGYGSSSR |
| | | RAPQTGIVDECCFRSCDKRRLEMYCAPLKPAKSA |
| 103 | E3R L54M | GPRTLCGAELVDALQFVCGDRGFYFNKPTGYGSSSR |
| | | RAPQTGIVDECCFRSCDMRRLEMYCAPLKPAKSA |
| 104 | E3R L54N | GPRTLCGAELVDALQFVCGDRGFYFNKPTGYGSSSR |
| | | RAPQTGIVDECCFRSCDNRRLEMYCAPLKPAKSA |
| 105 | E3R L54P | GPRTLCGAELVDALQFVCGDRGFYFNKPTGYGSSSR |
| | | RAPQTGIVDECCFRSCDPRRLEMYCAPLKPAKSA |
| 106 | E3R L54Q | GPRTLCGAELVDALQFVCGDRGFYFNKPTGYGSSSR |
| | | RAPQTGIVDECCFRSCDQRRLEMYCAPLKPAKSA |
| 107 | E3R L54R | GPRTLCGAELVDALQFVCGDRGFYFNKPTGYGSSSR |
| | | RAPQTGIVDECCFRSCDRRRLEMYCAPLKPAKSA |
| 108 | E3R L54S | GPRTLCGAELVDALQFVCGDRGFYFNKPTGYGSSSR |
| | | RAPQTGIVDECCFRSCDSRRLEMYCAPLKPAKSA |
| 109 | E3R L54T | GPRTLCGAELVDALQFVCGDRGFYFNKPTGYGSSSR |
| | | RAPQTGIVDECCFRSCDTRRLEMYCAPLKPAKSA |
| 110 | E3R L54V | GPRTLCGAELVDALQFVCGDRGFYFNKPTGYGSSSR |
| | | RAPQTGIVDECCFRSCDVRRLEMYCAPLKPAKSA |
| 111 | E3R L54W | GPRTLCGAELVDALQFVCGDRGFYFNKPTGYGSSSR |
| | | RAPQTGIVDECCFRSCDWRRLEMYCAPLKPAKSA |
| 112 | E3R L54Y | GPRTLCGAELVDALQFVCGDRGFYFNKPTGYGSSSR |
| | | RAPQTGIVDECCFRSCDYRRLEMYCAPLKPAKSA |
| 113 | Des1-3 | TLCGAELVDALQFVCGDRGFYFNKPTGYGSSSRRAP |
| | L54A | QTGIVDECCFRSCDARRLEMYCAPLKPAKSA |
| 114 | Des1-3 | TLCGAELVDALQFVCGDRGFYFNKPTGYGSSSRRAP |
| | L54C | QTGIVDECCFRSCDCRRLEMYCAPLKPAKSA |
| 115 | Des1-3 | TLCGAELVDALQFVCGDRGFYFNKPTGYGSSSRRAP |
| | L54D | QTGIVDECCFRSCDDRRLEMYCAPLKPAKSA |
| 116 | Des1-3 | TLCGAELVDALQFVCGDRGFYFNKPTGYGSSSRRAP |
| | L54E | QTGIVDECCFRSCDERRLEMYCAPLKPAKSA |
| 117 | Des1-3 | TLCGAELVDALQFVCGDRGFYFNKPTGYGSSSRRAP |
| | L54F | QTGIVDECCFRSCDFRRLEMYCAPLKPAKSA |
| 118 | Des1-3 | TLCGAELVDALQFVCGDRGFYFNKPTGYGSSSRRAP |
| | L54G | QTGIVDECCFRSCDGRRLEMYCAPLKPAKSA |
| 119 | Des1-3 | TLCGAELVDALQFVCGDRGFYFNKPTGYGSSSRRAP |
| | L54H | QTGIVDECCFRSCDHRRLEMYCAPLKPAKSA |
| 120 | Des1-3 L54I | TLCGAELVDALQFVCGDRGFYFNKPTGYGSSSRRAP |
| | | QTGIVDECCFRSCDIRRLEMYCAPLKPAKSA |
| 121 | Des1-3 | TLCGAELVDALQFVCGDRGFYFNKPTGYGSSSRRAP |
| | L54K | QTGIVDECCFRSCDKRRLEMYCAPLKPAKSA |
| 122 | Des1-3 | TLCGAELVDALQFVCGDRGFYFNKPTGYGSSSRRAP |
| | L54M | QTGIVDECCFRSCDMRRLEMYCAPLKPAKSA |
| 123 | Des1-3 | TLCGAELVDALQFVCGDRGFYFNKPTGYGSSSRRAP |
| | L54N | QTGIVDECCFRSCDNRRLEMYCAPLKPAKSA |
| 124 | Des1-3 | TLCGAELVDALQFVCGDRGFYFNKPTGYGSSSRRAP |
| | L54P | QTGIVDECCFRSCDPRRLEMYCAPLKPAKSA |
| 125 | Des1-3 | TLCGAELVDALQFVCGDRGFYFNKPTGYGSSSRRAP |
| | L54Q | QTGIVDECCFRSCDQRRLEMYCAPLKPAKSA |
| 126 | Des1-3 | TLCGAELVDALQFVCGDRGFYFNKPTGYGSSSRRAP |
| | L54R | QTGIVDECCFRSCDRRRLEMYCAPLKPAKSA |
| 127 | Des1-3 | TLCGAELVDALQFVCGDRGFYFNKPTGYGSSSRRAP |
| | L54S | QTGIVDECCFRSCDSRRLEMYCAPLKPAKSA |
| 128 | Des1-3 | TLCGAELVDALQFVCGDRGFYFNKPTGYGSSSRRAP |
| | L54T | QTGIVDECCFRSCDTRRLEMYCAPLKPAKSA |
| 129 | Des1-3 | TLCGAELVDALQFVCGDRGFYFNKPTGYGSSSRRAP |
| | L54V | QTGIVDECCFRSCDVRRLEMYCAPLKPAKSA |
| 130 | Des1-3 | TLCGAELVDALQFVCGDRGFYFNKPTGYGSSSRRAP |
| | L54W | QTGIVDECCFRSCDWRRLEMYCAPLKPAKSA |
| 131 | Des1-3 | TLCGAELVDALQFVCGDRGFYFNKPTGYGSSSRRAP |
| | L54Y | QTGIVDECCFRSCDYRRLEMYCAPLKPAKSA |
| 132 | F49A | GPETLCGAELVDALQFVCGDRGFYFNKPTGYGSSSR |
| | | RAPQTGIVDECCARSCDLRRLEMYCAPLKPAKSA |
| 133 | D12A | GPETLCGAELVAALQFVCGDRGFYFNKPTGYGSSSR |
| | | RAPQTGIVDECCFRSCDLRRLEMYCAPLKPAKSA |
| 134 | F49L | GPETLCGAELVDALQFVCGDRGFYFNKPTGYGSSSR |
| | | RAPQTGIVDECCLRSCDLRRLEMYCAPLKPAKSA |
| 135 | L54A | GPETLCGAELVDALQFVCGDRGFYFNKPTGYGSSSR |
| | | RAPQTGIVDECCFRSCDARRLEMYCAPLKPAKSA |
| 136 | F49L-D12A | GPETLCGAELVAALQFVCGDRGFYFNKPTGYGSSSR |
| | | RAPQTGIVDECCLRSCDLRRLEMYCAPLKPAKSA |
| 137 | F49L-L54A | GPETLCGAELVDALQFVCGDRGFYFNKPTGYGSSSR |
| | | RAPQTGIVDECCLRSCDARRLEMYCAPLKPAKSA |
| 138 | F49L- | GPETLCGAELVAALQFVCGDRGFYFNKPTGYGSSSR |
| | D12A-L54A | RAPQTGIVDECCLRSCDARRLEMYCAPLKPAKSA |
| 139 | E3R-F49L- | GPRTLCGAELVAALQFVCGDRGFYFNKPTGYGSSSR |
| | D12A | RAPQTGIVDECCLRSCDLRRLEMYCAPLKPAKSA |
| 140 | E3R-F49L- | GPRTLCGAELVDALQFVCGDRGFYFNKPTGYGSSSR |
| | L54A | RAPQTGIVDECCLRSCDARRLEMYCAPLKPAKSA |
| 141 | E3R-F49L- | GPRTLCGAELVAALQFVCGDRGFYFNKPTGYGSSSR |
| | D12A-L54A | RAPQTGIVDECCLRSCDARRLEMYCAPLKPAKSA |
| 142 | Des1-3- | TLCGAELVAALQFVCGDRGFYFNKPTGYGSSSRRAP |
| | F49L-D12A | QTGIVDECCLRSCDLRRLEMYCAPLKPAKSA |
| 143 | Des1-3- | TLCGAELVDALQFVCGDRGFYFNKPTGYGSSSRRAP |
| | F49L-L54A | QTGIVDECCLRSCDARRLEMYCAPLKPAKSA |
| 144 | Des1-3- | TLCGAELVAALQFVCGDRGFYFNKPTGYGSSSRRAP |
| | F49L- | QTGIVDECCLRSCDARRLEMYCAPLKPAKSA |
| | D12A-L54A | |
| 145 | D12C | GPETLCGAELVCALQFVCGDRGFYFNKPTGYGSSSR |
| | | RAPQTGIVDECCFRSCDLRRLEMYCAPLKPAKSA |
| 146 | D12E | GPETLCGAELVEALQFVCGDRGFYFNKPTGYGSSSRR |
| | | APQTGIVDECCFRSCDLRRLEMYCAPLKPAKSA |
| 147 | D12F | GPETLCGAELVFALQFVCGDRGFYFNKPTGYGSSSRR |
| | | APQTGIVDECCFRSCDLRRLEMYCAPLKPAKSA |
| 148 | D12G | GPETLCGAELVGALQFVCGDRGFYFNKPTGYGSSSR |
| | | RAPQTGIVDECCFRSCDLRRLEMYCAPLKPAKSA |
| 149 | D12H | GPETLCGAELVHALQFVCGDRGFYFNKPTGYGSSSR |
| | | RAPQTGIVDECCFRSCDLRRLEMYCAPLKPAKSA |
| 150 | D12I | GPETLCGAELVIALQFVCGDRGFYFNKPTGYGSSSRR |
| | | APQTGIVDECCFRSCDLRRLEMYCAPLKPAKSA |
| 151 | D12K | GPETLCGAELVKALQFVCGDRGFYFNKPTGYGSSSR |
| | | RAPQTGIVDECCFRSCDLRRLEMYCAPLKPAKSA |
| 152 | D12L | GPETLCGAELVLALQFVCGDRGFYFNKPTGYGSSSRR |
| | | APQTGIVDECCFRSCDLRRLEMYCAPLKPAKSA |
| 153 | D12M | GPETLCGAELVMALQFVCGDRGFYFNKPTGYGSSSR |
| | | RAPQTGIVDECCFRSCDLRRLEMYCAPLKPAKSA |
| 154 | D12N | GPETLCGAELVNALQFVCGDRGFYFNKPTGYGSSSR |
| | | RAPQTGIVDECCFRSCDLRRLEMYCAPLKPAKSA |
| 155 | D12P | GPETLCGAELVPALQFVCGDRGFYFNKPTGYGSSSRR |
| | | APQTGIVDECCFRSCDLRRLEMYCAPLKPAKSA |
| 156 | D12Q | GPETLCGAELVQALQFVCGDRGFYFNKPTGYGSSSR |
| | | RAPQTGIVDECCFRSCDLRRLEMYCAPLKPAKSA |
| 157 | D12R | GPETLCGAELVRALQFVCGDRGFYFNKPTGYGSSSR |
| | | RAPQTGIVDECCFRSCDLRRLEMYCAPLKPAKSA |
| 158 | D12S | GPETLCGAELVSALQFVCGDRGFYFNKPTGYGSSSRR |
| | | APQTGIVDECCFRSCDLRRLEMYCAPLKPAKSA |
| 159 | D12T | GPETLCGAELVTALQFVCGDRGFYFNKPTGYGSSSRR |
| | | APQTGIVDECCFRSCDLRRLEMYCAPLKPAKSA |
| 160 | D12V | GPETLCGAELVVALQFVCGDRGFYFNKPTGYGSSSR |
| | | RAPQTGIVDECCFRSCDLRRLEMYCAPLKPAKSA |
| 161 | D12W | GPETLCGAELVWALQFVCGDRGFYFNKPTGYGSSSR |
| | | RAPQTGIVDECCFRSCDLRRLEMYCAPLKPAKSA |
| 162 | D12Y | GPETLCGAELVYALQFVCGDRGFYFNKPTGYGSSSR |
| | | RAPQTGIVDECCFRSCDLRRLEMYCAPLKPAKSA |
| 163 | F49C | GPETLCGAELVDALQFVCGDRGFYFNKPTGYGSSSR |
| | | RAPQTGIVDECCCRSCDLRRLEMYCAPLKPAKSA |
| 164 | F49D | GPETLCGAELVDALQFVCGDRGFYFNKPTGYGSSSR |
| | | RAPQTGIVDECCDRSCDLRRLEMYCAPLKPAKSA |
| 165 | F49E | GPETLCGAELVDALQFVCGDRGFYFNKPTGYGSSSR |
| | | RAPQTGIVDECCERSCDLRRLEMYCAPLKPAKSA |
| 166 | F49G | GPETLCGAELVDALQFVCGDRGFYFNKPTGYGSSSR |
| | | RAPQTGIVDECCGRSCDLRRLEMYCAPLKPAKSA |
| 167 | F49H | GPETLCGAELVDALQFVCGDRGFYFNKPTGYGSSSR |
| | | RAPQTGIVDECCHRSCDLRRLEMYCAPLKPAKSA |
| 168 | F49I | GPETLCGAELVDALQFVCGDRGFYFNKPTGYGSSSR |
| | | RAPQTGIVDECCIRSCDLRRLEMYCAPLKPAKSA |
| 169 | F49K | GPETLCGAELVDALQFVCGDRGFYFNKPTGYGSSSR |
| | | RAPQTGIVDECCKRSCDLRRLEMYCAPLKPAKSA |
| 170 | F49M | GPETLCGAELVDALQFVCGDRGFYFNKPTGYGSSSR |
| | | RAPQTGIVDECCMRSCDLRRLEMYCAPLKPAKSA |
| 171 | F49N | GPETLCGAELVDALQFVCGDRGFYFNKPTGYGSSSR |
| | | RAPQTGIVDECCNRSCDLRRLEMYCAPLKPAKSA |
| 172 | F49P | GPETLCGAELVDALQFVCGDRGFYFNKPTGYGSSSR |
| | | RAPQTGIVDECCPRSCDLRRLEMYCAPLKPAKSA |
| 173 | F49Q | GPETLCGAELVDALQFVCGDRGFYFNKPTGYGSSSR |
| | | RAPQTGIVDECCQRSCDLRRLEMYCAPLKPAKSA |
| 174 | F49R | GPETLCGAELVDALQFVCGDRGFYFNKPTGYGSSSR |
| | | RAPQTGIVDECCRRSCDLRRLEMYCAPLKPAKSA |
| 175 | F49S | GPETLCGAELVDALQFVCGDRGFYFNKPTGYGSSSR |
| | | RAPQTGIVDECCSRSCDLRRLEMYCAPLKPAKSA |
| 176 | F49T | GPETLCGAELVDALQFVCGDRGFYFNKPTGYGSSSR |
| | | RAPQTGIVDECCTRSCDLRRLEMYCAPLKPAKSA |
| 177 | F49V | GPETLCGAELVDALQFVCGDRGFYFNKPTGYGSSSR |
| | | RAPQTGIVDECCVRSCDLRRLEMYCAPLKPAKSA |
| 178 | F49W | GPETLCGAELVDALQFVCGDRGFYFNKPTGYGSSSR |
| | | RAPQTGIVDECCWRSCDLRRLEMYCAPLKPAKSA |
| 179 | F49Y | GPETLCGAELVDALQFVCGDRGFYFNKPTGYGSSSR |
| | | RAPQTGIVDECCYRSCDLRRLEMYCAPLKPAKSA |
| 180 | L54C | GPETLCGAELVDALQFVCGDRGFYFNKPTGYGSSSR |
| | | RAPQTGIVDECCFRSCDCRRLEMYCAPLKPAKSA |
| 181 | L54D | GPETLCGAELVDALQFVCGDRGFYFNKPTGYGSSSR |
| | | RAPQTGIVDECCFRSCDDRRLEMYCAPLKPAKSA |
| 182 | L54E | GPETLCGAELVDALQFVCGDRGFYFNKPTGYGSSSR |
| | | RAPQTGIVDECCFRSCDERRLEMYCAPLKPAKSA |
| 183 | L54F | GPETLCGAELVDALQFVCGDRGFYFNKPTGYGSSSR |
| | | RAPQTGIVDECCFRSCDFRRLEMYCAPLKPAKSA |
| 184 | L54G | GPETLCGAELVDALQFVCGDRGFYFNKPTGYGSSSR |
| | | RAPQTGIVDECCFRSCDGRRLEMYCAPLKPAKSA |
| 185 | L54H | GPETLCGAELVDALQFVCGDRGFYFNKPTGYGSSSR |
| | | RAPQTGIVDECCFRSCDHRRLEMYCAPLKPAKSA |
| 186 | L54I | GPETLCGAELVDALQFVCGDRGFYFNKPTGYGSSSR |
| | | RAPQTGIVDECCFRSCDIRRLEMYCAPLKPAKSA |
| 187 | L54K | GPETLCGAELVDALQFVCGDRGFYFNKPTGYGSSSR |
| | | RAPQTGIVDECCFRSCDKRRLEMYCAPLKPAKSA |
| 188 | L54M | GPETLCGAELVDALQFVCGDRGFYFNKPTGYGSSSR |
| | | RAPQTGIVDECCFRSCDMRRLEMYCAPLKPAKSA |
| 189 | L54N | GPETLCGAELVDALQFVCGDRGFYFNKPTGYGSSSR |
| | | RAPQTGIVDECCFRSCDNRRLEMYCAPLKPAKSA |
| 190 | L54P | GPETLCGAELVDALQFVCGDRGFYFNKPTGYGSSSR |
| | | RAPQTGIVDECCFRSCDPRRLEMYCAPLKPAKSA |
| 191 | L54Q | GPETLCGAELVDALQFVCGDRGFYFNKPTGYGSSSR |
| | | RAPQTGIVDECCFRSCDQRRLEMYCAPLKPAKSA |
| 192 | L54R | GPETLCGAELVDALQFVCGDRGFYFNKPTGYGSSSR |
| | | RAPQTGIVDECCFRSCDRRRLEMYCAPLKPAKSA |
| 193 | L54S | GPETLCGAELVDALQFVCGDRGFYFNKPTGYGSSSR |
| | | RAPQTGIVDECCFRSCDSRRLEMYCAPLKPAKSA |
| 194 | L54T | GPETLCGAELVDALQFVCGDRGFYFNKPTGYGSSSR |
| | | RAPQTGIVDECCFRSCDTRRLEMYCAPLKPAKSA |
| 195 | L54V | GPETLCGAELVDALQFVCGDRGFYFNKPTGYGSSSR |
| | | RAPQTGIVDECCFRSCDVRRLEMYCAPLKPAKSA |
| 196 | L54W | GPETLCGAELVDALQFVCGDRGFYFNKPTGYGSSSR |
| | | RAPQTGIVDECCFRSCDWRRLEMYCAPLKPAKSA |
| 197 | L54Y | GPETLCGAELVDALQFVCGDRGFYFNKPTGYGSSSR |
| | | RAPQTGIVDECCFRSCDYRRLEMYCAPLKPAKSA |
| 198 | E3R L5A | GPRTACGAELVDALQFVCGDRGFYFNKPTGYGSSSR |
| | | RAPQTGIVDECCFRSCDLRRLEMYCAPLKPAKSA |
| 199 | E3R L5C | GPRTCCGAELVDALQFVCGDRGFYFNKPTGYGSSSR |
| | | RAPQTGIVDECCFRSCDLRRLEMYCAPLKPAKSA |
| 200 | E3R L5D | GPRTDCGAELVDALQFVCGDRGFYFNKPTGYGSSSR |
| | | RAPQTGIVDECCFRSCDLRRLEMYCAPLKPAKSA |
| 201 | E3R L5E | GPRTECGAELVDALQFVCGDRGFYFNKPTGYGSSSR |
| | | RAPQTGIVDECCFRSCDLRRLEMYCAPLKPAKSA |
| 202 | E3R L5F | GPRTFCGAELVDALQFVCGDRGFYFNKPTGYGSSSR |
| | | RAPQTGIVDECCFRSCDLRRLEMYCAPLKPAKSA |
| 203 | E3R L5G | GPRTGCGAELVDALQFVCGDRGFYFNKPTGYGSSSR |
| | | RAPQTGIVDECCFRSCDLRRLEMYCAPLKPAKSA |
| 204 | E3R L5H | GPRTHCGAELVDALQFVCGDRGFYFNKPTGYGSSSR |
| | | RAPQTGIVDECCFRSCDLRRLEMYCAPLKPAKSA |
| 205 | E3R L5I | GPRTICGAELVDALQFVCGDRGFYFNKPTGYGSSSRR |
| | | APQTGIVDECCFRSCDLRRLEMYCAPLKPAKSA |
| 206 | E3R L5K | GPRTKCGAELVDALQFVCGDRGFYFNKPTGYGSSSR |
| | | RAPQTGIVDECCFRSCDLRRLEMYCAPLKPAKSA |
| 207 | E3R L5M | GPRTMCGAELVDALQFVCGDRGFYFNKPTGYGSSSR |
| | | RAPQTGIVDECCFRSCDLRRLEMYCAPLKPAKSA |
| 208 | E3R L5N | GPRTNCGAELVDALQFVCGDRGFYFNKPTGYGSSSR |
| | | RAPQTGIVDECCFRSCDLRRLEMYCAPLKPAKSA |
| 209 | E3R L5P | GPRTPCGAELVDALQFVCGDRGFYFNKPTGYGSSSR |
| | | RAPQTGIVDECCFRSCDLRRLEMYCAPLKPAKSA |
| 210 | E3R L5Q | GPRTQCGAELVDALQFVCGDRGFYFNKPTGYGSSSR |
| | | RAPQTGIVDECCFRSCDLRRLEMYCAPLKPAKSA |
| 211 | E3R L5R | GPRTRCGAELVDALQFVCGDRGFYFNKPTGYGSSSR |
| | | RAPQTGIVDECCFRSCDLRRLEMYCAPLKPAKSA |
| 212 | E3R L5S | GPRTSCGAELVDALQFVCGDRGFYFNKPTGYGSSSR |
| | | RAPQTGIVDECCFRSCDLRRLEMYCAPLKPAKSA |
| 213 | E3R L5T | GPRTTCGAELVDALQFVCGDRGFYFNKPTGYGSSSR |
| | | RAPQTGIVDECCFRSCDLRRLEMYCAPLKPAKSA |
| 214 | E3R L5V | GPRTVCGAELVDALQFVCGDRGFYFNKPTGYGSSSR |
| | | RAPQTGIVDECCFRSCDLRRLEMYCAPLKPAKSA |
| 215 | E3R L5W | GPRTWCGAELVDALQFVCGDRGFYFNKPTGYGSSSR |
| | | RAPQTGIVDECCFRSCDLRRLEMYCAPLKPAKSA |
| 216 | E3R L5Y | GPRTYCGAELVDALQFVCGDRGFYFNKPTGYGSSSR |
| | | RAPQTGIVDECCFRSCDLRRLEMYCAPLKPAKSA |
| 217 | Des1-3 | TACGAELVDALQFVCGDRGFYFNKPTGYGSSSRRAP |
| | L5A | QTGIVDECCFRSCDLRRLEMYCAPLKPAKSA |
| 218 | Des1-3 L5C | TCCGAELVDALQFVCGDRGFYFNKPTGYGSSSRRAP |
| | | QTGIVDECCFRSCDLRRLEMYCAPLKPAKSA |
| 219 | Des1-3 | TDCGAELVDALQFVCGDRGFYFNKPTGYGSSSRRAP |
| | L5D | QTGIVDECCFRSCDLRRLEMYCAPLKPAKSA |
| 220 | Des1-3 L5E | TECGAELVDALQFVCGDRGFYFNKPTGYGSSSRRAP |
| | | QTGIVDECCFRSCDLRRLEMYCAPLKPAKSA |
| 221 | Des1-3 L5F | TFCGAELVDALQFVCGDRGFYFNKPTGYGSSSRRAP |
| | | QTGIVDECCFRSCDLRRLEMYCAPLKPAKSA |
| 222 | Des1-3 | TGCGAELVDALQFVCGDRGFYFNKPTGYGSSSRRAP |
| | L5G | QTGIVDECCFRSCDLRRLEMYCAPLKPAKSA |
| 223 | Des1-3 | THCGAELVDALQFVCGDRGFYFNKPTGYGSSSRRAP |
| | L5H | QTGIVDECCFRSCDLRRLEMYCAPLKPAKSA |
| 224 | Des1-3 L5I | TICGAELVDALQFVCGDRGFYFNKPTGYGSSSRRAPQ |
| | | TGIVDECCFRSCDLRRLEMYCAPLKPAKSA |
| 225 | Des1-3 | TKCGAELVDALQFVCGDRGFYFNKPTGYGSSSRRAP |
| | L5K | QTGIVDECCFRSCDLRRLEMYCAPLKPAKSA |
| 226 | Des1-3 | TMCGAELVDALQFVCGDRGFYFNKPTGYGSSSRRAP |
| | L5M | QTGIVDECCFRSCDLRRLEMYCAPLKPAKSA |
| 227 | Des1-3 | TNCGAELVDALQFVCGDRGFYFNKPTGYGSSSRRAP |
| | L5N | QTGIVDECCFRSCDLRRLEMYCAPLKPAKSA |
| 228 | Des1-3 L5P | TPCGAELVDALQFVCGDRGFYFNKPTGYGSSSRRAP |
| | | QTGIVDECCFRSCDLRRLEMYCAPLKPAKSA |
| 229 | Des1-3 | TQCGAELVDALQFVCGDRGFYFNKPTGYGSSSRRAP |
| | L5Q | QTGIVDECCFRSCDLRRLEMYCAPLKPAKSA |
| 230 | Des1-3 L5R | TRCGAELVDALQFVCGDRGFYFNKPTGYGSSSRRAP |
| | | QTGIVDECCFRSCDLRRLEMYCAPLKPAKSA |
| 231 | Des1-3 L5S | TSCGAELVDALQFVCGDRGFYFNKPTGYGSSSRRAP |
| | | QTGIVDECCFRSCDLRRLEMYCAPLKPAKSA |
| 232 | Des1-3 L5T | TTCGAELVDALQFVCGDRGFYFNKPTGYGSSSRRAP |
| | | QTGIVDECCFRSCDLRRLEMYCAPLKPAKSA |
| 233 | Des1-3 | TVCGAELVDALQFVCGDRGFYFNKPTGYGSSSRRAP |
| | L5V | QTGIVDECCFRSCDLRRLEMYCAPLKPAKSA |
| 234 | Des1-3 | TWCGAELVDALQFVCGDRGFYFNKPTGYGSSSRRAP |
| | L5W | QTGIVDECCFRSCDLRRLEMYCAPLKPAKSA |
| 235 | Des1-3 | TYCGAELVDALQFVCGDRGFYFNKPTGYGSSSRRAP |
| | L5Y | QTGIVDECCFRSCDLRRLEMYCAPLKPAKSA |
| 236 | E3R G7A | GPRTLCAAELVDALQFVCGDRGFYFNKPTGYGSSSR |
| | | RAPQTGIVDECCFRSCDLRRLEMYCAPLKPAKSA |
| 237 | E3R G7C | GPRTLCCAELVDALQFVCGDRGFYFNKPTGYGSSSR |
| | | RAPQTGIVDECCFRSCDLRRLEMYCAPLKPAKSA |
| 238 | E3R G7D | GPRTLCDAELVDALQFVCGDRGFYFNKPTGYGSSSR |
| | | RAPQTGIVDECCFRSCDLRRLEMYCAPLKPAKSA |
| 239 | E3R G7E | GPRTLCEAELVDALQFVCGDRGFYFNKPTGYGSSSR |
| | | RAPQTGIVDECCFRSCDLRRLEMYCAPLKPAKSA |
| 240 | E3R G7F | GPRTLCFAELVDALQFVCGDRGFYFNKPTGYGSSSRR |
| | | APQTGIVDECCFRSCDLRRLEMYCAPLKPAKSA |
| 241 | E3R G7H | GPRTLCHAELVDALQFVCGDRGFYFNKPTGYGSSSR |
| | | RAPQTGIVDECCFRSCDLRRLEMYCAPLKPAKSA |
| 242 | E3R G7I | GPRTLCIAELVDALQFVCGDRGFYFNKPTGYGSSSRR |
| | | APQTGIVDECCFRSCDLRRLEMYCAPLKPAKSA |
| 243 | E3R G7K | GPRTLCKAELVDALQFVCGDRGFYFNKPTGYGSSSR |
| | | RAPQTGIVDECCFRSCDLRRLEMYCAPLKPAKSA |
| 244 | E3R G7L | GPRTLCLAELVDALQFVCGDRGFYFNKPTGYGSSSR |
| | | RAPQTGIVDECCFRSCDLRRLEMYCAPLKPAKSA |
| 245 | E3R G7M | GPRTLCMAELVDALQFVCGDRGFYFNKPTGYGSSSR |
| | | RAPQTGIVDECCFRSCDLRRLEMYCAPLKPAKSA |
| 246 | E3R G7N | GPRTLCNAELVDALQFVCGDRGFYFNKPTGYGSSSR |
| | | RAPQTGIVDECCFRSCDLRRLEMYCAPLKPAKSA |
| 247 | E3R G7P | GPRTLCPAELVDALQFVCGDRGFYFNKPTGYGSSSRR |
| | | APQTGIVDECCFRSCDLRRLEMYCAPLKPAKSA |
| 248 | E3R G7Q | GPRTLCQAELVDALQFVCGDRGFYFNKPTGYGSSSR |
| | | RAPQTGIVDECCFRSCDLRRLEMYCAPLKPAKSA |
| 249 | E3R G7R | GPRTLCRAELVDALQFVCGDRGFYFNKPTGYGSSSR |
| | | RAPQTGIVDECCFRSCDLRRLEMYCAPLKPAKSA |
| 250 | E3R G7S | GPRTLCSAELVDALQFVCGDRGFYFNKPTGYGSSSRR |
| | | APQTGIVDECCFRSCDLRRLEMYCAPLKPAKSA |
| 251 | E3R G7T | GPRTLCTAELVDALQFVCGDRGFYFNKPTGYGSSSR |
| | | RAPQTGIVDECCFRSCDLRRLEMYCAPLKPAKSA |
| 252 | E3R G7V | GPRTLCVAELVDALQFVCGDRGFYFNKPTGYGSSSR |
| | | RAPQTGIVDECCFRSCDLRRLEMYCAPLKPAKSA |
| 253 | E3R G7W | GPRTLCWAELVDALQFVCGDRGFYFNKPTGYGSSSR |
| | | RAPQTGIVDECCFRSCDLRRLEMYCAPLKPAKSA |
| 254 | E3R G7Y | GPRTLCYAELVDALQFVCGDRGFYFNKPTGYGSSSR |
| | | RAPQTGIVDECCFRSCDLRRLEMYCAPLKPAKSA |
| 255 | Des1-3 | TLCAAELVDALQFVCGDRGFYFNKPTGYGSSSRRAP |
| | G7A | QTGIVDECCFRSCDLRRLEMYCAPLKPAKSA |
| 256 | Des1-3 | TLCCAELVDALQFVCGDRGFYFNKPTGYGSSSRRAP |
| | G7C | QTGIVDECCFRSCDLRRLEMYCAPLKPAKSA |
| 257 | Des1-3 | TLCDAELVDALQFVCGDRGFYFNKPTGYGSSSRRAP |
| | G7D | QTGIVDECCFRSCDLRRLEMYCAPLKPAKSA |
| 258 | Des1-3 | TLCEAELVDALQFVCGDRGFYFNKPTGYGSSSRRAP |
| | G7E | QTGIVDECCFRSCDLRRLEMYCAPLKPAKSA |
| 259 | Des1-3 G7F | TLCFAELVDALQFVCGDRGFYFNKPTGYGSSSRRAP |
| | | QTGIVDECCFRSCDLRRLEMYCAPLKPAKSA |
| 260 | Des1-3 | TLCHAELVDALQFVCGDRGFYFNKPTGYGSSSRRAP |
| | G7H | QTGIVDECCFRSCDLRRLEMYCAPLKPAKSA |
| 261 | Des1-3 G7I | TLCIAELVDALQFVCGDRGFYFNKPTGYGSSSRRAPQ |
| | | TGIVDECCFRSCDLRRLEMYCAPLKPAKSA |
| 262 | Des1-3 | TLCKAELVDALQFVCGDRGFYFNKPTGYGSSSRRAP |
| | G7K | QTGIVDECCFRSCDLRRLEMYCAPLKPAKSA |
| 263 | Des1-3 | TLCLAELVDALQFVCGDRGFYFNKPTGYGSSSRRAP |
| | G7L | QTGIVDECCFRSCDLRRLEMYCAPLKPAKSA |
| 264 | Des1-3 | TLCMAELVDALQFVCGDRGFYFNKPTGYGSSSRRAP |
| | G7M | QTGIVDECCFRSCDLRRLEMYCAPLKPAKSA |
| 265 | Des1-3 | TLCNAELVDALQFVCGDRGFYFNKPTGYGSSSRRAP |
| | G7N | QTGIVDECCFRSCDLRRLEMYCAPLKPAKSA |
| 266 | Des1-3 G7P | TLCPAELVDALQFVCGDRGFYFNKPTGYGSSSRRAP |
| | | QTGIVDECCFRSCDLRRLEMYCAPLKPAKSA |
| 267 | Des1-3 | TLCQAELVDALQFVCGDRGFYFNKPTGYGSSSRRAP |
| | G7Q | QTGIVDECCFRSCDLRRLEMYCAPLKPAKSA |
| 268 | Des1-3 | TLCRAELVDALQFVCGDRGFYFNKPTGYGSSSRRAP |
| | G7R | QTGIVDECCFRSCDLRRLEMYCAPLKPAKSA |
| 269 | Des1-3 | TLCSAELVDALQFVCGDRGFYFNKPTGYGSSSRRAP |
| | G7S | QTGIVDECCFRSCDLRRLEMYCAPLKPAKSA |
| 270 | Des1-3 | TLCTAELVDALQFVCGDRGFYFNKPTGYGSSSRRAP |
| | G7T | QTGIVDECCFRSCDLRRLEMYCAPLKPAKSA |
| 271 | Des1-3 | TLCVAELVDALQFVCGDRGFYFNKPTGYGSSSRRAP |
| | G7V | QTGIVDECCFRSCDLRRLEMYCAPLKPAKSA |
| 272 | Des1-3 | TLCWAELVDALQFVCGDRGFYFNKPTGYGSSSRRAP |
| | G7W | QTGIVDECCFRSCDLRRLEMYCAPLKPAKSA |
| 273 | Des1-3 | TLCYAELVDALQFVCGDRGFYFNKPTGYGSSSRRAP |
| | G7Y | QTGIVDECCFRSCDLRRLEMYCAPLKPAKSA |
| 274 | E3R L10A | GPRTLCGAEAVDALQFVCGDRGFYFNKPTGYGSSSR |
| | | RAPQTGIVDECCFRSCDLRRLEMYCAPLKPAKSA |
| 275 | E3R L10C | GPRTLCGAECVDALQFVCGDRGFYFNKPTGYGSSSR |
| | | RAPQTGIVDECCFRSCDLRRLEMYCAPLKPAKSA |
| 276 | E3R L10D | GPRTLCGAEDVDALQFVCGDRGFYFNKPTGYGSSSR |
| | | RAPQTGIVDECCFRSCDLRRLEMYCAPLKPAKSA |
| 277 | E3R L10E | GPRTLCGAEEVDALQFVCGDRGFYFNKPTGYGSSSR |
| | | RAPQTGIVDECCFRSCDLRRLEMYCAPLKPAKSA |
| 278 | E3R L1OF | GPRTLCGAEFVDALQFVCGDRGFYFNKPTGYGSSSR |
| | | RAPQTGIVDECCFRSCDLRRLEMYCAPLKPAKSA |
| 279 | E3R L10G | GPRTLCGAEGVDALQFVCGDRGFYFNKPTGYGSSSR |
| | | RAPQTGIVDECCFRSCDLRRLEMYCAPLKPAKSA |
| 280 | E3R L10H | GPRTLCGAEHVDALQFVCGDRGFYFNKPTGYGSSSR |
| | | RAPQTGIVDECCFRSCDLRRLEMYCAPLKPAKSA |
| 281 | E3R L10I | GPRTLCGAEIVDALQFVCGDRGFYFNKPTGYGSSSRR |
| | | APQTGIVDECCFRSCDLRRLEMYCAPLKPAKSA |
| 282 | E3R L10K | GPRTLCGAEKVDALQFVCGDRGFYFNKPTGYGSSSR |
| | | RAPQTGIVDECCFRSCDLRRLEMYCAPLKPAKSA |
| 283 | E3R L10M | GPRTLCGAEMVDALQFVCGDRGFYFNKPTGYGSSSR |
| | | RAPQTGIVDECCFRSCDLRRLEMYCAPLKPAKSA |
| 284 | E3R L10N | GPRTLCGAENVDALQFVCGDRGFYFNKPTGYGSSSR |
| | | RAPQTGIVDECCFRSCDLRRLEMYCAPLKPAKSA |
| 285 | E3R L10P | GPRTLCGAEPVDALQFVCGDRGFYFNKPTGYGSSSR |
| | | RAPQTGIVDECCFRSCDLRRLEMYCAPLKPAKSA |
| 286 | E3R L10Q | GPRTLCGAEQVDALQFVCGDRGFYFNKPTGYGSSSR |
| | | RAPQTGIVDECCFRSCDLRRLEMYCAPLKPAKSA |
| 287 | E3R L10R | GPRTLCGAERVDALQFVCGDRGFYFNKPTGYGSSSR |
| | | RAPQTGIVDECCFRSCDLRRLEMYCAPLKPAKSA |
| 288 | E3R L10S | GPRTLCGAESVDALQFVCGDRGFYFNKPTGYGSSSR |
| | | RAPQTGIVDECCFRSCDLRRLEMYCAPLKPAKSA |
| 289 | E3R L10T | GPRTLCGAETVDALQFVCGDRGFYFNKPTGYGSSSR |
| | | RAPQTGIVDECCFRSCDLRRLEMYCAPLKPAKSA |
| 290 | E3R L10V | GPRTLCGAEVVDALQFVCGDRGFYFNKPTGYGSSSR |
| | | RAPQTGIVDECCFRSCDLRRLEMYCAPLKPAKSA |
| 291 | E3R L10W | GPRTLCGAEWVDALQFVCGDRGFYFNKPTGYGSSSR |
| | | RAPQTGIVDECCFRSCDLRRLEMYCAPLKPAKSA |
| 292 | E3R L10Y | GPRTLCGAEYVDALQFVCGDRGFYFNKPTGYGSSSR |
| | | RAPQTGIVDECCFRSCDLRRLEMYCAPLKPAKSA |
| 293 | Des1-3 | TLCGAEAVDALQFVCGDRGFYFNKPTGYGSSSRRAP |
| | L10A | QTGIVDECCFRSCDLRRLEMYCAPLKPAKSA |
| 294 | Des1-3 | TLCGAECVDALQFVCGDRGFYFNKPTGYGSSSRRAP |
| | L10C | QTGIVDECCFRSCDLRRLEMYCAPLKPAKSA |
| 295 | Des1-3 | TLCGAEDVDALQFVCGDRGFYFNKPTGYGSSSRRAP |
| | L10D | QTGIVDECCFRSCDLRRLEMYCAPLKPAKSA |
| 296 | Des1-3 | TLCGAEEVDALQFVCGDRGFYFNKPTGYGSSSRRAP |
| | L10E | QTGIVDECCFRSCDLRRLEMYCAPLKPAKSA |
| 297 | Des1-3 | TLCGAEFVDALQFVCGDRGFYFNKPTGYGSSSRRAP |
| | L1OF | QTGIVDECCFRSCDLRRLEMYCAPLKPAKSA |
| 298 | Des1-3 | TLCGAEGVDALQFVCGDRGFYFNKPTGYGSSSRRAP |
| | L10G | QTGIVDECCFRSCDLRRLEMYCAPLKPAKSA |
| 299 | Des1-3 | TLCGAEHVDALQFVCGDRGFYFNKPTGYGSSSRRAP |
| | L10H | QTGIVDECCFRSCDLRRLEMYCAPLKPAKSA |
| 300 | Des1-3 | TLCGAEIVDALQFVCGDRGFYFNKPTGYGSSSRRAPQ |
| | L10I | TGIVDECCFRSCDLRRLEMYCAPLKPAKSA |
| 301 | Des1-3 | TLCGAEKVDALQFVCGDRGFYFNKPTGYGSSSRRAP |
| | L10K | QTGIVDECCFRSCDLRRLEMYCAPLKPAKSA |
| 302 | Des1-3 | TLCGAEMVDALQFVCGDRGFYFNKPTGYGSSSRRAP |
| | L10M | QTGIVDECCFRSCDLRRLEMYCAPLKPAKSA |
| 303 | Des1-3 | TLCGAENVDALQFVCGDRGFYFNKPTGYGSSSRRAP |
| | L10N | QTGIVDECCFRSCDLRRLEMYCAPLKPAKSA |
| 304 | Des1-3 | TLCGAEPVDALQFVCGDRGFYFNKPTGYGSSSRRAP |
| | L10P | QTGIVDECCFRSCDLRRLEMYCAPLKPAKSA |
| 305 | Des1-3 | TLCGAEQVDALQFVCGDRGFYFNKPTGYGSSSRRAP |
| | L10Q | QTGIVDECCFRSCDLRRLEMYCAPLKPAKSA |
| 306 | Des1-3 | TLCGAERVDALQFVCGDRGFYFNKPTGYGSSSRRAP |
| | L1OR | QTGIVDECCFRSCDLRRLEMYCAPLKPAKSA |
| 307 | Des1-3 | TLCGAESVDALQFVCGDRGFYFNKPTGYGSSSRRAP |
| | L10S | QTGIVDECCFRSCDLRRLEMYCAPLKPAKSA |
| 308 | Des1-3 | TLCGAETVDALQFVCGDRGFYFNKPTGYGSSSRRAP |
| | L10T | QTGIVDECCFRSCDLRRLEMYCAPLKPAKSA |
| 309 | Des1-3 | TLCGAEVVDALQFVCGDRGFYFNKPTGYGSSSRRAP |
| | L10V | QTGIVDECCFRSCDLRRLEMYCAPLKPAKSA |
| 310 | Des1-3 | TLCGAEWVDALQFVCGDRGFYFNKPTGYGSSSRRAP |
| | L10W | QTGIVDECCFRSCDLRRLEMYCAPLKPAKSA |
| 311 | Des1-3 | TLCGAEYVDALQFVCGDRGFYFNKPTGYGSSSRRAP |
| | L10Y | QTGIVDECCFRSCDLRRLEMYCAPLKPAKSA |
| 312 | E3R V17A | GPRTLCGAELVDALQFACGDRGFYFNKPTGYGSSSR |
| | | RAPQTGIVDECCFRSCDLRRLEMYCAPLKPAKSA |
| 313 | E3R V17C | GPRTLCGAELVDALQFCCGDRGFYFNKPTGYGSSSR |
| | | RAPQTGIVDECCFRSCDLRRLEMYCAPLKPAKSA |
| 314 | E3R V17D | GPRTLCGAELVDALQFDCGDRGFYFNKPTGYGSSSR |
| | | RAPQTGIVDECCFRSCDLRRLEMYCAPLKPAKSA |
| 315 | E3R V17E | GPRTLCGAELVDALQFECGDRGFYFNKPTGYGSSSR |
| | | RAPQTGIVDECCFRSCDLRRLEMYCAPLKPAKSA |
| 316 | E3R V17F | GPRTLCGAELVDALQFFCGDRGFYFNKPTGYGSSSRR |
| | | APQTGIVDECCFRSCDLRRLEMYCAPLKPAKSA |
| 317 | E3R V17G | GPRTLCGAELVDALQFGCGDRGFYFNKPTGYGSSSR |
| | | RAPQTGIVDECCFRSCDLRRLEMYCAPLKPAKSA |
| 318 | E3R V17H | GPRTLCGAELVDALQFHCGDRGFYFNKPTGYGSSSR |
| | | RAPQTGIVDECCFRSCDLRRLEMYCAPLKPAKSA |
| 319 | E3R V17I | GPRTLCGAELVDALQFICGDRGFYFNKPTGYGSSSRR |
| | | APQTGIVDECCFRSCDLRRLEMYCAPLKPAKSA |
| 320 | E3R V17K | GPRTLCGAELVDALQFKCGDRGFYFNKPTGYGSSSR |
| | | RAPQTGIVDECCFRSCDLRRLEMYCAPLKPAKSA |
| 321 | E3R V17L | GPRTLCGAELVDALQFLCGDRGFYFNKPTGYGSSSR |
| | | RAPQTGIVDECCFRSCDLRRLEMYCAPLKPAKSA |
| 322 | E3R V17M | GPRTLCGAELVDALQFMCGDRGFYFNKPTGYGSSSR |
| | | RAPQTGIVDECCFRSCDLRRLEMYCAPLKPAKSA |
| 323 | E3R V17N | GPRTLCGAELVDALQFNCGDRGFYFNKPTGYGSSSR |
| | | RAPQTGIVDECCFRSCDLRRLEMYCAPLKPAKSA |
| 324 | E3R V17P | GPRTLCGAELVDALQFPCGDRGFYFNKPTGYGSSSRR |
| | | APQTGIVDECCFRSCDLRRLEMYCAPLKPAKSA |
| 325 | E3R V17Q | GPRTLCGAELVDALQFQCGDRGFYFNKPTGYGSSSR |
| | | RAPQTGIVDECCFRSCDLRRLEMYCAPLKPAKSA |
| 326 | E3R V17R | GPRTLCGAELVDALQFRCGDRGFYFNKPTGYGSSSR |
| | | RAPQTGIVDECCFRSCDLRRLEMYCAPLKPAKSA |
| 327 | E3R V17S | GPRTLCGAELVDALQFSCGDRGFYFNKPTGYGSSSRR |
| | | APQTGIVDECCFRSCDLRRLEMYCAPLKPAKSA |
| 328 | E3R V17T | GPRTLCGAELVDALQFTCGDRGFYFNKPTGYGSSSR |
| | | RAPQTGIVDECCFRSCDLRRLEMYCAPLKPAKSA |
| 329 | E3R V17W | GPRTLCGAELVDALQFWCGDRGFYFNKPTGYGSSSR |
| | | RAPQTGIVDECCFRSCDLRRLEMYCAPLKPAKSA |
| 330 | E3R V17Y | GPRTLCGAELVDALQFYCGDRGFYFNKPTGYGSSSR |
| | | RAPQTGIVDECCFRSCDLRRLEMYCAPLKPAKSA |
| 331 | Des1-3 | TLCGAELVDALQFACGDRGFYFNKPTGYGSSSRRAP |
| | V17A | QTGIVDECCFRSCDLRRLEMYCAPLKPAKSA |
| 332 | Des1-3 | TLCGAELVDALQFCCGDRGFYFNKPTGYGSSSRRAP |
| | V17C | QTGIVDECCFRSCDLRRLEMYCAPLKPAKSA |
| 333 | Des1-3 | TLCGAELVDALQFDCGDRGFYFNKPTGYGSSSRRAP |
| | V17D | QTGIVDECCFRSCDLRRLEMYCAPLKPAKSA |
| 334 | Des1-3 | TLCGAELVDALQFECGDRGFYFNKPTGYGSSSRRAP |
| | V17E | QTGIVDECCFRSCDLRRLEMYCAPLKPAKSA |
| 335 | Des1-3 | TLCGAELVDALQFFCGDRGFYFNKPTGYGSSSRRAP |
| | V17F | QTGIVDECCFRSCDLRRLEMYCAPLKPAKSA |
| 336 | Des1-3 | TLCGAELVDALQFGCGDRGFYFNKPTGYGSSSRRAP |
| | V17G | QTGIVDECCFRSCDLRRLEMYCAPLKPAKSA |
| 337 | Des1-3 | TLCGAELVDALQFHCGDRGFYFNKPTGYGSSSRRAP |
| | V17H | QTGIVDECCFRSCDLRRLEMYCAPLKPAKSA |
| 338 | Des1-3 | TLCGAELVDALQFICGDRGFYFNKPTGYGSSSRRAPQ |
| | V17I | TGIVDECCFRSCDLRRLEMYCAPLKPAKSA |
| 339 | Des1-3 | TLCGAELVDALQFKCGDRGFYFNKPTGYGSSSRRAP |
| | V17K | QTGIVDECCFRSCDLRRLEMYCAPLKPAKSA |
| 340 | Des1-3 | TLCGAELVDALQFLCGDRGFYFNKPTGYGSSSRRAP |
| | V17L | QTGIVDECCFRSCDLRRLEMYCAPLKPAKSA |
| 341 | Des1-3 | TLCGAELVDALQFMCGDRGFYFNKPTGYGSSSRRAP |
| | V17M | QTGIVDECCFRSCDLRRLEMYCAPLKPAKSA |
| 342 | Des1-3 | TLCGAELVDALQFNCGDRGFYFNKPTGYGSSSRRAP |
| | V17N | QTGIVDECCFRSCDLRRLEMYCAPLKPAKSA |
| 343 | Des1-3 | TLCGAELVDALQFPCGDRGFYFNKPTGYGSSSRRAP |
| | V17P | QTGIVDECCFRSCDLRRLEMYCAPLKPAKSA |
| 344 | Des1-3 | TLCGAELVDALQFQCGDRGFYFNKPTGYGSSSRRAP |
| | V17Q | QTGIVDECCFRSCDLRRLEMYCAPLKPAKSA |
| 345 | Des1-3 | TLCGAELVDALQFRCGDRGFYFNKPTGYGSSSRRAP |
| | V17R | QTGIVDECCFRSCDLRRLEMYCAPLKPAKSA |
| 346 | Des1-3 | TLCGAELVDALQFSCGDRGFYFNKPTGYGSSSRRAP |
| | V17S | QTGIVDECCFRSCDLRRLEMYCAPLKPAKSA |
| 347 | Des1-3 | TLCGAELVDALQFTCGDRGFYFNKPTGYGSSSRRAP |
| | V17T | QTGIVDECCFRSCDLRRLEMYCAPLKPAKSA |
| 348 | Des1-3 | TLCGAELVDALQFWCGDRGFYFNKPTGYGSSSRRAP |
| | V17W | QTGIVDECCFRSCDLRRLEMYCAPLKPAKSA |
| 349 | Des1-3 | TLCGAELVDALQFYCGDRGFYFNKPTGYGSSSRRAP |
| | V17Y | QTGIVDECCFRSCDLRRLEMYCAPLKPAKSA |
| 350 | E3R G22A | GPRTLCGAELVDALQFVCGDRAFYFNKPTGYGSSSR |
| | | RAPQTGIVDECCFRSCDLRRLEMYCAPLKPAKSA |
| 351 | E3R G22C | GPRTLCGAELVDALQFVCGDRCFYFNKPTGYGSSSR |
| | | RAPQTGIVDECCFRSCDLRRLEMYCAPLKPAKSA |
| 352 | E3R G22D | GPRTLCGAELVDALQFVCGDRDFYFNKPTGYGSSSR |
| | | RAPQTGIVDECCFRSCDLRRLEMYCAPLKPAKSA |
| 353 | E3R G22E | GPRTLCGAELVDALQFVCGDREFYFNKPTGYGSSSR |
| | | RAPQTGIVDECCFRSCDLRRLEMYCAPLKPAKSA |
| 354 | E3R G22F | GPRTLCGAELVDALQFVCGDRFFYFNKPTGYGSSSRR |
| | | APQTGIVDECCFRSCDLRRLEMYCAPLKPAKSA |
| 355 | E3R G22H | GPRTLCGAELVDALQFVCGDRHFYFNKPTGYGSSSR |
| | | RAPQTGIVDECCFRSCDLRRLEMYCAPLKPAKSA |
| 356 | E3R G22I | GPRTLCGAELVDALQFVCGDRIFYFNKPTGYGSSSRR |
| | | APQTGIVDECCFRSCDLRRLEMYCAPLKPAKSA |
| 357 | E3R G22K | GPRTLCGAELVDALQFVCGDRKFYFNKPTGYGSSSR |
| | | RAPQTGIVDECCFRSCDLRRLEMYCAPLKPAKSA |
| 358 | E3R G22L | GPRTLCGAELVDALQFVCGDRLFYFNKPTGYGSSSR |
| | | RAPQTGIVDECCFRSCDLRRLEMYCAPLKPAKSA |
| 359 | E3R G22M | GPRTLCGAELVDALQFVCGDRMFYFNKPTGYGSSSR |
| | | RAPQTGIVDECCFRSCDLRRLEMYCAPLKPAKSA |
| 360 | E3R G22N | GPRTLCGAELVDALQFVCGDRNFYFNKPTGYGSSSR |
| | | RAPQTGIVDECCFRSCDLRRLEMYCAPLKPAKSA |
| 361 | E3R G22P | GPRTLCGAELVDALQFVCGDRPFYFNKPTGYGSSSRR |
| | | APQTGIVDECCFRSCDLRRLEMYCAPLKPAKSA |
| 362 | E3R G22Q | GPRTLCGAELVDALQFVCGDRQFYFNKPTGYGSSSR |
| | | RAPQTGIVDECCFRSCDLRRLEMYCAPLKPAKSA |
| 363 | E3R G22R | GPRTLCGAELVDALQFVCGDRRFYFNKPTGYGSSSR |
| | | RAPQTGIVDECCFRSCDLRRLEMYCAPLKPAKSA |
| 364 | E3R G22S | GPRTLCGAELVDALQFVCGDRSFYFNKPTGYGSSSRR |
| | | APQTGIVDECCFRSCDLRRLEMYCAPLKPAKSA |
| 365 | E3R G22T | GPRTLCGAELVDALQFVCGDRTFYFNKPTGYGSSSR |
| | | RAPQTGIVDECCFRSCDLRRLEMYCAPLKPAKSA |
| 366 | E3R G22V | GPRTLCGAELVDALQFVCGDRVFYFNKPTGYGSSSR |
| | | RAPQTGIVDECCFRSCDLRRLEMYCAPLKPAKSA |
| 367 | E3R G22W | GPRTLCGAELVDALQFVCGDRWFYFNKPTGYGSSSR |
| | | RAPQTGIVDECCFRSCDLRRLEMYCAPLKPAKSA |
| 368 | E3R G22Y | GPRTLCGAELVDALQFVCGDRYFYFNKPTGYGSSSR |
| | | RAPQTGIVDECCFRSCDLRRLEMYCAPLKPAKSA |
| 369 | Des1-3 | TLCGAELVDALQFVCGDRAFYFNKPTGYGSSSRRAP |
| | G22A | QTGIVDECCFRSCDLRRLEMYCAPLKPAKSA |
| 370 | Des1-3 | TLCGAELVDALQFVCGDRCFYFNKPTGYGSSSRRAP |
| | G22C | QTGIVDECCFRSCDLRRLEMYCAPLKPAKSA |
| 371 | Des1-3 | TLCGAELVDALQFVCGDRDFYFNKPTGYGSSSRRAP |
| | G22D | QTGIVDECCFRSCDLRRLEMYCAPLKPAKSA |
| 372 | Des1-3 | TLCGAELVDALQFVCGDREFYFNKPTGYGSSSRRAP |
| | G22E | QTGIVDECCFRSCDLRRLEMYCAPLKPAKSA |
| 373 | Des1-3 | TLCGAELVDALQFVCGDRFFYFNKPTGYGSSSRRAP |
| | G22F | QTGIVDECCFRSCDLRRLEMYCAPLKPAKSA |
| 374 | Des1-3 | TLCGAELVDALQFVCGDRHFYFNKPTGYGSSSRRAP |
| | G22H | QTGIVDECCFRSCDLRRLEMYCAPLKPAKSA |
| 375 | Des1-3 | TLCGAELVDALQFVCGDRIFYFNKPTGYGSSSRRAPQ |
| | G22I | TGIVDECCFRSCDLRRLEMYCAPLKPAKSA |
| 376 | Des1-3 | TLCGAELVDALQFVCGDRKFYFNKPTGYGSSSRRAP |
| | G22K | QTGIVDECCFRSCDLRRLEMYCAPLKPAKSA |
| 377 | Des1-3 | TLCGAELVDALQFVCGDRLFYFNKPTGYGSSSRRAP |
| | G22L | QTGIVDECCFRSCDLRRLEMYCAPLKPAKSA |
| 378 | Des1-3 | TLCGAELVDALQFVCGDRMFYFNKPTGYGSSSRRAP |
| | G22M | QTGIVDECCFRSCDLRRLEMYCAPLKPAKSA |
| 379 | Des1-3 | TLCGAELVDALQFVCGDRNFYFNKPTGYGSSSRRAP |
| | G22N | QTGIVDECCFRSCDLRRLEMYCAPLKPAKSA |
| 380 | Des1-3 | TLCGAELVDALQFVCGDRPFYFNKPTGYGSSSRRAP |
| | G22P | QTGIVDECCFRSCDLRRLEMYCAPLKPAKSA |
| 381 | Des1-3 | TLCGAELVDALQFVCGDRQFYFNKPTGYGSSSRRAP |
| | G22Q | QTGIVDECCFRSCDLRRLEMYCAPLKPAKSA |
| 382 | Des1-3 | TLCGAELVDALQFVCGDRRFYFNKPTGYGSSSRRAP |
| | G22R | QTGIVDECCFRSCDLRRLEMYCAPLKPAKSA |
| 383 | Des1-3 | TLCGAELVDALQFVCGDRSFYFNKPTGYGSSSRRAP |
| | G22S | QTGIVDECCFRSCDLRRLEMYCAPLKPAKSA |
| 384 | Des1-3 | TLCGAELVDALQFVCGDRTFYFNKPTGYGSSSRRAP |
| | G22T | QTGIVDECCFRSCDLRRLEMYCAPLKPAKSA |
| 385 | Des1-3 | TLCGAELVDALQFVCGDRVFYFNKPTGYGSSSRRAP |
| | G22V | QTGIVDECCFRSCDLRRLEMYCAPLKPAKSA |
| 386 | Des1-3 | TLCGAELVDALQFVCGDRWFYFNKPTGYGSSSRRAP |
| | G22W | QTGIVDECCFRSCDLRRLEMYCAPLKPAKSA |
| 387 | Des1-3 | TLCGAELVDALQFVCGDRYFYFNKPTGYGSSSRRAP |
| | G22Y | QTGIVDECCFRSCDLRRLEMYCAPLKPAKSA |
| 388 | E3R F23A | GPRTLCGAELVDALQFVCGDRGAYFNKPTGYGSSSR |
| | | RAPQTGIVDECCFRSCDLRRLEMYCAPLKPAKSA |
| 389 | E3R F23C | GPRTLCGAELVDALQFVCGDRGCYFNKPTGYGSSSR |
| | | RAPQTGIVDECCFRSCDLRRLEMYCAPLKPAKSA |
| 390 | E3R F23D | GPRTLCGAELVDALQFVCGDRGDYFNKPTGYGSSSR |
| | | RAPQTGIVDECCFRSCDLRRLEMYCAPLKPAKSA |
| 391 | E3R F23E | GPRTLCGAELVDALQFVCGDRGEYFNKPTGYGSSSR |
| | | RAPQTGIVDECCFRSCDLRRLEMYCAPLKPAKSA |
| 392 | E3R F23G | GPRTLCGAELVDALQFVCGDRGGYFNKPTGYGSSSR |
| | | RAPQTGIVDECCFRSCDLRRLEMYCAPLKPAKSA |
| 393 | E3R F23H | GPRTLCGAELVDALQFVCGDRGHYFNKPTGYGSSSR |
| | | RAPQTGIVDECCFRSCDLRRLEMYCAPLKPAKSA |
| 394 | E3R F23I | GPRTLCGAELVDALQFVCGDRGIYFNKPTGYGSSSRR |
| | | APQTGIVDECCFRSCDLRRLEMYCAPLKPAKSA |
| 395 | E3R F23K | GPRTLCGAELVDALQFVCGDRGKYFNKPTGYGSSSR |
| | | RAPQTGIVDECCFRSCDLRRLEMYCAPLKPAKSA |
| 396 | E3R F23L | GPRTLCGAELVDALQFVCGDRGLYFNKPTGYGSSSR |
| | | RAPQTGIVDECCFRSCDLRRLEMYCAPLKPAKSA |
| 397 | E3R F23M | GPRTLCGAELVDALQFVCGDRGMYFNKPTGYGSSSR |
| | | RAPQTGIVDECCFRSCDLRRLEMYCAPLKPAKSA |
| 398 | E3R F23N | GPRTLCGAELVDALQFVCGDRGNYFNKPTGYGSSSR |
| | | RAPQTGIVDECCFRSCDLRRLEMYCAPLKPAKSA |
| 399 | E3R F23P | GPRTLCGAELVDALQFVCGDRGPYFNKPTGYGSSSR |
| | | RAPQTGIVDECCFRSCDLRRLEMYCAPLKPAKSA |
| 400 | E3R F23Q | GPRTLCGAELVDALQFVCGDRGQYFNKPTGYGSSSR |
| | | RAPQTGIVDECCFRSCDLRRLEMYCAPLKPAKSA |
| 401 | E3R F23R | GPRTLCGAELVDALQFVCGDRGRYFNKPTGYGSSSR |
| | | RAPQTGIVDECCFRSCDLRRLEMYCAPLKPAKSA |
| 402 | E3R F23S | GPRTLCGAELVDALQFVCGDRGSYFNKPTGYGSSSR |
| | | RAPQTGIVDECCFRSCDLRRLEMYCAPLKPAKSA |
| 403 | E3R F23T | GPRTLCGAELVDALQFVCGDRGTYFNKPTGYGSSSR |
| | | RAPQTGIVDECCFRSCDLRRLEMYCAPLKPAKSA |
| 404 | E3R F23V | GPRTLCGAELVDALQFVCGDRGVYFNKPTGYGSSSR |
| | | RAPQTGIVDECCFRSCDLRRLEMYCAPLKPAKSA |
| 405 | E3R F23W | GPRTLCGAELVDALQFVCGDRGWYFNKPTGYGSSSR |
| | | RAPQTGIVDECCFRSCDLRRLEMYCAPLKPAKSA |
| 406 | E3R F23Y | GPRTLCGAELVDALQFVCGDRGYYFNKPTGYGSSSR |
| | | RAPQTGIVDECCFRSCDLRRLEMYCAPLKPAKSA |
| 407 | Des1-3 | TLCGAELVDALQFVCGDRGAYFNKPTGYGSSSRRAP |
| | F23A | QTGIVDECCFRSCDLRRLEMYCAPLKPAKSA |
| 408 | Des1-3 | TLCGAELVDALQFVCGDRGCYFNKPTGYGSSSRRAP |
| | F23C | QTGIVDECCFRSCDLRRLEMYCAPLKPAKSA |
| 409 | Des1-3 | TLCGAELVDALQFVCGDRGDYFNKPTGYGSSSRRAP |
| | F23D | QTGIVDECCFRSCDLRRLEMYCAPLKPAKSA |
| 410 | Des1-3 | TLCGAELVDALQFVCGDRGEYFNKPTGYGSSSRRAP |
| | F23E | QTGIVDECCFRSCDLRRLEMYCAPLKPAKSA |
| 411 | Des1-3 | TLCGAELVDALQFVCGDRGGYFNKPTGYGSSSRRAP |
| | F23G | QTGIVDECCFRSCDLRRLEMYCAPLKPAKSA |
| 412 | Des1-3 | TLCGAELVDALQFVCGDRGHYFNKPTGYGSSSRRAP |
| | F23H | QTGIVDECCFRSCDLRRLEMYCAPLKPAKSA |
| 413 | Des1-3 | TLCGAELVDALQFVCGDRGIYFNKPTGYGSSSRRAPQ |
| | F23I | TGIVDECCFRSCDLRRLEMYCAPLKPAKSA |
| 414 | Des1-3 | TLCGAELVDALQFVCGDRGKYFNKPTGYGSSSRRAP |
| | F23K | QTGIVDECCFRSCDLRRLEMYCAPLKPAKSA |
| 415 | Des1-3 | TLCGAELVDALQFVCGDRGLYFNKPTGYGSSSRRAP |
| | F23L | QTGIVDECCFRSCDLRRLEMYCAPLKPAKSA |
| 416 | Des1-3 | TLCGAELVDALQFVCGDRGMYFNKPTGYGSSSRRAP |
| | F23M | QTGIVDECCFRSCDLRRLEMYCAPLKPAKSA |
| 417 | Des1-3 | TLCGAELVDALQFVCGDRGNYFNKPTGYGSSSRRAP |
| | F23N | QTGIVDECCFRSCDLRRLEMYCAPLKPAKSA |
| 418 | Des1-3 | TLCGAELVDALQFVCGDRGPYFNKPTGYGSSSRRAP |
| | F23P | QTGIVDECCFRSCDLRRLEMYCAPLKPAKSA |
| 419 | Des1-3 | TLCGAELVDALQFVCGDRGQYFNKPTGYGSSSRRAP |
| | F23Q | QTGIVDECCFRSCDLRRLEMYCAPLKPAKSA |
| 420 | Des1-3 | TLCGAELVDALQFVCGDRGRYFNKPTGYGSSSRRAP |
| | F23R | QTGIVDECCFRSCDLRRLEMYCAPLKPAKSA |
| 421 | Des1-3 | TLCGAELVDALQFVCGDRGSYFNKPTGYGSSSRRAP |
| | F23S | QTGIVDECCFRSCDLRRLEMYCAPLKPAKSA |
| 422 | Des1-3 | TLCGAELVDALQFVCGDRGTYFNKPTGYGSSSRRAP |
| | F23T | QTGIVDECCFRSCDLRRLEMYCAPLKPAKSA |
| 423 | Des1-3 | TLCGAELVDALQFVCGDRGVYFNKPTGYGSSSRRAP |
| | F23V | QTGIVDECCFRSCDLRRLEMYCAPLKPAKSA |
| 424 | Des1-3 | TLCGAELVDALQFVCGDRGWYFNKPTGYGSSSRRAP |
| | F23W | QTGIVDECCFRSCDLRRLEMYCAPLKPAKSA |
| 425 | Des1-3 | TLCGAELVDALQFVCGDRGYYFNKPTGYGSSSRRAP |
| | F23Y | QTGIVDECCFRSCDLRRLEMYCAPLKPAKSA |
| 426 | E3R Y24A | GPRTLCGAELVDALQFVCGDRGFAFNKPTGYGSSSR |
| | | RAPQTGIVDECCFRSCDLRRLEMYCAPLKPAKSA |
| 427 | E3R Y24C | GPRTLCGAELVDALQFVCGDRGFCFNKPTGYGSSSR |
| | | RAPQTGIVDECCFRSCDLRRLEMYCAPLKPAKSA |
| 428 | E3R Y24D | GPRTLCGAELVDALQFVCGDRGFDFNKPTGYGSSSR |
| | | RAPQTGIVDECCFRSCDLRRLEMYCAPLKPAKSA |
| 429 | E3R Y24E | GPRTLCGAELVDALQFVCGDRGFEFNKPTGYGSSSR |
| | | RAPQTGIVDECCFRSCDLRRLEMYCAPLKPAKSA |
| 430 | E3R Y24F | GPRTLCGAELVDALQFVCGDRGFFFNKPTGYGSSSRR |
| | | APQTGIVDECCFRSCDLRRLEMYCAPLKPAKSA |
| 431 | E3R Y24G | GPRTLCGAELVDALQFVCGDRGFGFNKPTGYGSSSR |
| | | RAPQTGIVDECCFRSCDLRRLEMYCAPLKPAKSA |
| 432 | E3R Y24H | GPRTLCGAELVDALQFVCGDRGFHFNKPTGYGSSSR |
| | | RAPQTGIVDECCFRSCDLRRLEMYCAPLKPAKSA |
| 433 | E3R Y24I | GPRTLCGAELVDALQFVCGDRGFIFNKPTGYGSSSRR |
| | | APQTGIVDECCFRSCDLRRLEMYCAPLKPAKSA |
| 434 | E3R Y24K | GPRTLCGAELVDALQFVCGDRGFKFNKPTGYGSSSR |
| | | RAPQTGIVDECCFRSCDLRRLEMYCAPLKPAKSA |
| 435 | E3R Y24L | GPRTLCGAELVDALQFVCGDRGFLFNKPTGYGSSSR |
| | | RAPQTGIVDECCFRSCDLRRLEMYCAPLKPAKSA |
| 436 | E3R Y24M | GPRTLCGAELVDALQFVCGDRGFMFNKPTGYGSSSR |
| | | RAPQTGIVDECCFRSCDLRRLEMYCAPLKPAKSA |
| 437 | E3R Y24N | GPRTLCGAELVDALQFVCGDRGFNFNKPTGYGSSSR |
| | | RAPQTGIVDECCFRSCDLRRLEMYCAPLKPAKSA |
| 438 | E3R Y24P | GPRTLCGAELVDALQFVCGDRGFPFNKPTGYGSSSRR |
| | | APQTGIVDECCFRSCDLRRLEMYCAPLKPAKSA |
| 439 | E3R Y24Q | GPRTLCGAELVDALQFVCGDRGFQFNKPTGYGSSSR |
| | | RAPQTGIVDECCFRSCDLRRLEMYCAPLKPAKSA |
| 440 | E3R Y24R | GPRTLCGAELVDALQFVCGDRGFRFNKPTGYGSSSR |
| | | RAPQTGIVDECCFRSCDLRRLEMYCAPLKPAKSA |
| 441 | E3R Y24S | GPRTLCGAELVDALQFVCGDRGFSFNKPTGYGSSSRR |
| | | APQTGIVDECCFRSCDLRRLEMYCAPLKPAKSA |
| 442 | E3R Y24T | GPRTLCGAELVDALQFVCGDRGFTFNKPTGYGSSSR |
| | | RAPQTGIVDECCFRSCDLRRLEMYCAPLKPAKSA |
| 443 | E3R Y24V | GPRTLCGAELVDALQFVCGDRGFVFNKPTGYGSSSR |
| | | RAPQTGIVDECCFRSCDLRRLEMYCAPLKPAKSA |
| 444 | E3R Y24W | GPRTLCGAELVDALQFVCGDRGFWFNKPTGYGSSSR |
| | | RAPQTGIVDECCFRSCDLRRLEMYCAPLKPAKSA |
| 445 | Des1-3 | TLCGAELVDALQFVCGDRGFAFNKPTGYGSSSRRAP |
| | Y24A | QTGIVDECCFRSCDLRRLEMYCAPLKPAKSA |
| 446 | Des1-3 | TLCGAELVDALQFVCGDRGFCFNKPTGYGSSSRRAP |
| | Y24C | QTGIVDECCFRSCDLRRLEMYCAPLKPAKSA |
| 447 | Des1-3 | TLCGAELVDALQFVCGDRGFDFNKPTGYGSSSRRAP |
| | Y24D | QTGIVDECCFRSCDLRRLEMYCAPLKPAKSA |
| 448 | Des1-3 | TLCGAELVDALQFVCGDRGFEFNKPTGYGSSSRRAP |
| | Y24E | QTGIVDECCFRSCDLRRLEMYCAPLKPAKSA |
| 449 | Des1-3 | TLCGAELVDALQFVCGDRGFFFNKPTGYGSSSRRAP |
| | Y24F | QTGIVDECCFRSCDLRRLEMYCAPLKPAKSA |
| 450 | Des1-3 | TLCGAELVDALQFVCGDRGFGFNKPTGYGSSSRRAP |
| | Y24G | QTGIVDECCFRSCDLRRLEMYCAPLKPAKSA |
| 451 | Des1-3 | TLCGAELVDALQFVCGDRGFHFNKPTGYGSSSRRAP |
| | Y24H | QTGIVDECCFRSCDLRRLEMYCAPLKPAKSA |
| 452 | Des1-3 | TLCGAELVDALQFVCGDRGFIFNKPTGYGSSSRRAPQ |
| | Y24I | TGIVDECCFRSCDLRRLEMYCAPLKPAKSA |
| 453 | Des1-3 | TLCGAELVDALQFVCGDRGFKFNKPTGYGSSSRRAP |
| | Y24K | QTGIVDECCFRSCDLRRLEMYCAPLKPAKSA |
| 454 | Des1-3 | TLCGAELVDALQFVCGDRGFLFNKPTGYGSSSRRAP |
| | Y24L | QTGIVDECCFRSCDLRRLEMYCAPLKPAKSA |
| 455 | Des1-3 | TLCGAELVDALQFVCGDRGFMFNKPTGYGSSSRRAP |
| | Y24M | QTGIVDECCFRSCDLRRLEMYCAPLKPAKSA |
| 456 | Des1-3 | TLCGAELVDALQFVCGDRGFNFNKPTGYGSSSRRAP |
| | Y24N | QTGIVDECCFRSCDLRRLEMYCAPLKPAKSA |
| 457 | Des1-3 | TLCGAELVDALQFVCGDRGFPFNKPTGYGSSSRRAP |
| | Y24P | QTGIVDECCFRSCDLRRLEMYCAPLKPAKSA |
| 458 | Des1-3 | TLCGAELVDALQFVCGDRGFQFNKPTGYGSSSRRAP |
| | Y24Q | QTGIVDECCFRSCDLRRLEMYCAPLKPAKSA |
| 459 | Des1-3 | TLCGAELVDALQFVCGDRGFRFNKPTGYGSSSRRAP |
| | Y24R | QTGIVDECCFRSCDLRRLEMYCAPLKPAKSA |
| 460 | Des1-3 | TLCGAELVDALQFVCGDRGFSFNKPTGYGSSSRRAP |
| | Y24S | QTGIVDECCFRSCDLRRLEMYCAPLKPAKSA |
| 461 | Des1-3 | TLCGAELVDALQFVCGDRGFTFNKPTGYGSSSRRAP |
| | Y24T | QTGIVDECCFRSCDLRRLEMYCAPLKPAKSA |
| 462 | Des1-3 | TLCGAELVDALQFVCGDRGFVFNKPTGYGSSSRRAP |
| | Y24V | QTGIVDECCFRSCDLRRLEMYCAPLKPAKSA |
| 463 | Des1-3 | TLCGAELVDALQFVCGDRGFWFNKPTGYGSSSRRAP |
| | Y24W | QTGIVDECCFRSCDLRRLEMYCAPLKPAKSA |
| 464 | E3R F25A | GPRTLCGAELVDALQFVCGDRGFYANKPTGYGSSSR |
| | | RAPQTGIVDECCFRSCDLRRLEMYCAPLKPAKSA |
| 465 | E3R F25C | GPRTLCGAELVDALQFVCGDRGFYCNKPTGYGSSSR |
| | | RAPQTGIVDECCFRSCDLRRLEMYCAPLKPAKSA |
| 466 | E3R F25D | GPRTLCGAELVDALQFVCGDRGFYDNKPTGYGSSSR |
| | | RAPQTGIVDECCFRSCDLRRLEMYCAPLKPAKSA |
| 467 | E3R F25E | GPRTLCGAELVDALQFVCGDRGFYENKPTGYGSSSR |
| | | RAPQTGIVDECCFRSCDLRRLEMYCAPLKPAKSA |
| 468 | E3R F25G | GPRTLCGAELVDALQFVCGDRGFYGNKPTGYGSSSR |
| | | RAPQTGIVDECCFRSCDLRRLEMYCAPLKPAKSA |
| 469 | E3R F25H | GPRTLCGAELVDALQFVCGDRGFYHNKPTGYGSSSR |
| | | RAPQTGIVDECCFRSCDLRRLEMYCAPLKPAKSA |
| 470 | E3R F25I | GPRTLCGAELVDALQFVCGDRGFYINKPTGYGSSSRR |
| | | APQTGIVDECCFRSCDLRRLEMYCAPLKPAKSA |
| 471 | E3R F25K | GPRTLCGAELVDALQFVCGDRGFYKNKPTGYGSSSR |
| | | RAPQTGIVDECCFRSCDLRRLEMYCAPLKPAKSA |
| 472 | E3R F25L | GPRTLCGAELVDALQFVCGDRGFYLNKPTGYGSSSR |
| | | RAPQTGIVDECCFRSCDLRRLEMYCAPLKPAKSA |
| 473 | E3R F25M | GPRTLCGAELVDALQFVCGDRGFYMNKPTGYGSSSR |
| | | RAPQTGIVDECCFRSCDLRRLEMYCAPLKPAKSA |
| 474 | E3R F25N | GPRTLCGAELVDALQFVCGDRGFYNNKPTGYGSSSR |
| | | RAPQTGIVDECCFRSCDLRRLEMYCAPLKPAKSA |
| 475 | E3R F25P | GPRTLCGAELVDALQFVCGDRGFYPNKPTGYGSSSR |
| | | RAPQTGIVDECCFRSCDLRRLEMYCAPLKPAKSA |
| 476 | E3R F25Q | GPRTLCGAELVDALQFVCGDRGFYQNKPTGYGSSSR |
| | | RAPQTGIVDECCFRSCDLRRLEMYCAPLKPAKSA |
| 477 | E3R F25R | GPRTLCGAELVDALQFVCGDRGFYRNKPTGYGSSSR |
| | | RAPQTGIVDECCFRSCDLRRLEMYCAPLKPAKSA |
| 478 | E3R F25S | GPRTLCGAELVDALQFVCGDRGFYSNKPTGYGSSSR |
| | | RAPQTGIVDECCFRSCDLRRLEMYCAPLKPAKSA |
| 479 | E3R F25T | GPRTLCGAELVDALQFVCGDRGFYTNKPTGYGSSSR |
| | | RAPQTGIVDECCFRSCDLRRLEMYCAPLKPAKSA |
| 480 | E3R F25V | GPRTLCGAELVDALQFVCGDRGFYVNKPTGYGSSSR |
| | | RAPQTGIVDECCFRSCDLRRLEMYCAPLKPAKSA |
| 481 | E3R F25W | GPRTLCGAELVDALQFVCGDRGFYWNKPTGYGSSSR |
| | | RAPQTGIVDECCFRSCDLRRLEMYCAPLKPAKSA |
| 482 | E3R F25Y | GPRTLCGAELVDALQFVCGDRGFYYNKPTGYGSSSR |
| | | RAPQTGIVDECCFRSCDLRRLEMYCAPLKPAKSA |
| 483 | Des1-3 | TLCGAELVDALQFVCGDRGFYANKPTGYGSSSRRAP |
| | F25A | QTGIVDECCFRSCDLRRLEMYCAPLKPAKSA |
| 484 | Des1-3 | TLCGAELVDALQFVCGDRGFYCNKPTGYGSSSRRAP |
| | F25C | QTGIVDECCFRSCDLRRLEMYCAPLKPAKSA |
| 485 | Des1-3 | TLCGAELVDALQFVCGDRGFYDNKPTGYGSSSRRAP |
| | F25D | QTGIVDECCFRSCDLRRLEMYCAPLKPAKSA |
| 486 | Des1-3 | TLCGAELVDALQFVCGDRGFYENKPTGYGSSSRRAP |
| | F25E | QTGIVDECCFRSCDLRRLEMYCAPLKPAKSA |
| 487 | Des1-3 | TLCGAELVDALQFVCGDRGFYGNKPTGYGSSSRRAP |
| | F25G | QTGIVDECCFRSCDLRRLEMYCAPLKPAKSA |
| 488 | Des1-3 | TLCGAELVDALQFVCGDRGFYHNKPTGYGSSSRRAP |
| | F25H | QTGIVDECCFRSCDLRRLEMYCAPLKPAKSA |
| 489 | Des1-3 | TLCGAELVDALQFVCGDRGFYINKPTGYGSSSRRAPQ |
| | F25I | TGIVDECCFRSCDLRRLEMYCAPLKPAKSA |
| 490 | Des1-3 | TLCGAELVDALQFVCGDRGFYKNKPTGYGSSSRRAP |
| | F25K | QTGIVDECCFRSCDLRRLEMYCAPLKPAKSA |
| 491 | Des1-3 | TLCGAELVDALQFVCGDRGFYLNKPTGYGSSSRRAP |
| | F25L | QTGIVDECCFRSCDLRRLEMYCAPLKPAKSA |
| 492 | Des1-3 | TLCGAELVDALQFVCGDRGFYMNKPTGYGSSSRRAP |
| | F25M | QTGIVDECCFRSCDLRRLEMYCAPLKPAKSA |
| 493 | Des1-3 | TLCGAELVDALQFVCGDRGFYNNKPTGYGSSSRRAP |
| | F25N | QTGIVDECCFRSCDLRRLEMYCAPLKPAKSA |
| 494 | Des1-3 | TLCGAELVDALQFVCGDRGFYPNKPTGYGSSSRRAP |
| | F25P | QTGIVDECCFRSCDLRRLEMYCAPLKPAKSA |
| 495 | Des1-3 | TLCGAELVDALQFVCGDRGFYQNKPTGYGSSSRRAP |
| | F25Q | QTGIVDECCFRSCDLRRLEMYCAPLKPAKSA |
| 496 | Des1-3 | TLCGAELVDALQFVCGDRGFYRNKPTGYGSSSRRAP |
| | F25R | QTGIVDECCFRSCDLRRLEMYCAPLKPAKSA |
| 497 | Des1-3 | TLCGAELVDALQFVCGDRGFYSNKPTGYGSSSRRAP |
| | F25S | QTGIVDECCFRSCDLRRLEMYCAPLKPAKSA |
| 498 | Des1-3 | TLCGAELVDALQFVCGDRGFYTNKPTGYGSSSRRAP |
| | F25T | QTGIVDECCFRSCDLRRLEMYCAPLKPAKSA |
| 499 | Des1-3 | TLCGAELVDALQFVCGDRGFYVNKPTGYGSSSRRAP |
| | F25V | QTGIVDECCFRSCDLRRLEMYCAPLKPAKSA |
| 500 | Des1-3 | TLCGAELVDALQFVCGDRGFYWNKPTGYGSSSRRAP |
| | F25W | QTGIVDECCFRSCDLRRLEMYCAPLKPAKSA |
| 501 | Des1-3 | TLCGAELVDALQFVCGDRGFYYNKPTGYGSSSRRAP |
| | F25Y | QTGIVDECCFRSCDLRRLEMYCAPLKPAKSA |
| 502 | E3R S34A | GPRTLCGAELVDALQFVCGDRGFYFNKPTGYGSASR |
| | | RAPQTGIVDECCFRSCDLRRLEMYCAPLKPAKSA |
| 503 | E3R S34C | GPRTLCGAELVDALQFVCGDRGFYFNKPTGYGSCSR |
| | | RAPQTGIVDECCFRSCDLRRLEMYCAPLKPAKSA |
| 504 | E3R S34D | GPRTLCGAELVDALQFVCGDRGFYFNKPTGYGSDSR |
| | | RAPQTGIVDECCFRSCDLRRLEMYCAPLKPAKSA |
| 505 | E3R S34E | GPRTLCGAELVDALQFVCGDRGFYFNKPTGYGSESR |
| | | RAPQTGIVDECCFRSCDLRRLEMYCAPLKPAKSA |
| 506 | E3R S34F | GPRTLCGAELVDALQFVCGDRGFYFNKPTGYGSFSR |
| | | RAPQTGIVDECCFRSCDLRRLEMYCAPLKPAKSA |
| 507 | E3R S34G | GPRTLCGAELVDALQFVCGDRGFYFNKPTGYGSGSR |
| | | RAPQTGIVDECCFRSCDLRRLEMYCAPLKPAKSA |
| 508 | E3R S34H | GPRTLCGAELVDALQFVCGDRGFYFNKPTGYGSHSR |
| | | RAPQTGIVDECCFRSCDLRRLEMYCAPLKPAKSA |
| 509 | E3R S34I | GPRTLCGAELVDALQFVCGDRGFYFNKPTGYGSISRR |
| | | APQTGIVDECCFRSCDLRRLEMYCAPLKPAKSA |
| 510 | E3R S34K | GPRTLCGAELVDALQFVCGDRGFYFNKPTGYGSKSR |
| | | RAPQTGIVDECCFRSCDLRRLEMYCAPLKPAKSA |
| 511 | E3R S34L | GPRTLCGAELVDALQFVCGDRGFYFNKPTGYGSLSR |
| | | RAPQTGIVDECCFRSCDLRRLEMYCAPLKPAKSA |
| 512 | E3R S34M | GPRTLCGAELVDALQFVCGDRGFYFNKPTGYGSMSR |
| | | RAPQTGIVDECCFRSCDLRRLEMYCAPLKPAKSA |
| 513 | E3R S34N | GPRTLCGAELVDALQFVCGDRGFYFNKPTGYGSNSR |
| | | RAPQTGIVDECCFRSCDLRRLEMYCAPLKPAKSA |
| 514 | E3R S34P | GPRTLCGAELVDALQFVCGDRGFYFNKPTGYGSPSR |
| | | RAPQTGIVDECCFRSCDLRRLEMYCAPLKPAKSA |
| 515 | E3R S34Q | GPRTLCGAELVDALQFVCGDRGFYFNKPTGYGSQSR |
| | | RAPQTGIVDECCFRSCDLRRLEMYCAPLKPAKSA |
| 516 | E3R S34S | GPRTLCGAELVDALQFVCGDRGFYFNKPTGYGSSSR |
| | | RAPQTGIVDECCFRSCDLRRLEMYCAPLKPAKSA |
| 517 | E3R S34T | GPRTLCGAELVDALQFVCGDRGFYFNKPTGYGSTSR |
| | | RAPQTGIVDECCFRSCDLRRLEMYCAPLKPAKSA |
| 518 | E3R S34V | GPRTLCGAELVDALQFVCGDRGFYFNKPTGYGSVSR |
| | | RAPQTGIVDECCFRSCDLRRLEMYCAPLKPAKSA |
| 519 | E3R S34W | GPRTLCGAELVDALQFVCGDRGFYFNKPTGYGSWSR |
| | | RAPQTGIVDECCFRSCDLRRLEMYCAPLKPAKSA |
| 520 | E3R S34Y | GPRTLCGAELVDALQFVCGDRGFYFNKPTGYGSYSR |
| | | RAPQTGIVDECCFRSCDLRRLEMYCAPLKPAKSA |
| 521 | Des1-3 | TLCGAELVDALQFVCGDRGFYFNKPTGYGSASRRAP |
| | S34A | QTGIVDECCFRSCDLRRLEMYCAPLKPAKSA |
| 522 | Des1-3 | TLCGAELVDALQFVCGDRGFYFNKPTGYGSCSRRAP |
| | S34C | QTGIVDECCFRSCDLRRLEMYCAPLKPAKSA |
| 523 | Des1-3 | TLCGAELVDALQFVCGDRGFYFNKPTGYGSDSRRAP |
| | S34D | QTGIVDECCFRSCDLRRLEMYCAPLKPAKSA |
| 524 | Des1-3 | TLCGAELVDALQFVCGDRGFYFNKPTGYGSESRRAP |
| | S34E | QTGIVDECCFRSCDLRRLEMYCAPLKPAKSA |
| 525 | Des1-3 | TLCGAELVDALQFVCGDRGFYFNKPTGYGSFSRRAP |
| | S34F | QTGIVDECCFRSCDLRRLEMYCAPLKPAKSA |
| 526 | Des1-3 | TLCGAELVDALQFVCGDRGFYFNKPTGYGSGSRRAP |
| | S34G | QTGIVDECCFRSCDLRRLEMYCAPLKPAKSA |
| 527 | Des1-3 | TLCGAELVDALQFVCGDRGFYFNKPTGYGSHSRRAP |
| | S34H | QTGIVDECCFRSCDLRRLEMYCAPLKPAKSA |
| 528 | Des1-3 | TLCGAELVDALQFVCGDRGFYFNKPTGYGSISRRAPQ |
| | S34I | TGIVDECCFRSCDLRRLEMYCAPLKPAKSA |
| 529 | Des1-3 | TLCGAELVDALQFVCGDRGFYFNKPTGYGSKSRRAP |
| | S34K | QTGIVDECCFRSCDLRRLEMYCAPLKPAKSA |
| 530 | Des1-3 | TLCGAELVDALQFVCGDRGFYFNKPTGYGSLSRRAP |
| | S34L | QTGIVDECCFRSCDLRRLEMYCAPLKPAKSA |
| 531 | Des1-3 | TLCGAELVDALQFVCGDRGFYFNKPTGYGSMSRRAP |
| | S34M | QTGIVDECCFRSCDLRRLEMYCAPLKPAKSA |
| 532 | Des1-3 | TLCGAELVDALQFVCGDRGFYFNKPTGYGSNSRRAP |
| | S34N | QTGIVDECCFRSCDLRRLEMYCAPLKPAKSA |
| 533 | Des1-3 | TLCGAELVDALQFVCGDRGFYFNKPTGYGSPSRRAP |
| | S34P | QTGIVDECCFRSCDLRRLEMYCAPLKPAKSA |
| 534 | Des1-3 | TLCGAELVDALQFVCGDRGFYFNKPTGYGSQSRRAP |
| | S34Q | QTGIVDECCFRSCDLRRLEMYCAPLKPAKSA |
| 535 | Des1-3 | TLCGAELVDALQFVCGDRGFYFNKPTGYGSSSRRAP |
| | S34S | QTGIVDECCFRSCDLRRLEMYCAPLKPAKSA |
| 536 | Des1-3 | TLCGAELVDALQFVCGDRGFYFNKPTGYGSTSRRAP |
| | S34T | QTGIVDECCFRSCDLRRLEMYCAPLKPAKSA |
| 537 | Des1-3 | TLCGAELVDALQFVCGDRGFYFNKPTGYGSVSRRAP |
| | S34V | QTGIVDECCFRSCDLRRLEMYCAPLKPAKSA |
| 538 | Des1-3 | TLCGAELVDALQFVCGDRGFYFNKPTGYGSWSRRAP |
| | S34W | QTGIVDECCFRSCDLRRLEMYCAPLKPAKSA |
| 539 | Des1-3 | TLCGAELVDALQFVCGDRGFYFNKPTGYGSYSRRAP |
| | S34Y | QTGIVDECCFRSCDLRRLEMYCAPLKPAKSA |
| 540 | E3R L57A | GPRTLCGAELVDALQFVCGDRGFYFNKPTGYGSSSR |
| | | RAPQTGIVDECCFRSCDLRRAEMYCAPLKPAKSA |
| 541 | E3R L57C | GPRTLCGAELVDALQFVCGDRGFYFNKPTGYGSSSR |
| | | RAPQTGIVDECCFRSCDLRRCEMYCAPLKPAKSA |
| 542 | E3R L57D | GPRTLCGAELVDALQFVCGDRGFYFNKPTGYGSSSR |
| | | RAPQTGIVDECCFRSCDLRRDEMYCAPLKPAKSA |
| 543 | E3R L57E | GPRTLCGAELVDALQFVCGDRGFYFNKPTGYGSSSR |
| | | RAPQTGIVDECCFRSCDLRREEMYCAPLKPAKSA |
| 544 | E3R L57F | GPRTLCGAELVDALQFVCGDRGFYFNKPTGYGSSSR |
| | | RAPQTGIVDECCFRSCDLRRFEMYCAPLKPAKSA |
| 545 | E3R L57G | GPRTLCGAELVDALQFVCGDRGFYFNKPTGYGSSSR |
| | | RAPQTGIVDECCFRSCDLRRGEMYCAPLKPAKSA |
| 546 | E3R L57H | GPRTLCGAELVDALQFVCGDRGFYFNKPTGYGSSSR |
| | | RAPQTGIVDECCFRSCDLRRHEMYCAPLKPAKSA |
| 547 | E3R L57I | GPRTLCGAELVDALQFVCGDRGFYFNKPTGYGSSSR |
| | | RAPQTGIVDECCFRSCDLRRIEMYCAPLKPAKSA |
| 548 | E3R L57K | GPRTLCGAELVDALQFVCGDRGFYFNKPTGYGSSSR |
| | | RAPQTGIVDECCFRSCDLRRKEMYCAPLKPAKSA |
| 549 | E3R L57M | GPRTLCGAELVDALQFVCGDRGFYFNKPTGYGSSSR |
| | | RAPQTGIVDECCFRSCDLRRMEMYCAPLKPAKSA |
| 550 | E3R L57N | GPRTLCGAELVDALQFVCGDRGFYFNKPTGYGSSSR |
| | | RAPQTGIVDECCFRSCDLRRNEMYCAPLKPAKSA |
| 551 | E3R L57P | GPRTLCGAELVDALQFVCGDRGFYFNKPTGYGSSSR |
| | | RAPQTGIVDECCFRSCDLRRPEMYCAPLKPAKSA |
| 552 | E3R L57Q | GPRTLCGAELVDALQFVCGDRGFYFNKPTGYGSSSR |
| | | RAPQTGIVDECCFRSCDLRRQEMYCAPLKPAKSA |
| 553 | E3R L57R | GPRTLCGAELVDALQFVCGDRGFYFNKPTGYGSSSR |
| | | RAPQTGIVDECCFRSCDLRRREMYCAPLKPAKSA |
| 554 | E3R L57S | GPRTLCGAELVDALQFVCGDRGFYFNKPTGYGSSSR |
| | | RAPQTGIVDECCFRSCDLRRSEMYCAPLKPAKSA |
| 555 | E3R L57T | GPRTLCGAELVDALQFVCGDRGFYFNKPTGYGSSSR |
| | | RAPQTGIVDECCFRSCDLRRTEMYCAPLKPAKSA |
| 556 | E3R L57V | GPRTLCGAELVDALQFVCGDRGFYFNKPTGYGSSSR |
| | | RAPQTGIVDECCFRSCDLRRVEMYCAPLKPAKSA |
| 557 | E3R L57W | GPRTLCGAELVDALQFVCGDRGFYFNKPTGYGSSSR |
| | | RAPQTGIVDECCFRSCDLRRWEMYCAPLKPAKSA |
| 558 | E3R L57Y | GPRTLCGAELVDALQFVCGDRGFYFNKPTGYGSSSR |
| | | RAPQTGIVDECCFRSCDLRRYEMYCAPLKPAKSA |
| 559 | Des1-3 | TLCGAELVDALQFVCGDRGFYFNKPTGYGSSSRRAP |
| | L57A | QTGIVDECCFRSCDLRRAEMYCAPLKPAKSA |
| 560 | Des1-3 | TLCGAELVDALQFVCGDRGFYFNKPTGYGSSSRRAP |
| | L57C | QTGIVDECCFRSCDLRRCEMYCAPLKPAKSA |
| 561 | Des1-3 | TLCGAELVDALQFVCGDRGFYFNKPTGYGSSSRRAP |
| | L57D | QTGIVDECCFRSCDLRRDEMYCAPLKPAKSA |
| 562 | Des1-3 | TLCGAELVDALQFVCGDRGFYFNKPTGYGSSSRRAP |
| | L57E | QTGIVDECCFRSCDLRREEMYCAPLKPAKSA |
| 563 | Des1-3 | TLCGAELVDALQFVCGDRGFYFNKPTGYGSSSRRAP |
| | L57F | QTGIVDECCFRSCDLRRFEMYCAPLKPAKSA |
| 564 | Des1-3 | TLCGAELVDALQFVCGDRGFYFNKPTGYGSSSRRAP |
| | L57G | QTGIVDECCFRSCDLRRGEMYCAPLKPAKSA |
| 565 | Des1-3 | TLCGAELVDALQFVCGDRGFYFNKPTGYGSSSRRAP |
| | L57H | QTGIVDECCFRSCDLRRHEMYCAPLKPAKSA |
| 566 | Des1-3 | TLCGAELVDALQFVCGDRGFYFNKPTGYGSSSRRAP |
| | L57I | QTGIVDECCFRSCDLRRIEMYCAPLKPAKSA |
| 567 | Des1-3 | TLCGAELVDALQFVCGDRGFYFNKPTGYGSSSRRAP |
| | L57K | QTGIVDECCFRSCDLRRKEMYCAPLKPAKSA |
| 568 | Des1-3 | TLCGAELVDALQFVCGDRGFYFNKPTGYGSSSRRAP |
| | L57M | QTGIVDECCFRSCDLRRMEMYCAPLKPAKSA |
| 569 | Des1-3 | TLCGAELVDALQFVCGDRGFYFNKPTGYGSSSRRAP |
| | L57N | QTGIVDECCFRSCDLRRNEMYCAPLKPAKSA |
| 570 | Des1-3 | TLCGAELVDALQFVCGDRGFYFNKPTGYGSSSRRAP |
| | L57P | QTGIVDECCFRSCDLRRPEMYCAPLKPAKSA |
| 571 | Des1-3 | TLCGAELVDALQFVCGDRGFYFNKPTGYGSSSRRAP |
| | L57Q | QTGIVDECCFRSCDLRRQEMYCAPLKPAKSA |
| 572 | Des1-3 | TLCGAELVDALQFVCGDRGFYFNKPTGYGSSSRRAP |
| | L57R | QTGIVDECCFRSCDLRRREMYCAPLKPAKSA |
| 573 | Des1-3 | TLCGAELVDALQFVCGDRGFYFNKPTGYGSSSRRAP |
| | L57S | QTGIVDECCFRSCDLRRSEMYCAPLKPAKSA |
| 574 | Des1-3 | TLCGAELVDALQFVCGDRGFYFNKPTGYGSSSRRAP |
| | L57T | QTGIVDECCFRSCDLRRTEMYCAPLKPAKSA |
| 575 | Des1-3 | TLCGAELVDALQFVCGDRGFYFNKPTGYGSSSRRAP |
| | L57V | QTGIVDECCFRSCDLRRVEMYCAPLKPAKSA |
| 576 | Des1-3 | TLCGAELVDALQFVCGDRGFYFNKPTGYGSSSRRAP |
| | L57W | QTGIVDECCFRSCDLRRWEMYCAPLKPAKSA |
| 577 | Des1-3 | TLCGAELVDALQFVCGDRGFYFNKPTGYGSSSRRAP |
| | L57Y | QTGIVDECCFRSCDLRRYEMYCAPLKPAKSA |
| 578 | L5A | GPETACGAELVDALQFVCGDRGFYFNKPTGYGSSSR |
| | | RAPQTGIVDECCFRSCDLRRLEMYCAPLKPAKSA |
| 579 | L5C | GPETCCGAELVDALQFVCGDRGFYFNKPTGYGSSSR |
| | | RAPQTGIVDECCFRSCDLRRLEMYCAPLKPAKSA |
| 580 | L5D | GPETDCGAELVDALQFVCGDRGFYFNKPTGYGSSSR |
| | | RAPQTGIVDECCFRSCDLRRLEMYCAPLKPAKSA |
| 581 | L5E | GPETECGAELVDALQFVCGDRGFYFNKPTGYGSSSR |
| | | RAPQTGIVDECCFRSCDLRRLEMYCAPLKPAKSA |
| 582 | L5F | GPETFCGAELVDALQFVCGDRGFYFNKPTGYGSSSR |
| | | RAPQTGIVDECCFRSCDLRRLEMYCAPLKPAKSA |
| 583 | L5G | GPETGCGAELVDALQFVCGDRGFYFNKPTGYGSSSR |
| | | RAPQTGIVDECCFRSCDLRRLEMYCAPLKPAKSA |
| 584 | L5H | GPETHCGAELVDALQFVCGDRGFYFNKPTGYGSSSR |
| | | RAPQTGIVDECCFRSCDLRRLEMYCAPLKPAKSA |
| 585 | L5I | GPETICGAELVDALQFVCGDRGFYFNKPTGYGSSSRR |
| | | APQTGIVDECCFRSCDLRRLEMYCAPLKPAKSA |
| 586 | L5K | GPETKCGAELVDALQFVCGDRGFYFNKPTGYGSSSR |
| | | RAPQTGIVDECCFRSCDLRRLEMYCAPLKPAKSA |
| 587 | L5M | GPETMCGAELVDALQFVCGDRGFYFNKPTGYGSSSR |
| | | RAPQTGIVDECCFRSCDLRRLEMYCAPLKPAKSA |
| 588 | L5N | GPETNCGAELVDALQFVCGDRGFYFNKPTGYGSSSR |
| | | RAPQTGIVDECCFRSCDLRRLEMYCAPLKPAKSA |
| 589 | L5P | GPETPCGAELVDALQFVCGDRGFYFNKPTGYGSSSR |
| | | RAPQTGIVDECCFRSCDLRRLEMYCAPLKPAKSA |
| 590 | L5Q | GPETQCGAELVDALQFVCGDRGFYFNKPTGYGSSSR |
| | | RAPQTGIVDECCFRSCDLRRLEMYCAPLKPAKSA |
| 591 | L5R | GPETRCGAELVDALQFVCGDRGFYFNKPTGYGSSSR |
| | | RAPQTGIVDECCFRSCDLRRLEMYCAPLKPAKSA |
| 592 | L5S | GPETSCGAELVDALQFVCGDRGFYFNKPTGYGSSSR |
| | | RAPQTGIVDECCFRSCDLRRLEMYCAPLKPAKSA |
| 593 | L5T | GPETTCGAELVDALQFVCGDRGFYFNKPTGYGSSSR |
| | | RAPQTGIVDECCFRSCDLRRLEMYCAPLKPAKSA |
| 594 | L5V | GPETVCGAELVDALQFVCGDRGFYFNKPTGYGSSSR |
| | | RAPQTGIVDECCFRSCDLRRLEMYCAPLKPAKSA |
| 595 | L5W | GPETWCGAELVDALQFVCGDRGFYFNKPTGYGSSSR |
| | | RAPQTGIVDECCFRSCDLRRLEMYCAPLKPAKSA |
| 596 | L5Y | GPETYCGAELVDALQFVCGDRGFYFNKPTGYGSSSR |
| | | RAPQTGIVDECCFRSCDLRRLEMYCAPLKPAKSA |
| 597 | G7A | GPETLCAAELVDALQFVCGDRGFYFNKPTGYGSSSR |
| | | RAPQTGIVDECCFRSCDLRRLEMYCAPLKPAKSA |
| 598 | G7C | GPETLCCAELVDALQFVCGDRGFYFNKPTGYGSSSR |
| | | RAPQTGIVDECCFRSCDLRRLEMYCAPLKPAKSA |
| 599 | G7D | GPETLCDAELVDALQFVCGDRGFYFNKPTGYGSSSR |
| | | RAPQTGIVDECCFRSCDLRRLEMYCAPLKPAKSA |
| 600 | G7E | GPETLCEAELVDALQFVCGDRGFYFNKPTGYGSSSRR |
| | | APQTGIVDECCFRSCDLRRLEMYCAPLKPAKSA |
| 601 | G7F | GPETLCFAELVDALQFVCGDRGFYFNKPTGYGSSSRR |
| | | APQTGIVDECCFRSCDLRRLEMYCAPLKPAKSA |
| 602 | G7H | GPETLCHAELVDALQFVCGDRGFYFNKPTGYGSSSR |
| | | RAPQTGIVDECCFRSCDLRRLEMYCAPLKPAKSA |
| 603 | G7I | GPETLCIAELVDALQFVCGDRGFYFNKPTGYGSSSRR |
| | | APQTGIVDECCFRSCDLRRLEMYCAPLKPAKSA |
| 604 | G7K | GPETLCKAELVDALQFVCGDRGFYFNKPTGYGSSSR |
| | | RAPQTGIVDECCFRSCDLRRLEMYCAPLKPAKSA |
| 605 | G7L | GPETLCLAELVDALQFVCGDRGFYFNKPTGYGSSSRR |
| | | APQTGIVDECCFRSCDLRRLEMYCAPLKPAKSA |
| 60€ | G7M | GPETLCMAELVDALQFVCGDRGFYFNKPTGYGSSSR |
| | | RAPQTGIVDECCFRSCDLRRLEMYCAPLKPAKSA |
| 607 | G7N | GPETLCNAELVDALQFVCGDRGFYFNKPTGYGSSSR |
| | | RAPQTGIVDECCFRSCDLRRLEMYCAPLKPAKSA |
| 608 | G7P | GPETLCPAELVDALQFVCGDRGFYFNKPTGYGSSSRR |
| | | APQTGIVDECCFRSCDLRRLEMYCAPLKPAKSA |
| 609 | G7Q | GPETLCQAELVDALQFVCGDRGFYFNKPTGYGSSSR |
| | | RAPQTGIVDECCFRSCDLRRLEMYCAPLKPAKSA |
| 610 | G7R | GPETLCRAELVDALQFVCGDRGFYFNKPTGYGSSSR |
| | | RAPQTGIVDECCFRSCDLRRLEMYCAPLKPAKSA |
| 611 | G7S | GPETLCSAELVDALQFVCGDRGFYFNKPTGYGSSSRR |
| | | APQTGIVDECCFRSCDLRRLEMYCAPLKPAKSA |
| 612 | G7T | GPETLCTAELVDALQFVCGDRGFYFNKPTGYGSSSRR |
| | | APQTGIVDECCFRSCDLRRLEMYCAPLKPAKSA |
| 613 | G7V | GPETLCVAELVDALQFVCGDRGFYFNKPTGYGSSSR |
| | | RAPQTGIVDECCFRSCDLRRLEMYCAPLKPAKSA |
| 614 | G7W | GPETLCWAELVDALQFVCGDRGFYFNKPTGYGSSSR |
| | | RAPQTGIVDECCFRSCDLRRLEMYCAPLKPAKSA |
| 615 | G7Y | GPETLCYAELVDALQFVCGDRGFYFNKPTGYGSSSR |
| | | RAPQTGIVDECCFRSCDLRRLEMYCAPLKPAKSA |
| 616 | L10A | GPETLCGAEAVDALQFVCGDRGFYFNKPTGYGSSSR |
| | | RAPQTGIVDECCFRSCDLRRLEMYCAPLKPAKSA |
| 617 | L10C | GPETLCGAECVDALQFVCGDRGFYFNKPTGYGSSSR |
| | | RAPQTGIVDECCFRSCDLRRLEMYCAPLKPAKSA |
| 618 | L10D | GPETLCGAEDVDALQFVCGDRGFYFNKPTGYGSSSR |
| | | RAPQTGIVDECCFRSCDLRRLEMYCAPLKPAKSA |
| 619 | L10E | GPETLCGAEEVDALQFVCGDRGFYFNKPTGYGSSSR |
| | | RAPQTGIVDECCFRSCDLRRLEMYCAPLKPAKSA |
| 620 | L10F | GPETLCGAEFVDALQFVCGDRGFYFNKPTGYGSSSR |
| | | RAPQTGIVDECCFRSCDLRRLEMYCAPLKPAKSA |
| 621 | L10G | GPETLCGAEGVDALQFVCGDRGFYFNKPTGYGSSSR |
| | | RAPQTGIVDECCFRSCDLRRLEMYCAPLKPAKSA |
| 622 | L10H | GPETLCGAEHVDALQFVCGDRGFYFNKPTGYGSSSR |
| | | RAPQTGIVDECCFRSCDLRRLEMYCAPLKPAKSA |
| 623 | L10I | GPETLCGAEIVDALQFVCGDRGFYFNKPTGYGSSSRR |
| | | APQTGIVDECCFRSCDLRRLEMYCAPLKPAKSA |
| 624 | L10K | GPETLCGAEKVDALQFVCGDRGFYFNKPTGYGSSSR |
| | | RAPQTGIVDECCFRSCDLRRLEMYCAPLKPAKSA |
| 625 | L10M | GPETLCGAEMVDALQFVCGDRGFYFNKPTGYGSSSR |
| | | RAPQTGIVDECCFRSCDLRRLEMYCAPLKPAKSA |
| 626 | L10N | GPETLCGAENVDALQFVCGDRGFYFNKPTGYGSSSR |
| | | RAPQTGIVDECCFRSCDLRRLEMYCAPLKPAKSA |
| 627 | L10P | GPETLCGAEPVDALQFVCGDRGFYFNKPTGYGSSSR |
| | | RAPQTGIVDECCFRSCDLRRLEMYCAPLKPAKSA |
| 628 | L10Q | GPETLCGAEQVDALQFVCGDRGFYFNKPTGYGSSSR |
| | | RAPQTGIVDECCFRSCDLRRLEMYCAPLKPAKSA |
| 629 | L10R | GPETLCGAERVDALQFVCGDRGFYFNKPTGYGSSSR |
| | | RAPQTGIVDECCFRSCDLRRLEMYCAPLKPAKSA |
| 630 | L10S | GPETLCGAESVDALQFVCGDRGFYFNKPTGYGSSSR |
| | | RAPQTGIVDECCFRSCDLRRLEMYCAPLKPAKSA |
| 631 | L10T | GPETLCGAETVDALQFVCGDRGFYFNKPTGYGSSSR |
| | | RAPQTGIVDECCFRSCDLRRLEMYCAPLKPAKSA |
| 632 | L10V | GPETLCGAEVVDALQFVCGDRGFYFNKPTGYGSSSR |
| | | RAPQTGIVDECCFRSCDLRRLEMYCAPLKPAKSA |
| 633 | L10W | GPETLCGAEWVDALQFVCGDRGFYFNKPTGYGSSSR |
| | | RAPQTGIVDECCFRSCDLRRLEMYCAPLKPAKSA |
| 634 | L10Y | GPETLCGAEYVDALQFVCGDRGFYFNKPTGYGSSSR |
| | | RAPQTGIVDECCFRSCDLRRLEMYCAPLKPAKSA |
| 635 | V17A | GPETLCGAELVDALQFACGDRGFYFNKPTGYGSSSR |
| | | RAPQTGIVDECCFRSCDLRRLEMYCAPLKPAKSA |
| 636 | V17C | GPETLCGAELVDALQFCCGDRGFYFNKPTGYGSSSR |
| | | RAPQTGIVDECCFRSCDLRRLEMYCAPLKPAKSA |
| 637 | V17D | GPETLCGAELVDALQFDCGDRGFYFNKPTGYGSSSR |
| | | RAPQTGIVDECCFRSCDLRRLEMYCAPLKPAKSA |
| 638 | V17E | GPETLCGAELVDALQFECGDRGFYFNKPTGYGSSSRR |
| | | APQTGIVDECCFRSCDLRRLEMYCAPLKPAKSA |
| 639 | V17F | GPETLCGAELVDALQFFCGDRGFYFNKPTGYGSSSRR |
| | | APQTGIVDECCFRSCDLRRLEMYCAPLKPAKSA |
| 640 | V17G | GPETLCGAELVDALQFGCGDRGFYFNKPTGYGSSSR |
| | | RAPQTGIVDECCFRSCDLRRLEMYCAPLKPAKSA |
| 641 | V17H | GPETLCGAELVDALQFHCGDRGFYFNKPTGYGSSSR |
| | | RAPQTGIVDECCFRSCDLRRLEMYCAPLKPAKSA |
| 642 | V17I | GPETLCGAELVDALQFICGDRGFYFNKPTGYGSSSRR |
| | | APQTGIVDECCFRSCDLRRLEMYCAPLKPAKSA |
| 643 | V17K | GPETLCGAELVDALQFKCGDRGFYFNKPTGYGSSSR |
| | | RAPQTGIVDECCFRSCDLRRLEMYCAPLKPAKSA |
| 644 | V17L | GPETLCGAELVDALQFLCGDRGFYFNKPTGYGSSSRR |
| | | APQTGIVDECCFRSCDLRRLEMYCAPLKPAKSA |
| 645 | V17M | GPETLCGAELVDALQFMCGDRGFYFNKPTGYGSSSR |
| | | RAPQTGIVDECCFRSCDLRRLEMYCAPLKPAKSA |
| 646 | V17N | GPETLCGAELVDALQFNCGDRGFYFNKPTGYGSSSR |
| | | RAPQTGIVDECCFRSCDLRRLEMYCAPLKPAKSA |
| 647 | V17P | GPETLCGAELVDALQFPCGDRGFYFNKPTGYGSSSRR |
| | | APQTGIVDECCFRSCDLRRLEMYCAPLKPAKSA |
| 648 | V17Q | GPETLCGAELVDALQFQCGDRGFYFNKPTGYGSSSR |
| | | RAPQTGIVDECCFRSCDLRRLEMYCAPLKPAKSA |
| 649 | V17R | GPETLCGAELVDALQFRCGDRGFYFNKPTGYGSSSR |
| | | RAPQTGIVDECCFRSCDLRRLEMYCAPLKPAKSA |
| 650 | V17S | GPETLCGAELVDALQFSCGDRGFYFNKPTGYGSSSRR |
| | | APQTGIVDECCFRSCDLRRLEMYCAPLKPAKSA |
| 651 | V17T | GPETLCGAELVDALQFTCGDRGFYFNKPTGYGSSSRR |
| | | APQTGIVDECCFRSCDLRRLEMYCAPLKPAKSA |
| 652 | V17W | GPETLCGAELVDALQFWCGDRGFYFNKPTGYGSSSR |
| | | RAPQTGIVDECCFRSCDLRRLEMYCAPLKPAKSA |
| 653 | V17Y | GPETLCGAELVDALQFYCGDRGFYFNKPTGYGSSSR |
| | | RAPQTGIVDECCFRSCDLRRLEMYCAPLKPAKSA |
| 654 | G22A | GPETLCGAELVDALQFVCGDRAFYFNKPTGYGSSSR |
| | | RAPQTGIVDECCFRSCDLRRLEMYCAPLKPAKSA |
| 655 | G22C | GPETLCGAELVDALQFVCGDRCFYFNKPTGYGSSSR |
| | | RAPQTGIVDECCFRSCDLRRLEMYCAPLKPAKSA |
| 656 | G22D | GPETLCGAELVDALQFVCGDRDFYFNKPTGYGSSSR |
| | | RAPQTGIVDECCFRSCDLRRLEMYCAPLKPAKSA |
| 657 | G22E | GPETLCGAELVDALQFVCGDREFYFNKPTGYGSSSRR |
| | | APQTGIVDECCFRSCDLRRLEMYCAPLKPAKSA |
| 658 | G22F | GPETLCGAELVDALQFVCGDRFFYFNKPTGYGSSSRR |
| | | APQTGIVDECCFRSCDLRRLEMYCAPLKPAKSA |
| 659 | G22H | GPETLCGAELVDALQFVCGDRHFYFNKPTGYGSSSR |
| | | RAPQTGIVDECCFRSCDLRRLEMYCAPLKPAKSA |
| 660 | G22I | GPETLCGAELVDALQFVCGDRIFYFNKPTGYGSSSRR |
| | | APQTGIVDECCFRSCDLRRLEMYCAPLKPAKSA |
| 661 | G22K | GPETLCGAELVDALQFVCGDRKFYFNKPTGYGSSSR |
| | | RAPQTGIVDECCFRSCDLRRLEMYCAPLKPAKSA |
| 662 | G22L | GPETLCGAELVDALQFVCGDRLFYFNKPTGYGSSSRR |
| | | APQTGIVDECCFRSCDLRRLEMYCAPLKPAKSA |
| 663 | G22M | GPETLCGAELVDALQFVCGDRMFYFNKPTGYGSSSR |
| | | RAPQTGIVDECCFRSCDLRRLEMYCAPLKPAKSA |
| 664 | G22N | GPETLCGAELVDALQFVCGDRNFYFNKPTGYGSSSR |
| | | RAPQTGIVDECCFRSCDLRRLEMYCAPLKPAKSA |
| 665 | G22P | GPETLCGAELVDALQFVCGDRPFYFNKPTGYGSSSRR |
| | | APQTGIVDECCFRSCDLRRLEMYCAPLKPAKSA |
| 666 | G22Q | GPETLCGAELVDALQFVCGDRQFYFNKPTGYGSSSR |
| | | RAPQTGIVDECCFRSCDLRRLEMYCAPLKPAKSA |
| 667 | G22R | GPETLCGAELVDALQFVCGDRRFYFNKPTGYGSSSR |
| | | RAPQTGIVDECCFRSCDLRRLEMYCAPLKPAKSA |
| 668 | G22S | GPETLCGAELVDALQFVCGDRSFYFNKPTGYGSSSRR |
| | | APQTGIVDECCFRSCDLRRLEMYCAPLKPAKSA |
| 669 | G22T | GPETLCGAELVDALQFVCGDRTFYFNKPTGYGSSSRR |
| | | APQTGIVDECCFRSCDLRRLEMYCAPLKPAKSA |
| 670 | G22V | GPETLCGAELVDALQFVCGDRVFYFNKPTGYGSSSR |
| | | RAPQTGIVDECCFRSCDLRRLEMYCAPLKPAKSA |
| 671 | G22W | GPETLCGAELVDALQFVCGDRWFYFNKPTGYGSSSR |
| | | RAPQTGIVDECCFRSCDLRRLEMYCAPLKPAKSA |
| 672 | G22Y | GPETLCGAELVDALQFVCGDRYFYFNKPTGYGSSSR |
| | | RAPQTGIVDECCFRSCDLRRLEMYCAPLKPAKSA |
| 673 | F23A | GPETLCGAELVDALQFVCGDRGAYFNKPTGYGSSSR |
| | | RAPQTGIVDECCFRSCDLRRLEMYCAPLKPAKSA |
| 674 | F23C | GPETLCGAELVDALQFVCGDRGCYFNKPTGYGSSSR |
| | | RAPQTGIVDECCFRSCDLRRLEMYCAPLKPAKSA |
| 675 | F23D | GPETLCGAELVDALQFVCGDRGDYFNKPTGYGSSSR |
| | | RAPQTGIVDECCFRSCDLRRLEMYCAPLKPAKSA |
| 676 | F23E | GPETLCGAELVDALQFVCGDRGEYFNKPTGYGSSSR |
| | | RAPQTGIVDECCFRSCDLRRLEMYCAPLKPAKSA |
| 677 | F23G | GPETLCGAELVDALQFVCGDRGGYFNKPTGYGSSSR |
| | | RAPQTGIVDECCFRSCDLRRLEMYCAPLKPAKSA |
| 678 | F23H | GPETLCGAELVDALQFVCGDRGHYFNKPTGYGSSSR |
| | | RAPQTGIVDECCFRSCDLRRLEMYCAPLKPAKSA |
| 679 | F23I | GPETLCGAELVDALQFVCGDRGIYFNKPTGYGSSSRR |
| | | APQTGIVDECCFRSCDLRRLEMYCAPLKPAKSA |
| 680 | F23K | GPETLCGAELVDALQFVCGDRGKYFNKPTGYGSSSR |
| | | RAPQTGIVDECCFRSCDLRRLEMYCAPLKPAKSA |
| 681 | F23L | GPETLCGAELVDALQFVCGDRGLYFNKPTGYGSSSR |
| | | RAPQTGIVDECCFRSCDLRRLEMYCAPLKPAKSA |
| 682 | F23M | GPETLCGAELVDALQFVCGDRGMYFNKPTGYGSSSR |
| | | RAPQTGIVDECCFRSCDLRRLEMYCAPLKPAKSA |
| 683 | F23N | GPETLCGAELVDALQFVCGDRGNYFNKPTGYGSSSR |
| | | RAPQTGIVDECCFRSCDLRRLEMYCAPLKPAKSA |
| 684 | F23P | GPETLCGAELVDALQFVCGDRGPYFNKPTGYGSSSR |
| | | RAPQTGIVDECCFRSCDLRRLEMYCAPLKPAKSA |
| 685 | F23Q | GPETLCGAELVDALQFVCGDRGQYFNKPTGYGSSSR |
| | | RAPQTGIVDECCFRSCDLRRLEMYCAPLKPAKSA |
| 686 | F23R | GPETLCGAELVDALQFVCGDRGRYFNKPTGYGSSSR |
| | | RAPQTGIVDECCFRSCDLRRLEMYCAPLKPAKSA |
| 687 | F23S | GPETLCGAELVDALQFVCGDRGSYFNKPTGYGSSSR |
| | | RAPQTGIVDECCFRSCDLRRLEMYCAPLKPAKSA |
| 688 | F23T | GPETLCGAELVDALQFVCGDRGTYFNKPTGYGSSSR |
| | | RAPQTGIVDECCFRSCDLRRLEMYCAPLKPAKSA |
| 689 | F23V | GPETLCGAELVDALQFVCGDRGVYFNKPTGYGSSSR |
| | | RAPQTGIVDECCFRSCDLRRLEMYCAPLKPAKSA |
| 690 | F23W | GPETLCGAELVDALQFVCGDRGWYFNKPTGYGSSSR |
| | | RAPQTGIVDECCFRSCDLRRLEMYCAPLKPAKSA |
| 691 | F23Y | GPETLCGAELVDALQFVCGDRGYYFNKPTGYGSSSR |
| | | RAPQTGIVDECCFRSCDLRRLEMYCAPLKPAKSA |
| 692 | Y24A | GPETLCGAELVDALQFVCGDRGFAFNKPTGYGSSSR |
| | | RAPQTGIVDECCFRSCDLRRLEMYCAPLKPAKSA |
| 693 | Y24C | GPETLCGAELVDALQFVCGDRGFCFNKPTGYGSSSR |
| | | RAPQTGIVDECCFRSCDLRRLEMYCAPLKPAKSA |
| 694 | Y24D | GPETLCGAELVDALQFVCGDRGFDFNKPTGYGSSSR |
| | | RAPQTGIVDECCFRSCDLRRLEMYCAPLKPAKSA |
| 695 | Y24E | GPETLCGAELVDALQFVCGDRGFEFNKPTGYGSSSRR |
| | | APQTGIVDECCFRSCDLRRLEMYCAPLKPAKSA |
| 696 | Y24F | GPETLCGAELVDALQFVCGDRGFFFNKPTGYGSSSRR |
| | | APQTGIVDECCFRSCDLRRLEMYCAPLKPAKSA |
| 697 | Y24G | GPETLCGAELVDALQFVCGDRGFGFNKPTGYGSSSR |
| | | RAPQTGIVDECCFRSCDLRRLEMYCAPLKPAKSA |
| 698 | Y24H | GPETLCGAELVDALQFVCGDRGFHFNKPTGYGSSSR |
| | | RAPQTGIVDECCFRSCDLRRLEMYCAPLKPAKSA |
| 699 | Y24I | GPETLCGAELVDALQFVCGDRGFIFNKPTGYGSSSRR |
| | | APQTGIVDECCFRSCDLRRLEMYCAPLKPAKSA |
| 700 | Y24K | GPETLCGAELVDALQFVCGDRGFKFNKPTGYGSSSR |
| | | RAPQTGIVDECCFRSCDLRRLEMYCAPLKPAKSA |
| 701 | Y24L | GPETLCGAELVDALQFVCGDRGFLFNKPTGYGSSSRR |
| | | APQTGIVDECCFRSCDLRRLEMYCAPLKPAKSA |
| 702 | Y24M | GPETLCGAELVDALQFVCGDRGFMFNKPTGYGSSSR |
| | | RAPQTGIVDECCFRSCDLRRLEMYCAPLKPAKSA |
| 703 | Y24N | GPETLCGAELVDALQFVCGDRGFNFNKPTGYGSSSR |
| | | RAPQTGIVDECCFRSCDLRRLEMYCAPLKPAKSA |
| 704 | Y24P | GPETLCGAELVDALQFVCGDRGFPFNKPTGYGSSSRR |
| | | APQTGIVDECCFRSCDLRRLEMYCAPLKPAKSA |
| 705 | Y24Q | GPETLCGAELVDALQFVCGDRGFQFNKPTGYGSSSR |
| | | RAPQTGIVDECCFRSCDLRRLEMYCAPLKPAKSA |
| 706 | Y24R | GPETLCGAELVDALQFVCGDRGFRFNKPTGYGSSSR |
| | | RAPQTGIVDECCFRSCDLRRLEMYCAPLKPAKSA |
| 707 | Y24S | GPETLCGAELVDALQFVCGDRGFSFNKPTGYGSSSRR |
| | | APQTGIVDECCFRSCDLRRLEMYCAPLKPAKSA |
| 708 | Y24T | GPETLCGAELVDALQFVCGDRGFTFNKPTGYGSSSRR |
| | | APQTGIVDECCFRSCDLRRLEMYCAPLKPAKSA |
| 709 | Y24V | GPETLCGAELVDALQFVCGDRGFVFNKPTGYGSSSR |
| | | RAPQTGIVDECCFRSCDLRRLEMYCAPLKPAKSA |
| 710 | Y24W | GPETLCGAELVDALQFVCGDRGFWFNKPTGYGSSSR |
| | | RAPQTGIVDECCFRSCDLRRLEMYCAPLKPAKSA |
| 711 | F25A | GPETLCGAELVDALQFVCGDRGFYANKPTGYGSSSR |
| | | RAPQTGIVDECCFRSCDLRRLEMYCAPLKPAKSA |
| 712 | F25C | GPETLCGAELVDALQFVCGDRGFYCNKPTGYGSSSR |
| | | RAPQTGIVDECCFRSCDLRRLEMYCAPLKPAKSA |
| 713 | F25D | GPETLCGAELVDALQFVCGDRGFYDNKPTGYGSSSR |
| | | RAPQTGIVDECCFRSCDLRRLEMYCAPLKPAKSA |
| 714 | F25E | GPETLCGAELVDALQFVCGDRGFYENKPTGYGSSSR |
| | | RAPQTGIVDECCFRSCDLRRLEMYCAPLKPAKSA |
| 715 | F25G | GPETLCGAELVDALQFVCGDRGFYGNKPTGYGSSSR |
| | | RAPQTGIVDECCFRSCDLRRLEMYCAPLKPAKSA |
| 716 | F25H | GPETLCGAELVDALQFVCGDRGFYHNKPTGYGSSSR |
| | | RAPQTGIVDECCFRSCDLRRLEMYCAPLKPAKSA |
| 717 | F25I | GPETLCGAELVDALQFVCGDRGFYINKPTGYGSSSRR |
| | | APQTGIVDECCFRSCDLRRLEMYCAPLKPAKSA |
| 718 | F25K | GPETLCGAELVDALQFVCGDRGFYKNKPTGYGSSSR |
| | | RAPQTGIVDECCFRSCDLRRLEMYCAPLKPAKSA |
| 719 | F25L | GPETLCGAELVDALQFVCGDRGFYLNKPTGYGSSSR |
| | | RAPQTGIVDECCFRSCDLRRLEMYCAPLKPAKSA |
| 720 | F25M | GPETLCGAELVDALQFVCGDRGFYMNKPTGYGSSSR |
| | | RAPQTGIVDECCFRSCDLRRLEMYCAPLKPAKSA |
| 721 | F25N | GPETLCGAELVDALQFVCGDRGFYNNKPTGYGSSSR |
| | | RAPQTGIVDECCFRSCDLRRLEMYCAPLKPAKSA |
| 722 | F25P | GPETLCGAELVDALQFVCGDRGFYPNKPTGYGSSSR |
| | | RAPQTGIVDECCFRSCDLRRLEMYCAPLKPAKSA |
| 723 | F25Q | GPETLCGAELVDALQFVCGDRGFYQNKPTGYGSSSR |
| | | RAPQTGIVDECCFRSCDLRRLEMYCAPLKPAKSA |
| 724 | F25R | GPETLCGAELVDALQFVCGDRGFYRNKPTGYGSSSR |
| | | RAPQTGIVDECCFRSCDLRRLEMYCAPLKPAKSA |
| 725 | F25S | GPETLCGAELVDALQFVCGDRGFYSNKPTGYGSSSR |
| | | RAPQTGIVDECCFRSCDLRRLEMYCAPLKPAKSA |
| 726 | F25T | GPETLCGAELVDALQFVCGDRGFYTNKPTGYGSSSR |
| | | RAPQTGIVDECCFRSCDLRRLEMYCAPLKPAKSA |
| 727 | F25V | GPETLCGAELVDALQFVCGDRGFYVNKPTGYGSSSR |
| | | RAPQTGIVDECCFRSCDLRRLEMYCAPLKPAKSA |
| 728 | F25W | GPETLCGAELVDALQFVCGDRGFYWNKPTGYGSSSR |
| | | RAPQTGIVDECCFRSCDLRRLEMYCAPLKPAKSA |
| 729 | F25Y | GPETLCGAELVDALQFVCGDRGFYYNKPTGYGSSSR |
| | | RAPQTGIVDECCFRSCDLRRLEMYCAPLKPAKSA |
| 730 | S34A | GPETLCGAELVDALQFVCGDRGFYFNKPTGYGSASR |
| | | RAPQTGIVDECCFRSCDLRRLEMYCAPLKPAKSA |
| 731 | S34C | GPETLCGAELVDALQFVCGDRGFYFNKPTGYGSCSR |
| | | RAPQTGIVDECCFRSCDLRRLEMYCAPLKPAKSA |
| 732 | S34D | GPETLCGAELVDALQFVCGDRGFYFNKPTGYGSDSR |
| | | RAPQTGIVDECCFRSCDLRRLEMYCAPLKPAKSA |
| 733 | S34E | GPETLCGAELVDALQFVCGDRGFYFNKPTGYGSESR |
| | | RAPQTGIVDECCFRSCDLRRLEMYCAPLKPAKSA |
| 734 | S34F | GPETLCGAELVDALQFVCGDRGFYFNKPTGYGSFSR |
| | | RAPQTGIVDECCFRSCDLRRLEMYCAPLKPAKSA |
| 735 | S34G | GPETLCGAELVDALQFVCGDRGFYFNKPTGYGSGSR |
| | | RAPQTGIVDECCFRSCDLRRLEMYCAPLKPAKSA |
| 736 | S34H | GPETLCGAELVDALQFVCGDRGFYFNKPTGYGSHSR |
| | | RAPQTGIVDECCFRSCDLRRLEMYCAPLKPAKSA |
| 737 | S34I | GPETLCGAELVDALQFVCGDRGFYFNKPTGYGSISRR |
| | | APQTGIVDECCFRSCDLRRLEMYCAPLKPAKSA |
| 738 | S34K | GPETLCGAELVDALQFVCGDRGFYFNKPTGYGSKSR |
| | | RAPQTGIVDECCFRSCDLRRLEMYCAPLKPAKSA |
| 739 | S34L | GPETLCGAELVDALQFVCGDRGFYFNKPTGYGSLSR |
| | | RAPQTGIVDECCFRSCDLRRLEMYCAPLKPAKSA |
| 740 | S34M | GPETLCGAELVDALQFVCGDRGFYFNKPTGYGSMSR |
| | | RAPQTGIVDECCFRSCDLRRLEMYCAPLKPAKSA |
| 741 | S34N | GPETLCGAELVDALQFVCGDRGFYFNKPTGYGSNSR |
| | | RAPQTGIVDECCFRSCDLRRLEMYCAPLKPAKSA |
| 742 | S34P | GPETLCGAELVDALQFVCGDRGFYFNKPTGYGSPSR |
| | | RAPQTGIVDECCFRSCDLRRLEMYCAPLKPAKSA |
| 743 | S34Q | GPETLCGAELVDALQFVCGDRGFYFNKPTGYGSQSR |
| | | RAPQTGIVDECCFRSCDLRRLEMYCAPLKPAKSA |
| 744 | S34S | GPETLCGAELVDALQFVCGDRGFYFNKPTGYGSSSR |
| | | RAPQTGIVDECCFRSCDLRRLEMYCAPLKPAKSA |
| 745 | S34T | GPETLCGAELVDALQFVCGDRGFYFNKPTGYGSTSR |
| | | RAPQTGIVDECCFRSCDLRRLEMYCAPLKPAKSA |
| 746 | S34V | GPETLCGAELVDALQFVCGDRGFYFNKPTGYGSVSR |
| | | RAPQTGIVDECCFRSCDLRRLEMYCAPLKPAKSA |
| 747 | S34W | GPETLCGAELVDALQFVCGDRGFYFNKPTGYGSWSR |
| | | RAPQTGIVDECCFRSCDLRRLEMYCAPLKPAKSA |
| 748 | S34Y | GPETLCGAELVDALQFVCGDRGFYFNKPTGYGSYSR |
| | | RAPQTGIVDECCFRSCDLRRLEMYCAPLKPAKSA |
| 749 | L57A | GPETLCGAELVDALQFVCGDRGFYFNKPTGYGSSSR |
| | | RAPQTGIVDECCFRSCDLRRAEMYCAPLKPAKSA |
| 750 | L57C | GPETLCGAELVDALQFVCGDRGFYFNKPTGYGSSSR |
| | | RAPQTGIVDECCFRSCDLRRCEMYCAPLKPAKSA |
| 751 | L57D | GPETLCGAELVDALQFVCGDRGFYFNKPTGYGSSSR |
| | | RAPQTGIVDECCFRSCDLRRDEMYCAPLKPAKSA |
| 752 | L57E | GPETLCGAELVDALQFVCGDRGFYFNKPTGYGSSSR |
| | | RAPQTGIVDECCFRSCDLRREEMYCAPLKPAKSA |
| 753 | L57F | GPETLCGAELVDALQFVCGDRGFYFNKPTGYGSSSR |
| | | RAPQTGIVDECCFRSCDLRRFEMYCAPLKPAKSA |
| 754 | L57G | GPETLCGAELVDALQFVCGDRGFYFNKPTGYGSSSR |
| | | RAPQTGIVDECCFRSCDLRRGEMYCAPLKPAKSA |
| 755 | L57H | GPETLCGAELVDALQFVCGDRGFYFNKPTGYGSSSR |
| | | RAPQTGIVDECCFRSCDLRRHEMYCAPLKPAKSA |
| 756 | L57I | GPETLCGAELVDALQFVCGDRGFYFNKPTGYGSSSR |
| | | RAPQTGIVDECCFRSCDLRRIEMYCAPLKPAKSA |
| 757 | L57K | GPETLCGAELVDALQFVCGDRGFYFNKPTGYGSSSR |
| | | RAPQTGIVDECCFRSCDLRRKEMYCAPLKPAKSA |
| 758 | L57M | GPETLCGAELVDALQFVCGDRGFYFNKPTGYGSSSR |
| | | RAPQTGIVDECCFRSCDLRRMEMYCAPLKPAKSA |
| 759 | L57N | GPETLCGAELVDALQFVCGDRGFYFNKPTGYGSSSR |
| | | RAPQTGIVDECCFRSCDLRRNEMYCAPLKPAKSA |
| 760 | L57P | GPETLCGAELVDALQFVCGDRGFYFNKPTGYGSSSR |
| | | RAPQTGIVDECCFRSCDLRRPEMYCAPLKPAKSA |
| 761 | L57Q | GPETLCGAELVDALQFVCGDRGFYFNKPTGYGSSSR |
| | | RAPQTGIVDECCFRSCDLRRQEMYCAPLKPAKSA |
| 762 | L57R | GPETLCGAELVDALQFVCGDRGFYFNKPTGYGSSSR |
| | | RAPQTGIVDECCFRSCDLRRREMYCAPLKPAKSA |
| 763 | L57S | GPETLCGAELVDALQFVCGDRGFYFNKPTGYGSSSR |
| | | RAPQTGIVDECCFRSCDLRRSEMYCAPLKPAKSA |
| 764 | L57T | GPETLCGAELVDALQFVCGDRGFYFNKPTGYGSSSR |
| | | RAPQTGIVDECCFRSCDLRRTEMYCAPLKPAKSA |
| 765 | L57V | GPETLCGAELVDALQFVCGDRGFYFNKPTGYGSSSR |
| | | RAPQTGIVDECCFRSCDLRRVEMYCAPLKPAKSA |
| 766 | L57W | GPETLCGAELVDALQFVCGDRGFYFNKPTGYGSSSR |
| | | RAPQTGIVDECCFRSCDLRRWEMYCAPLKPAKSA |
| 767 | L57Y | GPETLCGAELVDALQFVCGDRGFYFNKPTGYGSSSR |
| | | RAPQTGIVDECCFRSCDLRRYEMYCAPLKPAKSA |
| 768 | E3K | GPKTLCGAELVDALQFVCGDRGFYFNKPTGYGSSSR |
| | | RAPQTGIVDECCFRSCDLRRLEMYCAPLKPAKSA |
| 769 | E3G | GPGTLCGAELVDALQFVCGDRGFYFNKPTGYGSSSR |
| | | RAPQTGIVDECCFRSCDLRRLEMYCAPLKPAKSA |
| 770 | E3K/F49L | GPKTLCGAELVDALQFVCGDRGFYFNKPTGYGSSSR |
| | | RAPQTGIVDECCLRSCDLRRLEMYCAPLKPAKSA |
| 771 | E3G/F49L | GPGTLCGAELVDALQFVCGDRGFYFNKPTGYGSSSR |
| | | RAPQTGIVDECCLRSCDLRRLEMYCAPLKPAKSA |

In some embodiments, the engineered polypeptide comprises or consists of SEQ ID NO: 1 (native human IGF-1). The engineered polypeptide may comprise or consist of 70 amino acids. The engineered polypeptide may have potent binding to IGF1R. The engineered polypeptide may have potent binding to the one or more IGFBPs. An affinity of native IGF-1 for IGF1R may be in a range of about 1 nanomolar (nM) to about 10 nM, as measured by an affinity assay.

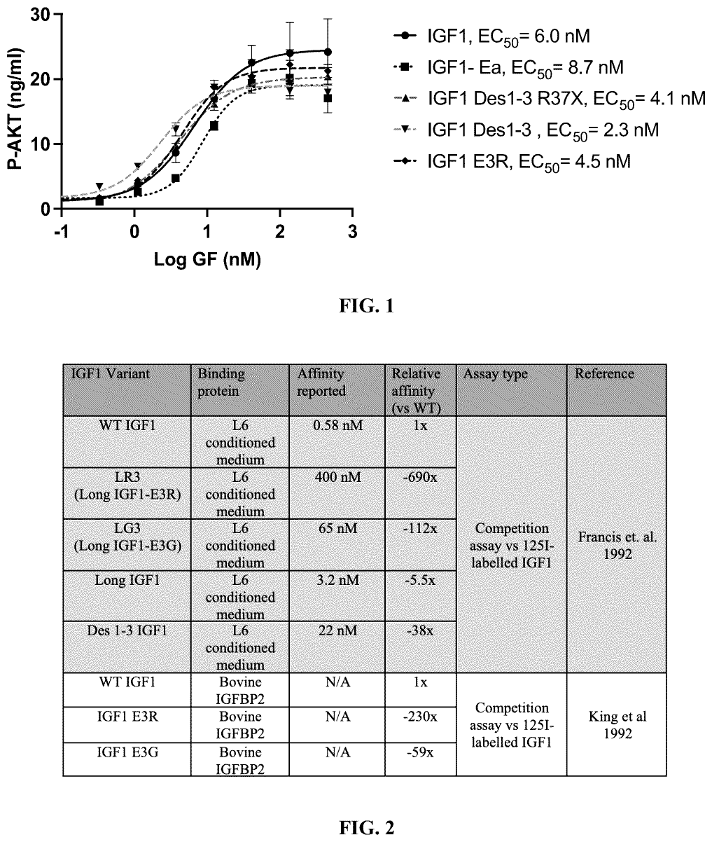

In one aspect, the engineered polypeptide is an IGF-1 variant. In some embodiments, the engineered polypeptide is a non-naturally occurring IGF-1 variant. In some embodiments, the IGF-1 variant has at least 60% sequence identity to SEQ ID NO: 1. In some embodiments, the IGF-1 variant has at least 65% sequence identity to SEQ ID NO: 1. In some embodiments, the IGF-1 variant has at least 70% sequence identity to SEQ ID NO: 1. In some embodiments, the IGF-1 variant has at least 75% sequence identity to SEQ ID NO: 1. In some embodiments, the IGF-1 variant has at least 80% sequence identity to SEQ ID NO: 1. In some embodiments, the IGF-1 variant has at least 85% sequence identity to SEQ ID NO: 1. In some embodiments, the IGF-1 variant has at least 90% sequence identity to SEQ ID NO: 1. In some embodiments, the IGF-1 variant has at least 91% sequence identity to SEQ ID NO: 1. In some embodiments, the IGF-1 variant has at least 92% sequence identity to SEQ ID NO: 1. In some embodiments, the IGF-1 variant has at least 93% sequence identity to SEQ ID NO: 1. In some embodiments, the IGF-1 variant has at least 94% sequence identity to SEQ ID NO: 1. In some embodiments, the IGF-1 variant has at least 95% sequence identity to SEQ ID NO: 1. In some embodiments, the IGF-1 variant has at least 96% sequence identity to SEQ ID NO: 1. In some embodiments, the IGF-1 variant has at least 97% sequence identity to SEQ ID NO: 1. In some embodiments, the IGF-1 variant has at least 98% sequence identity to SEQ ID NO: 1. In some embodiments, an affinity of the IGF-1 variant for IGF1R may be in a range of about 1 nanomolar (nM) to about 10 nM, as measured by an affinity assay. In some embodiments, the engineered polypeptide (e.g., IGF-1 variant) has an EC50 of greater than 1 nM, greater than 2 nM, greater than 3 nM, greater than 4 nM, or greater than 5 nM as determined via the assay described in connection with FIG. 1. In some embodiments, the EC50 of the engineered polypeptide is between 1 nM and 20 nM, between 1 nM and 15 nM, between 1 nM and 10 nM, between 2 nM and 10 nM, or between 3 nM and 5 nM. In some embodiments, the engineered polypeptide (e.g., IGF-1 variant) does not bind or binds very weakly to the insulin receptor. For instance, in some cases, the Kd of the interaction of the engineered polypeptide (e.g., IGF-1 variant) is >10-fold weaker than the binding of insulin to the insulin receptor.

In some embodiments, the IGF-1 variant comprises a truncation of the amino acid sequence of SEQ ID NO: 1. In some embodiments, the truncation is at the N-terminus of SEQ ID NO: 1. In some embodiments, the truncation is at the C-terminus of SEQ ID NO: 1. In some embodiments, the IGF-1 variant comprises a truncation that includes a deletion of at least about 1 amino acid. In some embodiments, the IGF-1 variant comprises a truncation that includes a deletion of at least about 2 amino acids. In some embodiments, the IGF-1 variant comprises a truncation that includes a deletion of at least about 3 amino acids. In some embodiments, the IGF-1 variant comprises a truncation that includes a deletion of at least about 4 amino acids. In some embodiments, the IGF-1 variant comprises a truncation that includes a deletion of at least about 5 amino acids. In some embodiments, the IGF-1 variant comprises a truncation that includes a deletion of at least about 6 amino acids. In some embodiments, the IGF-1 variant comprises a truncation that includes a deletion of at least about 7 amino acids. In some embodiments, the IGF-1 variant comprises a truncation that includes a deletion of at least about 8 amino acids. In some embodiments, the IGF-1 variant comprises a truncation that includes a deletion of at least about 9 amino acids. In some embodiments, the IGF-1 variant comprises a truncation that includes a deletion of at least about 10 amino acids. In some embodiments, the IGF-1 variant comprises a truncation that includes a deletion of at least about 11 amino acids. In some embodiments, the IGF-1 variant comprises a truncation that includes a deletion of at least about 12 amino acids.

In some embodiments, the IGF-1 variant is an extension of the amino acid sequence of SEQ ID NO: 1. In some embodiments, the extension is at the C-terminus of SEQ ID NO: 1. In some embodiments, the extension is at the N-terminus of SEQ ID NO: 1. In some embodiments, the extension has at least about 2 amino acids. In some embodiments, the extension has at least about 3 amino acids. In some embodiments, the extension has at least about 4 amino acids. In some embodiments, the extension has at least about 5 amino acids. In some embodiments, the extension has at least about 6 amino acids. In some embodiments, the extension has at least about 7 amino acids. In some embodiments, the extension has at least about 8 amino acids. In some embodiments, the extension has at least about 9 amino acids. In some embodiments, the extension has at least about 10 amino acids. In some embodiments, the extension has at least about 15 amino acids. In some embodiments, the extension has at least about 20 amino acids. In some embodiments, the extension has at least about 35 amino acids. In some embodiments, the extension has at least about 40 amino acids. In some embodiments, the extension has at least about 45 amino acids. In some embodiments, the extension has at least about 50 amino acids.

In some embodiments, the IGF-1 variant comprises one or more amino acid substitutions relative to wild-type IGF-1 (SEQ ID NO: 1). In some embodiments, the IGF-1 variant comprises an amino acid substitution at position 3 relative to wild-type IGF-1 (SEQ ID NO: 1). In some embodiments, the amino acid substitution at position 3 is an arginine. In some embodiments, the IGF-1 variant comprises an amino acid substitution at position 60 relative to wild-type IGF-1 (SEQ ID NO: 1). In some embodiments, the amino acid substitution at position 60 is a leucine. In some embodiments, the IGF-1 variant comprises an amino acid substitution at position 12 relative to wild-type IGF-1 (SEQ ID NO: 1). In some embodiments, the IGF-1 variant comprises an amino acid substitution at position 49 relative to wild-type IGF-1 (SEQ ID NO: 1). In some embodiments, the IGF-1 variant comprises an amino acid substitution at position 5 relative to wild-type IGF-1 (SEQ ID NO: 1). In some embodiments, the IGF-1 variant comprises an amino acid substitution at position 54 relative to wild-type IGF-1 (SEQ ID NO: 1). In some embodiments, the IGF-1 variant comprises an amino acid substitution at position 7 relative to wild-type IGF-1 (SEQ ID NO: 1). In some embodiments, the IGF-1 variant comprises an amino acid substitution at position 10 relative to wild-type IGF-1 (SEQ ID NO: 1). In some embodiments, the IGF-1 variant comprises an amino acid substitution at position 17 relative to wild-type IGF-1 (SEQ ID NO: 1). In some embodiments, the IGF-1 variant comprises an amino acid substitution at position 22 relative to wild-type IGF-1 (SEQ ID NO: 1). In some embodiments, the IGF-1 variant comprises an amino acid substitution at position 23 relative to wild-type IGF-1 (SEQ ID NO: 1). In some embodiments, the IGF-1 variant comprises an amino acid substitution at position 24 relative to wild-type IGF-1 (SEQ ID NO: 1). In some embodiments, the IGF-1 variant comprises an amino acid substitution at position 25 relative to wild-type IGF-1 (SEQ ID NO: 1). In some embodiments, the IGF-1 variant comprises an amino acid substitution at position 34 relative to wild-type IGF-1 (SEQ ID NO: 1). In some embodiments, the IGF-1 variant comprises an amino acid substitution at position 57 relative to wild-type IGF-1 (SEQ ID NO: 1). Position numbering can be based on alignment of the IGF-1 variant to SEQ ID NO: 1, with positions numbered from an N-terminus of SEQ ID NO: 1 to a C-terminus of SEQ ID NO: 1 starting with position 1 at the N-terminus of SEQ ID NO: 1. In some embodiments, the engineered polypeptide comprises IGF-1 Y60L. In some embodiments, the engineered polypeptide comprises an E3R mutation. In some embodiments, the engineered polypeptide comprises a D12A mutation. In some embodiments, the engineered polypeptide comprises a D12C mutation. In some embodiments, the engineered polypeptide comprises a D12E mutation. In some embodiments, the engineered polypeptide comprises a D12F mutation. In some embodiments, the engineered polypeptide comprises a D12G mutation. In some embodiments, the engineered polypeptide comprises a D12H mutation. In some embodiments, the engineered polypeptide comprises a D12I mutation. In some embodiments, the engineered polypeptide comprises a D12K mutation. In some embodiments, the engineered polypeptide comprises a D12L mutation. In some embodiments, the engineered polypeptide comprises a D12M mutation. In some embodiments, the engineered polypeptide comprises a D12N mutation. In some embodiments, the engineered polypeptide comprises a D12P mutation. In some embodiments, the engineered polypeptide comprises a D12Q mutation. In some embodiments, the engineered polypeptide comprises a D12R mutation. In some embodiments, the engineered polypeptide comprises a D12S mutation. In some embodiments, the engineered polypeptide comprises a D12T mutation. In some embodiments, the engineered polypeptide comprises a D12V mutation. In some embodiments, the engineered polypeptide comprises a D12W mutation. In some embodiments, the engineered polypeptide comprises a D12Y mutation. In some embodiments, the engineered polypeptide comprises a F49A mutation. In some embodiments, the engineered polypeptide comprises a F49C mutation. In some embodiments, the engineered polypeptide comprises an F49D mutation. In some embodiments, the engineered polypeptide comprises an F49E mutation. In some embodiments, the engineered polypeptide comprises an F49G mutation. In some embodiments, the engineered polypeptide comprises an F49H mutation. In some embodiments, the engineered polypeptide comprises an F49I mutation. In some embodiments, the engineered polypeptide comprises an F49K mutation. In some embodiments, the engineered polypeptide comprises an F49L mutation. In some embodiments, the engineered polypeptide comprises an F49M mutation. In some embodiments, the engineered polypeptide comprises an F49N mutation. In some embodiments, the engineered polypeptide comprises an F49P mutation. In some embodiments, the engineered polypeptide comprises an F49Q mutation. In some embodiments, the engineered polypeptide comprises an F49R mutation. In some embodiments, the engineered polypeptide comprises an F49S mutation. In some embodiments, the engineered polypeptide comprises an F49T mutation. In some embodiments, the engineered polypeptide comprises an F49V mutation. In some embodiments, the engineered polypeptide comprises an F49W mutation. In some embodiments, the engineered polypeptide comprises an F49Y mutation. In some embodiments, the engineered polypeptide comprises mutation at position 49 selected from the group consisting of A, C, D, E, G, H, I, K, L, M, N, P, Q, R, S, and T. In some embodiments, the engineered polypeptide comprises an L54A mutation. In some embodiments, the engineered polypeptide comprises an L54C mutation. In some embodiments, the engineered polypeptide comprises an L54D mutation. In some embodiments, the engineered polypeptide comprises an L54E mutation. In some embodiments, the engineered polypeptide comprises an L54F mutation. In some embodiments, the engineered polypeptide comprises an L54G mutation. In some embodiments, the engineered polypeptide comprises an L54H mutation. In some embodiments, the engineered polypeptide comprises an L54I mutation. In some embodiments, the engineered polypeptide comprises an L54K mutation. In some embodiments, the engineered polypeptide comprises an L54M mutation. In some embodiments, the engineered polypeptide comprises an L54N mutation. In some embodiments, the engineered polypeptide comprises an L54P mutation. In some embodiments, the engineered polypeptide comprises an L54Q mutation. In some embodiments, the engineered polypeptide comprises an L54R mutation. In some embodiments, the engineered polypeptide comprises an L54S mutation. In some embodiments, the engineered polypeptide comprises an L54T mutation. In some embodiments, the engineered polypeptide comprises an L54V mutation. In some embodiments, the engineered polypeptide comprises an L54W mutation. In some embodiments, the engineered polypeptide comprises an L54Y mutation. In some embodiments, the engineered polypeptide comprises L5A. In some embodiments, the engineered polypeptide comprises an L5A mutation. In some embodiments, the engineered polypeptide comprises an L5C mutation. In some embodiments, the engineered polypeptide comprises an L5D mutation. In some embodiments, the engineered polypeptide comprises an L5E mutation. In some embodiments, the engineered polypeptide comprises an L5F mutation. In some embodiments, the engineered polypeptide comprises an L5G mutation. In some embodiments, the engineered polypeptide comprises an L5H mutation. In some embodiments, the engineered polypeptide comprises an L5I mutation. In some embodiments, the engineered polypeptide comprises an L5K mutation. In some embodiments, the engineered polypeptide comprises an L5M mutation. In some embodiments, the engineered polypeptide comprises an L5N mutation. In some embodiments, the engineered polypeptide comprises an L5P mutation. In some embodiments, the engineered polypeptide comprises an L5Q mutation. In some embodiments, the engineered polypeptide comprises an L5R mutation. In some embodiments, the engineered polypeptide comprises an L5S mutation. In some embodiments, the engineered polypeptide comprises an L5T mutation. In some embodiments, the engineered polypeptide comprises an L5V mutation. In some embodiments, the engineered polypeptide comprises an L5W mutation. In some embodiments, the engineered polypeptide comprises an L5Y mutation. In some embodiments, the engineered polypeptide comprises a G7A mutation. In some embodiments, the engineered polypeptide comprises a G7C mutation. In some embodiments, the engineered polypeptide comprises a G7D mutation. In some embodiments, the engineered polypeptide comprises a G7E mutation. In some embodiments, the engineered polypeptide comprises a G7F mutation. In some embodiments, the engineered polypeptide comprises a G7H mutation. In some embodiments, the engineered polypeptide comprises a G7I mutation. In some embodiments, the engineered polypeptide comprises a G7K mutation. In some embodiments, the engineered polypeptide comprises a G7L mutation. In some embodiments, the engineered polypeptide comprises a G7M mutation. In some embodiments, the engineered polypeptide comprises a G7N mutation. In some embodiments, the engineered polypeptide comprises a G7P mutation. In some embodiments, the engineered polypeptide comprises a G7Q mutation. In some embodiments, the engineered polypeptide comprises a G7R mutation. In some embodiments, the engineered polypeptide comprises a G7S mutation. In some embodiments, the engineered polypeptide comprises a G7T mutation. In some embodiments, the engineered polypeptide comprises a G7V mutation. In some embodiments, the engineered polypeptide comprises a G7W mutation. In some embodiments, the engineered polypeptide comprises a G7Y mutation. In some embodiments, the engineered polypeptide comprises an L10A mutation. In some embodiments, the engineered polypeptide comprises an L10C mutation. In some embodiments, the engineered polypeptide comprises an L10D mutation. In some embodiments, the engineered polypeptide comprises an L10E mutation. In some embodiments, the engineered polypeptide comprises an L10F mutation. In some embodiments, the engineered polypeptide comprises an L10G mutation. In some embodiments, the engineered polypeptide comprises an L10H mutation. In some embodiments, the engineered polypeptide comprises an L10I mutation. In some embodiments, the engineered polypeptide comprises an L10K mutation. In some embodiments, the engineered polypeptide comprises an L10M mutation. In some embodiments, the engineered polypeptide comprises an L10N mutation. In some embodiments, the engineered polypeptide comprises an L10P mutation. In some embodiments, the engineered polypeptide comprises an L10Q mutation. In some embodiments, the engineered polypeptide comprises an L10R mutation. In some embodiments, the engineered polypeptide comprises an L10S mutation. In some embodiments, the engineered polypeptide comprises an L10T mutation. In some embodiments, the engineered polypeptide comprises an L10V mutation. In some embodiments, the engineered polypeptide comprises an L10W mutation. In some embodiments, the engineered polypeptide comprises an L10Y mutation. In some embodiments, the engineered polypeptide comprises a V17A mutation. In some embodiments, the engineered polypeptide comprises a V17C mutation. In some embodiments, the engineered polypeptide comprises a V17D mutation. In some embodiments, the engineered polypeptide comprises a V17E mutation. In some embodiments, the engineered polypeptide comprises a V17F mutation. In some embodiments, the engineered polypeptide comprises a V17G mutation. In some embodiments, the engineered polypeptide comprises a V17H mutation. In some embodiments, the engineered polypeptide comprises a V17I mutation. In some embodiments, the engineered polypeptide comprises a V17K mutation. In some embodiments, the engineered polypeptide comprises a V17L mutation. In some embodiments, the engineered polypeptide comprises a V17M mutation. In some embodiments, the engineered polypeptide comprises a V17N mutation. In some embodiments, the engineered polypeptide comprises a V17P mutation. In some embodiments, the engineered polypeptide comprises a V17Q mutation. In some embodiments, the engineered polypeptide comprises a V17R mutation. In some embodiments, the engineered polypeptide comprises a V17S mutation. In some embodiments, the engineered polypeptide comprises a V17T mutation. In some embodiments, the engineered polypeptide comprises a V17W mutation. In some embodiments, the engineered polypeptide comprises a V17Y mutation. In some embodiments, the engineered polypeptide comprises a G22A mutation. In some embodiments, the engineered polypeptide comprises a G22C mutation. In some embodiments, the engineered polypeptide comprises a G22D mutation. In some embodiments, the engineered polypeptide comprises a G22E mutation. In some embodiments, the engineered polypeptide comprises a G22F mutation. In some embodiments, the engineered polypeptide comprises a G22H mutation. In some embodiments, the engineered polypeptide comprises a G22I mutation. In some embodiments, the engineered polypeptide comprises a G22K mutation. In some embodiments, the engineered polypeptide comprises a G22L mutation. In some embodiments, the engineered polypeptide comprises a G22M mutation. In some embodiments, the engineered polypeptide comprises a G22N mutation. In some embodiments, the engineered polypeptide comprises a G22P mutation. In some embodiments, the engineered polypeptide comprises a G22Q mutation. In some embodiments, the engineered polypeptide comprises a G22R mutation. In some embodiments, the engineered polypeptide comprises a G22S mutation. In some embodiments, the engineered polypeptide comprises a G22T mutation. In some embodiments, the engineered polypeptide comprises a G22V mutation. In some embodiments, the engineered polypeptide comprises a G22W mutation. In some embodiments, the engineered polypeptide comprises a G22Y mutation. In some embodiments, the engineered polypeptide comprises an F23A mutation. In some embodiments, the engineered polypeptide comprises an F23C mutation. In some embodiments, the engineered polypeptide comprises an F23D mutation. In some embodiments, the engineered polypeptide comprises an F23E mutation. In some embodiments, the engineered polypeptide comprises an F23G mutation. In some embodiments, the engineered polypeptide comprises an F23H mutation. In some embodiments, the engineered polypeptide comprises an F23I mutation. In some embodiments, the engineered polypeptide comprises an F23K mutation. In some embodiments, the engineered polypeptide comprises an F23L mutation. In some embodiments, the engineered polypeptide comprises an F23M mutation. In some embodiments, the engineered polypeptide comprises an F23N mutation. In some embodiments, the engineered polypeptide comprises an F23P mutation. In some embodiments, the engineered polypeptide comprises an F23Q mutation. In some embodiments, the engineered polypeptide comprises an F23R mutation. In some embodiments, the engineered polypeptide comprises an F23S mutation. In some embodiments, the engineered polypeptide comprises an F23T mutation. In some embodiments, the engineered polypeptide comprises an F23V mutation. In some embodiments, the engineered polypeptide comprises an F23W mutation. In some embodiments, the engineered polypeptide comprises an F23Y mutation. In some embodiments, the engineered polypeptide comprises a Y24A mutation. In some embodiments, the engineered polypeptide comprises a Y24C mutation. In some embodiments, the engineered polypeptide comprises a Y24D mutation. In some embodiments, the engineered polypeptide comprises a Y24E mutation. In some embodiments, the engineered polypeptide comprises a Y24F mutation. In some embodiments, the engineered polypeptide comprises a Y24G mutation. In some embodiments, the engineered polypeptide comprises a Y24H mutation. In some embodiments, the engineered polypeptide comprises a Y24I mutation. In some embodiments, the engineered polypeptide comprises a Y24K mutation. In some embodiments, the engineered polypeptide comprises a Y24L mutation. In some embodiments, the engineered polypeptide comprises a Y24M mutation. In some embodiments, the engineered polypeptide comprises a Y24N mutation. In some embodiments, the engineered polypeptide comprises a Y24P mutation. In some embodiments, the engineered polypeptide comprises a Y24Q mutation. In some embodiments, the engineered polypeptide comprises a Y24R mutation. In some embodiments, the engineered polypeptide comprises a Y24S mutation. In some embodiments, the engineered polypeptide comprises a Y24T mutation. In some embodiments, the engineered polypeptide comprises a Y24V mutation. In some embodiments, the engineered polypeptide comprises a Y24W mutation. In some embodiments, the engineered polypeptide comprises an F25A mutation. In some embodiments, the engineered polypeptide comprises an F25C mutation. In some embodiments, the engineered polypeptide comprises an F25D mutation. In some embodiments, the engineered polypeptide comprises an F25E mutation. In some embodiments, the engineered polypeptide comprises an F25G mutation. In some embodiments, the engineered polypeptide comprises an F25H mutation. In some embodiments, the engineered polypeptide comprises an F25I mutation. In some embodiments, the engineered polypeptide comprises an F25K mutation. In some embodiments, the engineered polypeptide comprises an F25L mutation. In some embodiments, the engineered polypeptide comprises an F25M mutation. In some embodiments, the engineered polypeptide comprises an F25N mutation. In some embodiments, the engineered polypeptide comprises an F25P mutation. In some embodiments, the engineered polypeptide comprises an F25Q mutation. In some embodiments, the engineered polypeptide comprises an F25R mutation. In some embodiments, the engineered polypeptide comprises an F25S mutation. In some embodiments, the engineered polypeptide comprises an F25T mutation. In some embodiments, the engineered polypeptide comprises an F25V mutation. In some embodiments, the engineered polypeptide comprises an F25W mutation. In some embodiments, the engineered polypeptide comprises an F25Y mutation. In some embodiments, the engineered polypeptide comprises an S34A mutation. In some embodiments, the engineered polypeptide comprises an S34C mutation. In some embodiments, the engineered polypeptide comprises an S34D mutation. In some embodiments, the engineered polypeptide comprises an S34E mutation. In some embodiments, the engineered polypeptide comprises an S34F mutation. In some embodiments, the engineered polypeptide comprises an S34G mutation. In some embodiments, the engineered polypeptide comprises an S34H mutation. In some embodiments, the engineered polypeptide comprises an S34I mutation. In some embodiments, the engineered polypeptide comprises an S34K mutation. In some embodiments, the engineered polypeptide comprises an S34L mutation. In some embodiments, the engineered polypeptide comprises an S34M mutation. In some embodiments, the engineered polypeptide comprises an S34N mutation. In some embodiments, the engineered polypeptide comprises an S34P mutation. In some embodiments, the engineered polypeptide comprises an S34Q mutation. In some embodiments, the engineered polypeptide comprises an S34R mutation. In some embodiments, the engineered polypeptide comprises an S34T mutation. In some embodiments, the engineered polypeptide comprises an S34V mutation. In some embodiments, the engineered polypeptide comprises an S34W mutation. In some embodiments, the engineered polypeptide comprises an S34Y mutation. In some embodiments, the engineered polypeptide comprises an L57A mutation. In some embodiments, the engineered polypeptide comprises an L57C mutation. In some embodiments, the engineered polypeptide comprises an L57D mutation. In some embodiments, the engineered polypeptide comprises an L57E mutation. In some embodiments, the engineered polypeptide comprises an L57F mutation. In some embodiments, the engineered polypeptide comprises an L57G mutation. In some embodiments, the engineered polypeptide comprises an L57H mutation. In some embodiments, the engineered polypeptide comprises an L57I mutation. In some embodiments, the engineered polypeptide comprises an L57K mutation. In some embodiments, the engineered polypeptide comprises an L57M mutation. In some embodiments, the engineered polypeptide comprises an L57N mutation. In some embodiments, the engineered polypeptide comprises an L57P mutation. In some embodiments, the engineered polypeptide comprises an L57Q mutation. In some embodiments, the engineered polypeptide comprises an L57R mutation. In some embodiments, the engineered polypeptide comprises an L57S mutation. In some embodiments, the engineered polypeptide comprises an L57T mutation. In some embodiments, the engineered polypeptide comprises an L57V mutation. In some embodiments, the engineered polypeptide comprises an L57W mutation. In some embodiments, the engineered polypeptide comprises an L57Y mutation. In some embodiments, the engineered polypeptide comprises a native IGF-1 with a deletion at R37.

In some embodiments, the IGF-1 variant comprises a truncation of the amino acid sequence of SEQ ID NO: 1 and one or more amino acid substitutions relative to wild-type IGF-1 (SEQ ID NO: 1). In some embodiments, the IGF-1 variant comprises a truncation of 3 amino acids at the N-terminus of SEQ ID NO: 1 and an amino acid deletion at position 37 relative to wild-type IGF-1 (SEQ ID NO: 1). In some embodiments, the IGF-1 variant comprises a deletion relative to SEQ ID NO: 1. In some embodiments, the IGF-1 variant comprises an amino acid deletion at position 37 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 12 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 49 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 54 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 5 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 7 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 10 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 17 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 22 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 23 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 24 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 25 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 34 relative to wild-type IGF-1 (SEQ ID NO: 1). In some embodiments, the IGF-1 variant comprises a truncation of 3 amino acids at the N-terminus of SEQ ID NO: 1 and an amino acid substitution at position 57 relative to wild-type IGF-1 (SEQ ID NO: 1). In some embodiments, the engineered polypeptide comprises a D12A mutation. In some embodiments, the engineered polypeptide comprises a D12C mutation. In some embodiments, the engineered polypeptide comprises a D12E mutation. In some embodiments, the engineered polypeptide comprises a D12F mutation. In some embodiments, the engineered polypeptide comprises a D12G mutation. In some embodiments, the engineered polypeptide comprises a D12H mutation. In some embodiments, the engineered polypeptide comprises a D12I mutation. In some embodiments, the engineered polypeptide comprises a D12K mutation. In some embodiments, the engineered polypeptide comprises a D12L mutation. In some embodiments, the engineered polypeptide comprises a D12M mutation. In some embodiments, the engineered polypeptide comprises a D12N mutation. In some embodiments, the engineered polypeptide comprises a D12P mutation. In some embodiments, the engineered polypeptide comprises a D12Q mutation. In some embodiments, the engineered polypeptide comprises a D12R mutation. In some embodiments, the engineered polypeptide comprises a D12S mutation. In some embodiments, the engineered polypeptide comprises a D12T mutation. In some embodiments, the engineered polypeptide comprises a D12V mutation. In some embodiments, the engineered polypeptide comprises a D12W mutation. In some embodiments, the engineered polypeptide comprises a D12Y mutation. In some embodiments, the engineered polypeptide comprises a F49A mutation. In some embodiments, the engineered polypeptide comprises a F49C mutation. In some embodiments, the engineered polypeptide comprises an F49D mutation. In some embodiments, the engineered polypeptide comprises an F49E mutation. In some embodiments, the engineered polypeptide comprises an F49G mutation. In some embodiments, the engineered polypeptide comprises an F49H mutation. In some embodiments, the engineered polypeptide comprises an F49I mutation. In some embodiments, the engineered polypeptide comprises an F49K mutation. In some embodiments, the engineered polypeptide comprises an F49L mutation. In some embodiments, the engineered polypeptide comprises an F49M mutation. In some embodiments, the engineered polypeptide comprises an F49N mutation. In some embodiments, the engineered polypeptide comprises an F49P mutation. In some embodiments, the engineered polypeptide comprises an F49Q mutation. In some embodiments, the engineered polypeptide comprises an F49R mutation. In some embodiments, the engineered polypeptide comprises an F49S mutation. In some embodiments, the engineered polypeptide comprises an F49T mutation. In some embodiments, the engineered polypeptide comprises an F49V mutation. In some embodiments, the engineered polypeptide comprises an F49W mutation. In some embodiments, the engineered polypeptide comprises an F49Y mutation. In some embodiments, the engineered polypeptide comprises mutation at position 49 selected from the group consisting of A, C, D, E, G, H, I, K, L, M, N, P, Q, R, S, and T. In some embodiments, the engineered polypeptide comprises an L54A mutation. In some embodiments, the engineered polypeptide comprises an L54C mutation. In some embodiments, the engineered polypeptide comprises an L54D mutation. In some embodiments, the engineered polypeptide comprises an L54E mutation. In some embodiments, the engineered polypeptide comprises an L54F mutation. In some embodiments, the engineered polypeptide comprises an L54G mutation. In some embodiments, the engineered polypeptide comprises an L54H mutation. In some embodiments, the engineered polypeptide comprises an L54I mutation. In some embodiments, the engineered polypeptide comprises an L54K mutation. In some embodiments, the engineered polypeptide comprises an L54M mutation. In some embodiments, the engineered polypeptide comprises an L54N mutation. In some embodiments, the engineered polypeptide comprises an L54P mutation. In some embodiments, the engineered polypeptide comprises an L54Q mutation. In some embodiments, the engineered polypeptide comprises an L54R mutation. In some embodiments, the engineered polypeptide comprises an L54S mutation. In some embodiments, the engineered polypeptide comprises an L54T mutation. In some embodiments, the engineered polypeptide comprises an L54V mutation. In some embodiments, the engineered polypeptide comprises an L54W mutation. In some embodiments, the engineered polypeptide comprises an L54Y mutation. In some embodiments, the engineered polypeptide comprises L5A. In some embodiments, the engineered polypeptide comprises an L5A mutation. In some embodiments, the engineered polypeptide comprises an L5C mutation. In some embodiments, the engineered polypeptide comprises an L5D mutation. In some embodiments, the engineered polypeptide comprises an L5E mutation. In some embodiments, the engineered polypeptide comprises an L5F mutation. In some embodiments, the engineered polypeptide comprises an L5G mutation. In some embodiments, the engineered polypeptide comprises an L5H mutation. In some embodiments, the engineered polypeptide comprises an L5I mutation. In some embodiments, the engineered polypeptide comprises an L5K mutation. In some embodiments, the engineered polypeptide comprises an L5M mutation. In some embodiments, the engineered polypeptide comprises an L5N mutation. In some embodiments, the engineered polypeptide comprises an L5P mutation. In some embodiments, the engineered polypeptide comprises an L5Q mutation. In some embodiments, the engineered polypeptide comprises an L5R mutation. In some embodiments, the engineered polypeptide comprises an L5S mutation. In some embodiments, the engineered polypeptide comprises an L5T mutation. In some embodiments, the engineered polypeptide comprises an L5V mutation. In some embodiments, the engineered polypeptide comprises an L5W mutation. In some embodiments, the engineered polypeptide comprises an L5Y mutation. In some embodiments, the engineered polypeptide comprises a G7A mutation. In some embodiments, the engineered polypeptide comprises a G7C mutation. In some embodiments, the engineered polypeptide comprises a G7D mutation. In some embodiments, the engineered polypeptide comprises a G7E mutation. In some embodiments, the engineered polypeptide comprises a G7F mutation. In some embodiments, the engineered polypeptide comprises a G7H mutation. In some embodiments, the engineered polypeptide comprises a G7I mutation. In some embodiments, the engineered polypeptide comprises a G7K mutation. In some embodiments, the engineered polypeptide comprises a G7L mutation. In some embodiments, the engineered polypeptide comprises a G7M mutation. In some embodiments, the engineered polypeptide comprises a G7N mutation. In some embodiments, the engineered polypeptide comprises a G7P mutation. In some embodiments, the engineered polypeptide comprises a G7Q mutation. In some embodiments, the engineered polypeptide comprises a G7R mutation. In some embodiments, the engineered polypeptide comprises a G7S mutation. In some embodiments, the engineered polypeptide comprises a G7T mutation. In some embodiments, the engineered polypeptide comprises a G7V mutation. In some embodiments, the engineered polypeptide comprises a G7W mutation. In some embodiments, the engineered polypeptide comprises a G7Y mutation. In some embodiments, the engineered polypeptide comprises an L10A mutation. In some embodiments, the engineered polypeptide comprises an L10C mutation. In some embodiments, the engineered polypeptide comprises an L10D mutation. In some embodiments, the engineered polypeptide comprises an L10E mutation. In some embodiments, the engineered polypeptide comprises an L10F mutation. In some embodiments, the engineered polypeptide comprises an L10G mutation. In some embodiments, the engineered polypeptide comprises an L10H mutation. In some embodiments, the engineered polypeptide comprises an L10I mutation. In some embodiments, the engineered polypeptide comprises an L10K mutation. In some embodiments, the engineered polypeptide comprises an L10M mutation. In some embodiments, the engineered polypeptide comprises an L10N mutation. In some embodiments, the engineered polypeptide comprises an L10P mutation. In some embodiments, the engineered polypeptide comprises an L10Q mutation. In some embodiments, the engineered polypeptide comprises an L10R mutation. In some embodiments, the engineered polypeptide comprises an L10S mutation. In some embodiments, the engineered polypeptide comprises an L10T mutation. In some embodiments, the engineered polypeptide comprises an L10V mutation. In some embodiments, the engineered polypeptide comprises an L10W mutation. In some embodiments, the engineered polypeptide comprises an L10Y mutation. In some embodiments, the engineered polypeptide comprises a V17A mutation. In some embodiments, the engineered polypeptide comprises a V17C mutation. In some embodiments, the engineered polypeptide comprises a V17D mutation. In some embodiments, the engineered polypeptide comprises a V17E mutation. In some embodiments, the engineered polypeptide comprises a V17F mutation. In some embodiments, the engineered polypeptide comprises a V17G mutation. In some embodiments, the engineered polypeptide comprises a V17H mutation. In some embodiments, the engineered polypeptide comprises a V17I mutation. In some embodiments, the engineered polypeptide comprises a V17K mutation. In some embodiments, the engineered polypeptide comprises a V17L mutation. In some embodiments, the engineered polypeptide comprises a V17M mutation. In some embodiments, the engineered polypeptide comprises a V17N mutation. In some embodiments, the engineered polypeptide comprises a V17P mutation. In some embodiments, the engineered polypeptide comprises a V17Q mutation. In some embodiments, the engineered polypeptide comprises a V17R mutation. In some embodiments, the engineered polypeptide comprises a V17S mutation. In some embodiments, the engineered polypeptide comprises a V17T mutation. In some embodiments, the engineered polypeptide comprises a V17W mutation. In some embodiments, the engineered polypeptide comprises a V17Y mutation. In some embodiments, the engineered polypeptide comprises a G22A mutation. In some embodiments, the engineered polypeptide comprises a G22C mutation. In some embodiments, the engineered polypeptide comprises a G22D mutation. In some embodiments, the engineered polypeptide comprises a G22E mutation. In some embodiments, the engineered polypeptide comprises a G22F mutation. In some embodiments, the engineered polypeptide comprises a G22H mutation. In some embodiments, the engineered polypeptide comprises a G22I mutation. In some embodiments, the engineered polypeptide comprises a G22K mutation. In some embodiments, the engineered polypeptide comprises a G22L mutation. In some embodiments, the engineered polypeptide comprises a G22M mutation. In some embodiments, the engineered polypeptide comprises a G22N mutation. In some embodiments, the engineered polypeptide comprises a G22P mutation. In some embodiments, the engineered polypeptide comprises a G22Q mutation. In some embodiments, the engineered polypeptide comprises a G22R mutation. In some embodiments, the engineered polypeptide comprises a G22S mutation. In some embodiments, the engineered polypeptide comprises a G22T mutation. In some embodiments, the engineered polypeptide comprises a G22V mutation. In some embodiments, the engineered polypeptide comprises a G22W mutation. In some embodiments, the engineered polypeptide comprises a G22Y mutation. In some embodiments, the engineered polypeptide comprises an F23A mutation. In some embodiments, the engineered polypeptide comprises an F23C mutation. In some embodiments, the engineered polypeptide comprises an F23D mutation. In some embodiments, the engineered polypeptide comprises an F23E mutation. In some embodiments, the engineered polypeptide comprises an F23G mutation. In some embodiments, the engineered polypeptide comprises an F23H mutation. In some embodiments, the engineered polypeptide comprises an F23I mutation. In some embodiments, the engineered polypeptide comprises an F23K mutation. In some embodiments, the engineered polypeptide comprises an F23L mutation. In some embodiments, the engineered polypeptide comprises an F23M mutation. In some embodiments, the engineered polypeptide comprises an F23N mutation. In some embodiments, the engineered polypeptide comprises an F23P mutation. In some embodiments, the engineered polypeptide comprises an F23Q mutation. In some embodiments, the engineered polypeptide comprises an F23R mutation. In some embodiments, the engineered polypeptide comprises an F23S mutation. In some embodiments, the engineered polypeptide comprises an F23T mutation. In some embodiments, the engineered polypeptide comprises an F23V mutation. In some embodiments, the engineered polypeptide comprises an F23W mutation. In some embodiments, the engineered polypeptide comprises an F23Y mutation. In some embodiments, the engineered polypeptide comprises a Y24A mutation. In some embodiments, the engineered polypeptide comprises a Y24C mutation. In some embodiments, the engineered polypeptide comprises a Y24D mutation. In some embodiments, the engineered polypeptide comprises a Y24E mutation. In some embodiments, the engineered polypeptide comprises a Y24F mutation. In some embodiments, the engineered polypeptide comprises a Y24G mutation. In some embodiments, the engineered polypeptide comprises a Y24H mutation. In some embodiments, the engineered polypeptide comprises a Y24I mutation. In some embodiments, the engineered polypeptide comprises a Y24K mutation. In some embodiments, the engineered polypeptide comprises a Y24L mutation. In some embodiments, the engineered polypeptide comprises a Y24M mutation. In some embodiments, the engineered polypeptide comprises a Y24N mutation. In some embodiments, the engineered polypeptide comprises a Y24P mutation. In some embodiments, the engineered polypeptide comprises a Y24Q mutation. In some embodiments, the engineered polypeptide comprises a Y24R mutation. In some embodiments, the engineered polypeptide comprises a Y24S mutation. In some embodiments, the engineered polypeptide comprises a Y24T mutation. In some embodiments, the engineered polypeptide comprises a Y24V mutation. In some embodiments, the engineered polypeptide comprises a Y24W mutation. In some embodiments, the engineered polypeptide comprises an F25A mutation. In some embodiments, the engineered polypeptide comprises an F25C mutation. In some embodiments, the engineered polypeptide comprises an F25D mutation. In some embodiments, the engineered polypeptide comprises an F25E mutation. In some embodiments, the engineered polypeptide comprises an F25G mutation. In some embodiments, the engineered polypeptide comprises an F25H mutation. In some embodiments, the engineered polypeptide comprises an F25I mutation. In some embodiments, the engineered polypeptide comprises an F25K mutation. In some embodiments, the engineered polypeptide comprises an F25L mutation. In some embodiments, the engineered polypeptide comprises an F25M mutation. In some embodiments, the engineered polypeptide comprises an F25N mutation. In some embodiments, the engineered polypeptide comprises an F25P mutation. In some embodiments, the engineered polypeptide comprises an F25Q mutation. In some embodiments, the engineered polypeptide comprises an F25R mutation. In some embodiments, the engineered polypeptide comprises an F25S mutation. In some embodiments, the engineered polypeptide comprises an F25T mutation. In some embodiments, the engineered polypeptide comprises an F25V mutation. In some embodiments, the engineered polypeptide comprises an F25W mutation. In some embodiments, the engineered polypeptide comprises an F25Y mutation. In some embodiments, the engineered polypeptide comprises an S34A mutation. In some embodiments, the engineered polypeptide comprises an S34C mutation. In some embodiments, the engineered polypeptide comprises an S34D mutation. In some embodiments, the engineered polypeptide comprises an S34E mutation. In some embodiments, the engineered polypeptide comprises an S34F mutation. In some embodiments, the engineered polypeptide comprises an S34G mutation. In some embodiments, the engineered polypeptide comprises an S34H mutation. In some embodiments, the engineered polypeptide comprises an S34I mutation. In some embodiments, the engineered polypeptide comprises an S34K mutation. In some embodiments, the engineered polypeptide comprises an S34L mutation. In some embodiments, the engineered polypeptide comprises an S34M mutation. In some embodiments, the engineered polypeptide comprises an S34N mutation. In some embodiments, the engineered polypeptide comprises an S34P mutation. In some embodiments, the engineered polypeptide comprises an S34Q mutation. In some embodiments, the engineered polypeptide comprises an S34R mutation. In some embodiments, the engineered polypeptide comprises an S34T mutation. In some embodiments, the engineered polypeptide comprises an S34V mutation. In some embodiments, the engineered polypeptide comprises an S34W mutation. In some embodiments, the engineered polypeptide comprises an S34Y mutation. In some embodiments, the engineered polypeptide comprises an L57A mutation. In some embodiments, the engineered polypeptide comprises an L57C mutation. In some embodiments, the engineered polypeptide comprises an L57D mutation. In some embodiments, the engineered polypeptide comprises an L57E mutation. In some embodiments, the engineered polypeptide comprises an L57F mutation. In some embodiments, the engineered polypeptide comprises an L57G mutation. In some embodiments, the engineered polypeptide comprises an L57H mutation. In some embodiments, the engineered polypeptide comprises an L57I mutation. In some embodiments, the engineered polypeptide comprises an L57K mutation. In some embodiments, the engineered polypeptide comprises an L57M mutation. In some embodiments, the engineered polypeptide comprises an L57N mutation. In some embodiments, the engineered polypeptide comprises an L57P mutation. In some embodiments, the engineered polypeptide comprises an L57Q mutation. In some embodiments, the engineered polypeptide comprises an L57R mutation. In some embodiments, the engineered polypeptide comprises an L57S mutation. In some embodiments, the engineered polypeptide comprises an L57T mutation. In some embodiments, the engineered polypeptide comprises an L57V mutation. In some embodiments, the engineered polypeptide comprises an L57W mutation. In some embodiments, the engineered polypeptide comprises an L57Y mutation.

In some embodiments, the IGF-1 variant comprises the amino sequence of SEQ ID NO: 2. In some embodiments, the IGF-1 variant comprises the amino sequence of SEQ ID NO: 3. In some embodiments, the IGF-1 variant comprises the amino sequence of SEQ ID NO: 4. In some embodiments, the IGF-1 variant comprises the amino sequence of SEQ ID NO: 5. In some embodiments, the IGF-1 variant comprises the amino sequence of SEQ ID NO: 6. In some embodiments, the IGF-1 variant comprises the amino sequence of SEQ ID NO: 7. In some embodiments, the IGF-1 variant comprises the amino sequence of SEQ ID NO: 8. In some embodiments, the IGF-1 variant comprises the amino sequence of SEQ ID NO: 12. In some embodiments, the IGF-1 variant comprises the amino acid sequence of SEQ ID NO: 13. In some embodiments, the IGF-1 variant comprises the amino acid sequence of SEQ ID NO: 14. In some embodiments, the IGF-1 variant comprises the amino acid sequence of SEQ ID NO: 15. In some embodiments, the IGF-1 variant comprises the amino acid sequence of SEQ ID NO: 16. In some embodiments, the IGF-1 variant comprises the amino acid sequence of SEQ ID NO: 17. In some embodiments, the IGF-1 variant comprises the amino acid sequence of SEQ ID NO: 18. In some embodiments, the IGF-1 variant comprises the amino acid sequence of SEQ ID NO: 19. In some embodiments, the IGF-1 variant comprises the amino acid sequence of SEQ ID NO: 20. In some embodiments, the IGF-1 variant comprises the amino acid sequence of SEQ ID NO: 21. In some embodiments, the IGF-1 variant comprises the amino acid sequence of SEQ ID NO: 22. In some embodiments, the IGF-1 variant comprises the amino acid sequence of SEQ ID NO: 23. In some embodiments, the IGF-1 variant comprises the amino acid sequence of SEQ ID NO: 24. In some embodiments, the IGF-1 variant comprises the amino acid sequence of SEQ ID NO: 25. In some embodiments, the IGF-1 variant comprises the amino acid sequence of SEQ ID NO: 26. In some embodiments, the IGF-1 variant comprises the amino acid sequence of SEQ ID NO: 27. In some embodiments, the IGF-1 variant comprises the amino acid sequence of SEQ ID NO: 28. In some embodiments, the IGF-1 variant comprises the amino acid sequence of SEQ ID NO: 29. In some embodiments, the IGF-1 variant comprises the amino acid sequence of SEQ ID NO: 30. In some embodiments, the IGF-1 variant comprises the amino acid sequence of SEQ ID NO: 31. In some embodiments, the IGF-1 variant comprises the amino acid sequence of SEQ ID NO: 32. In some embodiments, the IGF-1 variant comprises the amino acid sequence of SEQ ID NO: 33. In some embodiments, the IGF-1 variant comprises the amino acid sequence of SEQ ID NO: 34. In some embodiments, the IGF-1 variant comprises the amino acid sequence of SEQ ID NO: 35. In some embodiments, the IGF-1 variant comprises the amino acid sequence of SEQ ID NO: 36. In some embodiments, the IGF-1 variant comprises the amino acid sequence of SEQ ID NO: 37. In some embodiments, the IGF-1 variant comprises the amino acid sequence of SEQ ID NO: 38. In some embodiments, the IGF-1 variant comprises the amino acid sequence of SEQ ID NO: 39. In some embodiments, the IGF-1 variant comprises the amino acid sequence of SEQ ID NO: 40. In some embodiments, the IGF-1 variant comprises the amino acid sequence of SEQ ID NO: 41. In some embodiments, the IGF-1 variant comprises the amino acid sequence of SEQ ID NO: 42. In some embodiments, the IGF-1 variant comprises the amino acid sequence of SEQ ID NO: 43. In some embodiments, the IGF-1 variant comprises the amino acid sequence of SEQ ID NO: 44. In some embodiments, the IGF-1 variant comprises the amino acid sequence of SEQ ID NO: 45. In some embodiments, the IGF-1 variant comprises the amino acid sequence of SEQ ID NO: 46. In some embodiments, the IGF-1 variant comprises the amino acid sequence of SEQ ID NO: 47. In some embodiments, the IGF-1 variant comprises the amino acid sequence of SEQ ID NO: 48. In some embodiments, the IGF-1 variant comprises the amino acid sequence of SEQ ID NO: 49. In some embodiments, the IGF-1 variant comprises the amino acid sequence of SEQ ID NO: 50. In some embodiments, the IGF-1 variant comprises the amino acid sequence of SEQ ID NO: 51. In some embodiments, the IGF-1 variant comprises the amino acid sequence of SEQ ID NO: 52. In some embodiments, the IGF-1 variant comprises the amino acid sequence of SEQ ID NO: 53. In some embodiments, the IGF-1 variant comprises the amino acid sequence of SEQ ID NO: 54. In some embodiments, the IGF-1 variant comprises the amino acid sequence of SEQ ID NO: 55. In some embodiments, the IGF-1 variant comprises the amino acid sequence of SEQ ID NO: 56. In some embodiments, the IGF-1 variant comprises the amino acid sequence of SEQ ID NO: 57. In some embodiments, the IGF-1 variant comprises the amino acid sequence of SEQ ID NO: 58. In some embodiments, the IGF-1 variant comprises the amino acid sequence of SEQ ID NO: 59. In some embodiments, the IGF-1 variant comprises the amino acid sequence of SEQ ID NO: 60. In some embodiments, the IGF-1 variant comprises the amino acid sequence of SEQ ID NO: 61. In some embodiments, the IGF-1 variant comprises the amino acid sequence of SEQ ID NO: 62. In some embodiments, the IGF-1 variant comprises the amino acid sequence of SEQ ID NO: 63. In some embodiments, the IGF-1 variant comprises the amino acid sequence of SEQ ID NO: 64. In some embodiments, the IGF-1 variant comprises the amino acid sequence of SEQ ID NO: 65. In some embodiments, the IGF-1 variant comprises the amino acid sequence of SEQ ID NO: 66. In some embodiments, the IGF-1 variant comprises the amino acid sequence of SEQ ID NO: 67. In some embodiments, the IGF-1 variant comprises the amino acid sequence of SEQ ID NO: 68. In some embodiments, the IGF-1 variant comprises the amino acid sequence of SEQ ID NO: 69. In some embodiments, the IGF-1 variant comprises the amino acid sequence of SEQ ID NO: 70. In some embodiments, the IGF-1 variant comprises the amino acid sequence of SEQ ID NO: 71. In some embodiments, the IGF-1 variant comprises the amino acid sequence of SEQ ID NO: 72. In some embodiments, the IGF-1 variant comprises the amino acid sequence of SEQ ID NO: 73. In some embodiments, the IGF-1 variant comprises the amino acid sequence of SEQ ID NO: 74. In some embodiments, the IGF-1 variant comprises the amino acid sequence of SEQ ID NO: 75. In some embodiments, the IGF-1 variant comprises the amino acid sequence of SEQ ID NO: 76. In some embodiments, the IGF-1 variant comprises the amino acid sequence of SEQ ID NO: 77. In some embodiments, the IGF-1 variant comprises the amino acid sequence of SEQ ID NO: 78. In some embodiments, the IGF-1 variant comprises the amino acid sequence of SEQ ID NO: 79. In some embodiments, the IGF-1 variant comprises the amino acid sequence of SEQ ID NO: 80. In some embodiments, the IGF-1 variant comprises the amino acid sequence of SEQ ID NO: 81. In some embodiments, the IGF-1 variant comprises the amino acid sequence of SEQ ID NO: 82. In some embodiments, the IGF-1 variant comprises the amino acid sequence of SEQ ID NO: 83. In some embodiments, the IGF-1 variant comprises the amino acid sequence of SEQ ID NO: 84. In some embodiments, the IGF-1 variant comprises the amino acid sequence of SEQ ID NO: 85. In some embodiments, the IGF-1 variant comprises the amino acid sequence of SEQ ID NO: 86. In some embodiments, the IGF-1 variant comprises the amino acid sequence of SEQ ID NO: 87. In some embodiments, the IGF-1 variant comprises the amino acid sequence of SEQ ID NO: 88. In some embodiments, the IGF-1 variant comprises the amino acid sequence of SEQ ID NO: 89. In some embodiments, the IGF-1 variant comprises the amino acid sequence of SEQ ID NO: 90. In some embodiments, the IGF-1 variant comprises the amino acid sequence of SEQ ID NO: 91. In some embodiments, the IGF-1 variant comprises the amino acid sequence of SEQ ID NO: 92. In some embodiments, the IGF-1 variant comprises the amino acid sequence of SEQ ID NO: 93. In some embodiments, the IGF-1 variant comprises the amino acid sequence of SEQ ID NO: 94. In some embodiments, the IGF-1 variant comprises the amino acid sequence of SEQ ID NO: 95. In some embodiments, the IGF-1 variant comprises the amino acid sequence of SEQ ID NO: 96. In some embodiments, the IGF-1 variant comprises the amino acid sequence of SEQ ID NO: 97. In some embodiments, the IGF-1 variant comprises the amino acid sequence of SEQ ID NO: 98. In some embodiments, the IGF-1 variant comprises the amino acid sequence of SEQ ID NO: 99. In some embodiments, the IGF-1 variant comprises the amino acid sequence of SEQ ID NO: 100. In some embodiments, the IGF-1 variant comprises the amino acid sequence of SEQ ID NO: 101. In some embodiments, the IGF-1 variant comprises the amino acid sequence of SEQ ID NO: 102. In some embodiments, the IGF-1 variant comprises the amino acid sequence of SEQ ID NO: 103. In some embodiments, the IGF-1 variant comprises the amino acid sequence of SEQ ID NO: 104. In some embodiments, the IGF-1 variant comprises the amino acid sequence of SEQ ID NO: 105. In some embodiments, the IGF-1 variant comprises the amino acid sequence of SEQ ID NO: 106. In some embodiments, the IGF-1 variant comprises the amino acid sequence of SEQ ID NO: 107. In some embodiments, the IGF-1 variant comprises the amino acid sequence of SEQ ID NO: 108. In some embodiments, the IGF-1 variant comprises the amino acid sequence of SEQ ID NO: 109. In some embodiments, the IGF-1 variant comprises the amino acid sequence of SEQ ID NO: 110. In some embodiments, the IGF-1 variant comprises the amino acid sequence of SEQ ID NO: 111. In some embodiments, the IGF-1 variant comprises the amino acid sequence of SEQ ID NO: 112. In some embodiments, the IGF-1 variant comprises the amino acid sequence of SEQ ID NO: 113. In some embodiments, the IGF-1 variant comprises the amino acid sequence of SEQ ID NO: 114. In some embodiments, the IGF-1 variant comprises the amino acid sequence of SEQ ID NO: 115. In some embodiments, the IGF-1 variant comprises the amino acid sequence of SEQ ID NO: 116. In some embodiments, the IGF-1 variant comprises the amino acid sequence of SEQ ID NO: 117. In some embodiments, the IGF-1 variant comprises the amino acid sequence of SEQ ID NO: 118. In some embodiments, the IGF-1 variant comprises the amino acid sequence of SEQ ID NO: 119. In some embodiments, the IGF-1 variant comprises the amino acid sequence of SEQ ID NO: 120. In some embodiments, the IGF-1 variant comprises the amino acid sequence of SEQ ID NO: 121. In some embodiments, the IGF-1 variant comprises the amino acid sequence of SEQ ID NO: 122. In some embodiments, the IGF-1 variant comprises the amino acid sequence of SEQ ID NO: 123. In some embodiments, the IGF-1 variant comprises the amino acid sequence of SEQ ID NO: 124. In some embodiments, the IGF-1 variant comprises the amino acid sequence of SEQ ID NO: 125. In some embodiments, the IGF-1 variant comprises the amino acid sequence of SEQ ID NO: 126. In some embodiments, the IGF-1 variant comprises the amino acid sequence of SEQ ID NO: 127. In some embodiments, the IGF-1 variant comprises the amino acid sequence of SEQ ID NO: 128. In some embodiments, the IGF-1 variant comprises the amino acid sequence of SEQ ID NO: 129. In some embodiments, the IGF-1 variant comprises the amino acid sequence of SEQ ID NO: 130. In some embodiments, the IGF-1 variant comprises the amino acid sequence of SEQ ID NO: 131. In some embodiments, the IGF-1 variant comprises the amino acid sequence of SEQ ID NO: 132. In some embodiments, the IGF-1 variant comprises the amino acid sequence of SEQ ID NO: 133. In some embodiments, the IGF-1 variant comprises the amino acid sequence of SEQ ID NO: 134. In some embodiments, the IGF-1 variant comprises the amino acid sequence of SEQ ID NO: 135. In some embodiments, the IGF-1 variant comprises the amino acid sequence of SEQ ID NO: 136. In some embodiments, the IGF-1 variant comprises the amino acid sequence of SEQ ID NO: 137. In some embodiments, the IGF-1 variant comprises the amino acid sequence of SEQ ID NO: 138. In some embodiments, the IGF-1 variant comprises the amino acid sequence of SEQ ID NO: 139. In some embodiments, the IGF-1 variant comprises the amino acid sequence of SEQ ID NO: 140. In some embodiments, the IGF-1 variant comprises the amino acid sequence of SEQ ID NO: 141. In some embodiments, the IGF-1 variant comprises the amino acid sequence of SEQ ID NO: 142. In some embodiments, the IGF-1 variant comprises the amino acid sequence of SEQ ID NO: 143. In some embodiments, the IGF-1 variant comprises the amino acid sequence of SEQ ID NO: 144. In some embodiments, the IGF-1 variant comprises the amino acid sequence of SEQ ID NO: 145. In some embodiments, the IGF-1 variant comprises the amino acid sequence of SEQ ID NO: 146. In some embodiments, the IGF-1 variant comprises the amino acid sequence of SEQ ID NO: 147. In some embodiments, the IGF-1 variant comprises the amino acid sequence of SEQ ID NO: 148. In some embodiments, the IGF-1 variant comprises the amino acid sequence of SEQ ID NO: 149. In some embodiments, the IGF-1 variant comprises the amino acid sequence of SEQ ID NO: 150. In some embodiments, the IGF-1 variant comprises the amino acid sequence of SEQ ID NO: 151. In some embodiments, the IGF-1 variant comprises the amino acid sequence of SEQ ID NO: 152. In some embodiments, the IGF-1 variant comprises the amino acid sequence of SEQ ID NO: 153. In some embodiments, the IGF-1 variant comprises the amino acid sequence of SEQ ID NO: 154. In some embodiments, the IGF-1 variant comprises the amino acid sequence of SEQ ID NO: 155. In some embodiments, the IGF-1 variant comprises the amino acid sequence of SEQ ID NO: 156. In some embodiments, the IGF-1 variant comprises the amino acid sequence of SEQ ID NO: 157. In some embodiments, the IGF-1 variant comprises the amino acid sequence of SEQ ID NO: 158. In some embodiments, the IGF-1 variant comprises the amino acid sequence of SEQ ID NO: 159. In some embodiments, the IGF-1 variant comprises the amino acid sequence of SEQ ID NO: 160. In some embodiments, the IGF-1 variant comprises the amino acid sequence of SEQ ID NO: 161. In some embodiments, the IGF-1 variant comprises the amino acid sequence of SEQ ID NO: 162. In some embodiments, the IGF-1 variant comprises the amino acid sequence of SEQ ID NO: 163. In some embodiments, the IGF-1 variant comprises the amino acid sequence of SEQ ID NO: 164. In some embodiments, the IGF-1 variant comprises the amino acid sequence of SEQ ID NO: 165. In some embodiments, the IGF-1 variant comprises the amino acid sequence of SEQ ID NO: 166. In some embodiments, the IGF-1 variant comprises the amino acid sequence of SEQ ID NO: 167. In some embodiments, the IGF-1 variant comprises the amino acid sequence of SEQ ID NO: 168. In some embodiments, the IGF-1 variant comprises the amino acid sequence of SEQ ID NO: 169. In some embodiments, the IGF-1 variant comprises the amino acid sequence of SEQ ID NO: 170. In some embodiments, the IGF-1 variant comprises the amino acid sequence of SEQ ID NO: 171. In some embodiments, the IGF-1 variant comprises the amino acid sequence of SEQ ID NO: 172. In some embodiments, the IGF-1 variant comprises the amino acid sequence of SEQ ID NO: 173. In some embodiments, the IGF-1 variant comprises the amino acid sequence of SEQ ID NO: 174. In some embodiments, the IGF-1 variant comprises the amino acid sequence of SEQ ID NO: 175. In some embodiments, the IGF-1 variant comprises the amino acid sequence of SEQ ID NO: 176. In some embodiments, the IGF-1 variant comprises the amino acid sequence of SEQ ID NO: 177. In some embodiments, the IGF-1 variant comprises the amino acid sequence of SEQ ID NO: 178. In some embodiments, the IGF-1 variant comprises the amino acid sequence of SEQ ID NO: 179. In some embodiments, the IGF-1 variant comprises the amino acid sequence of SEQ ID NO: 180. In some embodiments, the IGF-1 variant comprises the amino acid sequence of SEQ ID NO: 181. In some embodiments, the IGF-1 variant comprises the amino acid sequence of SEQ ID NO: 182. In some embodiments, the IGF-1 variant comprises the amino acid sequence of SEQ ID NO: 183. In some embodiments, the IGF-1 variant comprises the amino acid sequence of SEQ ID NO: 184. In some embodiments, the IGF-1 variant comprises the amino acid sequence of SEQ ID NO: 185. In some embodiments, the IGF-1 variant comprises the amino acid sequence of SEQ ID NO: 186. In some embodiments, the IGF-1 variant comprises the amino acid sequence of SEQ ID NO: 187. In some embodiments, the IGF-1 variant comprises the amino acid sequence of SEQ ID NO: 188. In some embodiments, the IGF-1 variant comprises the amino acid sequence of SEQ ID NO: 189. In some embodiments, the IGF-1 variant comprises the amino acid sequence of SEQ ID NO: 190. In some embodiments, the IGF-1 variant comprises the amino acid sequence of SEQ ID NO: 191. In some embodiments, the IGF-1 variant comprises the amino acid sequence of SEQ ID NO: 192. In some embodiments, the IGF-1 variant comprises the amino acid sequence of SEQ ID NO: 193. In some embodiments, the IGF-1 variant comprises the amino acid sequence of SEQ ID NO: 194. In some embodiments, the IGF-1 variant comprises the amino acid sequence of SEQ ID NO: 195. In some embodiments, the IGF-1 variant comprises the amino acid sequence of SEQ ID NO: 196. In some embodiments, the IGF-1 variant comprises the amino acid sequence of SEQ ID NO: 197. In some embodiments, the IGF-1 variant comprises the amino acid sequence of SEQ ID NO: 198. In some embodiments, the IGF-1 variant comprises the amino acid sequence of SEQ ID NO: 199. In some embodiments, the IGF-1 variant comprises the amino acid sequence of SEQ ID NO: 200. In some embodiments, the IGF-1 variant comprises the amino acid sequence of SEQ ID NO: 201. In some embodiments, the IGF-1 variant comprises the amino acid sequence of SEQ ID NO: 202. In some embodiments, the IGF-1 variant comprises the amino acid sequence of SEQ ID NO: 203. In some embodiments, the IGF-1 variant comprises the amino acid sequence of SEQ ID NO: 204. In some embodiments, the IGF-1 variant comprises the amino acid sequence of SEQ ID NO: 205. In some embodiments, the IGF-1 variant comprises the amino acid sequence of SEQ ID NO: 206. In some embodiments, the IGF-1 variant comprises the amino acid sequence of SEQ ID NO: 207. In some embodiments, the IGF-1 variant comprises the amino acid sequence of SEQ ID NO: 208. In some embodiments, the IGF-1 variant comprises the amino acid sequence of SEQ ID NO: 209. In some embodiments, the IGF-1 variant comprises the amino acid sequence of SEQ ID NO: 210. In some embodiments, the IGF-1 variant comprises the amino acid sequence of SEQ ID NO: 211. In some embodiments, the IGF-1 variant comprises the amino acid sequence of SEQ ID NO: 212. In some embodiments, the IGF-1 variant comprises the amino acid sequence of SEQ ID NO: 213. In some embodiments, the IGF-1 variant comprises the amino acid sequence of SEQ ID NO: 214. In some embodiments, the IGF-1 variant comprises the amino acid sequence of SEQ ID NO: 215. In some embodiments, the IGF-1 variant comprises the amino acid sequence of SEQ ID NO: 216. In some embodiments, the IGF-1 variant comprises the amino acid sequence of SEQ ID NO: 217. In some embodiments, the IGF-1 variant comprises the amino acid sequence of SEQ ID NO: 218. In some embodiments, the IGF-1 variant comprises the amino acid sequence of SEQ ID NO: 219. In some embodiments, the IGF-1 variant comprises the amino acid sequence of SEQ ID NO: 220. In some embodiments, the IGF-1 variant comprises the amino acid sequence of SEQ ID NO: 221. In some embodiments, the IGF-1 variant comprises the amino acid sequence of SEQ ID NO: 222. In some embodiments, the IGF-1 variant comprises the amino acid sequence of SEQ ID NO: 223. In some embodiments, the IGF-1 variant comprises the amino acid sequence of SEQ ID NO: 224. In some embodiments, the IGF-1 variant comprises the amino acid sequence of SEQ ID NO: 225. In some embodiments, the IGF-1 variant comprises the amino acid sequence of SEQ ID NO: 226. In some embodiments, the IGF-1 variant comprises the amino acid sequence of SEQ ID NO: 227. In some embodiments, the IGF-1 variant comprises the amino acid sequence of SEQ ID NO: 228. In some embodiments, the IGF-1 variant comprises the amino acid sequence of SEQ ID NO: 229. In some embodiments, the IGF-1 variant comprises the amino acid sequence of SEQ ID NO: 230. In some embodiments, the IGF-1 variant comprises the amino acid sequence of SEQ ID NO: 231. In some embodiments, the IGF-1 variant comprises the amino acid sequence of SEQ ID NO: 232. In some embodiments, the IGF-1 variant comprises the amino acid sequence of SEQ ID NO: 233. In some embodiments, the IGF-1 variant comprises the amino acid sequence of SEQ ID NO: 234. In some embodiments, the IGF-1 variant comprises the amino acid sequence of SEQ ID NO: 235. In some embodiments, the IGF-1 variant comprises the amino acid sequence of SEQ ID NO: 236. In some embodiments, the IGF-1 variant comprises the amino acid sequence of SEQ ID NO: 237. In some embodiments, the IGF-1 variant comprises the amino acid sequence of SEQ ID NO: 238. In some embodiments, the IGF-1 variant comprises the amino acid sequence of SEQ ID NO: 239. In some embodiments, the IGF-1 variant comprises the amino acid sequence of SEQ ID NO: 240. In some embodiments, the IGF-1 variant comprises the amino acid sequence of SEQ ID NO: 241. In some embodiments, the IGF-1 variant comprises the amino acid sequence of SEQ ID NO: 242. In some embodiments, the IGF-1 variant comprises the amino acid sequence of SEQ ID NO: 243. In some embodiments, the IGF-1 variant comprises the amino acid sequence of SEQ ID NO: 244. In some embodiments, the IGF-1 variant comprises the amino acid sequence of SEQ ID NO: 245. In some embodiments, the IGF-1 variant comprises the amino acid sequence of SEQ ID NO: 246. In some embodiments, the IGF-1 variant comprises the amino acid sequence of SEQ ID NO: 247. In some embodiments, the IGF-1 variant comprises the amino acid sequence of SEQ ID NO: 248. In some embodiments, the IGF-1 variant comprises the amino acid sequence of SEQ ID NO: 249. In some embodiments, the IGF-1 variant comprises the amino acid sequence of SEQ ID NO: 250. In some embodiments, the IGF-1 variant comprises the amino acid sequence of SEQ ID NO: 251. In some embodiments, the IGF-1 variant comprises the amino acid sequence of SEQ ID NO: 252. In some embodiments, the IGF-1 variant comprises the amino acid sequence of SEQ ID NO: 253. In some embodiments, the IGF-1 variant comprises the amino acid sequence of SEQ ID NO: 254. In some embodiments, the IGF-1 variant comprises the amino acid sequence of SEQ ID NO: 255. In some embodiments, the IGF-1 variant comprises the amino acid sequence of SEQ ID NO: 256. In some embodiments, the IGF-1 variant comprises the amino acid sequence of SEQ ID NO: 257. In some embodiments, the IGF-1 variant comprises the amino acid sequence of SEQ ID NO: 258. In some embodiments, the IGF-1 variant comprises the amino acid sequence of SEQ ID NO: 259. In some embodiments, the IGF-1 variant comprises the amino acid sequence of SEQ ID NO: 260. In some embodiments, the IGF-1 variant comprises the amino acid sequence of SEQ ID NO: 261. In some embodiments, the IGF-1 variant comprises the amino acid sequence of SEQ ID NO: 262. In some embodiments, the IGF-1 variant comprises the amino acid sequence of SEQ ID NO: 263. In some embodiments, the IGF-1 variant comprises the amino acid sequence of SEQ ID NO: 264. In some embodiments, the IGF-1 variant comprises the amino acid sequence of SEQ ID NO: 265. In some embodiments, the IGF-1 variant comprises the amino acid sequence of SEQ ID NO: 266. In some embodiments, the IGF-1 variant comprises the amino acid sequence of SEQ ID NO: 267. In some embodiments, the IGF-1 variant comprises the amino acid sequence of SEQ ID NO: 268. In some embodiments, the IGF-1 variant comprises the amino acid sequence of SEQ ID NO: 269. In some embodiments, the IGF-1 variant comprises the amino acid sequence of SEQ ID NO: 270. In some embodiments, the IGF-1 variant comprises the amino acid sequence of SEQ ID NO: 271. In some embodiments, the IGF-1 variant comprises the amino acid sequence of SEQ ID NO: 272. In some embodiments, the IGF-1 variant comprises the amino acid sequence of SEQ ID NO: 273. In some embodiments, the IGF-1 variant comprises the amino acid sequence of SEQ ID NO: 274. In some embodiments, the IGF-1 variant comprises the amino acid sequence of SEQ ID NO: 275. In some embodiments, the IGF-1 variant comprises the amino acid sequence of SEQ ID NO: 276. In some embodiments, the IGF-1 variant comprises the amino acid sequence of SEQ ID NO: 277. In some embodiments, the IGF-1 variant comprises the amino acid sequence of SEQ ID NO: 278. In some embodiments, the IGF-1 variant comprises the amino acid sequence of SEQ ID NO: 279. In some embodiments, the IGF-1 variant comprises the amino acid sequence of SEQ ID NO: 280. In some embodiments, the IGF-1 variant comprises the amino acid sequence of SEQ ID NO: 281. In some embodiments, the IGF-1 variant comprises the amino acid sequence of SEQ ID NO: 282. In some embodiments, the IGF-1 variant comprises the amino acid sequence of SEQ ID NO: 283. In some embodiments, the IGF-1 variant comprises the amino acid sequence of SEQ ID NO: 284. In some embodiments, the IGF-1 variant comprises the amino acid sequence of SEQ ID NO: 285. In some embodiments, the IGF-1 variant comprises the amino acid sequence of SEQ ID NO: 286. In some embodiments, the IGF-1 variant comprises the amino acid sequence of SEQ ID NO: 287. In some embodiments, the IGF-1 variant comprises the amino acid sequence of SEQ ID NO: 288. In some embodiments, the IGF-1 variant comprises the amino acid sequence of SEQ ID NO: 289. In some embodiments, the IGF-1 variant comprises the amino acid sequence of SEQ ID NO: 290. In some embodiments, the IGF-1 variant comprises the amino acid sequence of SEQ ID NO: 291. In some embodiments, the IGF-1 variant comprises the amino acid sequence of SEQ ID NO: 292. In some embodiments, the IGF-1 variant comprises the amino acid sequence of SEQ ID NO: 293. In some embodiments, the IGF-1 variant comprises the amino acid sequence of SEQ ID NO: 294. In some embodiments, the IGF-1 variant comprises the amino acid sequence of SEQ ID NO: 295. In some embodiments, the IGF-1 variant comprises the amino acid sequence of SEQ ID NO: 296. In some embodiments, the IGF-1 variant comprises the amino acid sequence of SEQ ID NO: 297. In some embodiments, the IGF-1 variant comprises the amino acid sequence of SEQ ID NO: 298. In some embodiments, the IGF-1 variant comprises the amino acid sequence of SEQ ID NO: 299. In some embodiments, the IGF-1 variant comprises the amino acid sequence of SEQ ID NO: 300. In some embodiments, the IGF-1 variant comprises the amino acid sequence of SEQ ID NO: 301. In some embodiments, the IGF-1 variant comprises the amino acid sequence of SEQ ID NO: 302. In some embodiments, the IGF-1 variant comprises the amino acid sequence of SEQ ID NO: 303. In some embodiments, the IGF-1 variant comprises the amino acid sequence of SEQ ID NO: 304. In some embodiments, the IGF-1 variant comprises the amino acid sequence of SEQ ID NO: 305. In some embodiments, the IGF-1 variant comprises the amino acid sequence of SEQ ID NO: 306. In some embodiments, the IGF-1 variant comprises the amino acid sequence of SEQ ID NO: 307. In some embodiments, the IGF-1 variant comprises the amino acid sequence of SEQ ID NO: 308. In some embodiments, the IGF-1 variant comprises the amino acid sequence of SEQ ID NO: 309. In some embodiments, the IGF-1 variant comprises the amino acid sequence of SEQ ID NO: 310. In some embodiments, the IGF-1 variant comprises the amino acid sequence of SEQ ID NO: 311. In some embodiments, the IGF-1 variant comprises the amino acid sequence of SEQ ID NO: 312. In some embodiments, the IGF-1 variant comprises the amino acid sequence of SEQ ID NO: 313. In some embodiments, the IGF-1 variant comprises the amino acid sequence of SEQ ID NO: 314. In some embodiments, the IGF-1 variant comprises the amino acid sequence of SEQ ID NO: 315. In some embodiments, the IGF-1 variant comprises the amino acid sequence of SEQ ID NO: 316. In some embodiments, the IGF-1 variant comprises the amino acid sequence of SEQ ID NO: 317. In some embodiments, the IGF-1 variant comprises the amino acid sequence of SEQ ID NO: 318. In some embodiments, the IGF-1 variant comprises the amino acid sequence of SEQ ID NO: 319. In some embodiments, the IGF-1 variant comprises the amino acid sequence of SEQ ID NO: 320. In some embodiments, the IGF-1 variant comprises the amino acid sequence of SEQ ID NO: 321. In some embodiments, the IGF-1 variant comprises the amino acid sequence of SEQ ID NO: 322. In some embodiments, the IGF-1 variant comprises the amino acid sequence of SEQ ID NO: 323. In some embodiments, the IGF-1 variant comprises the amino acid sequence of SEQ ID NO: 324. In some embodiments, the IGF-1 variant comprises the amino acid sequence of SEQ ID NO: 325. In some embodiments, the IGF-1 variant comprises the amino acid sequence of SEQ ID NO: 326. In some embodiments, the IGF-1 variant comprises the amino acid sequence of SEQ ID NO: 327. In some embodiments, the IGF-1 variant comprises the amino acid sequence of SEQ ID NO: 328. In some embodiments, the IGF-1 variant comprises the amino acid sequence of SEQ ID NO: 329. In some embodiments, the IGF-1 variant comprises the amino acid sequence of SEQ ID NO: 330. In some embodiments, the IGF-1 variant comprises the amino acid sequence of SEQ ID NO: 331. In some embodiments, the IGF-1 variant comprises the amino acid sequence of SEQ ID NO: 332. In some embodiments, the IGF-1 variant comprises the amino acid sequence of SEQ ID NO: 333. In some embodiments, the IGF-1 variant comprises the amino acid sequence of SEQ ID NO: 334. In some embodiments, the IGF-1 variant comprises the amino acid sequence of SEQ ID NO: 335. In some embodiments, the IGF-1 variant comprises the amino acid sequence of SEQ ID NO: 336. In some embodiments, the IGF-1 variant comprises the amino acid sequence of SEQ ID NO: 337. In some embodiments, the IGF-1 variant comprises the amino acid sequence of SEQ ID NO: 338. In some embodiments, the IGF-1 variant comprises the amino acid sequence of SEQ ID NO: 339. In some embodiments, the IGF-1 variant comprises the amino acid sequence of SEQ ID NO: 340. In some embodiments, the IGF-1 variant comprises the amino acid sequence of SEQ ID NO: 341. In some embodiments, the IGF-1 variant comprises the amino acid sequence of SEQ ID NO: 342. In some embodiments, the IGF-1 variant comprises the amino acid sequence of SEQ ID NO: 343. In some embodiments, the IGF-1 variant comprises the amino acid sequence of SEQ ID NO: 344. In some embodiments, the IGF-1 variant comprises the amino acid sequence of SEQ ID NO: 345. In some embodiments, the IGF-1 variant comprises the amino acid sequence of SEQ ID NO: 346. In some embodiments, the IGF-1 variant comprises the amino acid sequence of SEQ ID NO: 347. In some embodiments, the IGF-1 variant comprises the amino acid sequence of SEQ ID NO: 348. In some embodiments, the IGF-1 variant comprises the amino acid sequence of SEQ ID NO: 349. In some embodiments, the IGF-1 variant comprises the amino acid sequence of SEQ ID NO: 350. In some embodiments, the IGF-1 variant comprises the amino acid sequence of SEQ ID NO: 351. In some embodiments, the IGF-1 variant comprises the amino acid sequence of SEQ ID NO: 352. In some embodiments, the IGF-1 variant comprises the amino acid sequence of SEQ ID NO: 353. In some embodiments, the IGF-1 variant comprises the amino acid sequence of SEQ ID NO: 354. In some embodiments, the IGF-1 variant comprises the amino acid sequence of SEQ ID NO: 355. In some embodiments, the IGF-1 variant comprises the amino acid sequence of SEQ ID NO: 356. In some embodiments, the IGF-1 variant comprises the amino acid sequence of SEQ ID NO: 357. In some embodiments, the IGF-1 variant comprises the amino acid sequence of SEQ ID NO: 358. In some embodiments, the IGF-1 variant comprises the amino acid sequence of SEQ ID NO: 359. In some embodiments, the IGF-1 variant comprises the amino acid sequence of SEQ ID NO: 360. In some embodiments, the IGF-1 variant comprises the amino acid sequence of SEQ ID NO: 361. In some embodiments, the IGF-1 variant comprises the amino acid sequence of SEQ ID NO: 362. In some embodiments, the IGF-1 variant comprises the amino acid sequence of SEQ ID NO: 363. In some embodiments, the IGF-1 variant comprises the amino acid sequence of SEQ ID NO: 364. In some embodiments, the IGF-1 variant comprises the amino acid sequence of SEQ ID NO: 365. In some embodiments, the IGF-1 variant comprises the amino acid sequence of SEQ ID NO: 366. In some embodiments, the IGF-1 variant comprises the amino acid sequence of SEQ ID NO: 367. In some embodiments, the IGF-1 variant comprises the amino acid sequence of SEQ ID NO: 368. In some embodiments, the IGF-1 variant comprises the amino acid sequence of SEQ ID NO: 369. In some embodiments, the IGF-1 variant comprises the amino acid sequence of SEQ ID NO: 370. In some embodiments, the IGF-1 variant comprises the amino acid sequence of SEQ ID NO: 371. In some embodiments, the IGF-1 variant comprises the amino acid sequence of SEQ ID NO: 372. In some embodiments, the IGF-1 variant comprises the amino acid sequence of SEQ ID NO: 373. In some embodiments, the IGF-1 variant comprises the amino acid sequence of SEQ ID NO: 374. In some embodiments, the IGF-1 variant comprises the amino acid sequence of SEQ ID NO: 375. In some embodiments, the IGF-1 variant comprises the amino acid sequence of SEQ ID NO: 376. In some embodiments, the IGF-1 variant comprises the amino acid sequence of SEQ ID NO: 377. In some embodiments, the IGF-1 variant comprises the amino acid sequence of SEQ ID NO: 378. In some embodiments, the IGF-1 variant comprises the amino acid sequence of SEQ ID NO: 379. In some embodiments, the IGF-1 variant comprises the amino acid sequence of SEQ ID NO: 380. In some embodiments, the IGF-1 variant comprises the amino acid sequence of SEQ ID NO: 381. In some embodiments, the IGF-1 variant comprises the amino acid sequence of SEQ ID NO: 382. In some embodiments, the IGF-1 variant comprises the amino acid sequence of SEQ ID NO: 383. In some embodiments, the IGF-1 variant comprises the amino acid sequence of SEQ ID NO: 384. In some embodiments, the IGF-1 variant comprises the amino acid sequence of SEQ ID NO: 385. In some embodiments, the IGF-1 variant comprises the amino acid sequence of SEQ ID NO: 386. In some embodiments, the IGF-1 variant comprises the amino acid sequence of SEQ ID NO: 387. In some embodiments, the IGF-1 variant comprises the amino acid sequence of SEQ ID NO: 388. In some embodiments, the IGF-1 variant comprises the amino acid sequence of SEQ ID NO: 389. In some embodiments, the IGF-1 variant comprises the amino acid sequence of SEQ ID NO: 390. In some embodiments, the IGF-1 variant comprises the amino acid sequence of SEQ ID NO: 391. In some embodiments, the IGF-1 variant comprises the amino acid sequence of SEQ ID NO: 392. In some embodiments, the IGF-1 variant comprises the amino acid sequence of SEQ ID NO: 393. In some embodiments, the IGF-1 variant comprises the amino acid sequence of SEQ ID NO: 394. In some embodiments, the IGF-1 variant comprises the amino acid sequence of SEQ ID NO: 395. In some embodiments, the IGF-1 variant comprises the amino acid sequence of SEQ ID NO: 396. In some embodiments, the IGF-1 variant comprises the amino acid sequence of SEQ ID NO: 397. In some embodiments, the IGF-1 variant comprises the amino acid sequence of SEQ ID NO: 398. In some embodiments, the IGF-1 variant comprises the amino acid sequence of SEQ ID NO: 399. In some embodiments, the IGF-1 variant comprises the amino acid sequence of SEQ ID NO: 400. In some embodiments, the IGF-1 variant comprises the amino acid sequence of SEQ ID NO: 401. In some embodiments, the IGF-1 variant comprises the amino acid sequence of SEQ ID NO: 402. In some embodiments, the IGF-1 variant comprises the amino acid sequence of SEQ ID NO: 403. In some embodiments, the IGF-1 variant comprises the amino acid sequence of SEQ ID NO: 404. In some embodiments, the IGF-1 variant comprises the amino acid sequence of SEQ ID NO: 405. In some embodiments, the IGF-1 variant comprises the amino acid sequence of SEQ ID NO: 406. In some embodiments, the IGF-1 variant comprises the amino acid sequence of SEQ ID NO: 407. In some embodiments, the IGF-1 variant comprises the amino acid sequence of SEQ ID NO: 408. In some embodiments, the IGF-1 variant comprises the amino acid sequence of SEQ ID NO: 409. In some embodiments, the IGF-1 variant comprises the amino acid sequence of SEQ ID NO: 410. In some embodiments, the IGF-1 variant comprises the amino acid sequence of SEQ ID NO: 411. In some embodiments, the IGF-1 variant comprises the amino acid sequence of SEQ ID NO: 412. In some embodiments, the IGF-1 variant comprises the amino acid sequence of SEQ ID NO: 413. In some embodiments, the IGF-1 variant comprises the amino acid sequence of SEQ ID NO: 414. In some embodiments, the IGF-1 variant comprises the amino acid sequence of SEQ ID NO: 415. In some embodiments, the IGF-1 variant comprises the amino acid sequence of SEQ ID NO: 416. In some embodiments, the IGF-1 variant comprises the amino acid sequence of SEQ ID NO: 417. In some embodiments, the IGF-1 variant comprises the amino acid sequence of SEQ ID NO: 418. In some embodiments, the IGF-1 variant comprises the amino acid sequence of SEQ ID NO: 419. In some embodiments, the IGF-1 variant comprises the amino acid sequence of SEQ ID NO: 420. In some embodiments, the IGF-1 variant comprises the amino acid sequence of SEQ ID NO: 421. In some embodiments, the IGF-1 variant comprises the amino acid sequence of SEQ ID NO: 422. In some embodiments, the IGF-1 variant comprises the amino acid sequence of SEQ ID NO: 423. In some embodiments, the IGF-1 variant comprises the amino acid sequence of SEQ ID NO: 424. In some embodiments, the IGF-1 variant comprises the amino acid sequence of SEQ ID NO: 425. In some embodiments, the IGF-1 variant comprises the amino acid sequence of SEQ ID NO: 426. In some embodiments, the IGF-1 variant comprises the amino acid sequence of SEQ ID NO: 427. In some embodiments, the IGF-1 variant comprises the amino acid sequence of SEQ ID NO: 428. In some embodiments, the IGF-1 variant comprises the amino acid sequence of SEQ ID NO: 429. In some embodiments, the IGF-1 variant comprises the amino acid sequence of SEQ ID NO: 430. In some embodiments, the IGF-1 variant comprises the amino acid sequence of SEQ ID NO: 431. In some embodiments, the IGF-1 variant comprises the amino acid sequence of SEQ ID NO: 432. In some embodiments, the IGF-1 variant comprises the amino acid sequence of SEQ ID NO: 433. In some embodiments, the IGF-1 variant comprises the amino acid sequence of SEQ ID NO: 434. In some embodiments, the IGF-1 variant comprises the amino acid sequence of SEQ ID NO: 435. In some embodiments, the IGF-1 variant comprises the amino acid sequence of SEQ ID NO: 436. In some embodiments, the IGF-1 variant comprises the amino acid sequence of SEQ ID NO: 437. In some embodiments, the IGF-1 variant comprises the amino acid sequence of SEQ ID NO: 438. In some embodiments, the IGF-1 variant comprises the amino acid sequence of SEQ ID NO: 439. In some embodiments, the IGF-1 variant comprises the amino acid sequence of SEQ ID NO: 440. In some embodiments, the IGF-1 variant comprises the amino acid sequence of SEQ ID NO: 441. In some embodiments, the IGF-1 variant comprises the amino acid sequence of SEQ ID NO: 442. In some embodiments, the IGF-1 variant comprises the amino acid sequence of SEQ ID NO: 443. In some embodiments, the IGF-1 variant comprises the amino acid sequence of SEQ ID NO: 444. In some embodiments, the IGF-1 variant comprises the amino acid sequence of SEQ ID NO: 445. In some embodiments, the IGF-1 variant comprises the amino acid sequence of SEQ ID NO: 446. In some embodiments, the IGF-1 variant comprises the amino acid sequence of SEQ ID NO: 447. In some embodiments, the IGF-1 variant comprises the amino acid sequence of SEQ ID NO: 448. In some embodiments, the IGF-1 variant comprises the amino acid sequence of SEQ ID NO: 449. In some embodiments, the IGF-1 variant comprises the amino acid sequence of SEQ ID NO: 450. In some embodiments, the IGF-1 variant comprises the amino acid sequence of SEQ ID NO: 451. In some embodiments, the IGF-1 variant comprises the amino acid sequence of SEQ ID NO: 452. In some embodiments, the IGF-1 variant comprises the amino acid sequence of SEQ ID NO: 453. In some embodiments, the IGF-1 variant comprises the amino acid sequence of SEQ ID NO: 454. In some embodiments, the IGF-1 variant comprises the amino acid sequence of SEQ ID NO: 455. In some embodiments, the IGF-1 variant comprises the amino acid sequence of SEQ ID NO: 456. In some embodiments, the IGF-1 variant comprises the amino acid sequence of SEQ ID NO: 457. In some embodiments, the IGF-1 variant comprises the amino acid sequence of SEQ ID NO: 458. In some embodiments, the IGF-1 variant comprises the amino acid sequence of SEQ ID NO: 459. In some embodiments, the IGF-1 variant comprises the amino acid sequence of SEQ ID NO: 460. In some embodiments, the IGF-1 variant comprises the amino acid sequence of SEQ ID NO: 461. In some embodiments, the IGF-1 variant comprises the amino acid sequence of SEQ ID NO: 462. In some embodiments, the IGF-1 variant comprises the amino acid sequence of SEQ ID NO: 463. In some embodiments, the IGF-1 variant comprises the amino acid sequence of SEQ ID NO: 464. In some embodiments, the IGF-1 variant comprises the amino acid sequence of SEQ ID NO: 465. In some embodiments, the IGF-1 variant comprises the amino acid sequence of SEQ ID NO: 466. In some embodiments, the IGF-1 variant comprises the amino acid sequence of SEQ ID NO: 467. In some embodiments, the IGF-1 variant comprises the amino acid sequence of SEQ ID NO: 468. In some embodiments, the IGF-1 variant comprises the amino acid sequence of SEQ ID NO: 469. In some embodiments, the IGF-1 variant comprises the amino acid sequence of SEQ ID NO: 470. In some embodiments, the IGF-1 variant comprises the amino acid sequence of SEQ ID NO: 471. In some embodiments, the IGF-1 variant comprises the amino acid sequence of SEQ ID NO: 472. In some embodiments, the IGF-1 variant comprises the amino acid sequence of SEQ ID NO: 473. In some embodiments, the IGF-1 variant comprises the amino acid sequence of SEQ ID NO: 474. In some embodiments, the IGF-1 variant comprises the amino acid sequence of SEQ ID NO: 475. In some embodiments, the IGF-1 variant comprises the amino acid sequence of SEQ ID NO: 476. In some embodiments, the IGF-1 variant comprises the amino acid sequence of SEQ ID NO: 477. In some embodiments, the IGF-1 variant comprises the amino acid sequence of SEQ ID NO: 478. In some embodiments, the IGF-1 variant comprises the amino acid sequence of SEQ ID NO: 479. In some embodiments, the IGF-1 variant comprises the amino acid sequence of SEQ ID NO: 480. In some embodiments, the IGF-1 variant comprises the amino acid sequence of SEQ ID NO: 481. In some embodiments, the IGF-1 variant comprises the amino acid sequence of SEQ ID NO: 482. In some embodiments, the IGF-1 variant comprises the amino acid sequence of SEQ ID NO: 483. In some embodiments, the IGF-1 variant comprises the amino acid sequence of SEQ ID NO: 484. In some embodiments, the IGF-1 variant comprises the amino acid sequence of SEQ ID NO: 485. In some embodiments, the IGF-1 variant comprises the amino acid sequence of SEQ ID NO: 486. In some embodiments, the IGF-1 variant comprises the amino acid sequence of SEQ ID NO: 487. In some embodiments, the IGF-1 variant comprises the amino acid sequence of SEQ ID NO: 488. In some embodiments, the IGF-1 variant comprises the amino acid sequence of SEQ ID NO: 489. In some embodiments, the IGF-1 variant comprises the amino acid sequence of SEQ ID NO: 490. In some embodiments, the IGF-1 variant comprises the amino acid sequence of SEQ ID NO: 491. In some embodiments, the IGF-1 variant comprises the amino acid sequence of SEQ ID NO: 492. In some embodiments, the IGF-1 variant comprises the amino acid sequence of SEQ ID NO: 493. In some embodiments, the IGF-1 variant comprises the amino acid sequence of SEQ ID NO: 494. In some embodiments, the IGF-1 variant comprises the amino acid sequence of SEQ ID NO: 495. In some embodiments, the IGF-1 variant comprises the amino acid sequence of SEQ ID NO: 496. In some embodiments, the IGF-1 variant comprises the amino acid sequence of SEQ ID NO: 497. In some embodiments, the IGF-1 variant comprises the amino acid sequence of SEQ ID NO: 498. In some embodiments, the IGF-1 variant comprises the amino acid sequence of SEQ ID NO: 499. In some embodiments, the IGF-1 variant comprises the amino acid sequence of SEQ ID NO: 500. In some embodiments, the IGF-1 variant comprises the amino acid sequence of SEQ ID NO: 501. In some embodiments, the IGF-1 variant comprises the amino acid sequence of SEQ ID NO: 502. In some embodiments, the IGF-1 variant comprises the amino acid sequence of SEQ ID NO: 503. In some embodiments, the IGF-1 variant comprises the amino acid sequence of SEQ ID NO: 504. In some embodiments, the IGF-1 variant comprises the amino acid sequence of SEQ ID NO: 505. In some embodiments, the IGF-1 variant comprises the amino acid sequence of SEQ ID NO: 506. In some embodiments, the IGF-1 variant comprises the amino acid sequence of SEQ ID NO: 507. In some embodiments, the IGF-1 variant comprises the amino acid sequence of SEQ ID NO: 508. In some embodiments, the IGF-1 variant comprises the amino acid sequence of SEQ ID NO: 509. In some embodiments, the IGF-1 variant comprises the amino acid sequence of SEQ ID NO: 510. In some embodiments, the IGF-1 variant comprises the amino acid sequence of SEQ ID NO: 511. In some embodiments, the IGF-1 variant comprises the amino acid sequence of SEQ ID NO: 512. In some embodiments, the IGF-1 variant comprises the amino acid sequence of SEQ ID NO: 513. In some embodiments, the IGF-1 variant comprises the amino acid sequence of SEQ ID NO: 514. In some embodiments, the IGF-1 variant comprises the amino acid sequence of SEQ ID NO: 515. In some embodiments, the IGF-1 variant comprises the amino acid sequence of SEQ ID NO: 516. In some embodiments, the IGF-1 variant comprises the amino acid sequence of SEQ ID NO: 517. In some embodiments, the IGF-1 variant comprises the amino acid sequence of SEQ ID NO: 518. In some embodiments, the IGF-1 variant comprises the amino acid sequence of SEQ ID NO: 519. In some embodiments, the IGF-1 variant comprises the amino acid sequence of SEQ ID NO: 520. In some embodiments, the IGF-1 variant comprises the amino acid sequence of SEQ ID NO: 521. In some embodiments, the IGF-1 variant comprises the amino acid sequence of SEQ ID NO: 522. In some embodiments, the IGF-1 variant comprises the amino acid sequence of SEQ ID NO: 523. In some embodiments, the IGF-1 variant comprises the amino acid sequence of SEQ ID NO: 524. In some embodiments, the IGF-1 variant comprises the amino acid sequence of SEQ ID NO: 525. In some embodiments, the IGF-1 variant comprises the amino acid sequence of SEQ ID NO: 526. In some embodiments, the IGF-1 variant comprises the amino acid sequence of SEQ ID NO: 527. In some embodiments, the IGF-1 variant comprises the amino acid sequence of SEQ ID NO: 528. In some embodiments, the IGF-1 variant comprises the amino acid sequence of SEQ ID NO: 529. In some embodiments, the IGF-1 variant comprises the amino acid sequence of SEQ ID NO: 530. In some embodiments, the IGF-1 variant comprises the amino acid sequence of SEQ ID NO: 531. In some embodiments, the IGF-1 variant comprises the amino acid sequence of SEQ ID NO: 532. In some embodiments, the IGF-1 variant comprises the amino acid sequence of SEQ ID NO: 533. In some embodiments, the IGF-1 variant comprises the amino acid sequence of SEQ ID NO: 534. In some embodiments, the IGF-1 variant comprises the amino acid sequence of SEQ ID NO: 535. In some embodiments, the IGF-1 variant comprises the amino acid sequence of SEQ ID NO: 536. In some embodiments, the IGF-1 variant comprises the amino acid sequence of SEQ ID NO: 537. In some embodiments, the IGF-1 variant comprises the amino acid sequence of SEQ ID NO: 538. In some embodiments, the IGF-1 variant comprises the amino acid sequence of SEQ ID NO: 539. In some embodiments, the IGF-1 variant comprises the amino acid sequence of SEQ ID NO: 540. In some embodiments, the IGF-1 variant comprises the amino acid sequence of SEQ ID NO: 541. In some embodiments, the IGF-1 variant comprises the amino acid sequence of SEQ ID NO: 542. In some embodiments, the IGF-1 variant comprises the amino acid sequence of SEQ ID NO: 543. In some embodiments, the IGF-1 variant comprises the amino acid sequence of SEQ ID NO: 544. In some embodiments, the IGF-1 variant comprises the amino acid sequence of SEQ ID NO: 545. In some embodiments, the IGF-1 variant comprises the amino acid sequence of SEQ ID NO: 546. In some embodiments, the IGF-1 variant comprises the amino acid sequence of SEQ ID NO: 547. In some embodiments, the IGF-1 variant comprises the amino acid sequence of SEQ ID NO: 548. In some embodiments, the IGF-1 variant comprises the amino acid sequence of SEQ ID NO: 549. In some embodiments, the IGF-1 variant comprises the amino acid sequence of SEQ ID NO: 550. In some embodiments, the IGF-1 variant comprises the amino acid sequence of SEQ ID NO: 551. In some embodiments, the IGF-1 variant comprises the amino acid sequence of SEQ ID NO: 552. In some embodiments, the IGF-1 variant comprises the amino acid sequence of SEQ ID NO: 553. In some embodiments, the IGF-1 variant comprises the amino acid sequence of SEQ ID NO: 554. In some embodiments, the IGF-1 variant comprises the amino acid sequence of SEQ ID NO: 555. In some embodiments, the IGF-1 variant comprises the amino acid sequence of SEQ ID NO: 556. In some embodiments, the IGF-1 variant comprises the amino acid sequence of SEQ ID NO: 557. In some embodiments, the IGF-1 variant comprises the amino acid sequence of SEQ ID NO: 558. In some embodiments, the IGF-1 variant comprises the amino acid sequence of SEQ ID NO: 559. In some embodiments, the IGF-1 variant comprises the amino acid sequence of SEQ ID NO: 560. In some embodiments, the IGF-1 variant comprises the amino acid sequence of SEQ ID NO: 561. In some embodiments, the IGF-1 variant comprises the amino acid sequence of SEQ ID NO: 562. In some embodiments, the IGF-1 variant comprises the amino acid sequence of SEQ ID NO: 563. In some embodiments, the IGF-1 variant comprises the amino acid sequence of SEQ ID NO: 564. In some embodiments, the IGF-1 variant comprises the amino acid sequence of SEQ ID NO: 565. In some embodiments, the IGF-1 variant comprises the amino acid sequence of SEQ ID NO: 566. In some embodiments, the IGF-1 variant comprises the amino acid sequence of SEQ ID NO: 567. In some embodiments, the IGF-1 variant comprises the amino acid sequence of SEQ ID NO: 568. In some embodiments, the IGF-1 variant comprises the amino acid sequence of SEQ ID NO: 569. In some embodiments, the IGF-1 variant comprises the amino acid sequence of SEQ ID NO: 570. In some embodiments, the IGF-1 variant comprises the amino acid sequence of SEQ ID NO: 571. In some embodiments, the IGF-1 variant comprises the amino acid sequence of SEQ ID NO: 572. In some embodiments, the IGF-1 variant comprises the amino acid sequence of SEQ ID NO: 573. In some embodiments, the IGF-1 variant comprises the amino acid sequence of SEQ ID NO: 574. In some embodiments, the IGF-1 variant comprises the amino acid sequence of SEQ ID NO: 575. In some embodiments, the IGF-1 variant comprises the amino acid sequence of SEQ ID NO: 576. In some embodiments, the IGF-1 variant comprises the amino acid sequence of SEQ ID NO: 577. In some embodiments, the IGF-1 variant comprises the amino acid sequence of SEQ ID NO: 578. In some embodiments, the IGF-1 variant comprises the amino acid sequence of SEQ ID NO: 579. In some embodiments, the IGF-1 variant comprises the amino acid sequence of SEQ ID NO: 580. In some embodiments, the IGF-1 variant comprises the amino acid sequence of SEQ ID NO: 581. In some embodiments, the IGF-1 variant comprises the amino acid sequence of SEQ ID NO: 582. In some embodiments, the IGF-1 variant comprises the amino acid sequence of SEQ ID NO: 583. In some embodiments, the IGF-1 variant comprises the amino acid sequence of SEQ ID NO: 584. In some embodiments, the IGF-1 variant comprises the amino acid sequence of SEQ ID NO: 585. In some embodiments, the IGF-1 variant comprises the amino acid sequence of SEQ ID NO: 586. In some embodiments, the IGF-1 variant comprises the amino acid sequence of SEQ ID NO: 587. In some embodiments, the IGF-1 variant comprises the amino acid sequence of SEQ ID NO: 588. In some embodiments, the IGF-1 variant comprises the amino acid sequence of SEQ ID NO: 589. In some embodiments, the IGF-1 variant comprises the amino acid sequence of SEQ ID NO: 590. In some embodiments, the IGF-1 variant comprises the amino acid sequence of SEQ ID NO: 591. In some embodiments, the IGF-1 variant comprises the amino acid sequence of SEQ ID NO: 592. In some embodiments, the IGF-1 variant comprises the amino acid sequence of SEQ ID NO: 593. In some embodiments, the IGF-1 variant comprises the amino acid sequence of SEQ ID NO: 594. In some embodiments, the IGF-1 variant comprises the amino acid sequence of SEQ ID NO: 595. In some embodiments, the IGF-1 variant comprises the amino acid sequence of SEQ ID NO: 596. In some embodiments, the IGF-1 variant comprises the amino acid sequence of SEQ ID NO: 597. In some embodiments, the IGF-1 variant comprises the amino acid sequence of SEQ ID NO: 598. In some embodiments, the IGF-1 variant comprises the amino acid sequence of SEQ ID NO: 599. In some embodiments, the IGF-1 variant comprises the amino acid sequence of SEQ ID NO: 600. In some embodiments, the IGF-1 variant comprises the amino acid sequence of SEQ ID NO: 601. In some embodiments, the IGF-1 variant comprises the amino acid sequence of SEQ ID NO: 602. In some embodiments, the IGF-1 variant comprises the amino acid sequence of SEQ ID NO: 603. In some embodiments, the IGF-1 variant comprises the amino acid sequence of SEQ ID NO: 604. In some embodiments, the IGF-1 variant comprises the amino acid sequence of SEQ ID NO: 605. In some embodiments, the IGF-1 variant comprises the amino acid sequence of SEQ ID NO: 606. In some embodiments, the IGF-1 variant comprises the amino acid sequence of SEQ ID NO: 607. In some embodiments, the IGF-1 variant comprises the amino acid sequence of SEQ ID NO: 608. In some embodiments, the IGF-1 variant comprises the amino acid sequence of SEQ ID NO: 609. In some embodiments, the IGF-1 variant comprises the amino acid sequence of SEQ ID NO: 610. In some embodiments, the IGF-1 variant comprises the amino acid sequence of SEQ ID NO: 611. In some embodiments, the IGF-1 variant comprises the amino acid sequence of SEQ ID NO: 612. In some embodiments, the IGF-1 variant comprises the amino acid sequence of SEQ ID NO: 613. In some embodiments, the IGF-1 variant comprises the amino acid sequence of SEQ ID NO: 614. In some embodiments, the IGF-1 variant comprises the amino acid sequence of SEQ ID NO: 615. In some embodiments, the IGF-1 variant comprises the amino acid sequence of SEQ ID NO: 616. In some embodiments, the IGF-1 variant comprises the amino acid sequence of SEQ ID NO: 617. In some embodiments, the IGF-1 variant comprises the amino acid sequence of SEQ ID NO: 618. In some embodiments, the IGF-1 variant comprises the amino acid sequence of SEQ ID NO: 619. In some embodiments, the IGF-1 variant comprises the amino acid sequence of SEQ ID NO: 620. In some embodiments, the IGF-1 variant comprises the amino acid sequence of SEQ ID NO: 621. In some embodiments, the IGF-1 variant comprises the amino acid sequence of SEQ ID NO: 622. In some embodiments, the IGF-1 variant comprises the amino acid sequence of SEQ ID NO: 623. In some embodiments, the IGF-1 variant comprises the amino acid sequence of SEQ ID NO: 624. In some embodiments, the IGF-1 variant comprises the amino acid sequence of SEQ ID NO: 625. In some embodiments, the IGF-1 variant comprises the amino acid sequence of SEQ ID NO: 626. In some embodiments, the IGF-1 variant comprises the amino acid sequence of SEQ ID NO: 627. In some embodiments, the IGF-1 variant comprises the amino acid sequence of SEQ ID NO: 628. In some embodiments, the IGF-1 variant comprises the amino acid sequence of SEQ ID NO: 629. In some embodiments, the IGF-1 variant comprises the amino acid sequence of SEQ ID NO: 630. In some embodiments, the IGF-1 variant comprises the amino acid sequence of SEQ ID NO: 631. In some embodiments, the IGF-1 variant comprises the amino acid sequence of SEQ ID NO: 632. In some embodiments, the IGF-1 variant comprises the amino acid sequence of SEQ ID NO: 633. In some embodiments, the IGF-1 variant comprises the amino acid sequence of SEQ ID NO: 634. In some embodiments, the IGF-1 variant comprises the amino acid sequence of SEQ ID NO: 635. In some embodiments, the IGF-1 variant comprises the amino acid sequence of SEQ ID NO: 636. In some embodiments, the IGF-1 variant comprises the amino acid sequence of SEQ ID NO: 637. In some embodiments, the IGF-1 variant comprises the amino acid sequence of SEQ ID NO: 638. In some embodiments, the IGF-1 variant comprises the amino acid sequence of SEQ ID NO: 639. In some embodiments, the IGF-1 variant comprises the amino acid sequence of SEQ ID NO: 640. In some embodiments, the IGF-1 variant comprises the amino acid sequence of SEQ ID NO: 641. In some embodiments, the IGF-1 variant comprises the amino acid sequence of SEQ ID NO: 642. In some embodiments, the IGF-1 variant comprises the amino acid sequence of SEQ ID NO: 643. In some embodiments, the IGF-1 variant comprises the amino acid sequence of SEQ ID NO: 644. In some embodiments, the IGF-1 variant comprises the amino acid sequence of SEQ ID NO: 645. In some embodiments, the IGF-1 variant comprises the amino acid sequence of SEQ ID NO: 646. In some embodiments, the IGF-1 variant comprises the amino acid sequence of SEQ ID NO: 647. In some embodiments, the IGF-1 variant comprises the amino acid sequence of SEQ ID NO: 648. In some embodiments, the IGF-1 variant comprises the amino acid sequence of SEQ ID NO: 649. In some embodiments, the IGF-1 variant comprises the amino acid sequence of SEQ ID NO: 650. In some embodiments, the IGF-1 variant comprises the amino acid sequence of SEQ ID NO: 651. In some embodiments, the IGF-1 variant comprises the amino acid sequence of SEQ ID NO: 652. In some embodiments, the IGF-1 variant comprises the amino acid sequence of SEQ ID NO: 653. In some embodiments, the IGF-1 variant comprises the amino acid sequence of SEQ ID NO: 654. In some embodiments, the IGF-1 variant comprises the amino acid sequence of SEQ ID NO: 655. In some embodiments, the IGF-1 variant comprises the amino acid sequence of SEQ ID NO: 656. In some embodiments, the IGF-1 variant comprises the amino acid sequence of SEQ ID NO: 657. In some embodiments, the IGF-1 variant comprises the amino acid sequence of SEQ ID NO: 658. In some embodiments, the IGF-1 variant comprises the amino acid sequence of SEQ ID NO: 659. In some embodiments, the IGF-1 variant comprises the amino acid sequence of SEQ ID NO: 660. In some embodiments, the IGF-1 variant comprises the amino acid sequence of SEQ ID NO: 661. In some embodiments, the IGF-1 variant comprises the amino acid sequence of SEQ ID NO: 662. In some embodiments, the IGF-1 variant comprises the amino acid sequence of SEQ ID NO: 663. In some embodiments, the IGF-1 variant comprises the amino acid sequence of SEQ ID NO: 664. In some embodiments, the IGF-1 variant comprises the amino acid sequence of SEQ ID NO: 665. In some embodiments, the IGF-1 variant comprises the amino acid sequence of SEQ ID NO: 666. In some embodiments, the IGF-1 variant comprises the amino acid sequence of SEQ ID NO: 667. In some embodiments, the IGF-1 variant comprises the amino acid sequence of SEQ ID NO: 668. In some embodiments, the IGF-1 variant comprises the amino acid sequence of SEQ ID NO: 669. In some embodiments, the IGF-1 variant comprises the amino acid sequence of SEQ ID NO: 670. In some embodiments, the IGF-1 variant comprises the amino acid sequence of SEQ ID NO: 671. In some embodiments, the IGF-1 variant comprises the amino acid sequence of SEQ ID NO: 672. In some embodiments, the IGF-1 variant comprises the amino acid sequence of SEQ ID NO: 673. In some embodiments, the IGF-1 variant comprises the amino acid sequence of SEQ ID NO: 674. In some embodiments, the IGF-1 variant comprises the amino acid sequence of SEQ ID NO: 675. In some embodiments, the IGF-1 variant comprises the amino acid sequence of SEQ ID NO: 676. In some embodiments, the IGF-1 variant comprises the amino acid sequence of SEQ ID NO: 677. In some embodiments, the IGF-1 variant comprises the amino acid sequence of SEQ ID NO: 678. In some embodiments, the IGF-1 variant comprises the amino acid sequence of SEQ ID NO: 679. In some embodiments, the IGF-1 variant comprises the amino acid sequence of SEQ ID NO: 680. In some embodiments, the IGF-1 variant comprises the amino acid sequence of SEQ ID NO: 681. In some embodiments, the IGF-1 variant comprises the amino acid sequence of SEQ ID NO: 682. In some embodiments, the IGF-1 variant comprises the amino acid sequence of SEQ ID NO: 683. In some embodiments, the IGF-1 variant comprises the amino acid sequence of SEQ ID NO: 684. In some embodiments, the IGF-1 variant comprises the amino acid sequence of SEQ ID NO: 685. In some embodiments, the IGF-1 variant comprises the amino acid sequence of SEQ ID NO: 686. In some embodiments, the IGF-1 variant comprises the amino acid sequence of SEQ ID NO: 687. In some embodiments, the IGF-1 variant comprises the amino acid sequence of SEQ ID NO: 688. In some embodiments, the IGF-1 variant comprises the amino acid sequence of SEQ ID NO: 689. In some embodiments, the IGF-1 variant comprises the amino acid sequence of SEQ ID NO: 690. In some embodiments, the IGF-1 variant comprises the amino acid sequence of SEQ ID NO: 691. In some embodiments, the IGF-1 variant comprises the amino acid sequence of SEQ ID NO: 692. In some embodiments, the IGF-1 variant comprises the amino acid sequence of SEQ ID NO: 693. In some embodiments, the IGF-1 variant comprises the amino acid sequence of SEQ ID NO: 694. In some embodiments, the IGF-1 variant comprises the amino acid sequence of SEQ ID NO: 695. In some embodiments, the IGF-1 variant comprises the amino acid sequence of SEQ ID NO: 696. In some embodiments, the IGF-1 variant comprises the amino acid sequence of SEQ ID NO: 697. In some embodiments, the IGF-1 variant comprises the amino acid sequence of SEQ ID NO: 698. In some embodiments, the IGF-1 variant comprises the amino acid sequence of SEQ ID NO: 699. In some embodiments, the IGF-1 variant comprises the amino acid sequence of SEQ ID NO: 700. In some embodiments, the IGF-1 variant comprises the amino acid sequence of SEQ ID NO: 701. In some embodiments, the IGF-1 variant comprises the amino acid sequence of SEQ ID NO: 702. In some embodiments, the IGF-1 variant comprises the amino acid sequence of SEQ ID NO: 703. In some embodiments, the IGF-1 variant comprises the amino acid sequence of SEQ ID NO: 704. In some embodiments, the IGF-1 variant comprises the amino acid sequence of SEQ ID NO: 705. In some embodiments, the IGF-1 variant comprises the amino acid sequence of SEQ ID NO: 706. In some embodiments, the IGF-1 variant comprises the amino acid sequence of SEQ ID NO: 707. In some embodiments, the IGF-1 variant comprises the amino acid sequence of SEQ ID NO: 708. In some embodiments, the IGF-1 variant comprises the amino acid sequence of SEQ ID NO: 709. In some embodiments, the IGF-1 variant comprises the amino acid sequence of SEQ ID NO: 710. In some embodiments, the IGF-1 variant comprises the amino acid sequence of SEQ ID NO: 711. In some embodiments, the IGF-1 variant comprises the amino acid sequence of SEQ ID NO: 712. In some embodiments, the IGF-1 variant comprises the amino acid sequence of SEQ ID NO: 713. In some embodiments, the IGF-1 variant comprises the amino acid sequence of SEQ ID NO: 714. In some embodiments, the IGF-1 variant comprises the amino acid sequence of SEQ ID NO: 715. In some embodiments, the IGF-1 variant comprises the amino acid sequence of SEQ ID NO: 716. In some embodiments, the IGF-1 variant comprises the amino acid sequence of SEQ ID NO: 717. In some embodiments, the IGF-1 variant comprises the amino acid sequence of SEQ ID NO: 718. In some embodiments, the IGF-1 variant comprises the amino acid sequence of SEQ ID NO: 719. In some embodiments, the IGF-1 variant comprises the amino acid sequence of SEQ ID NO: 720. In some embodiments, the IGF-1 variant comprises the amino acid sequence of SEQ ID NO: 721. In some embodiments, the IGF-1 variant comprises the amino acid sequence of SEQ ID NO: 722. In some embodiments, the IGF-1 variant comprises the amino acid sequence of SEQ ID NO: 723. In some embodiments, the IGF-1 variant comprises the amino acid sequence of SEQ ID NO: 724. In some embodiments, the IGF-1 variant comprises the amino acid sequence of SEQ ID NO: 725. In some embodiments, the IGF-1 variant comprises the amino acid sequence of SEQ ID NO: 726. In some embodiments, the IGF-1 variant comprises the amino acid sequence of SEQ ID NO: 727. In some embodiments, the IGF-1 variant comprises the amino acid sequence of SEQ ID NO: 728. In some embodiments, the IGF-1 variant comprises the amino acid sequence of SEQ ID NO: 729. In some embodiments, the IGF-1 variant comprises the amino acid sequence of SEQ ID NO: 730. In some embodiments, the IGF-1 variant comprises the amino acid sequence of SEQ ID NO: 731. In some embodiments, the IGF-1 variant comprises the amino acid sequence of SEQ ID NO: 732. In some embodiments, the IGF-1 variant comprises the amino acid sequence of SEQ ID NO: 733. In some embodiments, the IGF-1 variant comprises the amino acid sequence of SEQ ID NO: 734. In some embodiments, the IGF-1 variant comprises the amino acid sequence of SEQ ID NO: 735. In some embodiments, the IGF-1 variant comprises the amino acid sequence of SEQ ID NO: 736. In some embodiments, the IGF-1 variant comprises the amino acid sequence of SEQ ID NO: 737. In some embodiments, the IGF-1 variant comprises the amino acid sequence of SEQ ID NO: 738. In some embodiments, the IGF-1 variant comprises the amino acid sequence of SEQ ID NO: 739. In some embodiments, the IGF-1 variant comprises the amino acid sequence of SEQ ID NO: 740. In some embodiments, the IGF-1 variant comprises the amino acid sequence of SEQ ID NO: 741. In some embodiments, the IGF-1 variant comprises the amino acid sequence of SEQ ID NO: 742. In some embodiments, the IGF-1 variant comprises the amino acid sequence of SEQ ID NO: 743. In some embodiments, the IGF-1 variant comprises the amino acid sequence of SEQ ID NO: 744. In some embodiments, the IGF-1 variant comprises the amino acid sequence of SEQ ID NO: 745. In some embodiments, the IGF-1 variant comprises the amino acid sequence of SEQ ID NO: 746. In some embodiments, the IGF-1 variant comprises the amino acid sequence of SEQ ID NO: 747. In some embodiments, the IGF-1 variant comprises the amino acid sequence of SEQ ID NO: 748. In some embodiments, the IGF-1 variant comprises the amino acid sequence of SEQ ID NO: 749. In some embodiments, the IGF-1 variant comprises the amino acid sequence of SEQ ID NO: 750. In some embodiments, the IGF-1 variant comprises the amino acid sequence of SEQ ID NO: 751. In some embodiments, the IGF-1 variant comprises the amino acid sequence of SEQ ID NO: 752. In some embodiments, the IGF-1 variant comprises the amino acid sequence of SEQ ID NO: 753. In some embodiments, the IGF-1 variant comprises the amino acid sequence of SEQ ID NO: 754. In some embodiments, the IGF-1 variant comprises the amino acid sequence of SEQ ID NO: 755. In some embodiments, the IGF-1 variant comprises the amino acid sequence of SEQ ID NO: 756. In some embodiments, the IGF-1 variant comprises the amino acid sequence of SEQ ID NO: 757. In some embodiments, the IGF-1 variant comprises the amino acid sequence of SEQ ID NO: 758. In some embodiments, the IGF-1 variant comprises the amino acid sequence of SEQ ID NO: 759. In some embodiments, the IGF-1 variant comprises the amino acid sequence of SEQ ID NO: 760. In some embodiments, the IGF-1 variant comprises the amino acid sequence of SEQ ID NO: 761. In some embodiments, the IGF-1 variant comprises the amino acid sequence of SEQ ID NO: 762. In some embodiments, the IGF-1 variant comprises the amino acid sequence of SEQ ID NO: 763. In some embodiments, the IGF-1 variant comprises the amino acid sequence of SEQ ID NO: 764. In some embodiments, the IGF-1 variant comprises the amino acid sequence of SEQ ID NO: 765. In some embodiments, the IGF-1 variant comprises the amino acid sequence of SEQ ID NO: 766. In some embodiments, the IGF-1 variant comprises the amino acid sequence of SEQ ID NO: 767. In some embodiments, the IGF-1 variant comprises the amino acid sequence of SEQ ID NO: 768. In some embodiments, the IGF-1 variant comprises the amino acid sequence of SEQ ID NO: 769. In some embodiments, the IGF-1 variant comprises the amino acid sequence of SEQ ID NO: 770. In some embodiments, the IGF-1 variant comprises the amino acid sequence of SEQ ID NO: 771.

The engineered polypeptide may comprise or consist of about 66 amino acids. The engineered polypeptide may comprise or consist of about 67 amino acids. The engineered polypeptide may comprise or consist of about 69 amino acids. The engineered polypeptide may comprise or consist of about 150 amino acids. The engineered polypeptide may comprise or consist of about 70 amino acids. The engineered polypeptide may comprise or consist of about 83 amino acids. The engineered polypeptide may comprise or consist of about 105 amino acids.

The engineered polypeptide may have a decreased systemic and/or local half-life upon administration to a healthy adult compared to native human IGF-1. In some embodiments, the systemic and/or local half-life of the engineered polypeptide upon administration to a healthy adult decreases at least 5% compared to native human IGF-1. In some embodiments, the systemic and/or local half-life of the engineered polypeptide upon administration to a healthy adult decreases at least 10% compared to native human IGF-1. In some embodiments, the systemic and/or local half-life of the engineered polypeptide upon administration to a healthy adult decreases at least 15% compared to native human IGF-1. In some embodiments, the systemic and/or local half-life of the engineered polypeptide upon administration to a healthy adult decreases at least 20% compared to native human IGF-1. In some embodiments, the systemic and/or local half-life of the engineered polypeptide upon administration to a healthy adult decreases at least 30% compared to native human IGF-1. In some embodiments, the systemic and/or local half-life of the engineered polypeptide upon administration to a healthy adult decreases at least 40% compared to native human IGF-1. In some embodiments, the systemic and/or local half-life of the engineered polypeptide upon administration to a healthy adult decreases at least 50% compared to native human IGF-1.

The engineered polypeptide may have reduced affinity to at least one IGFBP relative to native human IGF-1 (SEQ ID NO: 1) to the IGFBP. In some embodiments, the engineered polypeptide has reduced affinity to IGFBP1 relative to native human IGF-1 (SEQ ID NO: 1). In some embodiments, the engineered polypeptide has reduced affinity to IGFBP2 relative to native human IGF-1 (SEQ ID NO: 1). In some embodiments, the engineered polypeptide has reduced affinity to IGFBP3 relative to native human IGF-1 (SEQ ID NO: 1). In some embodiments, the engineered polypeptide has reduced affinity to IGFBP4 relative to native human IGF-1 (SEQ ID NO: 1). In some embodiments, the engineered polypeptide has reduced affinity to IGFBP5 relative to native human IGF-1 (SEQ ID NO: 1). In some embodiments, the engineered polypeptide has reduced affinity to IGFBP6 relative to native human IGF-1 (SEQ ID NO: 1). In some embodiments, the engineered polypeptide has reduced affinity to IGFBP3 and IGFBP2 relative to native human IGF-1 (SEQ ID NO: 1).

In some embodiments, the engineered polypeptide may further comprise a 13 amino acid sequence (MFPAMPLLSLFVN (SEQ ID NO: 13)) at its C-terminal or N-terminal sequence.

IGFBPs play a key role in extending the systemic half-life of Insulin-like Growth Factor 1 (IGF-1) in the human body. While the unbound form of wild-type IGF-1 has a systemic half-life of 10 to 20 minutes, the binding of IGF-1 to IGFBPs increases its half-life to several hours. This extended half-life is generally thought to increase the duration of IGF-1's biological effect. This is most clearly seen in Laron Syndrome, a form of dwarfism in which patients are unable to make, among other things, IGFBPs. Laron dwarfs given high doses of systemic IGF1 do not grow very much, in part because IGF-1 cannot persist in circulation without the dramatic half-life extension provided by IGFBP binding.

It is therefore surprising that, as described herein, when IGF1 variants that evade IGFBPs are locally administered (e.g., as an eye drop to treat meibomian gland dysfunction), that the opposite phenomenon is observed. For instance, the desired pharmacodynamic effects of wild-type IGF-1 treatment are seen at lower levels and across shorter time frames relative to IGF-1 variants (e.g., IGF1 LR3, IGF des 1-3, IGF E3R) that possess the ability to evade one or more IGFBPs. Stated differently, unexpectedly, relative to wild-type IGF-1 (SEQ ID NO: 1), variants that evade one or more IGF-1 binding proteins (e.g., IGFBP2) can exhibit extended and/or improved pharmacodynamic effects when administered locally, even though binding to one or more binding proteins has been understood as being important to extending half-life and function of IGF-1.

### Cell-Penetrating Peptides (CPPs) and Skin-Penetrating Peptides (SPPs)

Engineered polypeptides as disclosed herein may further comprise a cell-penetrating peptide (CPP) or a skin-penetrating peptide (SPP). The stratum corneum of skin generally comprises keratin-enriched dead cells floating in layered, lipid domains, and this functions as a barrier to the environment. This structure may inhibit absorption and transport of macromolecules to the dermis and beyond. CPPs or SPPs as disclosed herein may have high transduction efficiency, thereby enabling transdermal delivery. CPPs or SPPs as disclosed herein may modify the structure of a skin barrier to allow molecules that they are co-formulated with (even if not conjugated to) to enter and/or translocate across the skin.

The IGF-1 variant can be coupled to a cell-penetrating peptide (CPP) or skin-penetrating peptide (SPP) described herein. A CPP or SPP may comprise any one of SEQ ID NOS: 9-11. In some embodiments, a CPP or SPP comprises SEQ ID NO: 9. In some embodiments, a CPP or SPP comprises SEQ ID NO: 10. In some embodiments, a CPP or SPP comprises SEQ ID NO: 11. In some embodiments, an engineered polypeptide comprising any one of SEQ ID NOS: 2-8, 12, or any combination thereof is coupled to a CPP or SPP. In some embodiments, an engineered polypeptide comprising SEQ ID NO: 1 is coupled to a CPP or SPP. In some embodiments, an engineered polypeptide comprising SEQ ID NO: 2 is coupled to a CPP or SPP. In some embodiments, an engineered polypeptide comprising SEQ ID NO: 3 is coupled to a CPP or SPP. In some embodiments, an engineered polypeptide comprising SEQ ID NO: 4 is coupled to a CPP or SPP. In some embodiments, an engineered polypeptide comprising SEQ ID NO: 5 is coupled to a CPP or SPP. In some embodiments, an engineered polypeptide comprising SEQ ID NO: 6 is coupled to a CPP or SPP. In some embodiments, an engineered polypeptide comprising SEQ ID NO: 7 is coupled to a CPP or SPP. In some embodiments, an engineered polypeptide comprising SEQ ID NO: 8 is coupled to a CPP or SPP. In some embodiments, an engineered polypeptide comprising SEQ ID NO: 12 is coupled to a CPP or SPP.

Compositions or formulations delivered transdermally may be configured to better reach steady state level as compared to compositions or formulations not delivered by transdermal delivery. Compositions or formulations delivered transdermally may also be capable of bypassing hepatic metabolism, and reduce unwanted systemic side effects, thereby increasing patient compliance. CPPs or SPPs as disclosed herein may comprise or consist of about 5 to about 30 amino acids. CPPs or SPPs as disclosed herein may enable an engineered polypeptide to penetrate skin on an eyelid, thereby enabling the engineered polypeptide to become absorbed into the eyelid. SPPs as disclosed herein may comprise or consist of a hydrophobic peptide. CPPs as disclosed herein comprise or consist of a cationic peptide. The cationic peptide may have one or more charged amino acids, e.g., arginine. The CPP or SPP may present low cytotoxicity to cells, e.g., human cells. SPPs as disclosed herein may comprise a macromolecule transduction domain (MTD). The MTD may comprise or consist of MRAAAPAVAA (SEQ ID NO: 9). The MTD may be derived from a membrane translocation sequence (MTS) of a Kaposi fibroblast growth factor (FGF-4) signal peptide. CPPs as disclosed herein may comprise or consist of SEQ ID NO: 10. The engineered polypeptide of SEQ ID NO: 10 may be penetrate the epidermis and dermis of skin, and may be useful in treating antioxidant disorders. The engineered polypeptide of SEQ ID NO: 10 may be delivered via, e.g., a skin spray. CPPs as disclosed herein may comprise or consist of SEQ ID NO: 11. The engineered polypeptide of SEQ ID NO: 11 may be penetrate the epidermis and dermis of skin, and may be useful in treating antioxidant disorders. The engineered polypeptide of SEQ ID NO: 11 may be delivered via, e.g., a skin spray.

### Pharmaceutical Compositions

The present disclosure provides pharmaceutical compositions for treating a disease and disorder. The disease or disorder may include, for example, Meibomian glands or dry eye disease. The pharmaceutical composition may be applied topically, e.g., as eye drops or as a cream. The pharmaceutical composition may be applied to an eyelid. The pharmaceutical composition may be formulated for systemic delivery. The pharmaceutical composition may penetrate an eyelid.

The pharmaceutical composition may comprise a pharmaceutically acceptable carrier or adjuvant, such as, for example, a hyaluronate (or hyaluronic acid), an electrolyte, an ophthalmic demulcent, an excipient, an astringent, a vasoconstrictor and/or an emollient. Examples of pharmaceutically acceptable excipients may include the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone. Examples of electrolytes may include sodium chloride, potassium chloride, sodium bicarbonate, potassium bicarbonate, calcium chloride, magnesium chloride, trisodium citrate, hydrochloric acid, sodium hydroxide, and mixtures thereof. Pharmaceutical compositions as disclosed herein may comprise a solution having one or more electrolytes. For instance, in some embodiments, the electrolyte-containing solution comprises one or more of sodium chloride, potassium chloride, sodium bicarbonate, potassium bicarbonate, calcium chloride, magnesium chloride, trisodium citrate, hydrochloric acid, or sodium hydroxide. In some instances, the mole percent of sodium chloride is from about 40% to about 60%. In some instances, the mole percent of sodium chloride is about 40%, 45%, 50%, 55%, or 60%. In some instances, the mole percent of potassium chloride is about 1% to about 20%. In some instances, the mole percent of potassium chloride is about 1%, 2%, 3%, 4%, 5%, 10%, 15%, or 20%. In some instances, the mole percent of sodium bicarbonate is from about 1% to about 25%. In some instances, the mole percent of sodium bicarbonate is about 1%, 2%, 3%, 4%, 5%, 10%, 15%, 20%, or 25%. In some instances, the mole percent of potassium bicarbonate is about 0% to about 10%. In some instances, the mole percent of potassium bicarbonate is about 0%, 1%, 2%, 3%, 4%, 5%, 6%, 7%, 8%, 9%, or 10%. In some instances, the mole percent of calcium chloride is about 0% to 10% of In some instances, the mole percent of calcium chloride is about 0%, 1%, 2%, 3%, 4%, 5%, 6%, 7%, 8%, 9%, or 10%. In some instances, the mole percent of magnesium chloride is about 0% to 10% of In some instances, the mole percent of magnesium chloride is about 0%, 1%, 2%, 3%, 4%, 5%, 6%, 7%, 8%, 9%, or 10%. In some instances, the mole percent of trisodium citrate is about 0% to 10% of. In some instances, the mole percent of trisodium citrate is about 0%, 1%, 2%, 3%, 4%, 5%, 6%, 7%, 8%, 9%, or 10%. In some instances, the mole percent of hydrochloric acid is about 0% to about 30%. In some instances, the mole percent of hydrochloric acid is about 0%, 1%, 2%, 3%, 4%, 5%, 6%, 7%, 8%, 9%, or 10%, 15%, 20%, 25%, or 30%. In some instances, the mole percent of sodium hydroxide about 0% to about 30%. In some instances, the mole percent of sodium hydroxide about 0%, 1%, 2%, 3%, 4%, 5%, 6%, 7%, 8%, 9%, or 10%, 15%, 20%, 25%, or 30%.

In some embodiments, excipients may include one or more permeation enhancers. The term “permeation enhancer” or “penetration enhancer” may refer to a compound or mixture of compounds that interact with one or more skin constituents to promote drug penetration into and/or through the skin (e.g., through skin on an outer eyelid to one or more Meibomian glands). Any suitable permeation enhancer known in the art may be used in the pharmaceutical compositions described herein, including, for example, surfactants (e.g., ionic (anionic, cationic, zwitterionic) surfactants (such as sodium lauryl sulfate, sodium laureate, etc.) non-ionic surfactants (such as Tween-80, other polysorbates, etc.), and any combinations thereof), bile salts and derivatives thereof (e.g, sodium glyacolate, sodium deoxycholate, etc.), fatty acids and derivatives thereof (e.g, oleic acid, caprylic acid, esters of fatty acids such as isopropyl myrisate, etc.), chelating agents (e.g, EDTA, citric acid, etc.), sulphoxides (e.g, DMSO, DMA, DMF, etc.), polyols (e.g, diethylene glycol monoethyl ether, PG, polyethylene glycols (PEGs), glycerol, polyglycols, etc.), alcohols (e.g, alkanols, alkenols, glycols, etc.), hydrocarbons (e.g, alkanes, alkenes, halogenated alkanes, squalene, squalene, mineral oil, etc.), amines, amides (e.g, cyclic amides, acyclic amides, azones, pyrrolidones, urea and derivatives thereof, etc.), others (e.g, terpenes and terpenoids, essential oils (such as eucalyptus oil, peppermint oil, turpentine oil, etc.), phospholipids, cyclic oligosaccharides (such as cyclodextrins), amino acids and thioacyl derivatives of amino acids, alkyl amino esters and oxazolidinones, enzymes, ketones (such as macrocyclic ketones), etc.), hyaluronic acid, benzalkonium chloride, and any combinations thereof.

In some embodiments, the pharmaceutically acceptable excipient(s)s are adapted for transdermal delivery through an outer eyelid by formulating the excipient(s) to 1) achieve improved spreadability on the outer eyelid surface; and/or 2) avoid flow from the outer eyelid surface onto the corneal surface. In some embodiments, the pharmaceutically acceptable excipient(s) comprise characteristics and rheological features of improved spreadability, resulting in easier administration and spreading onto the eyelid surface, and absence of flow at the body temperature of a subject, particularly after being applied to the skin of a subject. In some embodiments, pharmaceutical compositions of the present disclosure are formulated such that the cohesiveness of the formulation does not significantly change after application to the skin (e.g., eyelids) of a subject. Examples of suitable additives that convey a suitable cohesiveness of the formulation may include, for example, additives that increase viscosity of the formulation such as waxes, paraffins, and elastomers. In some embodiments, the viscosity of the formulation does not significantly change when heated from room temperature to a temperature closer to the body temperature of a subject.

In some embodiments, the pharmaceutically acceptable formulation is an ointment comprising a water-miscible ointment base. In some embodiments, the pharmaceutically acceptable formulation is an ointment comprising a paraffinic ointment base. In some embodiments, the ointment comprises one or more of white soft paraffin, mineral oil, propylene glycol, ST cyclomethicone-5NF, labrasol, propylene carbonate, steareth 2, ST emulsifier 10, and ST elastomer-10. In some embodiments, the ointment comprises white soft paraffin, mineral oil, propylene glycol, ST cyclomethicone-5NF, labrasol, propylene carbonate, steareth 2, ST emulsifier 10, and ST elastomer-10.

In some embodiments, the pharmaceutically acceptable formulation is a cream comprising an oil-in-water base. In some embodiments, the pharmaceutically acceptable formulation is a cream comprising a water-in-oil base. In some embodiments, the cream comprises one or of white soft paraffin/petrolatum, mineral oil, propylene glycol, cyclomethicone, ST-cyclomethicone-5NF, emulsifier 10, ST-emulsifier, ST-elastomer-10, methylparaben, dibasic sodium phosphate, citric acid, propylparaben, and purified water. In some embodiments, the cream comprises white soft paraffin/petrolatum, mineral oil, propylene glycol, ST-cyclomethicone-5NF, ST-emulsifier, ST-elastomer-10, methylparaben, dibasic sodium phosphate, citric acid, propylparaben, and purified water. In some embodiments, the cream comprises white soft paraffin/petrolatum, mineral oil, propylene glycol, cyclomethicone, emulsifier 10, ST-elastomer-10, methylparaben, sodium phosphate dibasic anhydrous, citric acid anhydrous, propylparaben, and purified water.

Pharmaceutical compositions as disclosed herein may be administered to a subject. A therapeutically effective amount of a pharmaceutical composition as disclosed herein may be administered to a subject. The pharmaceutical composition may be administered intravenously, intraperitoneally, subcutaneously, intramuscularly, topically or intradermally. The pharmaceutical composition may be administered through any mode of delivery as disclosed herein, e.g., eye drops or cream. The eye drops may be administered via an eyedropper. The pharmaceutical composition may be administered to an eye of a subject or a portion thereof. The pharmaceutical composition may be administered to an eyelid, e.g., an outer eyelid.

In one aspect, the subject may suffer from a Holocrine gland dysfunction. In some embodiments, the Holocrine gland is a Meibomian gland. In some embodiments, the subject may suffer from a Sebaceous gland dysfunction. In some embodiments, the Sebaceous gland is a Meibomian gland. In some embodiments, the subject may suffer from a Meibomian gland dysfunction. Meibomian gland dysfunction can lead to altered tear film composition, ocular surface disease, ocular and eyelid discomfort, and evaporative dry eye. Symptoms of Meibomian gland dysfunction comprise dryness, burning, itching, redness, crusty discharge, watery eyes, blurred vision, and light sensitivity.

In some embodiments, an administration of pharmaceutical compositions as disclosed herein to the subject may result in an increase in a size of the meibomian gland as compared to subjects who do not receive the pharmaceutical composition. In some embodiments, an administration of pharmaceutical compositions as disclosed herein to the subject may result in a decrease in meibomian gland atrophy as compared to subjects who do not receive the pharmaceutical composition. In some embodiments, an administration of pharmaceutical compositions as disclosed herein to the subject may result in regeneration of meibomian glands as compared to subjects who do not receive the pharmaceutical composition. In some embodiments, an administration of pharmaceutical compositions as disclosed herein to the subject may result in a reversal of age-associated meibomian gland atrophy as compared to subjects who do not receive the pharmaceutical composition. In some embodiments, an administration of pharmaceutical compositions as disclosed herein to the subject may result in a median increase in the number of meibocytes within a Meibomian gland of the subject as compared to subjects who do not receive the pharmaceutical composition. In some embodiments, an administration of pharmaceutical compositions as disclosed herein to the subject may result in an increase in a function of one or more meibocytes as compared to subjects who do not receive the pharmaceutical composition. For example, in some instances, the increase in function may comprises increase phosphorylation of Akt (or increased duration in activity for phosphorylation or Akt), IGF1R itself or other downstream signaling molecules that are phosphorylated when IGF1R is activated. In some embodiments, an administration of pharmaceutical compositions as disclosed herein to the subject may result in an increase in corneal proliferation and/or repair as compared to subjects who do not receive the pharmaceutical composition. In some embodiments, an administration of pharmaceutical compositions as disclosed herein to the subject may result in an increase in corneal healing as compared to subjects who do not receive the pharmaceutical composition. In some embodiments, an administration of pharmaceutical compositions as disclosed herein to the subject may result in an increase in IGF1 receptor (IGF1R) activation in the meibomian glands as compared to subjects who do not receive the pharmaceutical composition. In some embodiments, an administration of pharmaceutical compositions as disclosed herein to the subject may result in an increase duration of IGF1R activation in the meibomian glands as compared to subjects who do not receive the pharmaceutical composition. In some embodiments, an administration of pharmaceutical compositions as disclosed herein to the subject may result in an increase of lipid content of the meibomian glands as compared to subjects who do not receive the pharmaceutical composition.

The administration of the pharmaceutical composition to the subject suffering from Meibomian gland dysfunction may result in a median increase in surface area or volume of meibomian glands of the subject as compared to subjects who do not receive the pharmaceutical composition. In some embodiments, the increase in surface area or volume of meibomian glands within the inner eyelid surface of the subject is at least 5% compared to subjects who do not receive the pharmaceutical composition. In some embodiments, the increase in surface area or volume of meibomian glands of the subject is at least 6% compared to subjects who do not receive the pharmaceutical composition. In some embodiments, the increase in surface area or volume of meibomian glands of the subject is at least 7% compared to subjects who do not receive the pharmaceutical composition. In some embodiments, the increase in surface area or volume of meibomian glands of the subject is at least 8% compared to subjects who do not receive the pharmaceutical composition. In some embodiments, the increase in surface area or volume of meibomian glands of the subject is at least 9% compared to subjects who do not receive the pharmaceutical composition. In some embodiments, the increase in surface area or volume of meibomian of the subject is at least 10% compared to subjects who do not receive the pharmaceutical composition. In some embodiments, the increase in surface area or volume of meibomian glands of the subject is at least 15% compared to subjects who do not receive the pharmaceutical composition. In some embodiments, the increase in surface area or volume of meibomian glands of the subject is at least 20% compared to subjects who do not receive the pharmaceutical composition. In some embodiments, the increase in surface area or volume of meibomian glands of the subject is at least 30% compared to subjects who do not receive the pharmaceutical composition. In some embodiments, the increase in surface area or volume of meibomian glands of the subject is at least 40% compared to subjects who do not receive the pharmaceutical composition. In some embodiments, the increase in surface area or volume of meibomian glands of the subject is at least 50% compared to subjects who do not receive the pharmaceutical composition. In some embodiments, the increase in surface area or volume of meibomian glands of the subject is at least 60% compared to subjects who do not receive the pharmaceutical composition.

The administration of the pharmaceutical composition to the subject suffering from Meibomian gland dysfunction may result in a median increase in lipid content within Meibomian glands as compared to subjects who do not receive the pharmaceutical composition. In some embodiments, the increase in lipid content within Meibomian glands is at least 5% compared to subjects who do not receive the pharmaceutical composition. In some embodiments, the increase in lipid content within Meibomian glands is at least 6% compared to subjects who do not receive the pharmaceutical composition. In some embodiments, the increase in lipid content within Meibomian glands is at least 7% compared to subjects who do not receive the pharmaceutical composition. In some embodiments, the increase in lipid content within Meibomian glands is at least 8% compared to subjects who do not receive the pharmaceutical composition. In some embodiments, the increase in lipid content within Meibomian glands is at least 9% compared to subjects who do not receive the pharmaceutical composition. In some embodiments, the increase in lipid content within Meibomian glands is at least 10% compared to subjects who do not receive the pharmaceutical composition. In some embodiments, the increase in lipid content within Meibomian glands is at least 15% compared to subjects who do not receive the pharmaceutical composition. In some embodiments, the increase in lipid content within Meibomian glands is at least 20% compared to subjects who do not receive the pharmaceutical composition. In some embodiments, the increase in lipid content within Meibomian glands is at least 30% compared to subjects who do not receive the pharmaceutical composition. In some embodiments, the increase in lipid content within Meibomian glands is at least 40% compared to subjects who do not receive the pharmaceutical composition. In some embodiments, the increase in lipid content within Meibomian glands is at least 50% compared to subjects who do not receive the pharmaceutical composition. In some embodiments, the increase in lipid content within Meibomian glands is at least 60% compared to subjects who do not receive the pharmaceutical composition.

The administration of the pharmaceutical composition to the subject suffering from Meibomian gland dysfunction may result in a median increase in lipid quality within Meibomian glands as compared to subjects who do not receive the pharmaceutical composition. The lipid quality may be assessed by having a lower melting point (e.g., being less waxy) that lipids (e.g., meibum) from subjects who did not receive the pharmaceutical composition. Stated differently lipids (e.g., meibum) with a lower average melting point may have improved quality relative to lipids (e.g., meibum) with a higher average melting point. In some embodiments, the increase in lipid quality within Meibomian glands is at least 5% compared to subjects who do not receive the pharmaceutical composition. In some embodiments, the increase in lipid quality within Meibomian glands is at least 6% compared to subjects who do not receive the pharmaceutical composition. In some embodiments, the increase in lipid quality within Meibomian glands is at least 7% compared to subjects who do not receive the pharmaceutical composition. In some embodiments, the increase in lipid quality within Meibomian glands is at least 8% compared to subjects who do not receive the pharmaceutical composition. In some embodiments, the increase in lipid quality within Meibomian glands is at least 9% compared to subjects who do not receive the pharmaceutical composition. In some embodiments, the increase in lipid quality within Meibomian glands is at least 10% compared to subjects who do not receive the pharmaceutical composition. In some embodiments, the increase in lipid quality within Meibomian glands is at least 15% compared to subjects who do not receive the pharmaceutical composition. In some embodiments, the increase in lipid quality within Meibomian glands is at least 20% compared to subjects who do not receive the pharmaceutical composition. In some embodiments, the increase in lipid quality within Meibomian glands is at least 30% compared to subjects who do not receive the pharmaceutical composition. In some embodiments, the increase in lipid quality within Meibomian glands is at least 40% compared to subjects who do not receive the pharmaceutical composition. In some embodiments, the increase in lipid quality within Meibomian glands is at least 50% compared to subjects who do not receive the pharmaceutical composition. In some embodiments, the increase in lipid quality within Meibomian glands is at least 60% compared to subjects who do not receive the pharmaceutical composition.

The administration of the pharmaceutical composition to the subject suffering from Meibomian gland dysfunction may result in an increase in release of lipid from acini of the Meibomian glands as compared to subjects who do not receive the pharmaceutical composition. In some embodiments, the increase in release of lipid from acini of the Meibomian glands is at least 5% compared to subjects who do not receive the pharmaceutical composition. In some embodiments, the increase in release of lipid from acini of the Meibomian glands is at least 10% compared to subjects who do not receive the pharmaceutical composition. In some embodiments, the increase in release of lipid from acini of the Meibomian glands is at least 15% compared to subjects who do not receive the pharmaceutical composition. In some embodiments, the increase in release of lipid from acini of the Meibomian glands is at least 20% compared to subjects who do not receive the pharmaceutical composition. In some embodiments, the increase in release of lipid from acini of the Meibomian glands is at least 30% compared to subjects who do not receive the pharmaceutical composition. In some embodiments, the increase in release of lipid from acini of the Meibomian glands is at least 40% compared to subjects who do not receive the pharmaceutical composition. In some embodiments, the increase in release of lipid from acini of the Meibomian glands is at least 50% compared to subjects who do not receive the pharmaceutical composition. In some embodiments, the increase in release of lipid from acini of the Meibomian glands is at least 75% compared to subjects who do not receive the pharmaceutical composition. In some embodiments, the increase in release of lipid from acini of the Meibomian glands is at least 100% compared to subjects who do not receive the pharmaceutical composition. In some embodiments, the increase in release of lipid from acini of the Meibomian glands is at least 150% compared to subjects who do not receive the pharmaceutical composition. In some embodiments, the increase in release of lipid from acini of the Meibomian glands is at least 200% compared to subjects who do not receive the pharmaceutical composition. In some embodiments, the increase in release of lipid from acini of the Meibomian glands is at least 300% compared to subjects who do not receive the pharmaceutical composition.

The administration of the pharmaceutical composition to the subject suffering from Meibomian gland dysfunction may result in an increase in one or more pharmacodynamic effects, such as an increase in duration of phosphorylation of Akt in meibocytes as compared to subjects who do not receive the pharmaceutical composition. In some embodiments, the increase in duration of phosphorylation of Akt in meibocytes is at least 5% compared to subjects who do not receive the pharmaceutical composition. In some embodiments, the increase in duration of phosphorylation of Akt in meibocytes is at least 10% compared to subjects who do not receive the pharmaceutical composition. In some embodiments, the increase in duration of phosphorylation of Akt in meibocytes is at least 15% compared to subjects who do not receive the pharmaceutical composition. In some embodiments, the increase in duration of phosphorylation of Akt in meibocytes is at least 20% compared to subjects who do not receive the pharmaceutical composition. In some embodiments, the increase in duration of phosphorylation of Akt in meibocytes is at least 30% compared to subjects who do not receive the pharmaceutical composition. In some embodiments, the increase in duration of phosphorylation of Akt in meibocytes is at least 40% compared to subjects who do not receive the pharmaceutical composition. In some embodiments, the increase in duration of phosphorylation of Akt in meibocytes is at least 50% compared to subjects who do not receive the pharmaceutical composition. In another embodiment, the change in one or more pharmacodynamic effects may be increased phosphorylation of any downstream target resulting from activation of the IGF1R.

The pharmaceutical composition may comprise an engineered polypeptide as disclosed herein. The engineered polypeptide may comprise the sequence of any one of SEQ ID NOS: 2-8 or 12. The engineered polypeptide may be an IGF-1 variant. The IGF-1 variant may comprise or consist of the sequence of any one of SEQ ID NOS: 2-8 or 12. The pharmaceutical composition may comprise an IGF-1 variant that has reduced affinity to at least one IGFBP relative to wild-type IGF-1 (SEQ ID NO: 1) to the IGFBP.

In some embodiments, the pharmaceutical composition does not comprise any additional phospholipidosis-inducing agent. In some embodiments, the pharmaceutical composition does not comprise any one of azithromycin or doxycycline.

In other embodiments, the pharmaceutical composition comprises a therapeutically effective amount of a non-IGF-1 growth factor. The pharmaceutical composition may comprise the non-IGF-1 growth factor alone or in combination with an IGF-1. For instance, in some embodiments, the non-IGF-1 growth factor is selected from group consisting of growth hormone, insulin-like growth factor 2 (IGF2), pappalysin, epidermal growth factor (EGF), basic fibroblast growth factor (bFGF), nerve growth factor (NGF), transforming growth factor-P (TGFb), brain-derived neurotrophic factor (BDNF), vascular endothelial growth factor (VEGF), fibroblast growth factor 17 (FGF17), fibroblast growth factor 6 (FGF6), fibroblast growth factor 9 (FGF9), fibroblast growth factor 10 (FGF10), Insulin, bone morphogenetic protein 2 (BMP2). Such pharmaceutical compositions may be used for treating various conditions, such a meibomian gland dysfunction. In some embodiments, the pharmaceutical composition is formulated for local administration (e.g., via an eyedrop).

### Method of Treatment, Administration, and Uses

Also provided herein are methods for treating a disease or a condition in a subject in need thereof. Also provided herein are methods for treating an eye disorder in a subject in need thereof. In some aspects, the method comprises administering a pharmaceutical composition provided herein. In some aspects, the method comprises administering an engineered polypeptide provided herein. In some aspects, the method comprises administering an IGF-1 variant provided herein. In some embodiments of the methods disclosed herein, the pharmaceutical composition is administered locally. In some embodiments, the subject is human. In some embodiments, a therapeutically effective amount of the pharmaceutical composition provided herein is administered.

Also provided herein is use of the pharmaceutical composition provided herein in treating an eye disorder.

The subject can have a certain disease or a condition in need of treatment provided by the present disclosure. The disease or condition can include dry eye disease, meibomian gland dysfunction, and/or Sjorgren's syndrome.

For treatment, the amount of the pharmaceutical composition provided herein administered is an amount effective in producing the desired effect, for example, treatment or amelioration of the effects and/or symptoms of an eye disorder in a subject in need thereof. An effective amount can be provided in one or a series of administrations of the pharmaceutical composition provided herein.

Subjects suffering from the eye disorder can be identified by any or a combination of diagnostic or prognostic assays known in the art.

Methods for treating a subject in need thereof can further comprise sequentially, separately, or simultaneously administering to the subject at least one additional therapy, e.g., artificial tears or punctal plugs.

In any case, the multiple therapeutic agents can be administered in any order or even simultaneously. If simultaneously, the multiple therapeutic agents can be provided in a single, unified form, or in multiple forms (by way of example only, either as a single pill or as two separate pills). One of the therapeutic agents can be given in multiple doses, or both can be given as multiple doses. If not simultaneous, the timing between the multiple doses can vary from more than zero weeks to less than four weeks. In addition, the combination methods, compositions and formulations are not to be limited to the use of only two agents.

### Kits

Provided herein is a kit that comprises an engineered polypeptide provided here and instructions for use. In some embodiments, the instructions for use recite a treatment of an eye disease or disorder.

The present disclosure also provides kits comprising a pharmaceutical composition as disclosed herein, and an eyedropper. The eyedropper may be configured for a delivery of the pharmaceutical composition as an eyedrop.

### EXAMPLES

### Example 1: In Vitro Potency of Wild-Type IGF-1 and IGF-1 Variants in the Presence and Absence of IGFBPs

### Materials and Methods

### In Vitro Potency Assay in DU145 Cells

15×10⁶ DU145 cells (ATCC #HTB-81) frozen down at passage 5 (P5) were reseeded in a T175 culture flask containing DMEM/F12 (1:1) medium (Gibco 11320-033) supplemented with 10% FBS (Gibco 10437-028) and 10 μg/mL Gentamicin (Gibco 15710-064) (“culture medium”) and expanded. At 80-90% confluency, cells were trypsinized, manually counted using a hemocytometer, resuspended at 3×10⁵ cells per mL in culture medium, and then reseeded in 24-well plates (Corning 3524) in 0.5 mL aliquots per well (150,000 cells/well). 14-16 hours after reseeding, the culture medium was removed, and following a wash with 1 mL PBS, cultured for 6 h in 225 μL serum-free culture medium supplemented with 0.2% BSA (Millipore #A3059). 25 μL aliquots of the following growth factor dilution series were added to the cells in duplicate for a final concentration in the well of: 457, 137, 41, 12.5, 3.7, 1.11, and 0.33 nM IGF-1 (SEQ ID NO: 1), IGF-1 Ea (SEQ ID NO: 7), IGF-1 Des1-3 R37X (SEQ ID NO: 6), IGF-1 Des1-3 (SEQ ID NO: 8), and IGF-1 E3R (SEQ ID NO: 4). After 15 min incubation at 37° C., medium was aspirated and cells lysed in 150 μL 1×PTR buffer (Extraction Buffer 5×PTR, Abcam ab193970) containing 1× Extraction Enhancer Buffer (Extraction Enhancer Buffer 50×, Abcam ab193971) and protease/phosphatase inhibitors (one Pierce mini tablet [#A32959] per 10 mL 1×PTR). After 15-30 min incubation on ice, plates were vortexed, and lysates transferred to Eppendorf tubes and stored at −80° C. for assessment of IGF1R-AKT signaling strength using the Human/Mouse/Rat Phospho-Akt (S473) Pan Specific DuoSet IC ELISA (RnD #DYC887B).

### Culture of IGFBP and IGF-1 in DU145 Cells

To measure the in vitro potency of IGF-1 and IGF-1 variants in the presence of IGFBP, DU145 cells were prepared as described above. During this time, IGFBPs in a 1:1, 2:1 or 4:1 molar ratio were incubated with 250 nM rhIGF1 (SEQ ID NO: 1), rhIGF1 LR3 (SEQ ID NO: 12), or rhIGF1 E3R (SEQ ID NO: 4). After 1 hour incubation at room temperature, complexes containing a final concentration of 25 nM of IGF1 variants were added to the cells in triplicate.

### Results

The dose-dependent effects of IGF-1 (SEQ ID NO: 1), IGF-1 Ea (SEQ ID NO: 7), IGF-1 Des1-3 R37X (SEQ ID NO: 6), IGF-1 Des1-3 (SEQ ID NO: 8), and IGF-1 E3R (SEQ ID NO: 4) on AKT S473 phosphorylation were measured in DU145 cells. The EC₅₀ value of IGF-1 was 6.0 nM. The EC₅₀ value of IGF-1 Ea was 8.7 nM. The EC₅₀ value of IGF-1 Des 1-3 R37X was 4.1 nM. The EC₅₀ value of IGF-1 Des 1-3 was 2.3 nM. The EC₅₀ value of IGF-1 E3R was 4.5 nM, as shown in FIG. 1. It suggests that wild-type IGF-1 and the tested IGF-1 variants have similar EC₅₀ values in vitro.

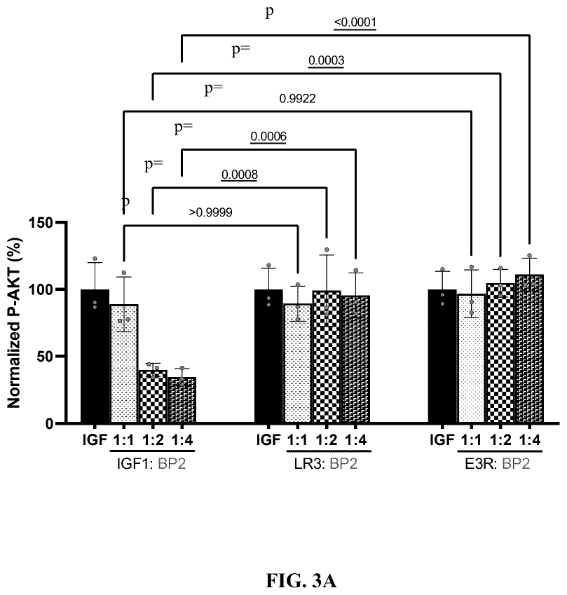

A literature search was conducted to determine differences in the ability of various IGF1 mutants to bind IGFBPs. Results of this search are shown in FIG. 2. In L6 rat myoblast conditioned media containing multiple binding proteins, competition assays report reduction in BP affinity for IGF-1 LR3 (SEQ ID NO: 12), LG3 (long IGF-1 E3G), long IGF-1, and IGF-1 Des 1-3 (SEQ ID NO: 3) to be 690 times, 112 times, 5.5 times and 38 times respectively (see Francis, G L et al., 8(3) J. Mol. Endocrinol. 213-223, 1992). In a separate study using bovine IGFBP2, IGF-1 E3R (SEQ ID NO: 4) and IGF E3G were reported to have reduction in binding affinity for IGFBP2 by 230 times and 59 times, respectively (see King, R. et al., 8 J. Mol. Endocrinol. 29-41, 1992). It suggests that some IGF-1 variants such as IGF-1 Des 1-3 (SEQ ID NO: 3), IGF-1 E3R (SEQ ID NO: 4), and IGF-1 LR3 (SEQ ID NO: 12) have reduced affinity for IGFBPs.

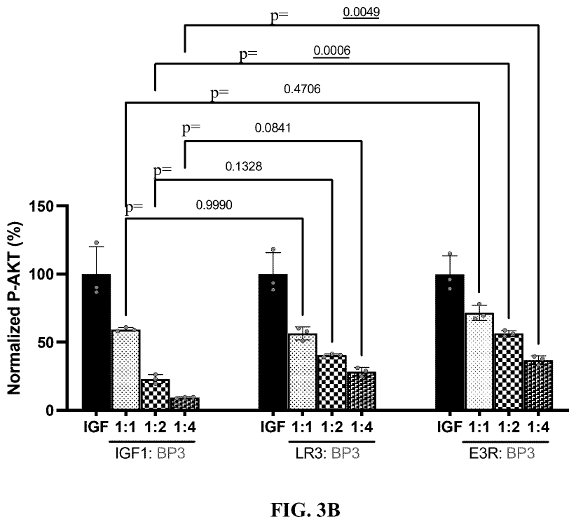

To determine the potency of IGF-1 variants in the presence of IGFBPs, pAKT levels measured in DU145 cells after preincubation of IGFBP2 or IGFBP3 with wild-type IGF-1 (SEQ ID NO: 1), IGF-1 LR3 (SEQ ID NO: 12), or IGF-1 E3R (SEQ ID NO: 4) at an IGF-1 proteins to IGFBPs ratio of 1:1, 1:2, or 1:4. FIGS. 3A and 3B suggest that IGF-1 LR3 (SEQ ID NO: 12) and IGF-1 E3R (SEQ ID NO: 4) are less inhibited by IGFBP2 and IGFBP3 compared to wild-type IGF-1 (SEQ ID NO: 1).

### Example 2: Wild-Type IGF-1 Enhances Growth of Spheroids

### Materials and Methods

In vitro potency assay (pAKT assay)

5×10⁶ immortalized human meibomian gland epithelial cells (IHMGECs: ATCC #CRL-3472) frozen down at passage 5 (P5) were reseeded in a T75 culture flask containing KSFM medium (Gibco: #10724-011) supplemented with 5 μg/l human recombinant EGF (Gibco #10450-013) and bovine pituitary extract (BPE) (Gibco #13028-014), MEM NEAA (Gibco #11140-050) Pen Strep (Gibco #15140-122) (“proliferation medium”) and expanded. At 90-95% confluency, cells were trypsinized, counted using an EVE automated cell counter from NanoEntek, resuspended at 4×10⁵ cells ml in culture medium, and then reseeded in 12-well plates (ThermoFisher #FB012928) in 1-ml aliquots per well. 36-42 hours after reseeding, the culture medium was removed, and following two washes with 1 ml PBS, cultured for 6 h in 450 μl DMEM/F12 (1:1) medium (Gibco: #11320-033) supplemented with Pen Strep (Gibco #15140-122) and 0.2% BSA (Millipore #A3059) (“serum starvation medium”). 50 μl aliquots of the following rh-IGF-1 (SEQ ID NO: 1) dilution series were added to the cells in duplicate: 10000, 1000, 100, 10, 1, 0.1, 0 nM. After 15 min incubation at 37° C., culture medium was aspirated, and cells lysed in 100 μl M-PER (Thermo Scientific #78501) containing protease/phosphatase inhibitors (one Pierce mini tablet [#A32961] per 10 ml M-PER). After 15-30 min incubation on ice, plates were vortexed, and lysates transferred to Eppendorf tubes and stored at −80° C. for assessment of IGFR1-AKT signaling strength using the phospho-Akt (S473) Pan Specific DuoSet IC ELISA (RnD #DYC887B-2). ELISAs were performed in 384-well plates to the manufacturers' recommendation with minor modifications. Briefly, wells of 384-well plates were coated with 25 μl 6 μg/ml phospho-Akt1 (S473) capture antibody (#841692) in PBS (25 μl per 384-well), sealed and placed overnight at RT. Wells were washed 4× with 100 μl PBS containing 0.05% Tween 20, and blocked with 50 μl PBS containing 2% BSA for 1-2 h at room temperature (RT). Wells were washed 4× with 100 μl PBS containing 0.05% Tween 20 and incubated with 25 μl of lysate or P-AKT standard for 2 h at RT. Wells were washed 4× with 100 μl PBS containing 0.05% Tween 20 and then incubated with 25 μl 100 ng/ml phospho-Akt1 (S473) detection antibody (#843081) diluted in PBS containing 1% BSA for 1 h at RT. Wells were washed 4× with 100 μl PBS containing 0.05% Tween 20 and incubated with 25 μl Streptavidin-HRP A (RnD #890803) diluted 1:200 in PBS containing 1% BSA for 20 min at RT. Wells were washed 4× with 100 μl PBS containing 0.05% Tween 20 and incubated with 25 μl TMB substrate solution (Abcam TMB ELISA Substrate High Sensitivity (ab171523; lot GR3427893-1)), and incubated for 10-20 min at RT. 12.5 μl stop solution (RnD #895926 from Ancillary Kit 2) was added and the OD₄₅₀ of each well measured using a BMG Labtech CLARIOstar® Plus Microplate Reader. EC₅₀ values were calculated using Prism 9.

### In Vitro Human Meibomian Gland Epithelial Cell Growth Assays

Live-Cell imaging assays. 5×10⁶ immortalized human meibomian gland epithelial cells (IHMGECs: ATCC #CRL-3472) frozen down at passage 5 (P5) were reseed in a T75 culture flask containing proliferation medium and expanded. At 90-95% confluency, cells were trypsinized, counted using an EVE automated cell counter from NanoEntek, resuspended at 1×10⁵ cells ml in proliferation medium, and then reseeded in two 12-well plates (ThermoFisher #FB012928) in 1-ml aliquots per well. 36 hours after reseeding, the proliferation medium was removed, and following two washes with 1 ml PBS, cultured in 500 μl serum starvation medium supplemented with 0, 0.1, 1, 10, 100 or 1000 nM human LR3-IGF-1 (PeproTech: #100-11R3) (4 independent wells for each condition). At 24-hour intervals, cells in the center of each well were imaged using an Eclipse Ti microscope (Nikon) with a 10× Plan Fluor Phl lens (Nikon) and an iXon Life 888 EMCCD camera.

CellTiter-Glo Luminescent Cell Viability Assay. 5×10⁶ immortalized human meibomian gland epithelial cells (IHMGECs: ATCC #CRL-3472) frozen down at passage 5 (P5) were reseeded in a T75 culture flask containing proliferation medium and expanded. At 90-95% confluency, cells were trypsinized, counted using an EVE automated cell counter from NanoEntek, resuspended at 1×10⁵ cells ml in proliferation medium, and then reseeded in two 12-well plates (ThermoFisher FB012928) in 1-ml aliquots per well. 36 hours after reseeding, the proliferation medium was removed, and following two washes with 1 ml PBS, cultured in 500 μl serum starvation medium supplemented with 0, 0.1, 1, 10, 100 or 1000 nM human IGF-1 (R&D 291-G1). 58 hours later, the medium on each well was replaced with 200 μl DMEM/F12 and an equal volume of CellTiter-Glo® Reagent. After 10 minutes on an orbital shaker two 100 μl aliquots of each 12 well were transferred to a 96-well flat-bottom black fluotrac plate (Greiner #655076) and luminescence measured at 560-580 nm using a BMG Labtech CLARIOstar® Plus Microplate Reader. As a measure of cell IGF1-mediated proliferation, RLU values of IGF1-treated IHMGEC were normalized to IHMGEC cultured in the absence of human IGF1.

### Results

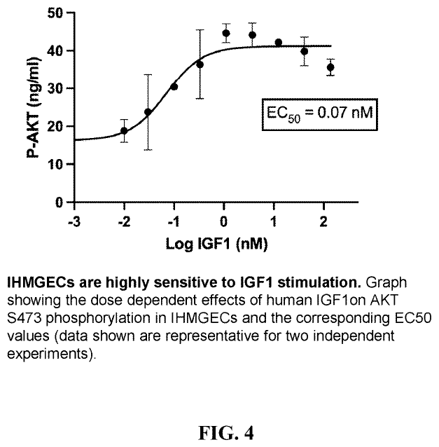

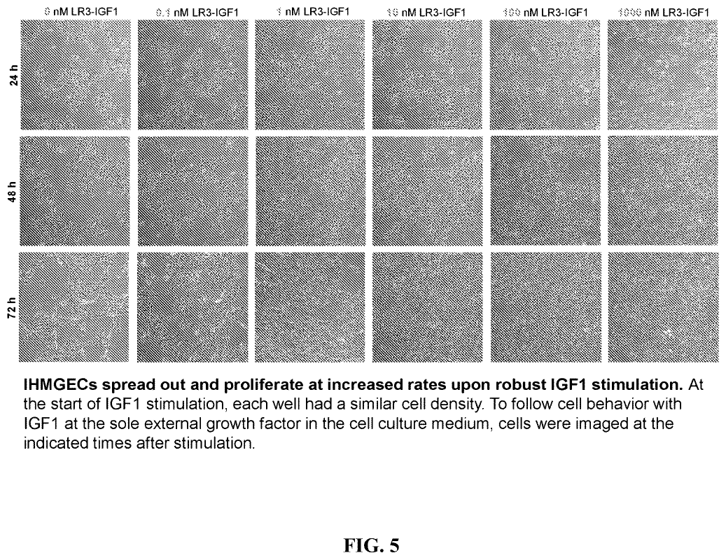

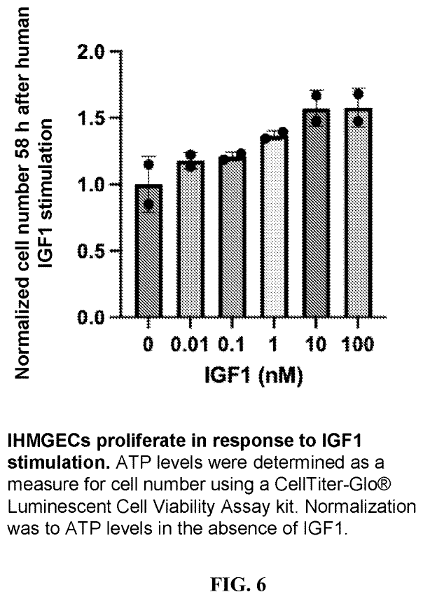

FIG. 4 depicts the dose-dependent effects of IGF-1 on AKT S473 phosphorylation in IHMGECs. The EC₅₀ value for IGF-1 is about 0.07 nanomolar (nM). FIG. 5 depicts the results of live cell imaging assays: IHMGECs spread out and proliferate at increased rates upon IGF-1 stimulation in a dose-dependent manner. FIG. 6 depicts the results of human IGF-1 stimulation in IGHMECs following the cell viability assay: the cells proliferate in response to IGF-1 in a dose-dependent matter.

### Example 3: IGF1 E3R Enhances Growth of Spheroids More Strongly than Wild-Type IGF-1

### Materials and Methods

### 3D Culture of IHGMGE Cells

2500 immortalized human meibomian gland epithelial cells (IHMGEC, ATCC, #CRL-3472) were seeded per well of a 24-well plate in a drop of 50 μL matrix (Matrigel, Corning #354230) covered by proliferation medium (Keratinocyte Serum Free Medium (Gibco, #17005042) supplemented with 50 μg/mL bovine pituitary extract (Gibco, #13028-014), 5 ng/mL recombinant human epidermal growth factor (EGF, Gibco, #10450-013), 10 μg/mL Gentamicin (Gibco, #15710-064) and 1% Pen/Strep (Gibco, #15140-122)). Spheroids were grown in proliferation medium for eight days, followed by a differentiation stage of seven days in presence of differentiation medium (DMEM:F12 medium (Gibco #11320-033) supplemented with 10 μg/mL Gentamicin (Gibco, #15710-064), 1% Pen/Strep (Gibco, #15140-122), 20 μM Rosiglitazone (Sigma Aldrich, #R2408), plus or minus various amounts of recombinant human insulin-like growth factor-1 (rhIGF1, PeproTech #100-11) or IGF1-E3R (SEQ ID NO: 12). N=2 per condition.

On day six of differentiation, spheroids were fixed in 4% PFA and processed for immunofluorescence using an antibody against Krt5 (Purified anti-Keratin 5 Polyclonal Chicken Antibody, Biolegend #905903) and detected with a AF594-labeled secondary anti-chicken antibody (Goat anti-Chicken IgY (H+L) Secondary Antibody, Alexa Fluo 594, Thermo #A-11042). For images of 2D and 3D spheroid cultures, brightfield images were collected with transmitted light.

### Measurement of Spheroids' Growth of IHGMGE Cells

IHMGE cells were cultured as described above. As a measure of proliferation, Fiji 2 by ImageJ was used to measure the diameter of each spheroid at day 7 of differentiation. To this end, a z-stack of 25 bright field images spanning a total of 1 mm were taken and maximum image projection was used to generate one final image that was used for measurement.

### Measurement of Lipid Production in the Spheroids

IHMGE cells were cultured as described above. On day six of differentiation, 1:3000 LipidTOX Green neutral lipid stain (Invitrogen, #H34475) was added to the wells. As a measure of lipid production, at day seven of differentiation a z-stack of 25 4× images spanning 1 mm were taken with a fluorescent microscope with filter sets appropriate for Alexa Fluor 488 dye or fluorescein. After maximum image projection, the mean signal per spheroid was calculated using QuPath software (https://qupath.github.io/).

### Quantification of IGFBPs in the Spheroids

IHMGE cells were cultured as described above. On day six of differentiation, replicates were pooled and lysed in 200 μl of 2× Extraction Buffer (5X PTR, Abcam #, ab193970) and vortexed extensively. Lysates were then frozen at −80 degrees. For analysis, protein quantitation was performed on each sample using Pierce Detergent Compatible Bradford Assay Kit (Thermo, #23246) and analyzed by Human IGF Signaling Array C1 Ray Biotech, #AAH-IGF-1-4) according to the manufacturer's instructions.

### Results

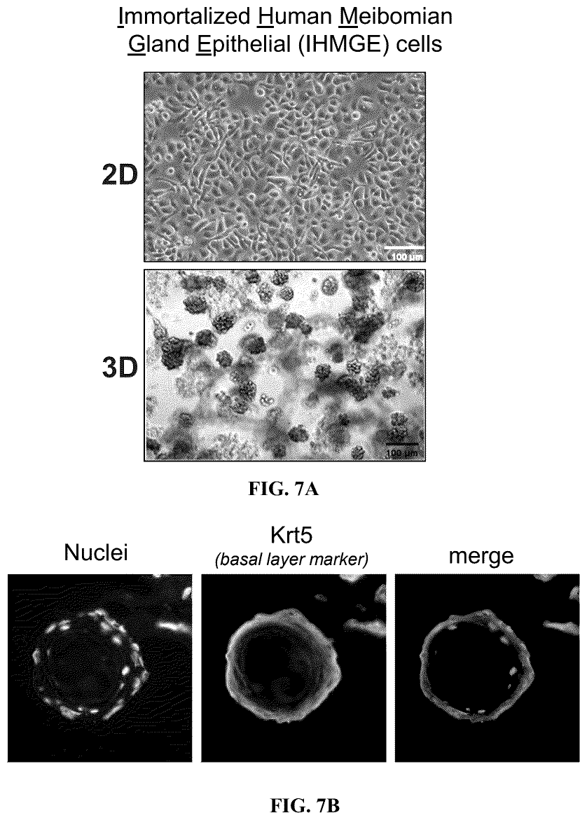

IHMGE cells grown in 3D culture under conditions promoted differentiation develop into spheroids structures resembling Meibomian gland Acini, as shown in FIGS. 7A and 7B. Spheroids expressed markers of the meibomian gland acini basal compartment including Krt5 and exhibit proliferation in the basal compartment, as shown in FIGS. 7A and 7B.

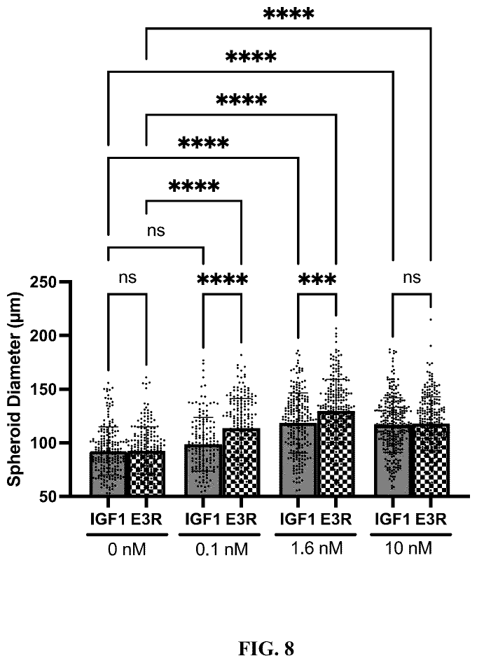

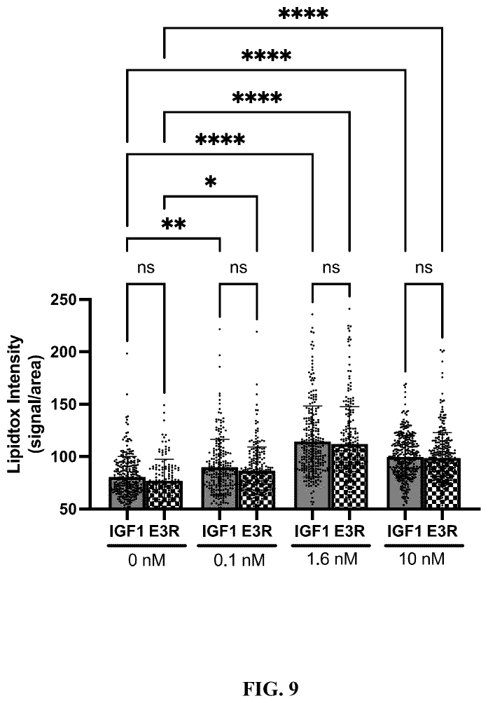

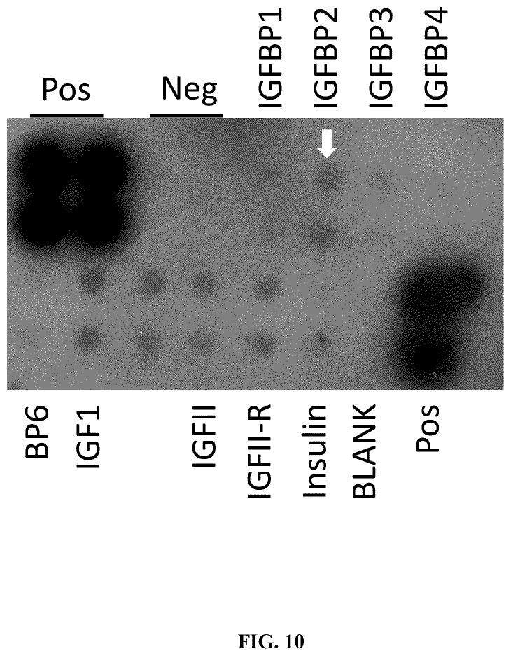

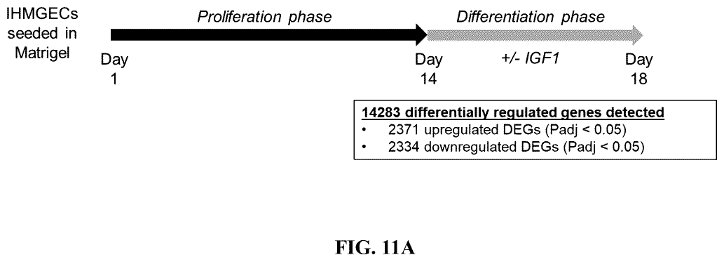

The perimeters of spheroids cultured in the presence of 0 nM, 0.1 nM, 1.6 nM, or 10 nM of IGF-1 or IGF-1 E3R were measured on day six of differentiation. FIG. 8 provides that spheroids grown in the presence of IGF-1 E3R were significantly larger than those grown with wild-type IGF-1. Furthermore, lipidtox intensity of spheroids cultured in the presence of 0 nM, 0.1 nM, 1.6 nM, or 10 nM of IGF-1 or IGF-1 E3R were measured on day seven of differentiation. IGF-1 and IGF-1 E3R proportionally increase Lipid content in spheroids, as shown in FIG. 9. Cells were lysed for quantification of IGFBPs. FIG. 10 provides that IGFBP2 was expressed higher compared to IGFBP1, IGFBP3, and IGFBP4 in IHMGE spheroid cultures.

### Example 4: IGF1 Treatment Upregulates Genes Involved in Fatty Acid Transport, Lipid Synthesis, and Meibogenesis

### Materials and Methods

2500 immortalized human meibomian gland epithelial cells (IHMGEC, ATCC, #CRL-3472) were seeded per well of a 24-well plate in a drop of 50 μL matrix (Geltrex, ThermoFisher #A1413201) covered by proliferation medium (Keratinocyte Serum Free Medium (Gibco, #17005042) supplemented with 50 μg/mL bovine pituitary extract (Gibco, #13028-014), 5 ng/mL recombinant human epidermal growth factor (EGF, Gibco, #10450-013), 10 μg/mL Gentamicin (Gibco, #15710-064) and 1% Pen/Strep (Gibco, #15140-122)). Spheroids were grown in proliferation medium for 14 days, followed by a differentiation stage of four days in presence of differentiation medium (DMEM:F12 medium (Gibco #11320-033) supplemented with 10 μg/mL Gentamicin (Gibco, #15710-064), 1% Pen/Strep (Gibco, #15140-122), 20 μM Rosiglitazone (Sigma Aldrich, #R2408), plus or minus 100 nM recombinant human insulin-like growth factor-1 (rhIGF1, PeproTech #100-11).

RNA extraction from 4-day differentiated spheroid cultures was performed by TRIzol extraction (Invitrogen #15596026) followed by an RNA clean-up step with RNA Clean & Concentrator-5 (Zymo Research #R1013) according to manufacturers' instructions. In brief, n=4 samples per condition. The droplets of matrix containing the spheroids were transferred to an Eppendorf tube and 500 μL TRIzol was added to lyse the cells, then 100 μL chloroform was added and the aqueous phase was transferred to an RNase-free tube and mixed with equal volume of 100% ethanol. The mixture was cleaned up with the Zymo-Spin IC Column, including a DNase I treatment, all according to protocol. RNA quality and quantity were assessed with a nanodrop (A260/A280=1.8-2, A260/A230>=1.8) prior to submitting total RNA samples to Novogene. At Novogene, RNA purity and integrity was confirmed. Then, a mRNA library with poly A enrichment was prepared and subsequently tested for its quality (Library QC). Next, a 150 bp paired-end sequencing strategy was used to sequence the library with the Illumina NovaSeq PE150 platform (6 G of raw data per sample), and the quality of the resulting data was also checked (Data QC).

Fastq files of paired-end RNA-Seq reads were aligned with STAR v. 2.6.0a (Dobin, A. et al., 29(1) Bioinformatics 15-21, 2013) against the reference genome hg38 (Schneider, V. A. et al., 27(5) Genome research 849-864, 2017). Gene level counts from read pairs were obtained using FeatureCounts v. 2.0.6 (Liao, Y. et al., 30(7) Bioinformatics 923-930, 2014) from the SubRead package. Differential expression analysis was performed using R package DESeq2 v.1.40.2 (Love, M. I. et al., 15(12) Genome biology 550, 2014). after removing genes with average raw counts <10. Gene Set Enrichment Analysis (GSEA) (Subramanian, A. et al., 102(43) Proc Natl Acad Sci USA. 15545-15550, 2005). was performed to detect enriched pathways against human gene sets from MSigDB (Liberzon A. et al., 27(12) Bioinformatics 1739-1740, 2011). using gene lists ranked in descending order by log 2FoldChange. Overrepresentation analysis was performed using the Fisher's exact test method for selected up-(log 2FoldChange >1, adjusted p-value <0.25) and down-regulated (log 2FoldChange <−1, adjusted p-value <0.25) genes against gene sets related to meibogenesis gathered from literature. Morpheus (Broad Institute) was used to generate heatmaps.

### Results

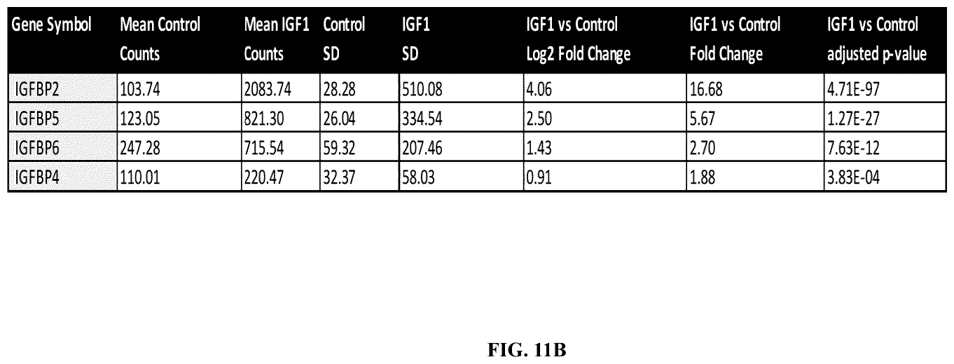

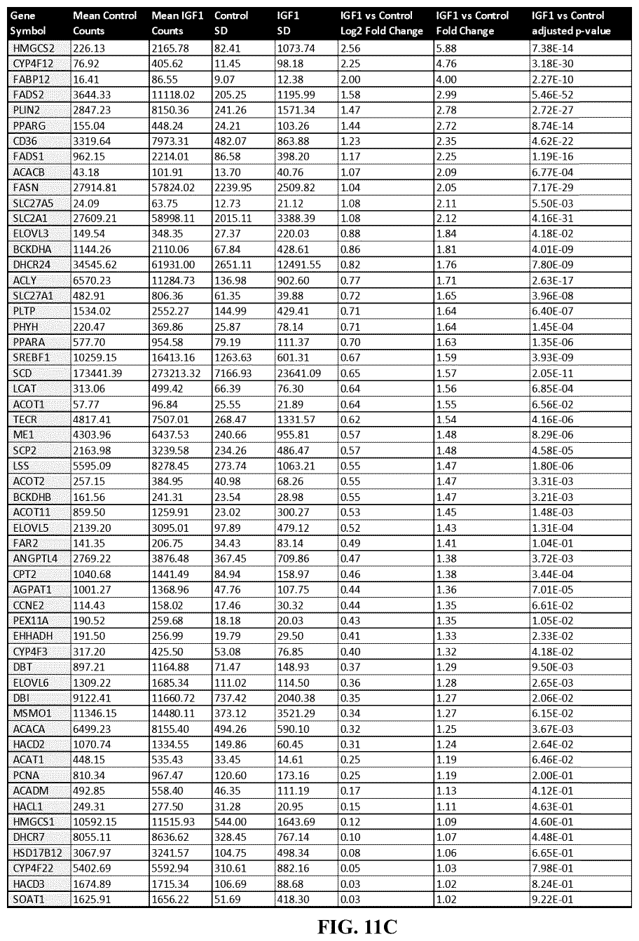

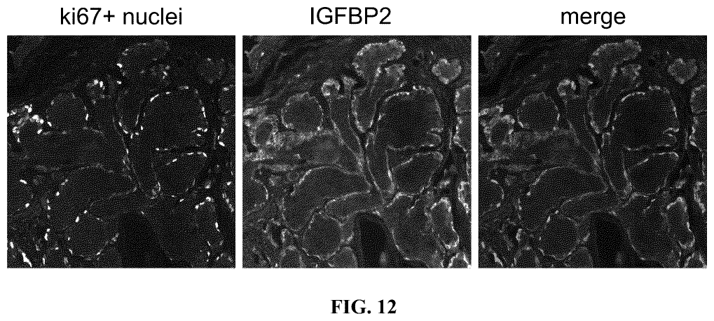

To perform transcriptional analysis of the effects of wild-type IGF1 treatment on IHMGE spheroids, IHMGE cells were seeded in Matrigel and differentiated for 4 days in the presence of rosiglitazone or rosiglitazone plus IGF1. RNA sequencing technology was used to determine the effects of wild-type IGF1 on gene expression in IHMGE spheroid. FIG. 11A illustrates the flowchart of the study design. Treatment of IHMGE spheroids with wild-type IGF1 significantly upregulates four IGFBPs, which are IGFBP2, IGFBP5, IGFBP4, and IGFBP6, as shown in FIG. 11B. Among them, IGFBP2 was the most significantly upregulated IGFBP in response to treatment with wild-type IGF1, as shown in FIG. 11B. Treatment of IHMGE spheroids with wtIGF1 significantly upregulates genes involved in fatty acid transport, lipid synthesis, and meibogenesis, as shown in FIG. 11C. Gene sets of interest were adapted from Butovich I., 163 Exp Eye Res. 2-16, 2017.

### Example 5: In Vivo Effects of IGF-1 Variants on Mouse Meibomian Glands

### Materials and Methods

### Animal Studies

Wild-type (WT) female C57BL/6J (Jackson Labs, Strain #000664) aged 2 months or 1-1.5 years of age were purchased. All animal studies were conducted in compliance with the relevant protocols.

### Dosing

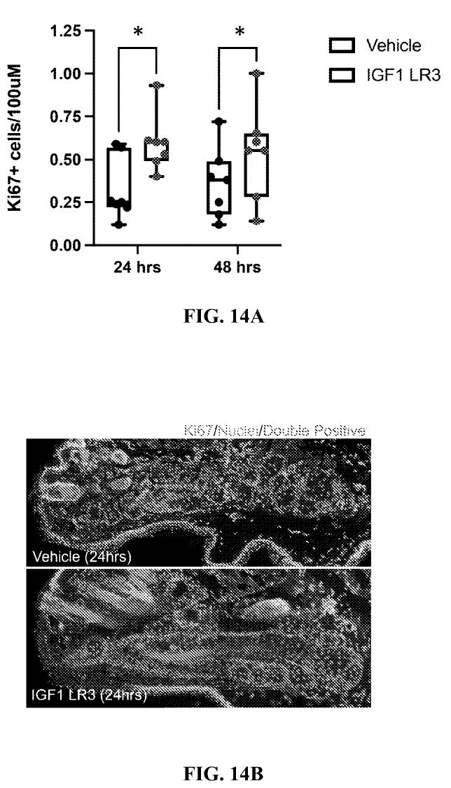

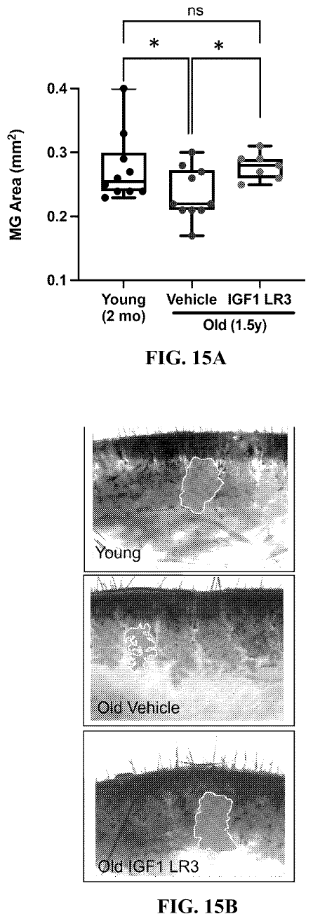

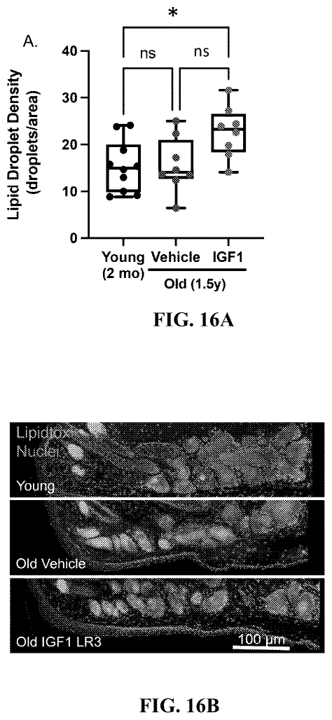

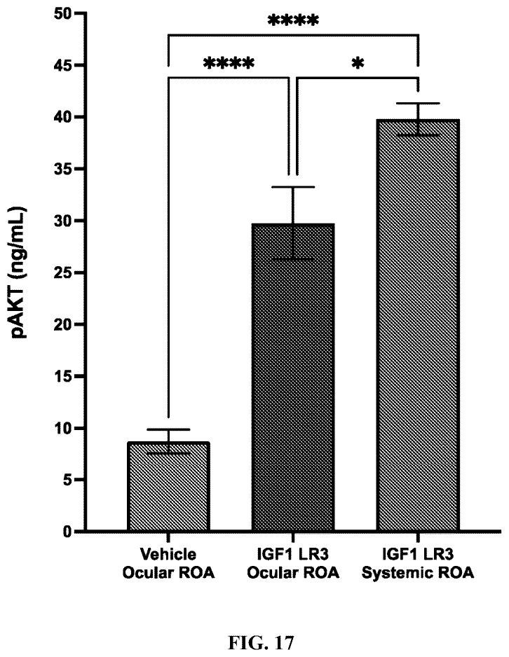

For dosing studies, mice were administered vehicle (Endotoxin-free PBS, EMD Millipore, TMS-012-A) or 10 milligram/kilogram (mg/kg) IGF-1 LR3 (SEQ ID NO: 12) resuspended in vehicle. Animals were dosed using insulin syringes (BD, 329424). Mice were given 2 intraperitoneal (IP) doses of IGF-1 LR3, 12 hours apart and sacrificed as described below at either 24 or 48 hours post dosing (results in FIGS. 14A and 14B). Mice were dosed daily IP with vehicle or 10 mg/kg IGF-1 LR3 for 5 days in a row followed by a 2 day holiday for a total of 4 weeks, then sacrificed as described below (results in FIGS. 15A, 15B, 16A, and 16B). Animals were dosed with a single IP dose of 10 mg/kg IGF-1 LR3 or given a 10 μl eyedrop of vehicle of IGF-1 LR3 at a concentration of 5 mg/ml, then sacrificed as described below (results in FIG. 17).

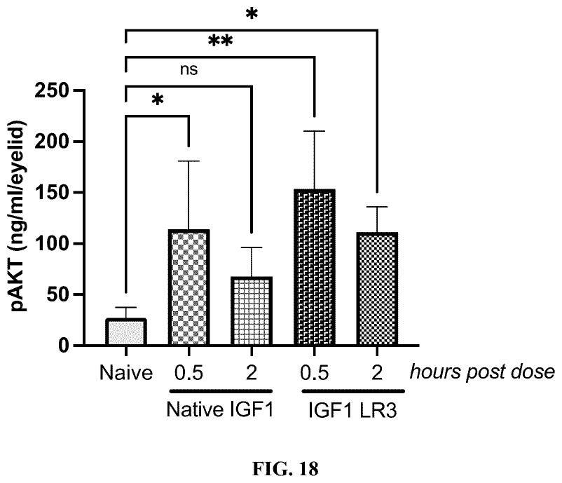

In FIG. 18, animals were administered a 10 μl eyedrop containing equimolar concentration based on molecular weight of either wild-type IGF-1 (SEQ ID NO: 1) at lmg/ml or IGF-1 LR3 (SEQ ID NO: 12) at 1.27 mg/ml then sacrificed as described previously at 0.5 hours or 2 hours post dosing. Eyelids were removed and frozen on dry ice then stored at −80° C. until analysis in P-AKT ELISA.

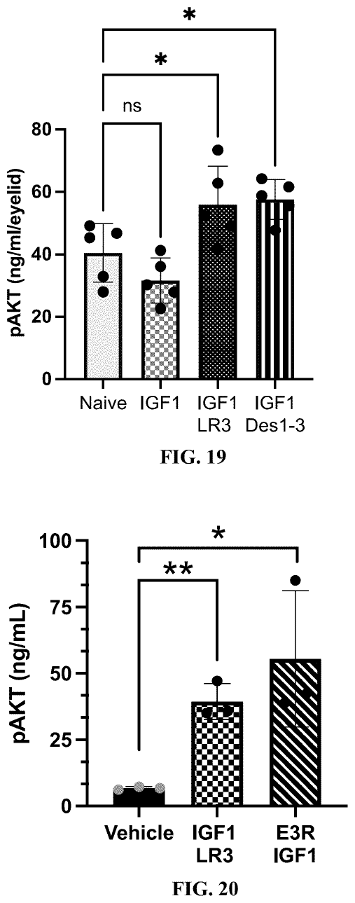

In FIG. 19, animals were administered a 10 μl eyedrop containing equimolar concentration based on molecular weight of either wild-type IGF-1 (SEQ ID NO: 1) at 1 mg/ml, IGF-1 LR3 (SEQ ID NO: 12) at 1.27 mg/ml, or rhIGF-1 Des1-3 (SEQ ID NO: 3) at 1 mg/ml then sacrificed as described previously at 2 hours post dosing. Eyelids were removed and frozen on dry ice then stored at −80° C. until analysis in P-AKT ELISA.

In FIG. 20, animals were administered a 10 μl eyedrop containing equimolar concentration based on molecular weight of either IGF-1 LR3 (SEQ ID NO: 12) at 1.27 mg/ml IGF-1 E3R (SEQ ID NO: 4) at 1 mg/ml then sacrificed as described previously at 1 hour post dosing. Eyelids were removed and frozen on dry ice then stored at −80° C. until analysis in P-AKT ELISA.

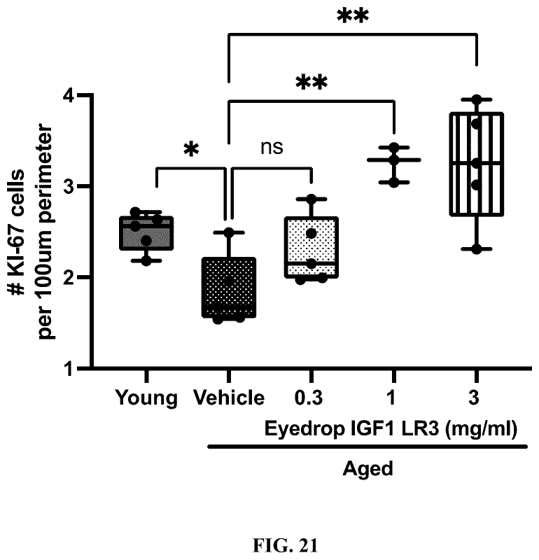

In FIG. 21, animals were administered a 10p eyedrop containing IGF-1 LR3 (SEQ ID NO: 12) at 0.3, 1 or 3 mg/ml every day for 2 weeks and sacrificed 24 hours after the last dose.

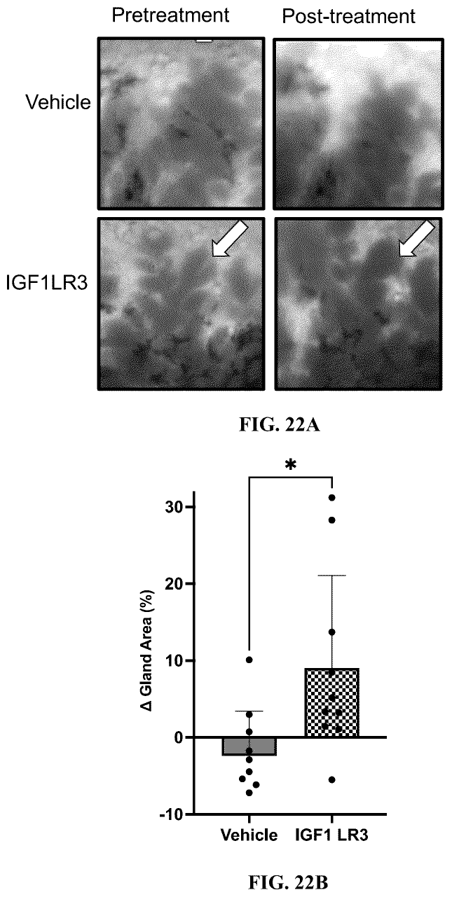

In FIGS. 22A and 22B, animals received a daily bilateral 10p dose of Vehicle (PBS) or 1 mg/ml IGF-1 LR3 (SEQ ID NO: 12) in each eye for 4 weeks.

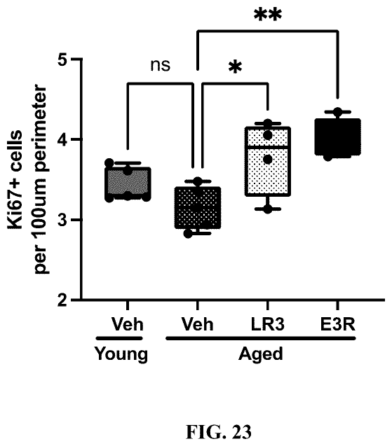

In FIG. 23, animals were administered a 10 μl eyedrop containing equimolar concentration based on molecular weight of either IGF-1 LR3 (SEQ ID NO: 12) at 1.27 mg/ml or IGF-1 E3R (SEQ ID NO: 4) at 1 mg/ml every day for 2 weeks and sacrificed 24 hours after the last dose.

### Tissue Collection, Processing and Analysis

Mice were sacrificed by cervical neck dislocation after Isoflurane anesthesia and the upper left and right eyelids removed. Eyelids were then trimmed to the central 4 mm of tissue, embedded in Tissue-Tek® O.C.T. Compound (Sakura Finetek, Torrance, CA) and then snap frozen over liquid nitrogen. Tissue blocks were then sectioned, 8 μm thick, using a Leica CM 1850 cryostat (Leica, Wetzlar, Germany). Tissue sections were then stored in an ultra-low freezer until processed for fluorescent microscopy.

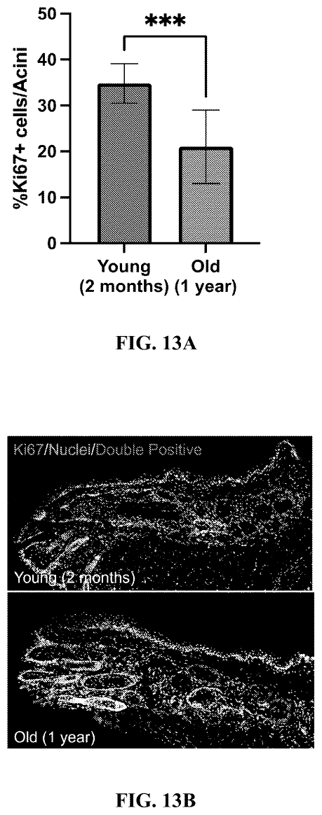

To measure basal proliferation rates or the effects of IGF-1LR3 on cell proliferation (FIGS. 13A, 13B, 14A, and 14B), tissue sections were stained with the cell proliferation marker, Ki67. Rabbit anti-Ki67 (Abcam, catalog #ab15580) were reacted with tissue sections for 1 hour at 37° C. in a humidified chamber. Sections were then washed in PBS (3×5 min) and secondary antibody (Goat Anti-Rabbit AlexaFluor546, Invitrogen) was applied to the sections for 1 hour and subsequently were rinsed with PBS (3×5 min), counterstained with DAPI (1:5000) and then mounted with a coverslip. Fluorescent imaging was performed on a Leica DMI6000B fully automated inverted fluorescence microscope (Leica Microsystems Inc., Buffalo Grove, IL) and tiled images over the eyelid tissue section and meibomian gland were collected using a low-light-level camera (QIClick, QImaging, British Columbia, Canada) and Leica 20× HC Plan Apo, 0.75 NA objective. Images were then stitched using Meta Imaging Series software. Quantification of Ki67 labeling was performed using QuPath, a bioimaging software analysis tool for high-throughput biomarker analysis. Initially, individual acini in each tissue section were annotated using the free-form region of interest tool, and the positive Ki67 stained nuclei within each acini identified using the positive cell detection tool. The number of Ki67 positive cells and the perimeter of each acinus was then recorded and the average number of label cells/100 μm of acinar perimeter calculated for each section. The average of three tissue sections was then determined the average for mouse calculated. Differences between treatment groups were then determined using a student's t-test (FIGS. 13A and 13B) or Two-Way Analysis of Variance, Tukey all pairwise multiple comparison procedures in both SigmaStat (Systat Software Inc., Point Richmond, CA) and GraphPad Prism (Insight Partners, NY, NY).

To assess changes in lipid synthesis (FIGS. 16A and 16B), cells were stained with the neutral lipid fluorescent probe, HCS LipidTox (Invitrogen, Carlsbad, CA). Cells were initially fixed in 2% paraformaldehyde in PBS and then rinsed and incubated in LipidTox solution (dilution 1:100) for 20 minutes at room temperature followed by DAPI staining. Fluorescent staining was then imaged using the Leica DM16000B inverted microscope and tiled images over the eyelid section collected and stitched together using Meta Imaging software. To quantify newly synthesized lipid droplets near basal acinar cells, the region of lipid staining in the meibomian gland was segmented using the Threshold sub-routine to identify pixels with intensity >50. The threshold region was then extracted using the duplicate plane function and newly synthesized individual lipid droplets, which showed high intensity fluorescent staining, were identified using the count sub-routine set to identify particles greater than 2 μm and less than 5 μm in diameter that had a threshold intensity greater than 1000. Individual acini were then manually outlined using the free-hand region tool and the area of the acinus and number of lipid droplets/acinus recorded. The number of lipid droplets/acinus were then normalized to an average acinar area of 4000 μm². The average number of lipid droplets in three individual sections were then calculated, followed by the average number/mouse. The difference in droplets/acini numbers per treatment group were then determined using One-Way Analysis of Variance, all pairwise multiple comparison with Tukey's method GraphPad Prism (Insight Partners, NY, NY).

To evaluate activation of IGF1R1 by IGF-1 LR3 (FIG. 17), phospho-AKT levels were measured using the phospho-Akt (S473) Pan Specific DuoSet IC ELISA (RnD #DYC887B-2). Eyelid tissue was flash frozen on dry ice and stored at −80C until later analysis. Samples were weighed and homogenized 1 ml/50 mg tissue in T-PER (Thermo Scientific, 78510) containing protease and phosphatase inhibitors (Thermo Scientific, A32959 using a NextAdvance Bullet Blender Gold (BB24AU) with Green, Navy or Red Eppendorf Lysis Kits (NextAdvance). Homogenates were then used directly in the assay. ELISAs were performed in 384-well plates to the manufacturers' recommendation with minor modifications. Briefly, wells of 384-well plates were coated with 25 μl 6 μg/ml phospho-Akt1 (S473) capture antibody (#841692) in PBS (25 μl per 384-well), sealed and placed overnight at RT. Wells were washed 4× with 100 μl PBS containing 0.05% Tween 20, and blocked with 50 μl PBS containing 2% BSA for 1-2 h at room temperature (RT). Wells were washed 4× with 100 μl PBS containing 0.05% Tween 20 and incubated with 25 μl of lysate or P-AKT standard for 2 h at RT. Wells were washed 4× with 100 μl PBS containing 0.05% Tween 20 and then incubated with 25 μl 100 nanograms per milliliter (ng/ml) phospho-Akt1 (S473) detection antibody (#843081) diluted in PBS containing 1% BSA for 1 h at RT. Wells were washed 4× with 100 μl PBS containing 0.05% Tween 20 and incubated with 25 μl Streptavidin-HRP A (RnD #890803) diluted 1:200 in PBS containing 1% BSA for 20 min at RT. Wells were washed 4× with 100 μl PBS containing 0.05% Tween 20 and incubated with 25 μl TMB substrate solution (Abcam TMB ELISA Substrate High Sensitivity (ab171523; lot GR3427893-1)), and incubated for 10-20 min at RT. 12.5 μl stop solution (RnD #895926 from Ancillary Kit 2) was added. OD450 data for standard curves and each sample was collected using a Promega GloMAX Discover Microplate Reader. Sample data was interpolated from standard curves, corrected for dilution factors, plotted and pAKT levels were plotted and compared statistically using One-way ANOVA and Tukey's method for multiple comparisons in GraphPad Prism (Insight Partners, NY, NY).

### Mouse Eyelid Meibography

Transillumination meibography (FIGS. 15A and 15B) was conducted before the start of the study and just prior to sacrifice using techniques previously published. Briefly, mice were anesthetized using isoflurane and placed under a binocular dissection microscope (Leica MZ16FA, Leica Microsystems, Heerbrugg, Switzerland) equipped with a monochromatic camera (DFC340FX, Leica Microsystems, Heerbrugg, Switzerland). A broad band, halogen light source (OSL2 and OSL2B, Thorlabs, Newton, NJ) was used to transilluminate the eyelid for meibography. The light was delivered by a special optical fiber (BFL200HS02, Thorlabs, Newton, NJ) that had an input connector comprised of a bundle of seven 200 μm-diameter fibers and an output connector comprised of a linear array of fibers attached to a diffuser and a prism with glue. All images were taken at 20× magnification. After sacrifice, eyelids were removed and ex vivo meibography performed by placing eyelids onto an LED back light plate and taking transillumination photographs using the dissection microscope. To quantify meibomian gland area, individual eyelid images were analyzed using Meta Imaging Series software (Molecular Devices, Downington, PA). Specifically, the region of individual meibomian glands were manually outlined and the area of the region of interest measured for each individual gland in each image. The average area for the glands in each eyelid were then calculated and differences between the groups statistically analyzed using One-tailed Mann-Whitney test, using GraphPad Prism for (Insight Partners, NY, NY).

### Immunohistochemistry Staining (IHC)

Eyelids from Wild-type (WT) female C57BL/6J (Jackson Labs, Strain #000664) were dissected. Tissue was fixed in 4% paraformaldehyde overnight and switched to 30% sucrose 24 hours later. Eyelids were then embedded in OCT medium and sectioned in 10 micron sections and adhered to glass slides. Tissue was then stained for ki67 using Rat anti-Ki67 at 1 ug/mL (Invitrogen #14-5698-82) followed by detection with an anti-rat Alexafluor-labeled secondary antibody and IGFBP2 using Rabbit anti-IGFBP2 at 1 ug/mL (Abcam #ab188200) followed by detection with an anti-rabbit Alexafluor-labeled secondary antibody. In between antibody incubations, slides were washed with PBS+0.1% Tween-20 3×. Slides were then coverslipped and imaged.

In FIGS. 21 and 23, animals were sacrificed as previously described, and eyelids were fixed in 4% paraformaldehyde overnight and switched to 30% sucrose 24 hours later. Eyelids were then embedded in OCT medium and sectioned in 10 micron sections and adhered to glass slides. Tissue was then stained for ki67 using Rat anti-Ki67 at 1 ug/mL (Invitrogen #14-5698-82) followed by detection with an anti-rat Alexafluor-labeled secondary antibody. In between antibody incubations, slides were washed with PBS+0.1% Tween-20 3×. Slides were then coverslipped and imaged. Cell nuclei were then stained with Hoescht and cover slipped for imaging. Random regions of interest (ROIs) were identified containing meibomian gland acini.

Acini perimeter was outlined based on Hoescht stain in Image J and number of ki67+ cells lining the perimeter were measured and represented as number of ki67+ cells per 100 μm of acini perimeter. Data was then quantified and analyzed in GraphPad Prism (Insight Partners, NY, NY).

### Results

As depicted in FIGS. 13A and 13B, young mice have increased proliferation in meibomian gland acini compared to old mice. Systemic IGF-1 LR3 treatment increased proliferation in meibomian gland acini in aged mice (FIGS. 14A and 14B); reversed atrophy and increased Meibomian gland area in aged mice (FIGS. 15A and 15B); increased lipid synthesis in Meibomian glands regardless of age (FIGS. 16A and 16B); and could be delivered systemically or by ocular drops to activate IGF1R in the eyelid (FIG. 17).

Next, the duration of effects of IGF-1 variations were investigated. IGF-1 LR3 (SEQ ID NO: 12) maintained significantly elevated pAKT (IGF1R activation) at 2 hours post dosing, whereas wild-type IGF-1 no longer showed significant pAKT at 2-hours post dosing, as shown in FIG. 18. IGF-1 LR3 (SEQ ID NO: 12) and another IGFBP-binding-deficient IGF-1 variant, IGF-1 Des1-3 (SEQ ID NO: 3), significantly elevated pAKT (IGF1R activation) at 2 hours post dosing compared to wild-type IGF-1, as shown in FIG. 19. It suggests that some IGF-1 variants with reduced affinity to IGFBPs prolong duration of IGF1R activation compared to wild-type IGF-1. Furthermore, IGF-1 LR3 (SEQ ID NO: 12) and IGF-1 E3R (SEQ ID NO: 4) have similar potency in vivo. IGF-1 LR3 (SEQ ID NO: 12) or an IGFBP-binding-deficient IGF-1 variant IGF-1 E3R (SEQ ID NO: 4) increased pAKT (IGF1R activation) to levels comparable to IGF-1 at 1 hour post dosing, as shown in FIG. 20.

We next tested the effects of IGF-1 LR3 and IGF-1 ER3 in aged mouse meibomian glands. IGF-1 LR3 (SEQ ID NO: 12) induced dose-responsive proliferation in basal cells of the meibomian gland, as shown in FIG. 21. Two weeks of daily eyedrop dosing of IGF-1 LR3 (SEQ ID NO: 12) at the indicated concentration of 0.3, 1 or 3 mg/ml induced basal cell proliferation as determined by IHC, as shown in FIG. 21. IGF-1 LR3 (SEQ ID NO: 12) regenerated atrophied Meibomian glands in aged mice. Daily treatment with IGF-1 LR3 (SEQ ID NO: 12) for 1 month increased area of Meibomian glands comparing pre- and post-treatment, as shown in FIGS. 22A and 22B. IGF-1 LR3 (SEQ ID NO: 12) and IGF-1 E3R (SEQ ID NO: 4) induced proliferation in basal cells of the Meibomian gland. Two weeks of daily eyedrop dosing of IGF-1 LR3 (SEQ ID NO: 12) or IGF-1 E3R (SEQ ID NO: 4) induced basal cell proliferation in aged mice as determined by IHC, as shown in FIG. 23.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 6: In Vitro Potency of Wild-Type IGF-1 and IGF-1 Variants in the Presence and Absence of IGFBPs

### Materials and Methods

### In Vitro Potency Assay in DU145 Cells

For potency experiments, 15×10⁶ DU145 cells (ATCC #HTB-81) frozen down at passage 5 (P5) were reseeded in a T175 culture flask containing DMEM/F12 (1:1) medium (Gibco 11320-033) supplemented with 10% FBS (Gibco 10437-028) and 10 μg/mL Gentamicin (Gibco 15710-064) [hereafter referred to as culture medium] and expanded. At 80-90% confluency, cells were trypsinized, manually counted using a hemocytometer, resuspended at 2.5×10⁵ cells per mL in culture medium, and then reseeded in 24-well plates (Corning 3524) in 0.5 mL aliquots per well (125,000 cells/well). 14-16 hours after reseeding, the culture medium was removed, and following a wash with 1 mL PBS, cultured for 6 h in 225 μL serum-free culture medium supplemented with 0.2% BSA (Millipore A3059). 25 μL aliquots of the following growth factor dilution series were added to the cells in duplicate for a final concentration in the well of: 457, 137, 41, 12.5, 3.7, 1.11, and 0.33 nM rhIGF1 (PeproTech #100-11, SEQ ID NO: 1), rhIGF1 E3R (SEQ ID NO: 4), linker IGF-1 (SEQ ID NO: 13), linker IGF-1 E3R (SEQ ID NO: 14), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), IGF-1 E3R A13C/L57C (SEQ ID NO: 19), or IGF-1 E3R L14C/L57C (SEQ ID NO: 20). After 15 min incubation at 37° C., medium was aspirated and cells lysed in 150 μL 1×PTR buffer (Extraction Buffer 5×PTR, Abcam ab193970) containing 1× Extraction Enhancer Buffer (Extraction Enhancer Buffer 50×, Abcam ab193971) and protease/phosphatase inhibitors (one Pierce mini tablet [A32959] per 10 mL 1×PTR). After 15-30 min incubation on ice, plates were vortexed, and lysates transferred to Eppendorf tubes and stored at −80° C. for assessment of IGF1R-AKT signaling strength using the Human/Mouse/Rat Phospho-Akt (S473) Pan Specific DuoSet IC ELISA (RnD #DYC887B). EC₅₀ fits were calculated using Graphpad Prism (Graphpad Software 2023).

### IGFBP2/3 Inhibition

The same protocol as above for potency experiments was followed except that rhIGF1 (PeproTech #100-11, SEQ ID NO: 1), rhIGF1 E3R (SEQ ID NO: 4), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), IGF-1 E3R A13C/L57C (SEQ ID NO: 19), or IGF-1 E3R L14C/L57C (SEQ ID NO: 20) (collectively called Growth Factor) were preincubated with IGFBP2 (R&D systems #674-B2) or IGFBP3 (R&D systems #675-B3) in a 1.5 mL Eppendorf tube at room temperature for 1.5 hrs before stimulating the cells. The preincubation was done with 250 nM growth factor (10×) and 0-, 1-, 2- or 4-fold molar excess of IGFBP2/3. Final concentration during stimulation was 25 nM Growth Factor with 0, 25 nM, 50 nM or 100 nM IGFBP2/3. Phosphorylated AKT levels were measured as above using the Human/Mouse/Rat Phospho-Akt (S473) Pan Specific DuoSet IC ELISA (RnD #DYC887B). Inhibition was determined by normalizing the total pAKT signal to the control group (0 nM IGFBP, 100%).

### Result

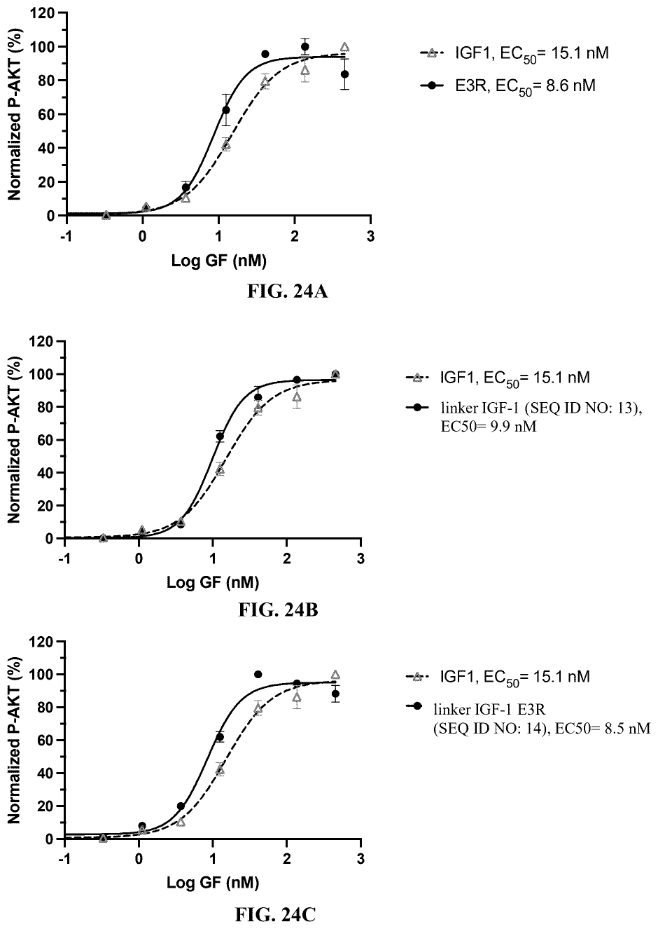

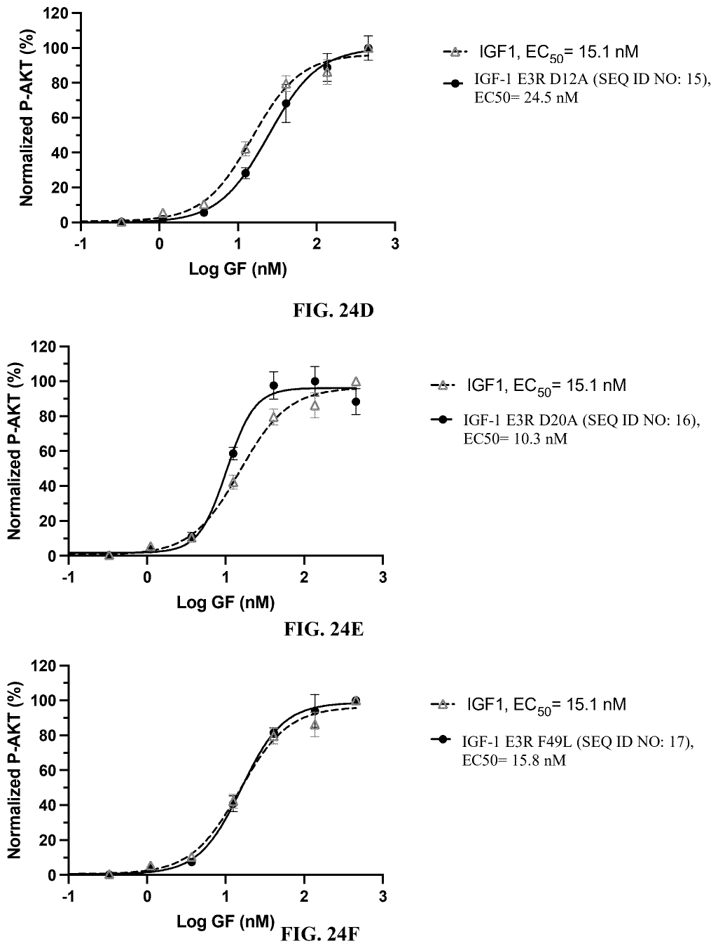

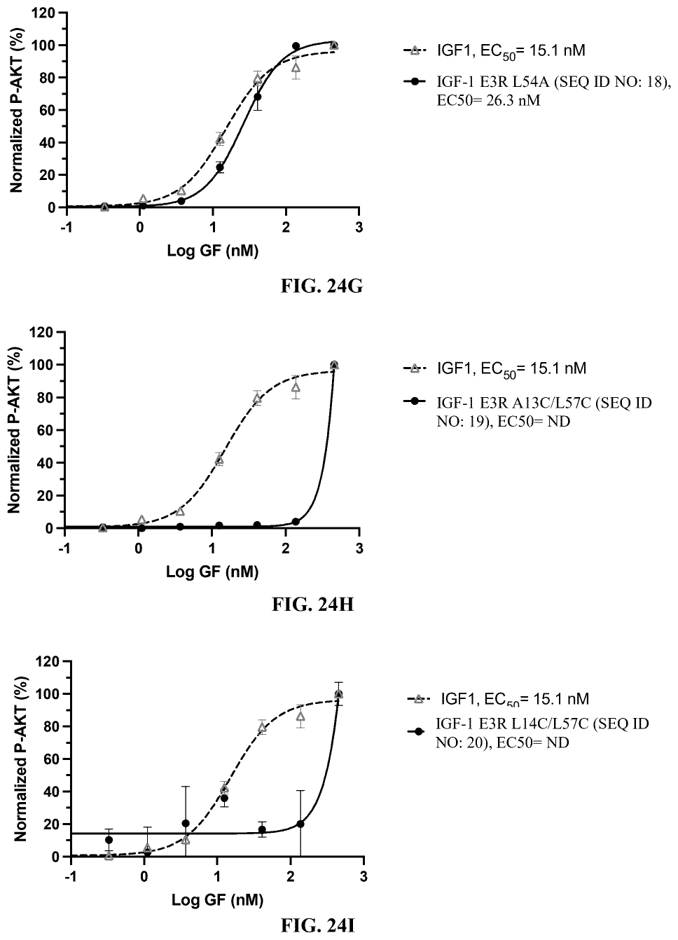

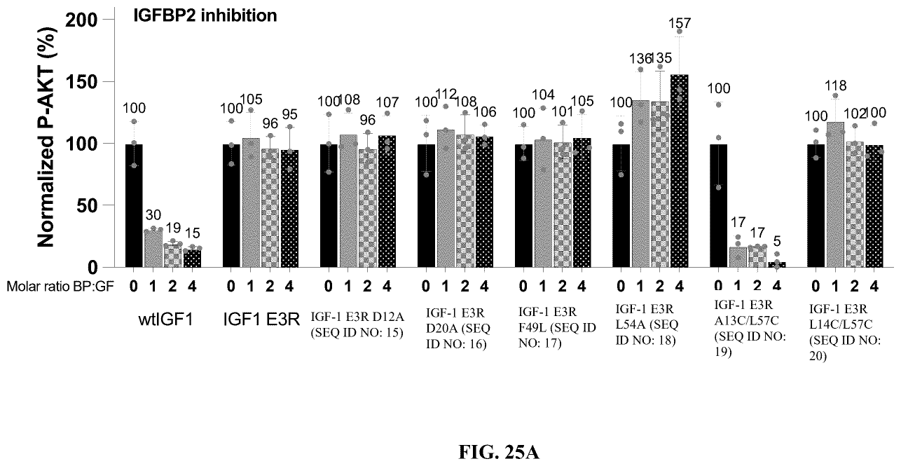

The dose-dependent effects of IGF-1 (SEQ ID NO: 1), IGF-1 E3R (SEQ ID NO: 4), linker IGF-1 (SEQ ID NO: 13), linker IGF-1 E3R (SEQ ID NO: 14), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), IGF-1 E3R A13C/L57C (SEQ ID NO: 19), and IGF-1 E3R L14C/L57C (SEQ ID NO: 20) on AKT S473 phosphorylation were measured in DU145 cells. FIG. 24A shows that the EC₅₀ value of IGF-1 E3R (SEQ ID NO: 4) was 8.6 nM. FIG. 24B shows that the EC₅₀ value of linker IGF-1 (SEQ ID NO: 13) was 9.9 nM. FIG. 24C shows that the EC₅₀ value of linker IGF-1 E3R (SEQ ID NO: 14) was 8.5 nM. FIG. 24D shows that the EC₅₀ value of IGF-1 E3R D12A (SEQ ID NO: 15) was 24.5 nM. FIG. 24E shows that the EC₅₀ value of IGF-1 E3R D20A (SEQ ID NO: 16) was 10.3 nM. FIG. 24F shows that the EC₅₀ value of IGF-1 E3R F49L (SEQ ID NO: 17) was 15.8 nM. FIG. 24G shows that the EC₅₀ value of IGF-1 E3R L54A (SEQ ID NO: 18) was 26.3 nM.

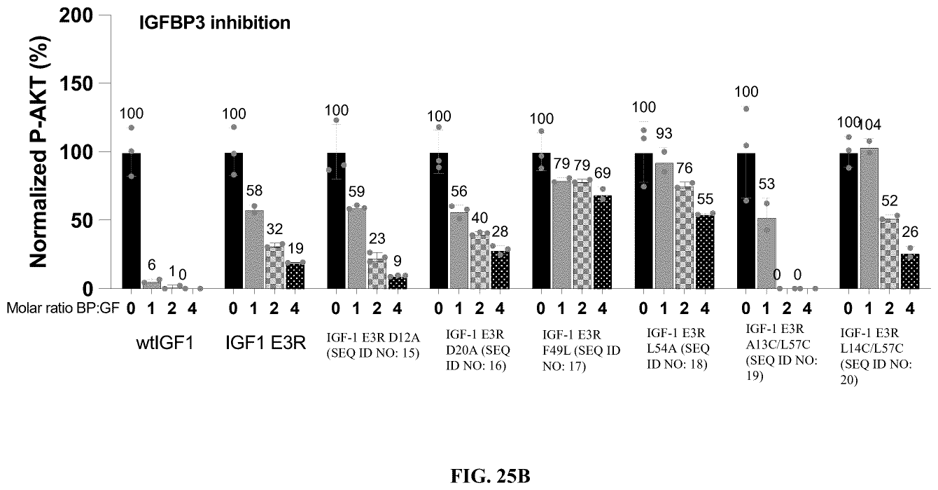

To determine the potency of IGF-1 variants in the presence of IGFBPs, pAKT levels were measured in DU145 cells after preincubation of IGFBP2 or IGFBP3 with wild-type IGF-1 (SEQ ID NO: 1), IGF-1 E3R (SEQ ID NO: 4), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), IGF-1 E3R A13C/L57C (SEQ ID NO: 19), or IGF-1 E3R L14C/L57C (SEQ ID NO: 20). FIGS. 25A and 25B demonstrate that IGF-1 E3R (SEQ ID NO: 4), IGF-1 E3R D12A (SEQ ID NO: 15), IGF-1 E3R D20A (SEQ ID NO: 16), IGF-1 E3R F49L (SEQ ID NO: 17), IGF-1 E3R L54A (SEQ ID NO: 18), or IGF-1 E3R L14C/L57C (SEQ ID NO: 20) are less inhibited by IGFBP2 and IGFBP3 compared to wild-type IGF-1 (SEQ ID NO: 1).

### Example 7: Potency of Key Variants Shows Synergy of IGF-1 E3R and IGF-1 F49L Mutations

### Materials and Methods

### In Vitro Potency Assay in DU145 Cells

For potency experiments, 15×10⁶ DU145 cells (ATCC, #HTB-81) frozen down at passage 5 (P5) were reseeded in a T175 culture flask containing DMEM/F12 (1:1) medium (Gibco, #11320-033) supplemented with 10% FBS (Gibco, #10437-028) and 10 μg/mL Gentamicin (Gibco, #15710-064) (hereafter referred to as culture medium) and expanded. At 80-90% confluency, cells were trypsinized, manually counted using a hemocytometer, resuspended at 2×10⁵ cells per mL in culture medium, and then reseeded in 24-well plates (Corning, #3524) in 0.5 mL aliquots per well (100,000 cells/well). 14-16 hours after reseeding, the culture medium was removed, and following a wash with 0.5 mL PBS, cultured for 6 h in 225 μL serum-free culture medium supplemented with 0.2% BSA (Millipore, #A3059). 25 μL aliquots of the following growth factor dilution series were added to the cells in duplicate for a final concentration in the well of: 457, 137, 41, 12.5, 3.7, 1.11, and 0.33 nM rhIGF1 (PeproTech #100-11), rhIGF1 E3R (SEQ ID NO: 4) or select IGF1 variant molecules as outlined in Table 2. After 15 min incubation at 37° C., medium was aspirated and cells lysed in 150 μL 1×PTR buffer (Extraction Buffer 5×PTR, Abcam #ab193970) containing 1× Extraction Enhancer Buffer (Extraction Enhancer Buffer 50×, Abcam #ab193971) and protease/phosphatase inhibitors (one Pierce mini tablet, #A32959, per 10 mL 1×PTR). After 15-30 min incubation on ice, plates were vortexed, and lysates transferred to Eppendorf tubes and stored at −80° C. for assessment of IGF1R-AKT signaling strength using the Human/Mouse/Rat Phospho-Akt (S473) Pan Specific DuoSet IC ELISA (RnD #DYC887B).

EC₅₀ fits were calculated using Graphpad Prism (Graphpad Software 2023). The curves shown are an average of 1-21 experiments with two biological replicates per experiment.

### IGFBP2/3 Inhibition Experiments

The same protocol as described above for in vitro potency assay in DU145 cells was followed, except that rhIGF1 (PeproTech #100-11) or select IGF1 variant molecules outlined in Table 2 were preincubated with IGFBP2 (R&D systems #674-B2) or IGFBP3 (R&D systems #675-B3) in a 1.5 mL Eppendorf tube at room temperature for 1.5 hrs prior to stimulating the cells. The preincubation was done with 250 nM growth factor (10×) and 4-fold molar excess of IGFBP2 or IGFBP3. Final concentration during stimulation was 25 nM Growth Factor with 100 nM IGFBP2 or IGFBP3. Phosphorylated AKT levels were measured using the Human/Mouse/Rat Phospho-Akt (S473) Pan Specific DuoSet IC ELISA (RnD #DYC887B). Inhibition was determined by normalizing the total pAKT signal to the control group (0 nM IGFBP, 100%). The bar diagram shown depicts the average of 5-28 biological replicates.

### Measurement of Spheroids' Growth of IHGMGE Cells

2500 immortalized human meibomian gland epithelial cells (IHMGEC, ATCC, #CRL-3472) were seeded per well of a 24-well plate in a drop of 50 μL matrix (Matrigel, Corning #354230) covered by proliferation medium (Keratinocyte Serum Free Medium (Gibco, #17005042) supplemented with 50 μg/mL bovine pituitary extract (Gibco, #13028-014), 5 ng/mL recombinant human epidermal growth factor (EGF, Gibco, #10450-013), 10 μg/mL Gentamicin (Gibco, #15710-064) and 1% Pen/Strep (Gibco, #15140-122)). Spheroids were grown in proliferation medium for eight days, followed by a differentiation stage of twelve days in presence of differentiation medium (DMEM:F12 medium (Gibco #11320-033) supplemented with 10 μg/mL Gentamicin (Gibco, #15710-064), 1% Pen/Strep (Gibco, #15140-122), 20 μM Rosiglitazone (Sigma Aldrich, #R2408), plus or minus 2 nM of recombinant human insulin-like growth factor-1 (rhIGF1, PeproTech #100-11) or 2 nM of rhIGF1 E3R (SEQ ID NO: 4), plus or minus 4 nM IGFBP2 (R&D systems #674-B2) or 4 nM IGFBP3 (R&D systems #675-B3). As a measure of proliferation, Fiji 2 by ImageJ was used to measure the diameter of each spheroid at day 12 of differentiation. To this end, a z-stack of 41 bright field images spanning a total of 1 mm were taken and maximum image projection was used to generate one final image that was used for measurement. N=3 (average of 3 wells, containing 45-78 spheroids per well).

### Results

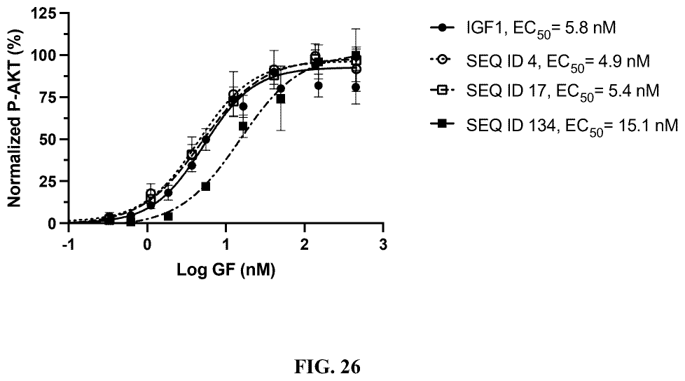

The dose-dependent effects of IGF-1 (SEQ ID NO: 1), IGF-1 E3R (SEQ ID NO: 4), IGF-1 E3R F49L (SEQ ID NO: 17), and IGF-1 F49L (SEQ ID NO: 134) on AKT S473 phosphorylation were measured in DU145 cells. FIG. 26 shows that the EC₅₀ value of IGF-1 was 5.8 nM; the EC₅₀ value of IGF-1 E3R (SEQ ID NO: 4) was 4.9 nM; the EC₅₀ value of IGF-1 E3R F49L (SEQ ID NO: 17) was 5.4 nM; and the EC₅₀ value of IGF-1 F49L (SEQ ID NO: 134) was 15.1 nM.

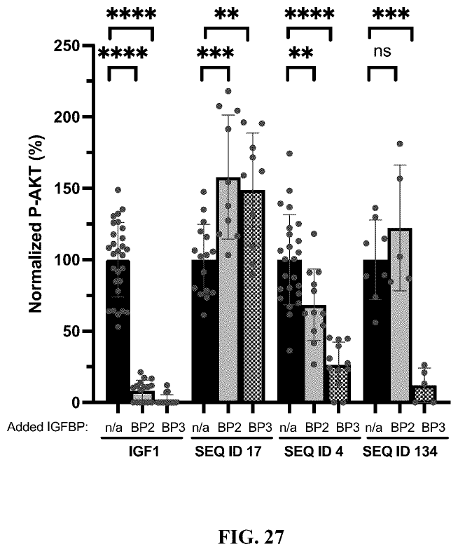

To determine the potency of IGF-1 variants in the presence of IGFBPs, pAKT levels measured in DU145 cells after preincubation of IGFBP2 or IGFBP3 with wild-type IGF-1 (SEQ ID NO: 1), IGF-1 E3R (SEQ ID NO: 4), IGF-1 E3R F49L (SEQ ID NO: 17), and IGF-1 F49L (SEQ ID NO: 134). FIG. 27 suggests that IGF-1 E3R (SEQ ID NO: 4) and IGF-1 F49L (SEQ ID NO: 134) are less inhibited by IGFBP2 and IGFBP3 compared to wild-type IGF-1 (SEQ ID NO: 1). Moreover, IGF-1 E3R F49L (SEQ ID NO: 17) exhibits a synergistic effect from the E3R and F49L mutations in IGF-1 in the presence of IGFBP2 or IGFBP3.

Table 2 shows the potency (EC₅₀ values) and IGFBP2- or IGFBP3-avoidance (as measured by P-AKT ELISA) of tested IGF-1 variants. EC50 values reflect the average of 1 to 21 individual experiments with two biological replicas per experiment. The IGFBP2 or IGFBP3 inhibition data reflect the mean+/−SD P-AKT activity in presence of these inhibitors compared to absence of inhibitors in 5 to 28 biological replicates.

TABLE 2
Potency and IGFBP Avoidance of Tested IGF-1 Variants

| | | | % Activity compared | |
|---|---|---|---|---|
| Sequence | | EC50 | to no IGFBP | |
| ID number | Name | (nM) | 4x BP2 | 4x BP3 |
| 1 | Wild-type | 5.8 | 8.5 | 1.5 |
| 4 | E3R | 4.9 | 69 | 25 |
| 15 | E3R D12A | 24.5 | 107 | 53 |
| 16 | E3R D20A | 10.3 | 106 | 39 |
| 17 | E3R F49L | 5.4 | 166 | 154 |
| 18 | E3R D54A | 26.3 | 157 | 55 |
| 28 | E3R D12L | unstable | | |
| 30 | E3R D12N | 8.7 | 291 | 171 |
| 33 | E3R D12R | 35.2 | 131 | 136 |
| 58 | E3R F49A | 8.2 | 157 | 191 |
| 60 | E3R F49D | 12.2 | 176 | 167 |
| 62 | E3R F49G | 18.3 | 228 | 166 |
| 64 | E3R F49I | 13.3 | 178 | 159 |
| 66 | E3R F49M | 7.6 | 215 | 163 |
| 67 | E3R F49N | 10.0 | 303 | 170 |
| 70 | E3R F49R | 5.2 | 142 | 154 |
| 72 | E3R F49T | 12.3 | 266 | 163 |
| 73 | E3R F49V | 345 | | |
| 74 | E3R F49W | 11.6 | 335 | 2.5 |
| 75 | E3R F49Y | 4.5 | 144 | 56 |
| 96 | E3R L54D | 15.9 | 238 | 70 |
| 104 | E3R L54N | 17.6 | 397 | 136 |
| 107 | E3R L54R | 12.4 | 281 | 58 |
| 132 | F49A | 15.1 | 130 | 16 |
| 133 | D12A | 25 | 0 | 0 |
| 134 | F49L | 7.5 | 130 | 10 |
| 135 | L54A | 11.8 | 21 | 3.5 |
| 139 | E3R-F49L-D12A | 17.7 | 357 | 342 |
| 140 | E3R-F49L-L54A | 11.4 | 175 | 239 |
| 141 | E3R-F49L-D12A-L54A | 68.1 | 456 | 344 |

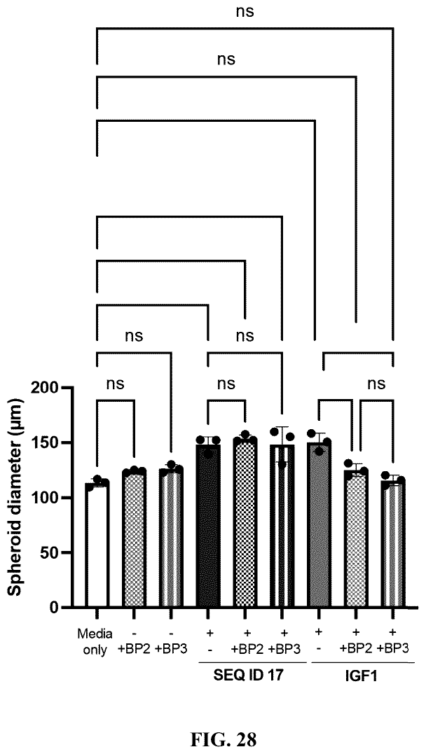

FIG. 28 provides that IGFBP2 and IGFBP3 inhibit spheroid growth in presence of IGF-1, while they have no effect on spheroid growth in presence of IGF-1 E3R F49L (SEQ ID NO: 17). It suggests that IGF-1 E3R F49L (SEQ ID NO: 17) avoids IGFBP2- and IGFBP3-mediated inhibition of meibomian gland spheroid growth.

### Example 8: In Vivo Effects of IGF-1 E3R F49L (SEQ ID NO: 17) on Meibomian Gland

### Materials and Methods

### Measurement of Proliferation Induction in the Meibomian Gland

Animals were administered a 10 μl eyedrop containing IGF-1 E3R F49L (SEQ ID NO: 17) at 1 mg/ml daily or twice daily for 2 weeks and sacrificed 24 hours after the last dose. Animals were sacrificed and eyelids were fixed in 4% paraformaldehyde overnight and switched to 30% sucrose 24 hours later. Eyelids were then embedded in OCT medium and sectioned in 10 micron sections and adhered to glass slides. Tissue was then stained for ki67 using Rat anti-Ki67 at 1 ug/mL (Invitrogen #14-5698-82) followed by detection with an anti-rat Alexafluor-labeled secondary antibody. In between antibody incubations, slides were washed with PBS+0.1% Tween-20 3×. Slides were then coverslipped and imaged. Cell nuclei were then stained with Hoescht and cover slipped for imaging. Random regions of interest (ROIs) were identified containing meibomian gland acini. Acini perimeter was outlined based on Hoescht stain in Image J and number of ki67+ cells lining the perimeter were measured and represented as number of ki67+ cells per 100 μm of acini perimeter. Data was then quantified and analyzed in Graphpad Prism (Insight Partners, NY, NY).

### Measurement of Meibomian Gland Density and Meibomian Gland Area

Animals received a daily bilateral 10 μl dose of Vehicle (PBS) or 1 mg/ml IGF-1 E3R F49L (SEQ ID NO: 17) in each eye for 4 weeks. Transillumination meibography was conducted before the start of the study and just prior to sacrifice using techniques previously published (Hwang, et al., Ocul Surf. (2021) 19:201-209). Briefly, mice were anesthetized using isoflurane and placed under a binocular dissection microscope (Leica MZ16FA, Leica Microsystems, Heerbrugg, Switzerland) equipped with a monochromatic camera (DFC340FX, Leica Microsystems, Heerbrugg, Switzerland. A broad band, halogen light source (OSL2 and OSL2B, Thorlabs, Newton, NJ) was used to transilluminate the eyelid for meibography. The light was delivered by a special optical fiber (BFL200HS02, Thorlabs, Newton, NJ) that had an input connector comprised of a bundle of seven 200 μm-diameter fibers and an output connector comprised of a linear array of fibers attached to a diffuser and a prism with glue. All images were taken at 20× magnification. After sacrifice, eyelids were removed and ex vivo meibography performed by placing eyelids onto an LED back light plate and taking transillumination photographs using the dissection microscope. To quantify meibomian gland area, individual eyelid images were analyzed using Meta Imaging Series software (Molecular Devices, Downington, PA). Specifically, the region of individual meibomian glands were manually outlined and the area of the region of interest measured for each individual gland in each image. The average area, density and intergland distance for two glands in each eyelid were then calculated, averaged and differences between the groups statistically analyzed using student's t-test using GraphPad Prism (Insight Partners, NY, NY).

Mice (5 mice/cage) were placed in standard, plastic mouse cage having two, 3000 rpm computer fans attached to opposites sides blowing air at 29.5 CPM into the cage. Cages were placed in an environmental chamber (ESPEC North America) that controlled air humidity and temperature set to 10% relative humidity and 25° C. with a 12 hr light/dark cycle. Using a computer sensor, humidity and temperature were recorded every 5 minutes throughout the experiment. At day 0, mice were anesthetized and tear volume and corneal fluorescein staining measured using the thread test and fluorescein staining. At day 1, animals were placed in the environmental chamber containing water and food ad lib. Mice were monitored daily for food and water and response to environmental conditions. At the end of the environmental exposure (Day 5 or Day 10) mice were anesthetized and tear volume and fluorescein staining measured prior to sacrifice. For the corneal fluorescein test, 50 μl of 0.05% fluorescein (BioGlo; HUB Pharmaceuticals, Rancho Cucamonga, CA, USA) in PBS was dropped on the cornea of the anesthetized mice, and they were manually forced to blink, to ensure that the fluorescein solution covered the entire surface. The corneal surface was then observed and photographed using high-resolution stereo fluorescence MZ10F microscope (Leica Microsystems) with a blue filter. All images were obtained using the LAS V4.5 Software under the same magnification (×20), exposure (0.124 seconds), gain (2.0×), saturation (1.00) and gamma (1.00) parameters. MetaMorph image analysis software (Molecular Device Inc, Downingtown, PA) was then used to calculate corneal erosion area and GFP intensity. MetaMorph uses an absolute threshold value that is applied across the whole image and any signal outside the upper- or lower-threshold limit is excluded from the analysis. The region of interest (area size: 243753) was saved and applied to the whole images. The original corneal fluorescence image was opened and separated by color. With green color image, the region was loaded in the center of the cornea. Threshold tool (threshold minimum value: 15) was opened and the measured data was exported to a Microsoft Excel (Microsoft, Seattle, WA) spreadsheet.

### Results

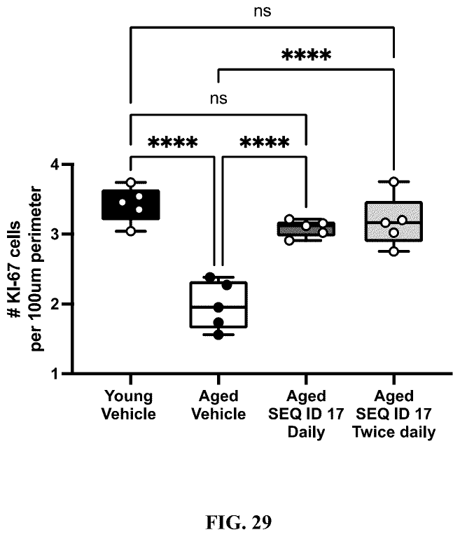

FIG. 29 shows that IGF-1 E3R F49L (SEQ ID NO: 17) treatment increased proliferation in meibomian gland acini in aged mice. Additionally, daily or twice daily eyedrop dosing of IGF-1 E3R F49L (SEQ ID NO: 17) has the same effect on proliferation induction in the meibomian gland.

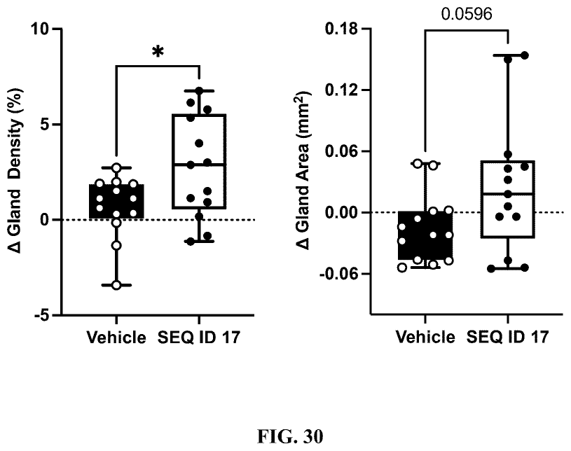

FIG. 30 shows that once daily eyedrop dosing of IGF-1 E3R F49L (SEQ ID NO: 17) at 1 mg/ml for one month increased meibomian gland density (left panel) and meibomian gland area (right panel) in aged mice compared to the treatment of vehicle control.

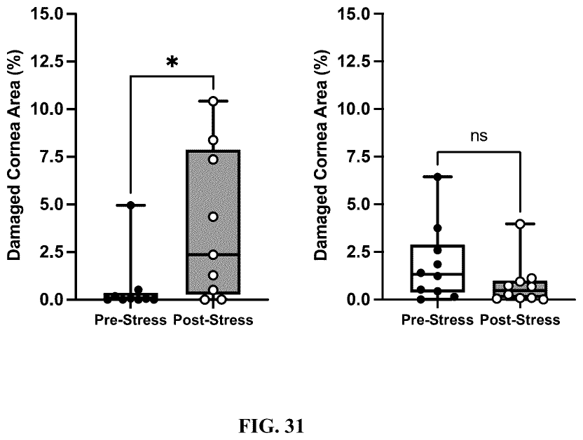

FIG. 31 shows that once daily eyedrop dosing of IGF-1 E3R F49L (SEQ ID NO: 17) (right panel) for one month reduced damaged cornea area in desiccation stress model mice compared to the treatment of vehicle control. It suggests that treatment of IGF-1 E3R F49L (SEQ ID NO: 17) enhances resistance to desiccation stress-induced corneal damage after cessation of dosing.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. An engineered polypeptide comprising an IGF-1 variant, wherein said engineered polypeptide has reduced affinity to at least one IGF binding protein (IGFBP) as compared to the affinity between wild-type IGF-1 (SEQ ID NO: 1) and the IGFBP.

Embodiment 2. The engineered polypeptide of Embodiment 1, wherein the engineered polypeptide comprises the amino acid sequence of any one of SEQ ID NOS: 2-771, or alternatively, any one of SEQ ID NOS: 2-767.

Embodiment 3. The engineered polypeptide of any preceding Embodiment, wherein the IGF-1 variant is a truncation.

Embodiment 4. The engineered polypeptide of any one of Embodiments 1-3, wherein the IGF-1 variant comprises or consists of the amino sequence of SEQ ID NO: 3.

Embodiment 5. The engineered polypeptide of any preceding Embodiment, wherein the IGF-1 variant comprises one or more amino acid substitutions relative to wild-type IGF-1 (SEQ ID NO: 1).

Embodiment 6. The engineered polypeptide of any one of Embodiments 1-3 or 5, wherein the IGF-1 variant comprises an amino acid deletion at position 37 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 7. The engineered polypeptide of any one of Embodiments 1-3, 5 or 6, wherein the IGF-1 variant comprises the amino sequence of SEQ ID NO: 6.

Embodiment 8. The engineered polypeptide of any one of Embodiments 1-3, wherein the IGF-1 variant comprises an amino acid substitution at position 60 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 9. The engineered polypeptide of any one of Embodiments 1-3, 5 or 8, wherein the IGF-1 variant comprises the amino sequence of SEQ ID NO: 2.

Embodiment 10. The engineered polypeptide of any one of Embodiments 1-3, wherein the IGF-1 variant comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 11. The engineered polypeptide of any one of Embodiments 1-3, 5 or 10, wherein the IGF-1 variant comprises the amino sequence of SEQ ID NO: 4.

Embodiment 12. The engineered polypeptide of any one of Embodiments 1-3 or 5, wherein the IGF-1 variant comprises the amino sequence of SEQ ID NO: 5.

Embodiment 13. The engineered polypeptide of any one of Embodiments 1-3, wherein the IGF-1 variant comprises the amino sequence of SEQ ID NO: 7.

Embodiment 14. The engineered polypeptide of any one of Embodiments 1-3, wherein the IGF-1 variant comprises the amino sequence of SEQ ID NO: 12.

Embodiment 15. The engineered polypeptide of any one of Embodiments 1-3, wherein the IGF-1 variant comprises an amino acid sequence selected from any one of SEQ ID NOS: 13-771, or alternatively any one of SEQ ID NOS: 13-767.

Embodiment 16. The engineered polypeptide of any one of Embodiments 1-3, wherein the IGF-1 variant comprises the amino sequence of SEQ ID NO: 16.

Embodiment 17. The engineered polypeptide of any one of Embodiments 1-3, wherein the IGF-1 variant comprises the amino sequence of SEQ ID NO: 17.

Embodiment 18. The engineered polypeptide of any one of Embodiments 1-3, wherein the IGF-1 variant comprises the amino sequence of SEQ ID NO: 18.

Embodiment 19. The engineered polypeptide of any one of Embodiments 1-3, wherein the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 20. The engineered polypeptide of Embodiment 19, wherein the amino acid substitution at position 3 is an arginine.

Embodiment 21. The engineered polypeptide of Embodiment 20, wherein the IGF-1 variant comprises an amino acid sequence selected from any one of SEQ ID NOS: 15 and 21-38.

Embodiment 22. The engineered polypeptide of any one of Embodiments 1-3, wherein the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 23. The engineered polypeptide of Embodiment 22, wherein the amino acid substitution at position 3 is an arginine.

Embodiment 24. The engineered polypeptide of Embodiment 23, wherein the IGF-1 variant comprises an amino acid sequence selected from any one of SEQ ID NOS: 58-75.

Embodiment 25. The engineered polypeptide of any one of Embodiments 1-3, wherein the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 26. The engineered polypeptide of Embodiment 25, wherein the amino acid substitution at position 3 is an arginine.

Embodiment 27. The engineered polypeptide of Embodiment 26, wherein the IGF-1 variant comprises an amino acid sequence selected from any one of SEQ ID NOS: 95-112.

Embodiment 28. The engineered polypeptide of any one of Embodiments 1-3, wherein the IGF-1 variant comprises (1) a truncation of the first three amino acid of the wild-type IGF-1 (SEQ ID NO: 1) and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 29. The engineered polypeptide of Embodiment 28, wherein the IGF-1 variant comprises an amino acid sequence selected from any one of SEQ ID NOS: 39-57.

Embodiment 30. The engineered polypeptide of any one of Embodiments 1-3, wherein the IGF-1 variant comprises (1) a truncation of the first three amino acid of the wild-type IGF-1 (SEQ ID NO: 1) and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 31. The engineered polypeptide of Embodiment 30, wherein the IGF-1 variant comprises an amino acid sequence selected from any one of SEQ ID NOS: 76-94.

Embodiment 32. The engineered polypeptide of any one of Embodiments 1-3, wherein the IGF-1 variant comprises (1) a truncation of the first three amino acid of the wild-type IGF-1 (SEQ ID NO: 1) and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 33. The engineered polypeptide of Embodiment 32, wherein the IGF-1 variant comprises an amino acid sequence selected from any one of SEQ ID NOS: 113-131.

Embodiment 34. The engineered polypeptide of Embodiment 5, wherein the one or more amino acid substitutions relative to wild-type IGF-1 (SEQ ID NO: 1) are at positions 12, 49, 54, or a combination thereof.

Embodiment 35. The engineered polypeptide of Embodiment 34, wherein the IGF-1 variant comprises an amino acid sequence selected from any one of SEQ ID NOS: 132-197.

Embodiment 36. The engineered polypeptide of any one of Embodiments 1-3, wherein the IGF-1 variant comprises an amino acid substitution at position 5 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 37. The engineered polypeptide of Embodiment 36, wherein the IGF-1 variant comprises an amino acid sequence selected from any one of SEQ ID NOS: 578-596.

Embodiment 38. The engineered polypeptide of Embodiment 36, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 39. The engineered polypeptide of Embodiment 38, wherein the amino acid substitution at position 3 is an arginine.

Embodiment 40. The engineered polypeptide of Embodiment 39, wherein the IGF-1 variant comprises an amino acid sequence selected from any one of SEQ ID NOS: 198-216.

Embodiment 41. The engineered polypeptide of Embodiment 36, wherein the IGF-1 variant further comprises a truncation of the first three amino acid of the wild-type IGF-1 (SEQ ID NO: 1).

Embodiment 42. The engineered polypeptide of Embodiment 41, wherein the IGF-1 variant comprises an amino acid sequence selected from any one of SEQ ID NOS: 217-235.

Embodiment 43. The engineered polypeptide of any one of Embodiments 1-3, wherein the IGF-1 variant comprises an amino acid substitution at position 7 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 44. The engineered polypeptide of Embodiment 43, wherein the IGF-1 variant comprises an amino acid sequence selected from any one of SEQ ID NOS: 597-615.

Embodiment 45. The engineered polypeptide of Embodiment 43, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 46. The engineered polypeptide of Embodiment 45, wherein the amino acid substitution at position 3 is an arginine.

Embodiment 47. The engineered polypeptide of Embodiment 46, wherein the IGF-1 variant comprises an amino acid sequence selected from any one of SEQ ID NOS: 236-254.

Embodiment 48. The engineered polypeptide of Embodiment 43, wherein the IGF-1 variant further comprises a truncation of the first three amino acid of the wild-type IGF-1 (SEQ ID NO: 1).

Embodiment 49. The engineered polypeptide of Embodiment 48, wherein the IGF-1 variant comprises an amino acid sequence selected from any one of SEQ ID NOS: 255-273.

Embodiment 50. The engineered polypeptide of any one of Embodiments 1-3, wherein the IGF-1 variant comprises an amino acid substitution at position 10 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 51. The engineered polypeptide of Embodiment 50, wherein the IGF-1 variant comprises an amino acid sequence selected from any one of SEQ ID NOS: 616-634.

Embodiment 52. The engineered polypeptide of Embodiment 50, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 53. The engineered polypeptide of Embodiment 52, wherein the amino acid substitution at position 3 is an arginine.

Embodiment 54. The engineered polypeptide of Embodiment 53, wherein the IGF-1 variant comprises an amino acid sequence selected from any one of SEQ ID NOS: 274-292.

Embodiment 55. The engineered polypeptide of Embodiment 50, wherein the IGF-1 variant further comprises a truncation of the first three amino acid of the wild-type IGF-1 (SEQ ID NO: 1).

Embodiment 56. The engineered polypeptide of Embodiment 55, wherein the IGF-1 variant comprises an amino acid sequence selected from any one of SEQ ID NOS: 293-311.

Embodiment 57. The engineered polypeptide of any one of Embodiments 1-3, wherein the IGF-1 variant comprises an amino acid substitution at position 17 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 58. The engineered polypeptide of Embodiment 57, wherein the IGF-1 variant comprises an amino acid sequence selected from any one of SEQ ID NOS: 635-653.

Embodiment 59. The engineered polypeptide of Embodiment 57, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 60. The engineered polypeptide of Embodiment 59, wherein the amino acid substitution at position 3 is an arginine.

Embodiment 61. The engineered polypeptide of Embodiment 60, wherein the IGF-1 variant comprises an amino acid sequence selected from any one of SEQ ID NOS: 312-330.

Embodiment 62. The engineered polypeptide of Embodiment 57, wherein the IGF-1 variant further comprises a truncation of the first three amino acid of the wild-type IGF-1 (SEQ ID NO: 1).

Embodiment 63. The engineered polypeptide of Embodiment 62, wherein the IGF-1 variant comprises an amino acid sequence selected from any one of SEQ ID NOS: 331-349.

Embodiment 64. The engineered polypeptide of any one of Embodiments 1-3, wherein the IGF-1 variant comprises an amino acid substitution at position 22 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 65. The engineered polypeptide of Embodiment 64, wherein the IGF-1 variant comprises an amino acid sequence selected from any one of SEQ ID NOS: 654-672.

Embodiment 66. The engineered polypeptide of Embodiment 64, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 67. The engineered polypeptide of Embodiment 66, wherein the amino acid substitution at position 3 is an arginine.

Embodiment 68. The engineered polypeptide of Embodiment 67, wherein the IGF-1 variant comprises an amino acid sequence selected from any one of SEQ ID NOS: 350-368.

Embodiment 69. The engineered polypeptide of Embodiment 64, wherein the IGF-1 variant further comprises a truncation of the first three amino acid of the wild-type IGF-1 (SEQ ID NO: 1).

Embodiment 70. The engineered polypeptide of Embodiment 69, wherein the IGF-1 variant comprises an amino acid sequence selected from any one of SEQ ID NOS: 369-387.

Embodiment 71. The engineered polypeptide of any one of Embodiments 1-3, wherein the IGF-1 variant comprises an amino acid substitution at position 23 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 72. The engineered polypeptide of Embodiment 71, wherein the IGF-1 variant comprises an amino acid sequence selected from any one of SEQ ID NOS: 673-691.

Embodiment 73. The engineered polypeptide of Embodiment 71, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 74. The engineered polypeptide of Embodiment 73, wherein the amino acid substitution at position 3 is an arginine.

Embodiment 75. The engineered polypeptide of Embodiment 74, wherein the IGF-1 variant comprises an amino acid sequence selected from any one of SEQ ID NOS: 388-406.

Embodiment 76. The engineered polypeptide of Embodiment 71, wherein the IGF-1 variant further comprises a truncation of the first three amino acid of the wild-type IGF-1 (SEQ ID NO: 1).

Embodiment 77. The engineered polypeptide of Embodiment 76, wherein the IGF-1 variant comprises an amino acid sequence selected from any one of SEQ ID NOS: 407-425.

Embodiment 78. The engineered polypeptide of any one of Embodiments 1-3, wherein the IGF-1 variant comprises an amino acid substitution at position 24 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 79. The engineered polypeptide of Embodiment 78, wherein the IGF-1 variant comprises an amino acid sequence selected from any one of SEQ ID NOS: 692-710.

Embodiment 80. The engineered polypeptide of Embodiment 78, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 81. The engineered polypeptide of Embodiment 80, wherein the amino acid substitution at position 3 is an arginine.

Embodiment 82. The engineered polypeptide of Embodiment 81, wherein the IGF-1 variant comprises an amino acid sequence selected from any one of SEQ ID NOS: 426-444.

Embodiment 83. The engineered polypeptide of Embodiment 78, wherein the IGF-1 variant further comprises a truncation of the first three amino acid of the wild-type IGF-1 (SEQ ID NO: 1).

Embodiment 84. The engineered polypeptide of Embodiment 83, wherein the IGF-1 variant comprises an amino acid sequence selected from any one of SEQ ID NOS: 445-463.

Embodiment 85. The engineered polypeptide of any one of Embodiments 1-3, wherein the IGF-1 variant comprises an amino acid substitution at position 25 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 86. The engineered polypeptide of Embodiment 85, wherein the IGF-1 variant comprises an amino acid sequence selected from any one of SEQ ID NOS: 711-729.

Embodiment 87. The engineered polypeptide of Embodiment 85, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 88. The engineered polypeptide of Embodiment 87, wherein the amino acid substitution at position 3 is an arginine.

Embodiment 89. The engineered polypeptide of Embodiment 88, wherein the IGF-1 variant comprises an amino acid sequence selected from any one of SEQ ID NOS: 464-482.

Embodiment 90. The engineered polypeptide of Embodiment 85, wherein the IGF-1 variant further comprises a truncation of the first three amino acid of the wild-type IGF-1 (SEQ ID NO: 1).

Embodiment 91. The engineered polypeptide of Embodiment 90, wherein the IGF-1 variant comprises an amino acid sequence selected from any one of SEQ ID NOS: 483-501.

Embodiment 92. The engineered polypeptide of any one of Embodiments 1-3, wherein the IGF-1 variant comprises an amino acid substitution at position 34 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 93. The engineered polypeptide of Embodiment 92, wherein the IGF-1 variant comprises an amino acid sequence selected from any one of SEQ ID NOS: 730-748.

Embodiment 94. The engineered polypeptide of Embodiment 92, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 95. The engineered polypeptide of Embodiment 94, wherein the amino acid substitution at position 3 is an arginine.

Embodiment 96. The engineered polypeptide of Embodiment 95, wherein the IGF-1 variant comprises an amino acid sequence selected from any one of SEQ ID NOS: 502-520.

Embodiment 97. The engineered polypeptide of Embodiment 92, wherein the IGF-1 variant further comprises a truncation of the first three amino acid of the wild-type IGF-1 (SEQ ID NO: 1).

Embodiment 98. The engineered polypeptide of Embodiment 97, wherein the IGF-1 variant comprises an amino acid sequence selected from any one of SEQ ID NOS: 521-539.

Embodiment 99. The engineered polypeptide of any one of Embodiments 1-3, wherein the IGF-1 variant comprises an amino acid substitution at position 57 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 100. The engineered polypeptide of Embodiment 99, wherein the IGF-1 variant comprises an amino acid sequence selected from any one of SEQ ID NOS: 749-767.

Embodiment 101. The engineered polypeptide of Embodiment 99, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 102. The engineered polypeptide of Embodiment 101, wherein the amino acid substitution at position 3 is an arginine.

Embodiment 103. The engineered polypeptide of Embodiment 102, wherein the IGF-1 variant comprises an amino acid sequence selected from any one of SEQ ID NOS: 540-558.

Embodiment 104. The engineered polypeptide of Embodiment 99, wherein the IGF-1 variant further comprises a truncation of the first three amino acid of the wild-type IGF-1 (SEQ ID NO: 1).

Embodiment 105. The engineered polypeptide of Embodiment 104, wherein the IGF-1 variant comprises an amino acid sequence selected from any one of SEQ ID NOS: 549-577.

Embodiment 106. The engineered polypeptide of any preceding Embodiment, wherein the IGF-1 variant is coupled to a cell-penetrating peptide (CPP) or skin-penetrating peptide (SPP).

Embodiment 107. The engineered polypeptide of Embodiment 106, wherein the IGF-1 variant is coupled to a cell-penetrating peptide selected from the group consisting of SEQ ID NO: 10 and SEQ ID NO: 11.

Embodiment 108. The engineered polypeptide of Embodiment 106, wherein the IGF-1 variant is coupled to the skin-penetrating peptide of SEQ ID NO: 9.

Embodiment 109. The engineered polypeptide of Embodiment 108, wherein the IGF-1 variant comprises the amino acid sequence of SEQ ID NO: 8.

Embodiment 110. The engineered polypeptide of any one of Embodiments 1-109, wherein the at least one IGFBP comprises IGFBP2.

Embodiment 111. The engineered polypeptide of any one of Embodiments 1-109, wherein the at least one IGFBP comprises IGFBP3.

Embodiment 112. The engineered polypeptide of any one of Embodiments 1-109, wherein the at least one IGFBP comprises IGFBP1.

Embodiment 113. The engineered polypeptide of any one of Embodiments 1-109, wherein the at least one IGFBP comprises IGFBP4.

Embodiment 114. The engineered polypeptide of any one of Embodiments 1-109, wherein the at least one IGFBP comprises IGFBP5.

Embodiment 115. The engineered polypeptide of any one of Embodiments 1-109, wherein the at least one IGFBP comprises IGFBP6.

Embodiment 116. A pharmaceutical composition comprising a therapeutically effective amount of an IGF-1 variant that has reduced affinity to at least one IGF binding protein (IGFBP) as compared to the affinity for the interaction between wild-type IGF-1 (SEQ ID NO: 1) and the IGFBP, wherein the pharmaceutical composition is formulated for local administration.

Embodiment 117. The pharmaceutical composition of Embodiment 116, wherein the pharmaceutical composition is formulated for local administration to an eye or eyelid.

Embodiment 118. A pharmaceutical composition comprising a therapeutically effective amount of an IGF-1 variant, wherein a local administration of the pharmaceutical composition to an eye or eyelid results in one or more of
(a) an increase in a size of meibomian glands;
(b) a decrease in meibomian gland atrophy;
(c) a reversal of age-associated meibomian gland atrophy;
(d) an increase in a function of one or more meibocytes;
(e) an increase in corneal epithelial cell proliferation;
(f) an increase in meibocyte proliferation;
(g) an increase in meibocyte differentiation;
(h) an increase in corneal healing rate;
(i) an increase in IGFT receptor (IGFTR) activation in the meibomian glands;
(j) an increase duration of IGF1R activation in the meibomian glands; and
(k) an increase of lipid content of the meibomian glands.

Embodiment 119. A pharmaceutical composition comprising a therapeutically effective amount of an IGF-1 variant, wherein a local administration of the pharmaceutical composition to an eye or eyelid results in one or more of
(a) an increase in a size of meibomian glands;
(b) a decrease in meibomian gland atrophy;
(c) a reversal of age-associated meibomian gland atrophy;
(d) an increase in a function of one or more meibocytes;
(e) an increase in corneal epithelial cell proliferation;
(f) an increase in corneal healing rate;
(g) an increase in IGF1 receptor (IGF1R) activation in the meibomian glands;
(h) an increase duration of IGF1R activation in the meibomian glands; and
(i) an increase of lipid content of the meibomian glands.

Embodiment 120. The pharmaceutical composition of Embodiment 118 or 119, wherein the IGF-1 variant has reduced affinity to at least one IGF binding protein relative to wild-type IGF-1 to the IGFBP.

Embodiment 121. A non-naturally occurring IGF-1 variant that has reduced affinity to at least one IGF binding protein (IGFP) as compared to the affinity for the interaction between wild-type IGF-1 (SEQ ID NO: 1) and the IGFBP.

Embodiment 122. A pharmaceutical composition comprising a therapeutically effective amount of the non-naturally occurring IGF-1 variant of Embodiment 121.

Embodiment 123. The pharmaceutical composition of any one of Embodiments 116-120 and 122, further comprising one or more pharmaceutically acceptable excipients.

Embodiment 124. The pharmaceutical composition of Embodiment 123, wherein the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone.

Embodiment 125. The pharmaceutical composition of any one of Embodiments 116-120 and 122-124, wherein the pharmaceutical composition is formulated for delivery via an eyedrop.

Embodiment 126. The pharmaceutical composition of any one of Embodiments 116-120 and 122-124, wherein the pharmaceutical composition comprises a cream for administration to one or both eyelids of a subject.

Embodiment 127. The pharmaceutical composition or IGF-1 variant of any one of Embodiments 116-126, wherein the IGF-1 variant has at least 80%, at least 85%, at least 90%, at least 95%, or at least 98% sequence identity to SEQ ID NO: 1.

Embodiment 128. The pharmaceutical composition or IGF-1 variant of any one of Embodiments 116-127, wherein the pharmaceutical composition or IGF-1 variant, when administered to subjects suffering from Meibomian gland dysfunction, results in a median increase in surface area or volume of meibomian glands within the inner eyelid surface of the subject as compared to subjects who do not receive the pharmaceutical composition or IGF-1 variant.

Embodiment 129. The pharmaceutical composition or IGF-1 variant of any one of Embodiments 116-128, wherein the pharmaceutical composition or IGF-1 variant, when administered to subjects suffering from Meibomian gland dysfunction, results in a median increase in lipid content within a Meibomian gland of the subject as compared to subjects who do not receive the pharmaceutical composition or IGF-1 variant.

Embodiment 130. The pharmaceutical composition or IGF-1 variant of any one of Embodiments 116-129, wherein the pharmaceutical composition or IGF-1 variant, when administered to subjects suffering from Meibomian gland dysfunction results, in a median increase in lipid quality within a Meibomian gland of the subject as compared to subjects who do not receive the pharmaceutical composition or IGF-1 variant.

Embodiment 131. The pharmaceutical composition or IGF-1 variant of any one of Embodiments 116-130, wherein the pharmaceutical composition or IGF-1 variant, when administered to subjects suffering from Meibomian gland dysfunction results, in a median increase in the number of meibocytes within a Meibomian gland of the subject as compared to subjects who do not receive the pharmaceutical composition or IGF-1 variant.

Embodiment 132. The pharmaceutical composition or IGF-1 variant of any one of Embodiments 116-131, wherein the pharmaceutical composition or IGF-1 variant, when administered to subjects suffering from Meibomian gland dysfunction, results in a median increase in release of lipid from acini of a Meibomian gland of subjects as compared to a subjects who do not receive the pharmaceutical composition or IGF-1 variant.

Embodiment 133. The pharmaceutical composition or IGF-1 variant of any one of Embodiments 116-132, wherein the pharmaceutical composition or IGF-1 variant, when administered to subjects suffering from meibomian gland dysfunction results in a median increase in duration of phosphorylation of Akt in meibocytes relative to subjects who do not receive the pharmaceutical composition or IGF-1 variant.

Embodiment 134. The pharmaceutical composition or IGF-1 variant of any one of Embodiments 116-133, wherein the IGF-1 variant is a truncation.

Embodiment 135. The pharmaceutical composition or IGF-1 variant of any one of Embodiments 116-134, wherein the IGF-1 variant comprises or consists of the amino sequence of SEQ ID NO: 3.

Embodiment 136. The pharmaceutical composition or IGF-1 variant of any one of Embodiments 1-134, wherein the IGF-1 variant comprises one or more amino acid substitutions relative to wild-type IGF-1 (SEQ ID NO: 1).

Embodiment 137. The pharmaceutical composition or IGF-1 variant of any one of Embodiments 116-134 and 136, wherein the IGF-1 variant comprises an amino acid deletion at position 37 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 138. The pharmaceutical composition or IGF-1 variant of any one of Embodiments 116-134, 136, and 137, wherein the IGF-1 variant comprises the amino sequence of SEQ ID NO: 6.

Embodiment 139. The pharmaceutical composition or IGF-1 variant of any one of Embodiments 116-133, wherein the IGF-1 variant comprises an amino acid substitution at position 60 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 140. The pharmaceutical composition or IGF-1 variant of any one of Embodiments 116-133, 136, and 139, wherein the IGF-1 variant comprises the amino sequence of SEQ ID NO: 2.

Embodiment 141. The pharmaceutical composition or IGF-1 variant of any one of Embodiments 116-133, wherein the IGF-1 variant comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 142. The pharmaceutical composition or IGF-1 variant of any one of Embodiments 116-133, 136, and 141, wherein the IGF-1 variant comprises the amino sequence of SEQ ID NO: 4.

Embodiment 143. The pharmaceutical composition or IGF-1 variant of any one of Embodiments 116-133 and 136, wherein the IGF-1 variant comprises the amino sequence of SEQ ID NO: 5.

Embodiment 144. The pharmaceutical composition or IGF-1 variant of any one of Embodiments 116-133, wherein the IGF-1 variant comprises the amino sequence of SEQ ID NO: 7.

Embodiment 145. The pharmaceutical composition or IGF-1 variant of any one of Embodiments 116-133, wherein the IGF-1 variant comprises the amino sequence of SEQ ID NO: 12.

Embodiment 146. The pharmaceutical composition or IGF-1 variant of any one of Embodiments 116-133, wherein the IGF-1 variant comprises an amino acid sequence selected from any one of SEQ ID NOS: 13-771, or alternatively any one of SEQ ID NOS: 13-767.

Embodiment 147. The pharmaceutical composition or IGF-1 variant of any one of Embodiments 116-133, wherein the IGF-1 variant comprises the amino sequence of SEQ ID NO: 16.

Embodiment 148. The pharmaceutical composition or IGF-1 variant of any one of Embodiments 116-133, wherein the IGF-1 variant comprises the amino sequence of SEQ ID NO: 17.

Embodiment 149. The pharmaceutical composition or IGF-1 variant of any one of Embodiments 1-133, wherein the IGF-1 variant comprises the amino sequence of SEQ ID NO: 18.

Embodiment 150. The pharmaceutical composition or IGF-1 variant of any one of Embodiments 116-133, wherein the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 151. The pharmaceutical composition or IGF-1 variant of Embodiment 150, wherein the amino acid substitution at position 3 is an arginine.

Embodiment 152. The pharmaceutical composition or IGF-1 variant of Embodiment 151, wherein the IGF-1 variant comprises an amino acid sequence selected from any one of SEQ ID NOS: 15 and 21-38.

Embodiment 153. The pharmaceutical composition or IGF-1 variant of any one of Embodiments 116-133, wherein the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 154. The pharmaceutical composition or IGF-1 variant of Embodiment 153, wherein the amino acid substitution at position 3 is an arginine.

Embodiment 155. The pharmaceutical composition or IGF-1 variant of Embodiment 154, wherein the IGF-1 variant comprises an amino acid sequence selected from any one of SEQ ID NOS: 58-75.

Embodiment 156. The pharmaceutical composition or IGF-1 variant of any one of Embodiments 116-133, wherein the IGF-1 variant comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 157. The pharmaceutical composition or IGF-1 variant of Embodiment 156, wherein the amino acid substitution at position 3 is an arginine.

Embodiment 158. The pharmaceutical composition or IGF-1 variant of Embodiment 157, wherein the IGF-1 variant comprises an amino acid sequence selected from any one of SEQ ID NOS: 95-112.

Embodiment 159. The pharmaceutical composition or IGF-1 variant of any one of Embodiments 116-133, wherein the IGF-1 variant comprises (1) a truncation of the first three amino acid of the wild-type IGF-1 (SEQ ID NO: 1) and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 160. The pharmaceutical composition or IGF-1 variant of Embodiment 159, wherein the IGF-1 variant comprises an amino acid sequence selected from any one of SEQ ID NOS: 39-57.

Embodiment 161. The pharmaceutical composition or IGF-1 variant of any one of Embodiments 116-133, wherein the IGF-1 variant comprises (1) a truncation of the first three amino acid of the wild-type IGF-1 (SEQ ID NO: 1) and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 162. The pharmaceutical composition or IGF-1 variant of Embodiment 161, wherein the IGF-1 variant comprises an amino acid sequence selected from any one of SEQ ID NOS: 76-94.

Embodiment 163. The pharmaceutical composition or IGF-1 variant of any one of Embodiments 1-133, wherein the IGF-1 variant comprises (1) a truncation of the first three amino acid of the wild-type IGF-1 (SEQ ID NO: 1) and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 164. The pharmaceutical composition or IGF-1 variant of Embodiment 163, wherein the IGF-1 variant comprises an amino acid sequence selected from any one of SEQ ID NOS: 113-131.

Embodiment 165. The pharmaceutical composition or IGF-1 variant of Embodiment 136, wherein the one or more amino acid substitutions relative to wild-type IGF-1 (SEQ ID NO: 1) are at positions 12, 49, 54, or a combination thereof.

Embodiment 166. The pharmaceutical composition or IGF-1 variant of Embodiment 165, wherein the IGF-1 variant comprises an amino acid sequence selected from any one of SEQ ID NOS: 132-197.

Embodiment 167. The pharmaceutical composition or IGF-1 variant of any one of Embodiments 116-133, wherein the IGF-1 variant comprises an amino acid substitution at position 5 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 168. The pharmaceutical composition or IGF-1 variant of Embodiment 167, wherein the IGF-1 variant comprises an amino acid sequence selected from any one of SEQ ID NOS: 578-596.

Embodiment 169. The pharmaceutical composition or IGF-1 variant of Embodiment 167, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 170. The pharmaceutical composition or IGF-1 variant of Embodiment 169, wherein the amino acid substitution at position 3 is an arginine.

Embodiment 171. The pharmaceutical composition or IGF-1 variant of Embodiment 170, wherein the IGF-1 variant comprises an amino acid sequence selected from any one of SEQ ID NOS: 198-216.

Embodiment 172. The pharmaceutical composition or IGF-1 variant of Embodiment 167, wherein the IGF-1 variant further comprises a truncation of the first three amino acid of the wild-type IGF-1 (SEQ ID NO: 1).

Embodiment 173. The pharmaceutical composition or IGF-1 variant of Embodiment 172, wherein the IGF-1 variant comprises an amino acid sequence selected from any one of SEQ ID NOS: 217-235.

Embodiment 174. The pharmaceutical composition or IGF-1 variant of any one of Embodiments 116-133, wherein the IGF-1 variant comprises an amino acid substitution at position 7 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 175. The pharmaceutical composition or IGF-1 variant of Embodiment 174, wherein the IGF-1 variant comprises an amino acid sequence selected from any one of SEQ ID NOS: 597-615.

Embodiment 176. The pharmaceutical composition or IGF-1 variant of Embodiment 174, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 177. The pharmaceutical composition or IGF-1 variant of Embodiment 169, wherein the amino acid substitution at position 3 is an arginine.

Embodiment 178. The pharmaceutical composition or IGF-1 variant of Embodiment 177, wherein the IGF-1 variant comprises an amino acid sequence selected from any one of SEQ ID NOS: 236-254.

Embodiment 179. The pharmaceutical composition or IGF-1 variant of Embodiment 174, wherein the IGF-1 variant further comprises a truncation of the first three amino acid of the wild-type IGF-1 (SEQ ID NO: 1).

Embodiment 180. The pharmaceutical composition or IGF-1 variant of Embodiment 179, wherein the IGF-1 variant comprises an amino acid sequence selected from any one of SEQ ID NOS: 255-273.

Embodiment 181. The pharmaceutical composition or IGF-1 variant of any one of Embodiments 116-133, wherein the IGF-1 variant comprises an amino acid substitution at position 10 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 182. The pharmaceutical composition or IGF-1 variant of Embodiment 181, wherein the IGF-1 variant comprises an amino acid sequence selected from any one of SEQ ID NOS: 616-634.

Embodiment 183. The pharmaceutical composition or IGF-1 variant of Embodiment 181, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 184. The pharmaceutical composition or IGF-1 variant of Embodiment 183, wherein the amino acid substitution at position 3 is an arginine.

Embodiment 185. The pharmaceutical composition or IGF-1 variant of Embodiment 184, wherein the IGF-1 variant comprises an amino acid sequence selected from any one of SEQ ID NOS: 274-292.

Embodiment 186. The pharmaceutical composition or IGF-1 variant of Embodiment 181, wherein the IGF-1 variant further comprises a truncation of the first three amino acid of the wild-type IGF-1 (SEQ ID NO: 1).

Embodiment 187. The pharmaceutical composition or IGF-1 variant of Embodiment 186, wherein the IGF-1 variant comprises an amino acid sequence selected from any one of SEQ ID NOS: 293-311.

Embodiment 188. The pharmaceutical composition or IGF-1 variant of any one of Embodiments 116-133, wherein the IGF-1 variant comprises an amino acid substitution at position 17 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 189. The pharmaceutical composition or IGF-1 variant of Embodiment 188, wherein the IGF-1 variant comprises an amino acid sequence selected from any one of SEQ ID NOS: 635-653.

Embodiment 190. The pharmaceutical composition or IGF-1 variant of Embodiment 188, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 191. The pharmaceutical composition or IGF-1 variant of Embodiment 190, wherein the amino acid substitution at position 3 is an arginine.

Embodiment 192. The pharmaceutical composition or IGF-1 variant of Embodiment 191, wherein the IGF-1 variant comprises an amino acid sequence selected from any one of SEQ ID NOS: 312-330.

Embodiment 193. The pharmaceutical composition or IGF-1 variant of Embodiment 188, wherein the IGF-1 variant further comprises a truncation of the first three amino acid of the wild-type IGF-1 (SEQ ID NO: 1).

Embodiment 194. The pharmaceutical composition or IGF-1 variant of Embodiment 193, wherein the IGF-1 variant comprises an amino acid sequence selected from any one of SEQ ID NOS: 331-349.

Embodiment 195. The pharmaceutical composition or IGF-1 variant of any one of Embodiments 116-133, wherein the IGF-1 variant comprises an amino acid substitution at position 22 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 196. The pharmaceutical composition or IGF-1 variant of Embodiment 195, wherein the IGF-1 variant comprises an amino acid sequence selected from any one of SEQ ID NOS: 654-672.

Embodiment 197. The pharmaceutical composition or IGF-1 variant of Embodiment 195, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 198. The pharmaceutical composition or IGF-1 variant of Embodiment 197, wherein the amino acid substitution at position 3 is an arginine.

Embodiment 199. The pharmaceutical composition or IGF-1 variant of Embodiment 198, wherein the IGF-1 variant comprises an amino acid sequence selected from any one of SEQ ID NOS: 350-368.

Embodiment 200. The pharmaceutical composition or IGF-1 variant of Embodiment 195, wherein the IGF-1 variant further comprises a truncation of the first three amino acid of the wild-type IGF-1 (SEQ ID NO: 1).

Embodiment 201. The pharmaceutical composition or IGF-1 variant of Embodiment 200, wherein the IGF-1 variant comprises an amino acid sequence selected from any one of SEQ ID NOS: 369-387.

Embodiment 202. The pharmaceutical composition or IGF-1 variant of any one of Embodiments 116-133, wherein the IGF-1 variant comprises an amino acid substitution at position 23 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 203. The pharmaceutical composition or IGF-1 variant of Embodiment 202, wherein the IGF-1 variant comprises an amino acid sequence selected from any one of SEQ ID NOS: 673-691.

Embodiment 204. The pharmaceutical composition or IGF-1 variant of Embodiment 202, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 205. The pharmaceutical composition or IGF-1 variant of Embodiment 204, wherein the amino acid substitution at position 3 is an arginine.

Embodiment 206. The pharmaceutical composition or IGF-1 variant of Embodiment 205, wherein the IGF-1 variant comprises an amino acid sequence selected from any one of SEQ ID NOS: 388-406.

Embodiment 207. The pharmaceutical composition or IGF-1 variant of Embodiment 202, wherein the IGF-1 variant further comprises a truncation of the first three amino acid of the wild-type IGF-1 (SEQ ID NO: 1).

Embodiment 208. The pharmaceutical composition or IGF-1 variant of Embodiment 207, wherein the IGF-1 variant comprises an amino acid sequence selected from any one of SEQ ID NOS: 407-425.

Embodiment 209. The pharmaceutical composition or IGF-1 variant of any one of Embodiments 116-133, wherein the IGF-1 variant comprises an amino acid substitution at position 24 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 210. The pharmaceutical composition or IGF-1 variant of Embodiment 209, wherein the IGF-1 variant comprises an amino acid sequence selected from any one of SEQ ID NOS: 692-710.

Embodiment 211. The pharmaceutical composition or IGF-1 variant of Embodiment 209, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 212. The pharmaceutical composition or IGF-1 variant of Embodiment 211, wherein the amino acid substitution at position 3 is an arginine.

Embodiment 213. The pharmaceutical composition or IGF-1 variant of Embodiment 212, wherein the IGF-1 variant comprises an amino acid sequence selected from any one of SEQ ID NOS: 426-444.

Embodiment 214. The pharmaceutical composition or IGF-1 variant of Embodiment 209, wherein the IGF-1 variant further comprises a truncation of the first three amino acid of the wild-type IGF-1 (SEQ ID NO: 1).

Embodiment 215. The pharmaceutical composition or IGF-1 variant of Embodiment 214, wherein the IGF-1 variant comprises an amino acid sequence selected from any one of SEQ ID NOS: 445-463.

Embodiment 216. The pharmaceutical composition or IGF-1 variant of any one of Embodiments 116-133, wherein the IGF-1 variant comprises an amino acid substitution at position 25 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 217. The pharmaceutical composition or IGF-1 variant of Embodiment 216, wherein the IGF-1 variant comprises an amino acid sequence selected from any one of SEQ ID NOS: 711-729.

Embodiment 218. The pharmaceutical composition or IGF-1 variant of Embodiment 216, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 219. The pharmaceutical composition or IGF-1 variant of Embodiment 218, wherein the amino acid substitution at position 3 is an arginine.

Embodiment 220. The pharmaceutical composition or IGF-1 variant of Embodiment 219, wherein the IGF-1 variant comprises an amino acid sequence selected from any one of SEQ ID NOS: 464-482.

Embodiment 221. The pharmaceutical composition or IGF-1 variant of Embodiment 216, wherein the IGF-1 variant further comprises a truncation of the first three amino acid of the wild-type IGF-1 (SEQ ID NO: 1).

Embodiment 222. The pharmaceutical composition or IGF-1 variant of Embodiment 221, wherein the IGF-1 variant comprises an amino acid sequence selected from any one of SEQ ID NOS: 483-501.

Embodiment 223. The pharmaceutical composition or IGF-1 variant of any one of Embodiments 116-133, wherein the IGF-1 variant comprises an amino acid substitution at position 34 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 224. The pharmaceutical composition or IGF-1 variant of Embodiment 223, wherein the IGF-1 variant comprises an amino acid sequence selected from any one of SEQ ID NOS: 730-748.

Embodiment 225. The pharmaceutical composition or IGF-1 variant of Embodiment 223, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 226. The pharmaceutical composition or IGF-1 variant of Embodiment 225, wherein the amino acid substitution at position 3 is an arginine.

Embodiment 227. The pharmaceutical composition or IGF-1 variant of Embodiment 226, wherein the IGF-1 variant comprises an amino acid sequence selected from any one of SEQ ID NOS: 502-520.

Embodiment 228. The pharmaceutical composition or IGF-1 variant of Embodiment 223, wherein the IGF-1 variant further comprises a truncation of the first three amino acid of the wild-type IGF-1 (SEQ ID NO: 1).

Embodiment 229. The pharmaceutical composition or IGF-1 variant of Embodiment 228, wherein the IGF-1 variant comprises an amino acid sequence selected from any one of SEQ ID NOS: 521-539.

Embodiment 230. The pharmaceutical composition or IGF-1 variant of any one of Embodiments 116-133, wherein the IGF-1 variant comprises an amino acid substitution at position 57 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 231. The pharmaceutical composition or IGF-1 variant of Embodiment 230, wherein the IGF-1 variant comprises an amino acid sequence selected from any one of SEQ ID NOS: 749-767.

Embodiment 232. The pharmaceutical composition or IGF-1 variant of Embodiment 230, wherein the IGF-1 variant further comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 233. The pharmaceutical composition or IGF-1 variant of Embodiment 232, wherein the amino acid substitution at position 3 is an arginine.

Embodiment 234. The pharmaceutical composition or IGF-1 variant of Embodiment 233, wherein the IGF-1 variant comprises an amino acid sequence selected from any one of SEQ ID NOS: 540-558.

Embodiment 235. The pharmaceutical composition or IGF-1 variant of Embodiment 230, wherein the IGF-1 variant further comprises a truncation of the first three amino acid of the wild-type IGF-1 (SEQ ID NO: 1).

Embodiment 236. The pharmaceutical composition or IGF-1 variant of Embodiment 235, wherein the IGF-1 variant comprises an amino acid sequence selected from any one of SEQ ID NOS: 549-577.

Embodiment 237. The pharmaceutical composition or IGF-1 variant of any one of Embodiments 116-236, wherein the IGF-1 variant is coupled to a cell-penetrating peptide (CPP) or skin-penetrating peptide (SPP).

Embodiment 238. The pharmaceutical composition or IGF-1 variant of Embodiment 237, wherein the IGF-1 variant is coupled to a cell-penetrating peptide selected from the group consisting of SEQ ID NO: 10 and SEQ ID NO: 11.

Embodiment 239. The pharmaceutical composition or IGF-1 variant of Embodiment 237, wherein the IGF-1 variant is coupled to the skin-penetrating peptide of SEQ ID NO: 9.

Embodiment 240. The pharmaceutical composition or IGF-1 variant of Embodiment 239, wherein the IGF-1 variant comprises the amino acid sequence of SEQ ID NO: 8.

Embodiment 241. The pharmaceutical composition or IGF-1 variant of any one of Embodiments 116-117 and 120-240, wherein the at least one IGFBP comprises IGFBP2.

Embodiment 242. The pharmaceutical composition or IGF-1 variant of any one of Embodiments 116-117 and 120-241, wherein the at least one IGFBP comprises IGFBP3.

Embodiment 243. The pharmaceutical composition or IGF-1 variant of any one of Embodiments 116-117 and 120-242, wherein the at least one IGFBP comprises IGFBP1.

Embodiment 244. The pharmaceutical composition or IGF-1 variant of any one of Embodiments 116-117 and 120-243, wherein the at least one IGFBP comprises IGFBP4.

Embodiment 245. The pharmaceutical composition or IGF-1 variant of any one of Embodiments 116-117 and 120-244, wherein the at least one IGFBP comprises IGFBP5.

Embodiment 246. The pharmaceutical composition or IGF-1 variant of any one of Embodiments 116-117 and 120-245, wherein the at least one IGFBP comprises IGFBP6.

Embodiment 247. The pharmaceutical composition or IGF-1 variant of any one of Embodiments 116-246, wherein administration of the pharmaceutical composition or IGF-1 variant to an eye or eyelid of the subject results in an increase in a size of the meibomian glands.

Embodiment 248. The pharmaceutical composition or IGF-1 variant of any one of Embodiments 116-247, wherein administration of the pharmaceutical composition or IGF-1 variant to an eye or eyelid of the subject results in an increase in the number of meibocytes in the meibomian glands.

Embodiment 249. The pharmaceutical composition or IGF-1 variant of any one of Embodiments 116-248, wherein administration of the pharmaceutical composition or IGF-1 variant to an eye or eyelid of the subject results in a decrease in meibomian gland atrophy.

Embodiment 250. The pharmaceutical composition or IGF-1 variant of any one of Embodiments 116-249, wherein administration of the pharmaceutical composition or IGF-1 variant to an eye or eyelid of the subject results in a reversal of age-associated meibomian gland atrophy.

Embodiment 251. The pharmaceutical composition or IGF-1 variant of any one of Embodiments 116-250, wherein administration of the pharmaceutical composition or IGF-1 variant to an eye or eyelid of the subject results in an increase in a function of one or more meibocytes.

Embodiment 252. The pharmaceutical composition or IGF-1 variant of any one of Embodiments 116-251, wherein administration of the pharmaceutical composition or IGF-1 variant to an eye or eyelid of the subject results in an increase in corneal epithelial cell proliferation.

Embodiment 253. The pharmaceutical composition or IGF-1 variant of any one of Embodiments 116-252, wherein administration of the pharmaceutical composition or IGF-1 variant to an eye or eyelid of the subject results in an increase in meibocyte proliferation

Embodiment 254. The pharmaceutical composition or IGF-1 variant of any one of Embodiments 116-253, wherein administration of the pharmaceutical composition or IGF-1 variant to an eye or eyelid of the subject results in an increase in corneal healing.

Embodiment 255. The pharmaceutical composition or IGF-1 variant of any one of Embodiments 116-254, wherein administration of the pharmaceutical composition or IGF-1 variant to an eye or eyelid of the subject results in an increase in IGF1 receptor (IGF1R) activation in the meibomian glands.

Embodiment 256. The pharmaceutical composition or IGF-1 variant of any one of Embodiments 116-255, wherein administration of the pharmaceutical composition or IGF-1 variant to an eye or eyelid of the subject results in an increase duration of IGF1R activation in the meibomian glands.

Embodiment 257. The pharmaceutical composition or IGF-1 variant of any one of Embodiments 116-256, wherein administration of the pharmaceutical composition or IGF-1 variant to an eye or eyelid of the subject results in an increase of lipid content of the meibomian glands.

Embodiment 258. The pharmaceutical composition or IGF-1 variant of any one of Embodiments 116-257, wherein the pharmaceutical composition or IGF-1 variant, when administered to spheroids of IHGMGE cells, results in, on average, an increase in lipid content in the spheroids.

Embodiment 259. A pharmaceutical composition comprising: a therapeutically effective amount of an IGF-1 variant comprising a polypeptide comprising an amino acid sequence of any one of SEQ ID NOS: 2-8 and 12-771 (or alternatively any one of SEQ ID NOS: 2-8 and 12-767); and one or more pharmaceutically acceptable excipients.

Embodiment 260. The pharmaceutical composition of Embodiment 259, wherein the pharmaceutical composition is a solution for delivery as an eyedrop.

Embodiment 261. The pharmaceutical composition of Embodiment 259, wherein the pharmaceutical composition comprises a cream for administration to one or both eyelids.

Embodiment 262. The pharmaceutical composition of Embodiment 259, wherein the pharmaceutical composition is formulated for systemic delivery.

Embodiment 263. The pharmaceutical composition of any one of Embodiments 259-262, wherein the polypeptide is a human IGF-1R agonist.

Embodiment 264. The pharmaceutical composition of any one of Embodiments 259-263, wherein administration of the pharmaceutical composition to subjects suffering from meibomian gland dysfunction results in a median increase in surface area or volume of meibomian glands within the inner eyelid surface.

Embodiment 265. The pharmaceutical composition of any one of Embodiments 259-264, wherein administration of the pharmaceutical composition to subjects suffering from meibomian gland dysfunction results in a median increase in lipid content within meibomian glands.

Embodiment 266. The pharmaceutical composition of any one of Embodiments 259-265, wherein administration of the pharmaceutical composition to subjects suffering from meibomian gland dysfunction results in a median increase in release of lipid from acini of the meibomian glands.

Embodiment 267. The pharmaceutical composition of any one of Embodiments 259-266, wherein administration of the pharmaceutical composition to subjects suffering from meibomian gland dysfunction results in a median increase in release of lipid from acini of the meibomian glands.

Embodiment 268. The pharmaceutical composition of any one of Embodiments 259-267, wherein administration of the pharmaceutical composition to subjects suffering from meibomian gland dysfunction results in a median increase in phosphorylation of Akt in meibocytes.

Embodiment 269. The pharmaceutical composition of any one of Embodiments 259-268, wherein the pharmaceutical composition does not comprise any additional phospholipidosis-inducing agent.

Embodiment 270. The pharmaceutical composition of any one of Embodiments 259-269, wherein the pharmaceutical composition does not comprise any one of azithromycin or doxycycline.

Embodiment 271. The pharmaceutical composition of any one of Embodiments 259-270, wherein the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone.

Embodiment 272. The pharmaceutical composition of any one of Embodiments 259-271, wherein the polypeptide comprises an amino acid sequence selected from any one of SEQ ID NOS: 2-12.

Embodiment 273. The pharmaceutical composition of any one of Embodiments 259-271, wherein the polypeptide comprises an amino acid sequence selected from any one of SEQ ID NOS: 13-771 (or alternatively any one of SEQ ID NOS: 13-767).

Embodiment 274. The pharmaceutical composition of any one of Embodiments 259-271, wherein the polypeptide comprises the amino acid sequence of SEQ ID NO: 16.

Embodiment 275. The pharmaceutical composition of any one of Embodiments 259-271, wherein the polypeptide comprises the amino acid sequence of SEQ ID NO: 17.

Embodiment 276. The pharmaceutical composition of any one of Embodiments 259-271, wherein the polypeptide comprises the amino acid sequence of SEQ ID NO: 18.

Embodiment 277. The pharmaceutical composition of any one of Embodiments 259-271, wherein the polypeptide comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 278. The pharmaceutical composition of Embodiment 277, wherein the amino acid substitution at position 3 is an arginine.

Embodiment 279. The pharmaceutical composition of Embodiment 278, wherein the polypeptide comprises an amino acid sequence selected from any one of SEQ ID NOS: 15 and 21-38.

Embodiment 280. The pharmaceutical composition of any one of Embodiments 259-271, wherein the polypeptide comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 281. The pharmaceutical composition of Embodiment 280, wherein the amino acid substitution at position 3 is an arginine.

Embodiment 282. The pharmaceutical composition of Embodiment 281, wherein the polypeptide comprises an amino acid sequence selected from any one of SEQ ID NOS: 58-75.

Embodiment 283. The pharmaceutical composition of any one of Embodiments 259-271, wherein the polypeptide comprises (1) an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 284. The pharmaceutical composition of Embodiment 283, wherein the amino acid substitution at position 3 is an arginine.

Embodiment 285. The pharmaceutical composition of Embodiment 284, wherein the polypeptide comprises an amino acid sequence selected from any one of SEQ ID NOS: 95-112.

Embodiment 286. The pharmaceutical composition of any one of Embodiments 259-271, wherein the polypeptide comprises (1) a truncation of the first three amino acid of the wild-type IGF-1 (SEQ ID NO: 1) and (2) an amino acid substitution at position 12 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 287. The pharmaceutical composition of Embodiment 286, wherein the polypeptide comprises an amino acid sequence selected from any one of SEQ ID NOS: 39-57.

Embodiment 288. The pharmaceutical composition of any one of Embodiments 259-271, wherein the polypeptide comprises (1) a truncation of the first three amino acid of the wild-type IGF-1 (SEQ ID NO: 1) and (2) an amino acid substitution at position 49 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 289. The pharmaceutical composition of Embodiment 288, wherein the polypeptide comprises an amino acid sequence selected from any one of SEQ ID NOS: 76-94.

Embodiment 290. The pharmaceutical composition of any one of Embodiments 259-271, wherein the polypeptide comprises (1) a truncation of the first three amino acid of the wild-type IGF-1 (SEQ ID NO: 1) and (2) an amino acid substitution at position 54 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 291. The pharmaceutical composition of Embodiment 290, wherein the polypeptide comprises an amino acid sequence selected from any one of SEQ ID NOS: 113-131.

Embodiment 292. The pharmaceutical composition of any one of Embodiments 259-271, wherein the polypeptide comprises one or more amino acid substitutions relative to wild-type IGF-1 (SEQ ID NO: 1), wherein the one or more amino acid substitutions relative to wild-type IGF-1 (SEQ ID NO: 1) are at positions 12, 49, 54, or a combination thereof.

Embodiment 293. The pharmaceutical composition of Embodiment 292, wherein the polypeptide comprises an amino acid sequence selected from any one of SEQ ID NOS: 132-197.

Embodiment 294. The pharmaceutical composition of any one of Embodiments 259-271, wherein the polypeptide comprises an amino acid substitution at position 5 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 295. The pharmaceutical composition of Embodiment 294, wherein the polypeptide comprises an amino acid sequence selected from any one of SEQ ID NOS: 578-596.

Embodiment 296. The pharmaceutical composition of Embodiment 294, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 297. The pharmaceutical composition of Embodiment 296, wherein the amino acid substitution at position 3 is an arginine.

Embodiment 298. The pharmaceutical composition of Embodiment 297, wherein the polypeptide comprises an amino acid sequence selected from any one of SEQ ID NOS: 198-216.

Embodiment 299. The pharmaceutical composition of Embodiment 294, wherein the polypeptide further comprises a truncation of the first three amino acid of the wild-type IGF-1 (SEQ ID NO: 1).

Embodiment 300. The pharmaceutical composition of Embodiment 294, wherein the polypeptide comprises an amino acid sequence selected from any one of SEQ ID NOS: 217-235.

Embodiment 301. The pharmaceutical composition of any one of Embodiments 259-271, wherein the polypeptide comprises an amino acid substitution at position 7 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 302. The pharmaceutical composition of Embodiment 300, wherein the polypeptide comprises an amino acid sequence selected from any one of SEQ ID NOS: 597-615.

Embodiment 303. The pharmaceutical composition of Embodiment 300, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 304. The pharmaceutical composition of Embodiment 303, wherein the amino acid substitution at position 3 is an arginine.

Embodiment 305. The pharmaceutical composition of Embodiment 304, wherein the polypeptide comprises an amino acid sequence selected from any one of SEQ ID NOS: 236-254.

Embodiment 306. The pharmaceutical composition of Embodiment 300, wherein the polypeptide further comprises a truncation of the first three amino acid of the wild-type IGF-1 (SEQ ID NO: 1).

Embodiment 307. The pharmaceutical composition of Embodiment 306, wherein the polypeptide comprises an amino acid sequence selected from any one of SEQ ID NOS: 255-273.

Embodiment 308. The pharmaceutical composition of any one of Embodiments 259-271, wherein the polypeptide comprises an amino acid substitution at position 10 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 309. The pharmaceutical composition of Embodiment 308, wherein the polypeptide comprises an amino acid sequence selected from any one of SEQ ID NOS: 616-634.

Embodiment 310. The pharmaceutical composition of Embodiment 304, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 311. The pharmaceutical composition of Embodiment 310, wherein the amino acid substitution at position 3 is an arginine.

Embodiment 312. The pharmaceutical composition of Embodiment 311, wherein the polypeptide comprises an amino acid sequence selected from any one of SEQ ID NOS: 274-292.

Embodiment 313. The pharmaceutical composition of Embodiment 308, wherein the polypeptide further comprises a truncation of the first three amino acid of the wild-type IGF-1 (SEQ ID NO: 1).

Embodiment 314. The pharmaceutical composition of Embodiment 313, wherein the polypeptide comprises an amino acid sequence selected from any one of SEQ ID NOS: 293-311.

Embodiment 315. The pharmaceutical composition of any one of Embodiments 259-271, wherein the polypeptide comprises an amino acid substitution at position 17 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 316. The pharmaceutical composition of Embodiment 315, wherein the polypeptide comprises an amino acid sequence selected from any one of SEQ ID NOS: 635-653.

Embodiment 317. The pharmaceutical composition of Embodiment 315, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 318. The pharmaceutical composition of Embodiment 317, wherein the amino acid substitution at position 3 is an arginine.

Embodiment 319. The pharmaceutical composition of Embodiment 318, wherein the polypeptide comprises an amino acid sequence selected from any one of SEQ ID NOS: 312-330.

Embodiment 320. The pharmaceutical composition of Embodiment 315, wherein the polypeptide further comprises a truncation of the first three amino acid of the wild-type IGF-1 (SEQ ID NO: 1).

Embodiment 321. The pharmaceutical composition of Embodiment 320, wherein the polypeptide comprises an amino acid sequence selected from any one of SEQ ID NOS: 331-349.

Embodiment 322. The pharmaceutical composition of any one of Embodiments 259-271, wherein the polypeptide comprises an amino acid substitution at position 22 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 323. The pharmaceutical composition of Embodiment 322, wherein the polypeptide comprises an amino acid sequence selected from any one of SEQ ID NOS: 654-672.

Embodiment 324. The pharmaceutical composition of Embodiment 322, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 325. The pharmaceutical composition of Embodiment 324, wherein the amino acid substitution at position 3 is an arginine.

Embodiment 326. The pharmaceutical composition of Embodiment 325, wherein the polypeptide comprises an amino acid sequence selected from any one of SEQ ID NOS: 350-368.

Embodiment 327. The pharmaceutical composition of Embodiment 322, wherein the polypeptide further comprises a truncation of the first three amino acid of the wild-type IGF-1 (SEQ ID NO: 1).

Embodiment 328. The pharmaceutical composition of Embodiment 327, wherein the polypeptide comprises an amino acid sequence selected from any one of SEQ ID NOS: 369-387.

Embodiment 329. The pharmaceutical composition of any one of Embodiments 259-271, wherein the polypeptide comprises an amino acid substitution at position 23 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 330. The pharmaceutical composition of Embodiment 329, wherein the polypeptide comprises an amino acid sequence selected from any one of SEQ ID NOS: 673-691.

Embodiment 331. The pharmaceutical composition of Embodiment 329, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 332. The pharmaceutical composition of Embodiment 331, wherein the amino acid substitution at position 3 is an arginine.

Embodiment 333. The pharmaceutical composition of Embodiment 332, wherein the polypeptide comprises an amino acid sequence selected from any one of SEQ ID NOS: 388-406.

Embodiment 334. The pharmaceutical composition of Embodiment 329, wherein the polypeptide further comprises a truncation of the first three amino acid of the wild-type IGF-1 (SEQ ID NO: 1).

Embodiment 335. The pharmaceutical composition of Embodiment 334, wherein the polypeptide comprises an amino acid sequence selected from any one of SEQ ID NOS: 407-425.

Embodiment 336. The pharmaceutical composition of any one of Embodiments 259-271, wherein the polypeptide comprises an amino acid substitution at position 24 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 337. The pharmaceutical composition of Embodiment 336, wherein the polypeptide comprises an amino acid sequence selected from any one of SEQ ID NOS: 692-710.

Embodiment 338. The pharmaceutical composition of Embodiment 336, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 339. The pharmaceutical composition of Embodiment 338, wherein the amino acid substitution at position 3 is an arginine.

Embodiment 340. The pharmaceutical composition of Embodiment 339, wherein the polypeptide comprises an amino acid sequence selected from any one of SEQ ID NOS: 426-444.

Embodiment 341. The pharmaceutical composition of Embodiment 336, wherein the polypeptide further comprises a truncation of the first three amino acid of the wild-type IGF-1 (SEQ ID NO: 1).

Embodiment 342. The pharmaceutical composition of Embodiment 341, wherein the polypeptide comprises an amino acid sequence selected from any one of SEQ ID NOS: 445-463.

Embodiment 343. The pharmaceutical composition of any one of Embodiments 259-271, wherein the polypeptide comprises an amino acid substitution at position 25 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 344. The pharmaceutical composition of Embodiment 343, wherein the polypeptide comprises an amino acid sequence selected from any one of SEQ ID NOS: 711-729.

Embodiment 345. The pharmaceutical composition of Embodiment 339, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 346. The pharmaceutical composition of Embodiment 345, wherein the amino acid substitution at position 3 is an arginine.

Embodiment 347. The pharmaceutical composition of Embodiment 346, wherein the polypeptide comprises an amino acid sequence selected from any one of SEQ ID NOS: 464-482.

Embodiment 348. The pharmaceutical composition of Embodiment 343, wherein the polypeptide further comprises a truncation of the first three amino acid of the wild-type IGF-1 (SEQ ID NO: 1).

Embodiment 349. The pharmaceutical composition of Embodiment 348, wherein the polypeptide comprises an amino acid sequence selected from any one of SEQ ID NOS: 483-501.

Embodiment 350. The pharmaceutical composition of any one of Embodiments 259-271, wherein the polypeptide comprises an amino acid substitution at position 34 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 351. The pharmaceutical composition of Embodiment 350, wherein the polypeptide comprises an amino acid sequence selected from any one of SEQ ID NOS: 730-748.

Embodiment 352. The pharmaceutical composition of Embodiment 350, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 353. The pharmaceutical composition of Embodiment 352, wherein the amino acid substitution at position 3 is an arginine.

Embodiment 354. The pharmaceutical composition of Embodiment 353, wherein the polypeptide comprises an amino acid sequence selected from any one of SEQ ID NOS: 502-520.

Embodiment 355. The pharmaceutical composition of Embodiment 350, wherein the polypeptide further comprises a truncation of the first three amino acid of the wild-type IGF-1 (SEQ ID NO: 1).

Embodiment 356. The pharmaceutical composition of Embodiment 355, wherein the polypeptide comprises an amino acid sequence selected from any one of SEQ ID NOS: 521-539.

Embodiment 357. The pharmaceutical composition of any one of Embodiments 255-267, wherein the polypeptide comprises an amino acid substitution at position 57 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 358. The pharmaceutical composition of Embodiment 357, wherein the polypeptide comprises an amino acid sequence selected from any one of SEQ ID NOS: 749-767.

Embodiment 359. The pharmaceutical composition of Embodiment 358, wherein the polypeptide comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus.

Embodiment 360. The pharmaceutical composition of Embodiment 359, wherein the amino acid substitution at position 3 is an arginine.

Embodiment 361. The pharmaceutical composition of Embodiment 353, wherein the polypeptide comprises an amino acid sequence selected from any one of SEQ ID NOS: 540-558.

Embodiment 362. The pharmaceutical composition of Embodiment 361, wherein the polypeptide further comprises a truncation of the first three amino acid of the wild-type IGF-1 (SEQ ID NO: 1).

Embodiment 363. The pharmaceutical composition of Embodiment 362, wherein the polypeptide comprises an amino acid sequence selected from any one of SEQ ID NOS: 549-577.

Embodiment 364. The pharmaceutical composition of any one of Embodiments 259-363, wherein the polypeptide further comprises a cell-penetrating peptide (CPP) or skin-penetrating peptide (SPP).

Embodiment 365. The pharmaceutical composition of Embodiment 364, wherein the polypeptide comprises a cell-penetrating peptide selected from the group consisting of SEQ ID NO: 10 and SEQ ID NO: 11.

Embodiment 366. The pharmaceutical composition of any one of Embodiments 259-365, wherein the polypeptide comprises the skin-penetrating peptide of SEQ ID NO: 9.

Embodiment 367. A kit comprising: the pharmaceutical composition or IGF-1 variant of any one of Embodiments 1-366; and an eyedropper for delivery of the pharmaceutical composition or IGF-1 variant as an eyedrop.

Embodiment 368. A method of treating a disease or disorder in a subject in need thereof, the method comprising administering to the subject a pharmaceutical composition comprising a non-naturally occurring IGF-1 variant that has reduced affinity to an IGF binding protein (IGFBP) relative to the affinity for the interaction between wild-type IGF-1 (SEQ ID NO: 1) and the IGFBP.

Embodiment 369. A method for treating an eye disorder in a subject in need thereof, the method comprising administering a pharmaceutical composition to a subject having an eye disorder, wherein the pharmaceutical composition comprises a therapeutically effective amount of an IGF-1 variant that has reduced affinity to an IGF binding protein (IGFBP) relative to the affinity for the interaction between wild-type IGF-1 (SEQ ID NO: 1) and the IGFBP.

Embodiment 370. A method for local administration of an IGF-1 variant to a subject, the method comprising administering an IGF-1 variant to a subject, wherein the IGF-1 variant has reduced affinity to an IGF binding protein (IGFBP) relative to the affinity of an interaction between the wild-type IGF-1 (SEQ ID NO: 1) and the IGFBP.

Embodiment 371. The method of any one of Embodiments 368-370, wherein the pharmaceutical composition is the pharmaceutical composition or IGF-1 variant of any one of Embodiments 1-117 and 119-362.

Embodiment 372. The method of any one of Embodiments 368-371, wherein the pharmaceutical composition or IGF-1 variant is administered to an eye or eyelid of the subject.

Embodiment 373. The method of Embodiment 372, wherein the pharmaceutical composition or IGF-1 variant is administered to the eye of the subject via an eyedropper.

Embodiment 374. The method of any one of Embodiments 368-373, wherein the pharmaceutical composition or IGF-1 variant is administered to an outer eyelid of the subject.

Embodiment 375. The method of Embodiment 370, wherein the pharmaceutical composition or IGF-1 variant is a cream.

Embodiment 376. The method of any one of Embodiments 368-375, wherein the pharmaceutical composition or IGF-1 variant is administered to a subject suffering from Meibomian gland dysfunction.

Embodiment 377. The method of any one of Embodiments 368-376, wherein administering the pharmaceutical composition or IGF-1 variant to the subject results in an increase in surface area or volume of meibomian glands within the inner eyelid surface of the subject.

Embodiment 378. The method of any one of Embodiments 368-377, wherein administering the pharmaceutical composition to the subject results in an increase in lipid content within a Meibomian gland of the subject.

Embodiment 379. The method of any one of Embodiments 368-378, wherein administering the pharmaceutical composition to the subject results in an increase in the number of meibocytes in a Meibomian gland of the subject.

Embodiment 380. The method of any one of Embodiments 368-379, wherein administering the pharmaceutical composition to the subject results in an increase in release of lipid from acini of a Meibomian gland of the subject.

Embodiment 381. The method of any one of Embodiments 368-380, wherein administering the pharmaceutical composition to the subject results in an increase in duration of phosphorylation of Akt in meibocytes.

Embodiment 382. The method of any one of Embodiments 368-381, wherein the method does not comprise administration of any additional phospholipidosis-inducing agent.

Embodiment 383. The method of any one of Embodiments 368-382, wherein the method does not comprise administration of one or both of azithromycin and doxycycline.

Embodiment 384. The method of any one of Embodiments 369 or 371-383, wherein the eye disorder comprises dry eye disease.

Embodiment 385. The method of any one of Embodiments 369 or 371-384, wherein the eye disorder comprises meibomian gland dysfunction.

Embodiment 386. The method of any one of Embodiments 369 or 371-385, wherein the eye disorder comprises Sjorgren's syndrome.

Embodiment 387. A pharmaceutical composition comprising the engineered polypeptide of any one of Embodiments 1-115.

Embodiment 388. A kit comprising: the engineered polypeptide of any one of Embodiments 1-115; and instructions for use.

Embodiment 389. The kit of Embodiment 388, wherein the instructions for use recite a treatment of an eye disease or disorder.

Embodiment 390. A method of treating a disease or disorder in a subject in need thereof, the method comprising administering the engineered polypeptide of any one of Embodiments 1-115 or the pharmaceutical composition of any one of Embodiments 116-366.

Embodiment 391. The method of Embodiment 390, wherein the disease or disorder is an eye disorder.

Embodiment 392. The method of any one of Embodiment 391, wherein the eye disorder comprises dry eye disease.

Embodiment 393. The method of any one of Embodiment 391, wherein the eye disorder comprises meibomian gland dysfunction.

Embodiment 394. The method of any one of Embodiment 391, wherein the eye disorder comprises Sjorgren's syndrome.

Embodiment 395. The method of any one of Embodiments 390-394, wherein the method does not comprise administration of one or both of azithromycin and doxycycline.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. An engineered polypeptide comprising SEQ ID NO: 17.

2. A pharmaceutical composition comprising the engineered polypeptide of claim 1.

3. The pharmaceutical composition of claim 2, wherein a local administration of the pharmaceutical composition to an eye or an eyelid results in one or more of:
(a) an increase in a size of meibomian glands;
(b) a decrease in meibomian gland atrophy;
(c) a reversal of age-associated meibomian gland atrophy;
(d) an increase in a function of one or more meibocytes;
(e) an increase in corneal epithelial cell proliferation;
(f) an increase in meibocyte proliferation;
(g) an increase in corneal healing rate;
(h) an increase in IGF1 receptor (IGF1R) activation in the meibomian glands;
(i) an increase duration of IGF1R activation in the meibomian glands; and
(j) an increase of lipid content of the meibomian glands.

4. The pharmaceutical composition of claim 2, further comprising one or more pharmaceutically acceptable excipients.

5. The pharmaceutical composition of claim 4, wherein the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone.

6. The pharmaceutical composition of claim 2, wherein the pharmaceutical composition is formulated for delivery via an eyedrop.

7. The pharmaceutical composition of claim 2, wherein the pharmaceutical composition comprises a cream for administration to one or both eyelids of a subject.

8. The pharmaceutical composition of claim 2, wherein the pharmaceutical composition does not comprise administration of one or both of azithromycin and doxycycline.

9. A kit comprising:
(a) the pharmaceutical composition of claim 2; and
(b) an eyedropper for delivery of the pharmaceutical composition as an eyedrop.

10. The kit of claim 9, further comprising instructions for use.

11. The kit of claim 10, wherein the instructions for use comprise administering the eyedropper to a subject having a disease or disorder.

12. The kit of claim 11, wherein the disease or disorder is dry eye disease.

13. The kit of claim 11, wherein the disease or disorder is Meibomian gland dysfunction.

14. The kit of claim 11, wherein the disease or disorder is Sjorgren's syndrome.

15. A method of treating a disease or disorder in a subject in need thereof, the method comprising administering to the subject the pharmaceutical composition of claim 2.


SEQUENCE LISTING (772 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, synthetic construct
GPETLCGAELVDALQFVCGDRGFYFNKPTGYGSSSRRAPQTGIVDECCFRSCDLRRLEMLCAPLKPAKSA

SEQ ID NO: 3 - protein, 67 aa, synthetic construct
TLCGAELVDALQFVCGDRGFYFNKPTGYGSSSRRAPQTGIVDECCFRSCDLRRLEMYCAPLKPAKSA

SEQ ID NO: 4 - protein, 70 aa, synthetic construct
GPRTLCGAELVDALQFVCGDRGFYFNKPTGYGSSSRRAPQTGIVDECCFRSCDLRRLEMYCAPLKPAKSA

SEQ ID NO: 5 - protein, 69 aa, synthetic construct
GPETLCGAELVDALQFVCGDRGFYFNKPTGYGSSSRAPQTGIVDECCFRSCDLRRLEMYCAPLKPAKSA

SEQ ID NO: 6 - protein, 66 aa, synthetic construct
TLCGAELVDALQFVCGDRGFYFNKPTGYGSSSRAPQTGIVDECCFRSCDLRRLEMYCAPLKPAKSA

SEQ ID NO: 7 - protein, 105 aa, synthetic construct
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 8 - protein, 77 aa, synthetic construct
MRAAAPAVAATLCGAELVDALQFVCGDRGFYFNKPTGYGSSSRRAPQTGIVDECCFRSCDLRRLEMYCAPLKPAKSA

SEQ ID NO: 9 - protein, 10 aa, synthetic construct
MRAAAPAVAA

SEQ ID NO: 10 - protein, 15 aa, synthetic construct
CGRKKRRQRRRPPQC

SEQ ID NO: 11 - protein, 9 aa, synthetic construct
RRRRRRRRR

SEQ ID NO: 12 - protein, 83 aa, synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 13 - protein, 83 aa, synthetic construct
GGGGSGGGGSGGGGPETLCGAELVDALQFVCGDRGFYFNKPTGYGSSSRRAPQTGIVDECCFRSCDLRRLEMYCAPLKPAKSA

SEQ ID NO: 14 - protein, 83 aa, synthetic construct
GGGGSGGGGSGGGGPRTLCGAELVDALQFVCGDRGFYFNKPTGYGSSSRRAPQTGIVDECCFRSCDLRRLEMYCAPLKPAKSA

SEQ ID NO: 15 - protein, 70 aa, synthetic construct
GPRTLCGAELVAALQFVCGDRGFYFNKPTGYGSSSRRAPQTGIVDECCFRSCDLRRLEMYCAPLKPAKSA

SEQ ID NO: 16 - protein, 70 aa, synthetic construct
GPRTLCGAELVDALQFVCGARGFYFNKPTGYGSSSRRAPQTGIVDECCFRSCDLRRLEMYCAPLKPAKSA

SEQ ID NO: 17 - protein, 70 aa, synthetic construct
GPRTLCGAELVDALQFVCGDRGFYFNKPTGYGSSSRRAPQTGIVDECCLRSCDLRRLEMYCAPLKPAKSA

SEQ ID NO: 18 - protein, 70 aa, synthetic construct
GPRTLCGAELVDALQFVCGDRGFYFNKPTGYGSSSRRAPQTGIVDECCFRSCDARRLEMYCAPLKPAKSA

SEQ ID NO: 19 - protein, 70 aa, synthetic construct
GPRTLCGAELVDCLQFVCGDRGFYFNKPTGYGSSSRRAPQTGIVDECCFRSCDLRRCEMYCAPLKPAKSA

SEQ ID NO: 20 - protein, 70 aa, synthetic construct
GPRTLCGAELVDACQFVCGDRGFYFNKPTGYGSSSRRAPQTGIVDECCFRSCDLRRCEMYCAPLKPAKSA

SEQ ID NO: 21 - protein, 70 aa, synthetic construct
GPRTLCGAELVCALQFVCGDRGFYFNKPTGYGSSSRRAPQTGIVDECCFRSCDLRRLEMYCAPLKPAKSA

SEQ ID NO: 22 - protein, 70 aa, synthetic construct
GPRTLCGAELVEALQFVCGDRGFYFNKPTGYGSSSRRAPQTGIVDECCFRSCDLRRLEMYCAPLKPAKSA

SEQ ID NO: 23 - protein, 70 aa, synthetic construct
GPRTLCGAELVFALQFVCGDRGFYFNKPTGYGSSSRRAPQTGIVDECCFRSCDLRRLEMYCAPLKPAKSA

SEQ ID NO: 24 - protein, 70 aa, synthetic construct
GPRTLCGAELVGALQFVCGDRGFYFNKPTGYGSSSRRAPQTGIVDECCFRSCDLRRLEMYCAPLKPAKSA

SEQ ID NO: 25 - protein, 70 aa, synthetic construct
GPRTLCGAELVHALQFVCGDRGFYFNKPTGYGSSSRRAPQTGIVDECCFRSCDLRRLEMYCAPLKPAKSA

SEQ ID NO: 26 - protein, 70 aa, synthetic construct
GPRTLCGAELVIALQFVCGDRGFYFNKPTGYGSSSRRAPQTGIVDECCFRSCDLRRLEMYCAPLKPAKSA

SEQ ID NO: 27 - protein, 70 aa, synthetic construct
GPRTLCGAELVKALQFVCGDRGFYFNKPTGYGSSSRRAPQTGIVDECCFRSCDLRRLEMYCAPLKPAKSA

SEQ ID NO: 28 - protein, 70 aa, synthetic construct
GPRTLCGAELVLALQFVCGDRGFYFNKPTGYGSSSRRAPQTGIVDECCFRSCDLRRLEMYCAPLKPAKSA

SEQ ID NO: 29 - protein, 70 aa, synthetic construct
GPRTLCGAELVMALQFVCGDRGFYFNKPTGYGSSSRRAPQTGIVDECCFRSCDLRRLEMYCAPLKPAKSA

SEQ ID NO: 30 - protein, 70 aa, synthetic construct
GPRTLCGAELVNALQFVCGDRGFYFNKPTGYGSSSRRAPQTGIVDECCFRSCDLRRLEMYCAPLKPAKSA

SEQ ID NO: 31 - protein, 70 aa, synthetic construct
GPRTLCGAELVPALQFVCGDRGFYFNKPTGYGSSSRRAPQTGIVDECCFRSCDLRRLEMYCAPLKPAKSA

SEQ ID NO: 32 - protein, 70 aa, synthetic construct
GPRTLCGAELVQALQFVCGDRGFYFNKPTGYGSSSRRAPQTGIVDECCFRSCDLRRLEMYCAPLKPAKSA

SEQ ID NO: 33 - protein, 70 aa, synthetic construct
GPRTLCGAELVRALQFVCGDRGFYFNKPTGYGSSSRRAPQTGIVDECCFRSCDLRRLEMYCAPLKPAKSA

SEQ ID NO: 34 - protein, 70 aa, synthetic construct
GPRTLCGAELVSALQFVCGDRGFYFNKPTGYGSSSRRAPQTGIVDECCFRSCDLRRLEMYCAPLKPAKSA

SEQ ID NO: 35 - protein, 70 aa, synthetic construct
GPRTLCGAELVTALQFVCGDRGFYFNKPTGYGSSSRRAPQTGIVDECCFRSCDLRRLEMYCAPLKPAKSA

SEQ ID NO: 36 - protein, 70 aa, synthetic construct
GPRTLCGAELVVALQFVCGDRGFYFNKPTGYGSSSRRAPQTGIVDECCFRSCDLRRLEMYCAPLKPAKSA

SEQ ID NO: 37 - protein, 70 aa, synthetic construct
GPRTLCGAELVWALQFVCGDRGFYFNKPTGYGSSSRRAPQTGIVDECCFRSCDLRRLEMYCAPLKPAKSA

SEQ ID NO: 38 - protein, 70 aa, synthetic construct
GPRTLCGAELVYALQFVCGDRGFYFNKPTGYGSSSRRAPQTGIVDECCFRSCDLRRLEMYCAPLKPAKSA

SEQ ID NO: 39 - protein, 67 aa, synthetic construct
TLCGAELVAALQFVCGDRGFYFNKPTGYGSSSRRAPQTGIVDECCFRSCDLRRLEMYCAPLKPAKSA

SEQ ID NO: 40 - protein, 67 aa, synthetic construct
TLCGAELVCALQFVCGDRGFYFNKPTGYGSSSRRAPQTGIVDECCFRSCDLRRLEMYCAPLKPAKSA

SEQ ID NO: 41 - protein, 67 aa, synthetic construct
TLCGAELVEALQFVCGDRGFYFNKPTGYGSSSRRAPQTGIVDECCFRSCDLRRLEMYCAPLKPAKSA

SEQ ID NO: 42 - protein, 67 aa, synthetic construct
TLCGAELVFALQFVCGDRGFYFNKPTGYGSSSRRAPQTGIVDECCFRSCDLRRLEMYCAPLKPAKSA

SEQ ID NO: 43 - protein, 67 aa, synthetic construct
TLCGAELVGALQFVCGDRGFYFNKPTGYGSSSRRAPQTGIVDECCFRSCDLRRLEMYCAPLKPAKSA

SEQ ID NO: 44 - protein, 67 aa, synthetic construct
TLCGAELVHALQFVCGDRGFYFNKPTGYGSSSRRAPQTGIVDECCFRSCDLRRLEMYCAPLKPAKSA

SEQ ID NO: 45 - protein, 67 aa, synthetic construct
TLCGAELVIALQFVCGDRGFYFNKPTGYGSSSRRAPQTGIVDECCFRSCDLRRLEMYCAPLKPAKSA

SEQ ID NO: 46 - protein, 67 aa, synthetic construct
TLCGAELVKALQFVCGDRGFYFNKPTGYGSSSRRAPQTGIVDECCFRSCDLRRLEMYCAPLKPAKSA

SEQ ID NO: 47 - protein, 67 aa, synthetic construct
TLCGAELVLALQFVCGDRGFYFNKPTGYGSSSRRAPQTGIVDECCFRSCDLRRLEMYCAPLKPAKSA

SEQ ID NO: 48 - protein, 67 aa, synthetic construct
TLCGAELVMALQFVCGDRGFYFNKPTGYGSSSRRAPQTGIVDECCFRSCDLRRLEMYCAPLKPAKSA

SEQ ID NO: 49 - protein, 67 aa, synthetic construct
TLCGAELVNALQFVCGDRGFYFNKPTGYGSSSRRAPQTGIVDECCFRSCDLRRLEMYCAPLKPAKSA

SEQ ID NO: 50 - protein, 67 aa, synthetic construct
TLCGAELVPALQFVCGDRGFYFNKPTGYGSSSRRAPQTGIVDECCFRSCDLRRLEMYCAPLKPAKSA

SEQ ID NO: 51 - protein, 67 aa, synthetic construct
TLCGAELVQALQFVCGDRGFYFNKPTGYGSSSRRAPQTGIVDECCFRSCDLRRLEMYCAPLKPAKSA

SEQ ID NO: 52 - protein, 67 aa, synthetic construct
TLCGAELVRALQFVCGDRGFYFNKPTGYGSSSRRAPQTGIVDECCFRSCDLRRLEMYCAPLKPAKSA

SEQ ID NO: 53 - protein, 67 aa, synthetic construct
TLCGAELVSALQFVCGDRGFYFNKPTGYGSSSRRAPQTGIVDECCFRSCDLRRLEMYCAPLKPAKSA

SEQ ID NO: 54 - protein, 67 aa, synthetic construct
TLCGAELVTALQFVCGDRGFYFNKPTGYGSSSRRAPQTGIVDECCFRSCDLRRLEMYCAPLKPAKSA

SEQ ID NO: 55 - protein, 67 aa, synthetic construct
TLCGAELVVALQFVCGDRGFYFNKPTGYGSSSRRAPQTGIVDECCFRSCDLRRLEMYCAPLKPAKSA

SEQ ID NO: 56 - protein, 67 aa, synthetic construct
TLCGAELVWALQFVCGDRGFYFNKPTGYGSSSRRAPQTGIVDECCFRSCDLRRLEMYCAPLKPAKSA

SEQ ID NO: 57 - protein, 67 aa, synthetic construct
TLCGAELVYALQFVCGDRGFYFNKPTGYGSSSRRAPQTGIVDECCFRSCDLRRLEMYCAPLKPAKSA

SEQ ID NO: 58 - protein, 70 aa, synthetic construct
GPRTLCGAELVDALQFVCGDRGFYFNKPTGYGSSSRRAPQTGIVDECCARSCDLRRLEMYCAPLKPAKSA

SEQ ID NO: 59 - protein, 70 aa, synthetic construct
GPRTLCGAELVDALQFVCGDRGFYFNKPTGYGSSSRRAPQTGIVDECCCRSCDLRRLEMYCAPLKPAKSA

SEQ ID NO: 60 - protein, 70 aa, synthetic construct
GPRTLCGAELVDALQFVCGDRGFYFNKPTGYGSSSRRAPQTGIVDECCDRSCDLRRLEMYCAPLKPAKSA

SEQ ID NO: 61 - protein, 70 aa, synthetic construct
GPRTLCGAELVDALQFVCGDRGFYFNKPTGYGSSSRRAPQTGIVDECCERSCDLRRLEMYCAPLKPAKSA

SEQ ID NO: 62 - protein, 70 aa, synthetic construct
GPRTLCGAELVDALQFVCGDRGFYFNKPTGYGSSSRRAPQTGIVDECCGRSCDLRRLEMYCAPLKPAKSA

SEQ ID NO: 63 - protein, 70 aa, synthetic construct
GPRTLCGAELVDALQFVCGDRGFYFNKPTGYGSSSRRAPQTGIVDECCHRSCDLRRLEMYCAPLKPAKSA

SEQ ID NO: 64 - protein, 70 aa, synthetic construct
GPRTLCGAELVDALQFVCGDRGFYFNKPTGYGSSSRRAPQTGIVDECCIRSCDLRRLEMYCAPLKPAKSA

SEQ ID NO: 65 - protein, 70 aa, synthetic construct
GPRTLCGAELVDALQFVCGDRGFYFNKPTGYGSSSRRAPQTGIVDECCKRSCDLRRLEMYCAPLKPAKSA

SEQ ID NO: 66 - protein, 70 aa, synthetic construct
GPRTLCGAELVDALQFVCGDRGFYFNKPTGYGSSSRRAPQTGIVDECCMRSCDLRRLEMYCAPLKPAKSA

SEQ ID NO: 67 - protein, 70 aa, synthetic construct
GPRTLCGAELVDALQFVCGDRGFYFNKPTGYGSSSRRAPQTGIVDECCNRSCDLRRLEMYCAPLKPAKSA

SEQ ID NO: 68 - protein, 70 aa, synthetic construct
GPRTLCGAELVDALQFVCGDRGFYFNKPTGYGSSSRRAPQTGIVDECCPRSCDLRRLEMYCAPLKPAKSA

SEQ ID NO: 69 - protein, 70 aa, synthetic construct
GPRTLCGAELVDALQFVCGDRGFYFNKPTGYGSSSRRAPQTGIVDECCQRSCDLRRLEMYCAPLKPAKSA

SEQ ID NO: 70 - protein, 70 aa, synthetic construct
GPRTLCGAELVDALQFVCGDRGFYFNKPTGYGSSSRRAPQTGIVDECCRRSCDLRRLEMYCAPLKPAKSA

SEQ ID NO: 71 - protein, 70 aa, synthetic construct
GPRTLCGAELVDALQFVCGDRGFYFNKPTGYGSSSRRAPQTGIVDECCSRSCDLRRLEMYCAPLKPAKSA

SEQ ID NO: 72 - protein, 70 aa, synthetic construct
GPRTLCGAELVDALQFVCGDRGFYFNKPTGYGSSSRRAPQTGIVDECCTRSCDLRRLEMYCAPLKPAKSA

SEQ ID NO: 73 - protein, 70 aa, synthetic construct
GPRTLCGAELVDALQFVCGDRGFYFNKPTGYGSSSRRAPQTGIVDECCVRSCDLRRLEMYCAPLKPAKSA

SEQ ID NO: 74 - protein, 70 aa, synthetic construct
GPRTLCGAELVDALQFVCGDRGFYFNKPTGYGSSSRRAPQTGIVDECCWRSCDLRRLEMYCAPLKPAKSA

SEQ ID NO: 75 - protein, 70 aa, synthetic construct
GPRTLCGAELVDALQFVCGDRGFYFNKPTGYGSSSRRAPQTGIVDECCYRSCDLRRLEMYCAPLKPAKSA

SEQ ID NO: 76 - protein, 67 aa, synthetic construct
TLCGAELVDALQFVCGDRGFYFNKPTGYGSSSRRAPQTGIVDECCARSCDLRRLEMYCAPLKPAKSA

SEQ ID NO: 77 - protein, 67 aa, synthetic construct
TLCGAELVDALQFVCGDRGFYFNKPTGYGSSSRRAPQTGIVDECCCRSCDLRRLEMYCAPLKPAKSA

SEQ ID NO: 78 - protein, 67 aa, synthetic construct
TLCGAELVDALQFVCGDRGFYFNKPTGYGSSSRRAPQTGIVDECCDRSCDLRRLEMYCAPLKPAKSA

SEQ ID NO: 79 - protein, 67 aa, synthetic construct
TLCGAELVDALQFVCGDRGFYFNKPTGYGSSSRRAPQTGIVDECCERSCDLRRLEMYCAPLKPAKSA

SEQ ID NO: 80 - protein, 67 aa, synthetic construct
TLCGAELVDALQFVCGDRGFYFNKPTGYGSSSRRAPQTGIVDECCGRSCDLRRLEMYCAPLKPAKSA

SEQ ID NO: 81 - protein, 67 aa, synthetic construct
TLCGAELVDALQFVCGDRGFYFNKPTGYGSSSRRAPQTGIVDECCHRSCDLRRLEMYCAPLKPAKSA

SEQ ID NO: 82 - protein, 67 aa, synthetic construct
TLCGAELVDALQFVCGDRGFYFNKPTGYGSSSRRAPQTGIVDECCIRSCDLRRLEMYCAPLKPAKSA

SEQ ID NO: 83 - protein, 67 aa, synthetic construct
TLCGAELVDALQFVCGDRGFYFNKPTGYGSSSRRAPQTGIVDECCKRSCDLRRLEMYCAPLKPAKSA

SEQ ID NO: 84 - protein, 67 aa, synthetic construct
TLCGAELVDALQFVCGDRGFYFNKPTGYGSSSRRAPQTGIVDECCLRSCDLRRLEMYCAPLKPAKSA

SEQ ID NO: 85 - protein, 67 aa, synthetic construct
TLCGAELVDALQFVCGDRGFYFNKPTGYGSSSRRAPQTGIVDECCMRSCDLRRLEMYCAPLKPAKSA

SEQ ID NO: 86 - protein, 67 aa, synthetic construct
TLCGAELVDALQFVCGDRGFYFNKPTGYGSSSRRAPQTGIVDECCNRSCDLRRLEMYCAPLKPAKSA

SEQ ID NO: 87 - protein, 67 aa, synthetic construct
TLCGAELVDALQFVCGDRGFYFNKPTGYGSSSRRAPQTGIVDECCPRSCDLRRLEMYCAPLKPAKSA

SEQ ID NO: 88 - protein, 67 aa, synthetic construct
TLCGAELVDALQFVCGDRGFYFNKPTGYGSSSRRAPQTGIVDECCQRSCDLRRLEMYCAPLKPAKSA

SEQ ID NO: 89 - protein, 67 aa, synthetic construct
TLCGAELVDALQFVCGDRGFYFNKPTGYGSSSRRAPQTGIVDECCRRSCDLRRLEMYCAPLKPAKSA

SEQ ID NO: 90 - protein, 67 aa, synthetic construct
TLCGAELVDALQFVCGDRGFYFNKPTGYGSSSRRAPQTGIVDECCSRSCDLRRLEMYCAPLKPAKSA

SEQ ID NO: 91 - protein, 67 aa, synthetic construct
TLCGAELVDALQFVCGDRGFYFNKPTGYGSSSRRAPQTGIVDECCTRSCDLRRLEMYCAPLKPAKSA

SEQ ID NO: 92 - protein, 67 aa, synthetic construct
TLCGAELVDALQFVCGDRGFYFNKPTGYGSSSRRAPQTGIVDECCVRSCDLRRLEMYCAPLKPAKSA

SEQ ID NO: 93 - protein, 67 aa, synthetic construct
TLCGAELVDALQFVCGDRGFYFNKPTGYGSSSRRAPQTGIVDECCWRSCDLRRLEMYCAPLKPAKSA

SEQ ID NO: 94 - protein, 67 aa, synthetic construct
TLCGAELVDALQFVCGDRGFYFNKPTGYGSSSRRAPQTGIVDECCYRSCDLRRLEMYCAPLKPAKSA

SEQ ID NO: 95 - protein, 70 aa, synthetic construct
GPRTLCGAELVDALQFVCGDRGFYFNKPTGYGSSSRRAPQTGIVDECCFRSCDCRRLEMYCAPLKPAKSA

SEQ ID NO: 96 - protein, 70 aa, synthetic construct
GPRTLCGAELVDALQFVCGDRGFYFNKPTGYGSSSRRAPQTGIVDECCFRSCDDRRLEMYCAPLKPAKSA

SEQ ID NO: 97 - protein, 70 aa, synthetic construct
GPRTLCGAELVDALQFVCGDRGFYFNKPTGYGSSSRRAPQTGIVDECCFRSCDERRLEMYCAPLKPAKSA

SEQ ID NO: 98 - protein, 70 aa, synthetic construct
GPRTLCGAELVDALQFVCGDRGFYFNKPTGYGSSSRRAPQTGIVDECCFRSCDFRRLEMYCAPLKPAKSA

SEQ ID NO: 99 - protein, 70 aa, synthetic construct
GPRTLCGAELVDALQFVCGDRGFYFNKPTGYGSSSRRAPQTGIVDECCFRSCDGRRLEMYCAPLKPAKSA

SEQ ID NO: 100 - protein, 70 aa, synthetic construct
GPRTLCGAELVDALQFVCGDRGFYFNKPTGYGSSSRRAPQTGIVDECCFRSCDHRRLEMYCAPLKPAKSA

SEQ ID NO: 101 - protein, 70 aa, synthetic construct
GPRTLCGAELVDALQFVCGDRGFYFNKPTGYGSSSRRAPQTGIVDECCFRSCDIRRLEMYCAPLKPAKSA

SEQ ID NO: 102 - protein, 70 aa, synthetic construct
GPRTLCGAELVDALQFVCGDRGFYFNKPTGYGSSSRRAPQTGIVDECCFRSCDKRRLEMYCAPLKPAKSA

SEQ ID NO: 103 - protein, 70 aa, synthetic construct
GPRTLCGAELVDALQFVCGDRGFYFNKPTGYGSSSRRAPQTGIVDECCFRSCDMRRLEMYCAPLKPAKSA

SEQ ID NO: 104 - protein, 70 aa, synthetic construct
GPRTLCGAELVDALQFVCGDRGFYFNKPTGYGSSSRRAPQTGIVDECCFRSCDNRRLEMYCAPLKPAKSA

SEQ ID NO: 105 - protein, 70 aa, synthetic construct
GPRTLCGAELVDALQFVCGDRGFYFNKPTGYGSSSRRAPQTGIVDECCFRSCDPRRLEMYCAPLKPAKSA

SEQ ID NO: 106 - protein, 70 aa, synthetic construct
GPRTLCGAELVDALQFVCGDRGFYFNKPTGYGSSSRRAPQTGIVDECCFRSCDQRRLEMYCAPLKPAKSA

SEQ ID NO: 107 - protein, 70 aa, synthetic construct
GPRTLCGAELVDALQFVCGDRGFYFNKPTGYGSSSRRAPQTGIVDECCFRSCDRRRLEMYCAPLKPAKSA

SEQ ID NO: 108 - protein, 70 aa, synthetic construct
GPRTLCGAELVDALQFVCGDRGFYFNKPTGYGSSSRRAPQTGIVDECCFRSCDSRRLEMYCAPLKPAKSA

SEQ ID NO: 109 - protein, 70 aa, synthetic construct
GPRTLCGAELVDALQFVCGDRGFYFNKPTGYGSSSRRAPQTGIVDECCFRSCDTRRLEMYCAPLKPAKSA

SEQ ID NO: 110 - protein, 70 aa, synthetic construct
GPRTLCGAELVDALQFVCGDRGFYFNKPTGYGSSSRRAPQTGIVDECCFRSCDVRRLEMYCAPLKPAKSA

SEQ ID NO: 111 - protein, 70 aa, synthetic construct
GPRTLCGAELVDALQFVCGDRGFYFNKPTGYGSSSRRAPQTGIVDECCFRSCDWRRLEMYCAPLKPAKSA

SEQ ID NO: 112 - protein, 70 aa, synthetic construct
GPRTLCGAELVDALQFVCGDRGFYFNKPTGYGSSSRRAPQTGIVDECCFRSCDYRRLEMYCAPLKPAKSA

SEQ ID NO: 113 - protein, 67 aa, synthetic construct
TLCGAELVDALQFVCGDRGFYFNKPTGYGSSSRRAPQTGIVDECCFRSCDARRLEMYCAPLKPAKSA

SEQ ID NO: 114 - protein, 67 aa, synthetic construct
TLCGAELVDALQFVCGDRGFYFNKPTGYGSSSRRAPQTGIVDECCFRSCDCRRLEMYCAPLKPAKSA

SEQ ID NO: 115 - protein, 67 aa, synthetic construct
TLCGAELVDALQFVCGDRGFYFNKPTGYGSSSRRAPQTGIVDECCFRSCDDRRLEMYCAPLKPAKSA

SEQ ID NO: 116 - protein, 67 aa, synthetic construct
TLCGAELVDALQFVCGDRGFYFNKPTGYGSSSRRAPQTGIVDECCFRSCDERRLEMYCAPLKPAKSA

SEQ ID NO: 117 - protein, 67 aa, synthetic construct
TLCGAELVDALQFVCGDRGFYFNKPTGYGSSSRRAPQTGIVDECCFRSCDFRRLEMYCAPLKPAKSA

SEQ ID NO: 118 - protein, 67 aa, synthetic construct
TLCGAELVDALQFVCGDRGFYFNKPTGYGSSSRRAPQTGIVDECCFRSCDGRRLEMYCAPLKPAKSA

SEQ ID NO: 119 - protein, 67 aa, synthetic construct
TLCGAELVDALQFVCGDRGFYFNKPTGYGSSSRRAPQTGIVDECCFRSCDHRRLEMYCAPLKPAKSA

SEQ ID NO: 120 - protein, 67 aa, synthetic construct
TLCGAELVDALQFVCGDRGFYFNKPTGYGSSSRRAPQTGIVDECCFRSCDIRRLEMYCAPLKPAKSA

SEQ ID NO: 121 - protein, 67 aa, synthetic construct
TLCGAELVDALQFVCGDRGFYFNKPTGYGSSSRRAPQTGIVDECCFRSCDKRRLEMYCAPLKPAKSA

SEQ ID NO: 122 - protein, 67 aa, synthetic construct
TLCGAELVDALQFVCGDRGFYFNKPTGYGSSSRRAPQTGIVDECCFRSCDMRRLEMYCAPLKPAKSA

SEQ ID NO: 123 - protein, 67 aa, synthetic construct
TLCGAELVDALQFVCGDRGFYFNKPTGYGSSSRRAPQTGIVDECCFRSCDNRRLEMYCAPLKPAKSA

SEQ ID NO: 124 - protein, 67 aa, synthetic construct
TLCGAELVDALQFVCGDRGFYFNKPTGYGSSSRRAPQTGIVDECCFRSCDPRRLEMYCAPLKPAKSA

SEQ ID NO: 125 - protein, 67 aa, synthetic construct
TLCGAELVDALQFVCGDRGFYFNKPTGYGSSSRRAPQTGIVDECCFRSCDQRRLEMYCAPLKPAKSA

SEQ ID NO: 126 - protein, 67 aa, synthetic construct
TLCGAELVDALQFVCGDRGFYFNKPTGYGSSSRRAPQTGIVDECCFRSCDRRRLEMYCAPLKPAKSA

SEQ ID NO: 127 - protein, 67 aa, synthetic construct
TLCGAELVDALQFVCGDRGFYFNKPTGYGSSSRRAPQTGIVDECCFRSCDSRRLEMYCAPLKPAKSA

SEQ ID NO: 128 - protein, 67 aa, synthetic construct
TLCGAELVDALQFVCGDRGFYFNKPTGYGSSSRRAPQTGIVDECCFRSCDTRRLEMYCAPLKPAKSA

SEQ ID NO: 129 - protein, 67 aa, synthetic construct
TLCGAELVDALQFVCGDRGFYFNKPTGYGSSSRRAPQTGIVDECCFRSCDVRRLEMYCAPLKPAKSA

SEQ ID NO: 130 - protein, 67 aa, synthetic construct
TLCGAELVDALQFVCGDRGFYFNKPTGYGSSSRRAPQTGIVDECCFRSCDWRRLEMYCAPLKPAKSA

SEQ ID NO: 131 - protein, 67 aa, synthetic construct
TLCGAELVDALQFVCGDRGFYFNKPTGYGSSSRRAPQTGIVDECCFRSCDYRRLEMYCAPLKPAKSA

SEQ ID NO: 132 - protein, 70 aa, synthetic construct
GPETLCGAELVDALQFVCGDRGFYFNKPTGYGSSSRRAPQTGIVDECCARSCDLRRLEMYCAPLKPAKSA

SEQ ID NO: 133 - protein, 70 aa, synthetic construct
GPETLCGAELVAALQFVCGDRGFYFNKPTGYGSSSRRAPQTGIVDECCFRSCDLRRLEMYCAPLKPAKSA

SEQ ID NO: 134 - protein, 70 aa, synthetic construct
GPETLCGAELVDALQFVCGDRGFYFNKPTGYGSSSRRAPQTGIVDECCLRSCDLRRLEMYCAPLKPAKSA

SEQ ID NO: 135 - protein, 70 aa, synthetic construct
GPETLCGAELVDALQFVCGDRGFYFNKPTGYGSSSRRAPQTGIVDECCFRSCDARRLEMYCAPLKPAKSA

SEQ ID NO: 136 - protein, 70 aa, synthetic construct
GPETLCGAELVAALQFVCGDRGFYFNKPTGYGSSSRRAPQTGIVDECCLRSCDLRRLEMYCAPLKPAKSA

SEQ ID NO: 137 - protein, 70 aa, synthetic construct
GPETLCGAELVDALQFVCGDRGFYFNKPTGYGSSSRRAPQTGIVDECCLRSCDARRLEMYCAPLKPAKSA

SEQ ID NO: 138 - protein, 70 aa, synthetic construct
GPETLCGAELVAALQFVCGDRGFYFNKPTGYGSSSRRAPQTGIVDECCLRSCDARRLEMYCAPLKPAKSA

SEQ ID NO: 139 - protein, 70 aa, synthetic construct
GPRTLCGAELVAALQFVCGDRGFYFNKPTGYGSSSRRAPQTGIVDECCLRSCDLRRLEMYCAPLKPAKSA

SEQ ID NO: 140 - protein, 70 aa, synthetic construct
GPRTLCGAELVDALQFVCGDRGFYFNKPTGYGSSSRRAPQTGIVDECCLRSCDARRLEMYCAPLKPAKSA

SEQ ID NO: 141 - protein, 70 aa, synthetic construct
GPRTLCGAELVAALQFVCGDRGFYFNKPTGYGSSSRRAPQTGIVDECCLRSCDARRLEMYCAPLKPAKSA

SEQ ID NO: 142 - protein, 67 aa, synthetic construct
TLCGAELVAALQFVCGDRGFYFNKPTGYGSSSRRAPQTGIVDECCLRSCDLRRLEMYCAPLKPAKSA

SEQ ID NO: 143 - protein, 67 aa, synthetic construct
TLCGAELVDALQFVCGDRGFYFNKPTGYGSSSRRAPQTGIVDECCLRSCDARRLEMYCAPLKPAKSA

SEQ ID NO: 144 - protein, 67 aa, synthetic construct
TLCGAELVAALQFVCGDRGFYFNKPTGYGSSSRRAPQTGIVDECCLRSCDARRLEMYCAPLKPAKSA

SEQ ID NO: 145 - protein, 70 aa, synthetic construct
GPETLCGAELVCALQFVCGDRGFYFNKPTGYGSSSRRAPQTGIVDECCFRSCDLRRLEMYCAPLKPAKSA

SEQ ID NO: 146 - protein, 70 aa, synthetic construct
GPETLCGAELVEALQFVCGDRGFYFNKPTGYGSSSRRAPQTGIVDECCFRSCDLRRLEMYCAPLKPAKSA

SEQ ID NO: 147 - protein, 70 aa, synthetic construct
GPETLCGAELVFALQFVCGDRGFYFNKPTGYGSSSRRAPQTGIVDECCFRSCDLRRLEMYCAPLKPAKSA

SEQ ID NO: 148 - protein, 70 aa, synthetic construct
GPETLCGAELVGALQFVCGDRGFYFNKPTGYGSSSRRAPQTGIVDECCFRSCDLRRLEMYCAPLKPAKSA

SEQ ID NO: 149 - protein, 70 aa, synthetic construct
GPETLCGAELVHALQFVCGDRGFYFNKPTGYGSSSRRAPQTGIVDECCFRSCDLRRLEMYCAPLKPAKSA

SEQ ID NO: 150 - protein, 70 aa, synthetic construct
GPETLCGAELVIALQFVCGDRGFYFNKPTGYGSSSRRAPQTGIVDECCFRSCDLRRLEMYCAPLKPAKSA

SEQ ID NO: 151 - protein, 70 aa, synthetic construct
GPETLCGAELVKALQFVCGDRGFYFNKPTGYGSSSRRAPQTGIVDECCFRSCDLRRLEMYCAPLKPAKSA

SEQ ID NO: 152 - protein, 70 aa, synthetic construct
GPETLCGAELVLALQFVCGDRGFYFNKPTGYGSSSRRAPQTGIVDECCFRSCDLRRLEMYCAPLKPAKSA

SEQ ID NO: 153 - protein, 70 aa, synthetic construct
GPETLCGAELVMALQFVCGDRGFYFNKPTGYGSSSRRAPQTGIVDECCFRSCDLRRLEMYCAPLKPAKSA

SEQ ID NO: 154 - protein, 70 aa, synthetic construct
GPETLCGAELVNALQFVCGDRGFYFNKPTGYGSSSRRAPQTGIVDECCFRSCDLRRLEMYCAPLKPAKSA

SEQ ID NO: 155 - protein, 70 aa, synthetic construct
GPETLCGAELVPALQFVCGDRGFYFNKPTGYGSSSRRAPQTGIVDECCFRSCDLRRLEMYCAPLKPAKSA

SEQ ID NO: 156 - protein, 70 aa, synthetic construct
GPETLCGAELVQALQFVCGDRGFYFNKPTGYGSSSRRAPQTGIVDECCFRSCDLRRLEMYCAPLKPAKSA

SEQ ID NO: 157 - protein, 70 aa, synthetic construct
GPETLCGAELVRALQFVCGDRGFYFNKPTGYGSSSRRAPQTGIVDECCFRSCDLRRLEMYCAPLKPAKSA

SEQ ID NO: 158 - protein, 70 aa, synthetic construct
GPETLCGAELVSALQFVCGDRGFYFNKPTGYGSSSRRAPQTGIVDECCFRSCDLRRLEMYCAPLKPAKSA

SEQ ID NO: 159 - protein, 70 aa, synthetic construct
GPETLCGAELVTALQFVCGDRGFYFNKPTGYGSSSRRAPQTGIVDECCFRSCDLRRLEMYCAPLKPAKSA

SEQ ID NO: 160 - protein, 70 aa, synthetic construct
GPETLCGAELVVALQFVCGDRGFYFNKPTGYGSSSRRAPQTGIVDECCFRSCDLRRLEMYCAPLKPAKSA

SEQ ID NO: 161 - protein, 70 aa, synthetic construct
GPETLCGAELVWALQFVCGDRGFYFNKPTGYGSSSRRAPQTGIVDECCFRSCDLRRLEMYCAPLKPAKSA

SEQ ID NO: 162 - protein, 70 aa, synthetic construct
GPETLCGAELVYALQFVCGDRGFYFNKPTGYGSSSRRAPQTGIVDECCFRSCDLRRLEMYCAPLKPAKSA

SEQ ID NO: 163 - protein, 70 aa, synthetic construct
GPETLCGAELVDALQFVCGDRGFYFNKPTGYGSSSRRAPQTGIVDECCCRSCDLRRLEMYCAPLKPAKSA

SEQ ID NO: 164 - protein, 70 aa, synthetic construct
GPETLCGAELVDALQFVCGDRGFYFNKPTGYGSSSRRAPQTGIVDECCDRSCDLRRLEMYCAPLKPAKSA

SEQ ID NO: 165 - protein, 70 aa, synthetic construct
GPETLCGAELVDALQFVCGDRGFYFNKPTGYGSSSRRAPQTGIVDECCERSCDLRRLEMYCAPLKPAKSA

SEQ ID NO: 166 - protein, 70 aa, synthetic construct
GPETLCGAELVDALQFVCGDRGFYFNKPTGYGSSSRRAPQTGIVDECCGRSCDLRRLEMYCAPLKPAKSA

SEQ ID NO: 167 - protein, 70 aa, synthetic construct
GPETLCGAELVDALQFVCGDRGFYFNKPTGYGSSSRRAPQTGIVDECCHRSCDLRRLEMYCAPLKPAKSA

SEQ ID NO: 168 - protein, 70 aa, synthetic construct
GPETLCGAELVDALQFVCGDRGFYFNKPTGYGSSSRRAPQTGIVDECCIRSCDLRRLEMYCAPLKPAKSA

SEQ ID NO: 169 - protein, 70 aa, synthetic construct
GPETLCGAELVDALQFVCGDRGFYFNKPTGYGSSSRRAPQTGIVDECCKRSCDLRRLEMYCAPLKPAKSA

SEQ ID NO: 170 - protein, 70 aa, synthetic construct
GPETLCGAELVDALQFVCGDRGFYFNKPTGYGSSSRRAPQTGIVDECCMRSCDLRRLEMYCAPLKPAKSA

SEQ ID NO: 171 - protein, 70 aa, synthetic construct
GPETLCGAELVDALQFVCGDRGFYFNKPTGYGSSSRRAPQTGIVDECCNRSCDLRRLEMYCAPLKPAKSA

SEQ ID NO: 172 - protein, 70 aa, synthetic construct
GPETLCGAELVDALQFVCGDRGFYFNKPTGYGSSSRRAPQTGIVDECCPRSCDLRRLEMYCAPLKPAKSA

SEQ ID NO: 173 - protein, 70 aa, synthetic construct
GPETLCGAELVDALQFVCGDRGFYFNKPTGYGSSSRRAPQTGIVDECCQRSCDLRRLEMYCAPLKPAKSA

SEQ ID NO: 174 - protein, 70 aa, synthetic construct
GPETLCGAELVDALQFVCGDRGFYFNKPTGYGSSSRRAPQTGIVDECCRRSCDLRRLEMYCAPLKPAKSA

SEQ ID NO: 175 - protein, 70 aa, synthetic construct
GPETLCGAELVDALQFVCGDRGFYFNKPTGYGSSSRRAPQTGIVDECCSRSCDLRRLEMYCAPLKPAKSA

SEQ ID NO: 176 - protein, 70 aa, synthetic construct
GPETLCGAELVDALQFVCGDRGFYFNKPTGYGSSSRRAPQTGIVDECCTRSCDLRRLEMYCAPLKPAKSA

SEQ ID NO: 177 - protein, 70 aa, synthetic construct
GPETLCGAELVDALQFVCGDRGFYFNKPTGYGSSSRRAPQTGIVDECCVRSCDLRRLEMYCAPLKPAKSA

SEQ ID NO: 178 - protein, 70 aa, synthetic construct
GPETLCGAELVDALQFVCGDRGFYFNKPTGYGSSSRRAPQTGIVDECCWRSCDLRRLEMYCAPLKPAKSA

SEQ ID NO: 179 - protein, 70 aa, synthetic construct
GPETLCGAELVDALQFVCGDRGFYFNKPTGYGSSSRRAPQTGIVDECCYRSCDLRRLEMYCAPLKPAKSA

SEQ ID NO: 180 - protein, 70 aa, synthetic construct
GPETLCGAELVDALQFVCGDRGFYFNKPTGYGSSSRRAPQTGIVDECCFRSCDCRRLEMYCAPLKPAKSA

SEQ ID NO: 181 - protein, 70 aa, synthetic construct
GPETLCGAELVDALQFVCGDRGFYFNKPTGYGSSSRRAPQTGIVDECCFRSCDDRRLEMYCAPLKPAKSA

SEQ ID NO: 182 - protein, 70 aa, synthetic construct
GPETLCGAELVDALQFVCGDRGFYFNKPTGYGSSSRRAPQTGIVDECCFRSCDERRLEMYCAPLKPAKSA

SEQ ID NO: 183 - protein, 70 aa, synthetic construct
GPETLCGAELVDALQFVCGDRGFYFNKPTGYGSSSRRAPQTGIVDECCFRSCDFRRLEMYCAPLKPAKSA

SEQ ID NO: 184 - protein, 70 aa, synthetic construct
GPETLCGAELVDALQFVCGDRGFYFNKPTGYGSSSRRAPQTGIVDECCFRSCDGRRLEMYCAPLKPAKSA

SEQ ID NO: 185 - protein, 70 aa, synthetic construct
GPETLCGAELVDALQFVCGDRGFYFNKPTGYGSSSRRAPQTGIVDECCFRSCDHRRLEMYCAPLKPAKSA

SEQ ID NO: 186 - protein, 70 aa, synthetic construct
GPETLCGAELVDALQFVCGDRGFYFNKPTGYGSSSRRAPQTGIVDECCFRSCDIRRLEMYCAPLKPAKSA

SEQ ID NO: 187 - protein, 70 aa, synthetic construct
GPETLCGAELVDALQFVCGDRGFYFNKPTGYGSSSRRAPQTGIVDECCFRSCDKRRLEMYCAPLKPAKSA

SEQ ID NO: 188 - protein, 70 aa, synthetic construct
GPETLCGAELVDALQFVCGDRGFYFNKPTGYGSSSRRAPQTGIVDECCFRSCDMRRLEMYCAPLKPAKSA

SEQ ID NO: 189 - protein, 70 aa, synthetic construct
GPETLCGAELVDALQFVCGDRGFYFNKPTGYGSSSRRAPQTGIVDECCFRSCDNRRLEMYCAPLKPAKSA

SEQ ID NO: 190 - protein, 70 aa, synthetic construct
GPETLCGAELVDALQFVCGDRGFYFNKPTGYGSSSRRAPQTGIVDECCFRSCDPRRLEMYCAPLKPAKSA

SEQ ID NO: 191 - protein, 70 aa, synthetic construct
GPETLCGAELVDALQFVCGDRGFYFNKPTGYGSSSRRAPQTGIVDECCFRSCDQRRLEMYCAPLKPAKSA

SEQ ID NO: 192 - protein, 70 aa, synthetic construct
GPETLCGAELVDALQFVCGDRGFYFNKPTGYGSSSRRAPQTGIVDECCFRSCDRRRLEMYCAPLKPAKSA

SEQ ID NO: 193 - protein, 70 aa, synthetic construct
GPETLCGAELVDALQFVCGDRGFYFNKPTGYGSSSRRAPQTGIVDECCFRSCDSRRLEMYCAPLKPAKSA

SEQ ID NO: 194 - protein, 70 aa, synthetic construct
GPETLCGAELVDALQFVCGDRGFYFNKPTGYGSSSRRAPQTGIVDECCFRSCDTRRLEMYCAPLKPAKSA

SEQ ID NO: 195 - protein, 70 aa, synthetic construct
GPETLCGAELVDALQFVCGDRGFYFNKPTGYGSSSRRAPQTGIVDECCFRSCDVRRLEMYCAPLKPAKSA

SEQ ID NO: 196 - protein, 70 aa, synthetic construct
GPETLCGAELVDALQFVCGDRGFYFNKPTGYGSSSRRAPQTGIVDECCFRSCDWRRLEMYCAPLKPAKSA

SEQ ID NO: 197 - protein, 70 aa, synthetic construct
GPETLCGAELVDALQFVCGDRGFYFNKPTGYGSSSRRAPQTGIVDECCFRSCDYRRLEMYCAPLKPAKSA

SEQ ID NO: 198 - protein, 70 aa, synthetic construct
GPRTACGAELVDALQFVCGDRGFYFNKPTGYGSSSRRAPQTGIVDECCFRSCDLRRLEMYCAPLKPAKSA

SEQ ID NO: 199 - protein, 70 aa, synthetic construct
GPRTCCGAELVDALQFVCGDRGFYFNKPTGYGSSSRRAPQTGIVDECCFRSCDLRRLEMYCAPLKPAKSA

SEQ ID NO: 200 - protein, 70 aa, synthetic construct
GPRTDCGAELVDALQFVCGDRGFYFNKPTGYGSSSRRAPQTGIVDECCFRSCDLRRLEMYCAPLKPAKSA

SEQ ID NO: 201 - protein, 70 aa, synthetic construct
GPRTECGAELVDALQFVCGDRGFYFNKPTGYGSSSRRAPQTGIVDECCFRSCDLRRLEMYCAPLKPAKSA

SEQ ID NO: 202 - protein, 70 aa, synthetic construct
GPRTFCGAELVDALQFVCGDRGFYFNKPTGYGSSSRRAPQTGIVDECCFRSCDLRRLEMYCAPLKPAKSA

SEQ ID NO: 203 - protein, 70 aa, synthetic construct
GPRTGCGAELVDALQFVCGDRGFYFNKPTGYGSSSRRAPQTGIVDECCFRSCDLRRLEMYCAPLKPAKSA

SEQ ID NO: 204 - protein, 70 aa, synthetic construct
GPRTHCGAELVDALQFVCGDRGFYFNKPTGYGSSSRRAPQTGIVDECCFRSCDLRRLEMYCAPLKPAKSA

SEQ ID NO: 205 - protein, 70 aa, synthetic construct
GPRTICGAELVDALQFVCGDRGFYFNKPTGYGSSSRRAPQTGIVDECCFRSCDLRRLEMYCAPLKPAKSA

SEQ ID NO: 206 - protein, 70 aa, synthetic construct
GPRTKCGAELVDALQFVCGDRGFYFNKPTGYGSSSRRAPQTGIVDECCFRSCDLRRLEMYCAPLKPAKSA

SEQ ID NO: 207 - protein, 70 aa, synthetic construct
GPRTMCGAELVDALQFVCGDRGFYFNKPTGYGSSSRRAPQTGIVDECCFRSCDLRRLEMYCAPLKPAKSA

SEQ ID NO: 208 - protein, 70 aa, synthetic construct
GPRTNCGAELVDALQFVCGDRGFYFNKPTGYGSSSRRAPQTGIVDECCFRSCDLRRLEMYCAPLKPAKSA

SEQ ID NO: 209 - protein, 70 aa, synthetic construct
GPRTPCGAELVDALQFVCGDRGFYFNKPTGYGSSSRRAPQTGIVDECCFRSCDLRRLEMYCAPLKPAKSA

SEQ ID NO: 210 - protein, 70 aa, synthetic construct
GPRTQCGAELVDALQFVCGDRGFYFNKPTGYGSSSRRAPQTGIVDECCFRSCDLRRLEMYCAPLKPAKSA

SEQ ID NO: 211 - protein, 70 aa, synthetic construct
GPRTRCGAELVDALQFVCGDRGFYFNKPTGYGSSSRRAPQTGIVDECCFRSCDLRRLEMYCAPLKPAKSA

SEQ ID NO: 212 - protein, 70 aa, synthetic construct
GPRTSCGAELVDALQFVCGDRGFYFNKPTGYGSSSRRAPQTGIVDECCFRSCDLRRLEMYCAPLKPAKSA

SEQ ID NO: 213 - protein, 70 aa, synthetic construct
GPRTTCGAELVDALQFVCGDRGFYFNKPTGYGSSSRRAPQTGIVDECCFRSCDLRRLEMYCAPLKPAKSA

SEQ ID NO: 214 - protein, 70 aa, synthetic construct
GPRTVCGAELVDALQFVCGDRGFYFNKPTGYGSSSRRAPQTGIVDECCFRSCDLRRLEMYCAPLKPAKSA

SEQ ID NO: 215 - protein, 70 aa, synthetic construct
GPRTWCGAELVDALQFVCGDRGFYFNKPTGYGSSSRRAPQTGIVDECCFRSCDLRRLEMYCAPLKPAKSA

SEQ ID NO: 216 - protein, 70 aa, synthetic construct
GPRTYCGAELVDALQFVCGDRGFYFNKPTGYGSSSRRAPQTGIVDECCFRSCDLRRLEMYCAPLKPAKSA

SEQ ID NO: 217 - protein, 67 aa, synthetic construct
TACGAELVDALQFVCGDRGFYFNKPTGYGSSSRRAPQTGIVDECCFRSCDLRRLEMYCAPLKPAKSA

SEQ ID NO: 218 - protein, 67 aa, synthetic construct
TCCGAELVDALQFVCGDRGFYFNKPTGYGSSSRRAPQTGIVDECCFRSCDLRRLEMYCAPLKPAKSA

SEQ ID NO: 219 - protein, 67 aa, synthetic construct
TDCGAELVDALQFVCGDRGFYFNKPTGYGSSSRRAPQTGIVDECCFRSCDLRRLEMYCAPLKPAKSA

SEQ ID NO: 220 - protein, 67 aa, synthetic construct
TECGAELVDALQFVCGDRGFYFNKPTGYGSSSRRAPQTGIVDECCFRSCDLRRLEMYCAPLKPAKSA

SEQ ID NO: 221 - protein, 67 aa, synthetic construct
TFCGAELVDALQFVCGDRGFYFNKPTGYGSSSRRAPQTGIVDECCFRSCDLRRLEMYCAPLKPAKSA

SEQ ID NO: 222 - protein, 67 aa, synthetic construct
TGCGAELVDALQFVCGDRGFYFNKPTGYGSSSRRAPQTGIVDECCFRSCDLRRLEMYCAPLKPAKSA

SEQ ID NO: 223 - protein, 67 aa, synthetic construct
THCGAELVDALQFVCGDRGFYFNKPTGYGSSSRRAPQTGIVDECCFRSCDLRRLEMYCAPLKPAKSA

SEQ ID NO: 224 - protein, 67 aa, synthetic construct
TICGAELVDALQFVCGDRGFYFNKPTGYGSSSRRAPQTGIVDECCFRSCDLRRLEMYCAPLKPAKSA

SEQ ID NO: 225 - protein, 67 aa, synthetic construct
TKCGAELVDALQFVCGDRGFYFNKPTGYGSSSRRAPQTGIVDECCFRSCDLRRLEMYCAPLKPAKSA

SEQ ID NO: 226 - protein, 67 aa, synthetic construct
TMCGAELVDALQFVCGDRGFYFNKPTGYGSSSRRAPQTGIVDECCFRSCDLRRLEMYCAPLKPAKSA

SEQ ID NO: 227 - protein, 67 aa, synthetic construct
TNCGAELVDALQFVCGDRGFYFNKPTGYGSSSRRAPQTGIVDECCFRSCDLRRLEMYCAPLKPAKSA

SEQ ID NO: 228 - protein, 67 aa, synthetic construct
TPCGAELVDALQFVCGDRGFYFNKPTGYGSSSRRAPQTGIVDECCFRSCDLRRLEMYCAPLKPAKSA

SEQ ID NO: 229 - protein, 67 aa, synthetic construct
TQCGAELVDALQFVCGDRGFYFNKPTGYGSSSRRAPQTGIVDECCFRSCDLRRLEMYCAPLKPAKSA

SEQ ID NO: 230 - protein, 67 aa, synthetic construct
TRCGAELVDALQFVCGDRGFYFNKPTGYGSSSRRAPQTGIVDECCFRSCDLRRLEMYCAPLKPAKSA

SEQ ID NO: 231 - protein, 67 aa, synthetic construct
TSCGAELVDALQFVCGDRGFYFNKPTGYGSSSRRAPQTGIVDECCFRSCDLRRLEMYCAPLKPAKSA

SEQ ID NO: 232 - protein, 67 aa, synthetic construct
TTCGAELVDALQFVCGDRGFYFNKPTGYGSSSRRAPQTGIVDECCFRSCDLRRLEMYCAPLKPAKSA

SEQ ID NO: 233 - protein, 67 aa, synthetic construct
TVCGAELVDALQFVCGDRGFYFNKPTGYGSSSRRAPQTGIVDECCFRSCDLRRLEMYCAPLKPAKSA

SEQ ID NO: 234 - protein, 67 aa, synthetic construct
TWCGAELVDALQFVCGDRGFYFNKPTGYGSSSRRAPQTGIVDECCFRSCDLRRLEMYCAPLKPAKSA

SEQ ID NO: 235 - protein, 67 aa, synthetic construct
TYCGAELVDALQFVCGDRGFYFNKPTGYGSSSRRAPQTGIVDECCFRSCDLRRLEMYCAPLKPAKSA

SEQ ID NO: 236 - protein, 70 aa, synthetic construct
GPRTLCAAELVDALQFVCGDRGFYFNKPTGYGSSSRRAPQTGIVDECCFRSCDLRRLEMYCAPLKPAKSA

SEQ ID NO: 237 - protein, 70 aa, synthetic construct
GPRTLCCAELVDALQFVCGDRGFYFNKPTGYGSSSRRAPQTGIVDECCFRSCDLRRLEMYCAPLKPAKSA

SEQ ID NO: 238 - protein, 70 aa, synthetic construct
GPRTLCDAELVDALQFVCGDRGFYFNKPTGYGSSSRRAPQTGIVDECCFRSCDLRRLEMYCAPLKPAKSA

SEQ ID NO: 239 - protein, 70 aa, synthetic construct
GPRTLCEAELVDALQFVCGDRGFYFNKPTGYGSSSRRAPQTGIVDECCFRSCDLRRLEMYCAPLKPAKSA

SEQ ID NO: 240 - protein, 70 aa, synthetic construct
GPRTLCFAELVDALQFVCGDRGFYFNKPTGYGSSSRRAPQTGIVDECCFRSCDLRRLEMYCAPLKPAKSA

SEQ ID NO: 241 - protein, 70 aa, synthetic construct
GPRTLCHAELVDALQFVCGDRGFYFNKPTGYGSSSRRAPQTGIVDECCFRSCDLRRLEMYCAPLKPAKSA

SEQ ID NO: 242 - protein, 70 aa, synthetic construct
GPRTLCIAELVDALQFVCGDRGFYFNKPTGYGSSSRRAPQTGIVDECCFRSCDLRRLEMYCAPLKPAKSA

SEQ ID NO: 243 - protein, 70 aa, synthetic construct
GPRTLCKAELVDALQFVCGDRGFYFNKPTGYGSSSRRAPQTGIVDECCFRSCDLRRLEMYCAPLKPAKSA

SEQ ID NO: 244 - protein, 70 aa, synthetic construct
GPRTLCLAELVDALQFVCGDRGFYFNKPTGYGSSSRRAPQTGIVDECCFRSCDLRRLEMYCAPLKPAKSA

SEQ ID NO: 245 - protein, 70 aa, synthetic construct
GPRTLCMAELVDALQFVCGDRGFYFNKPTGYGSSSRRAPQTGIVDECCFRSCDLRRLEMYCAPLKPAKSA

SEQ ID NO: 246 - protein, 70 aa, synthetic construct
GPRTLCNAELVDALQFVCGDRGFYFNKPTGYGSSSRRAPQTGIVDECCFRSCDLRRLEMYCAPLKPAKSA

SEQ ID NO: 247 - protein, 70 aa, synthetic construct
GPRTLCPAELVDALQFVCGDRGFYFNKPTGYGSSSRRAPQTGIVDECCFRSCDLRRLEMYCAPLKPAKSA

SEQ ID NO: 248 - protein, 70 aa, synthetic construct
GPRTLCQAELVDALQFVCGDRGFYFNKPTGYGSSSRRAPQTGIVDECCFRSCDLRRLEMYCAPLKPAKSA

SEQ ID NO: 249 - protein, 70 aa, synthetic construct
GPRTLCRAELVDALQFVCGDRGFYFNKPTGYGSSSRRAPQTGIVDECCFRSCDLRRLEMYCAPLKPAKSA

SEQ ID NO: 250 - protein, 70 aa, synthetic construct
GPRTLCSAELVDALQFVCGDRGFYFNKPTGYGSSSRRAPQTGIVDECCFRSCDLRRLEMYCAPLKPAKSA

SEQ ID NO: 251 - protein, 70 aa, synthetic construct
GPRTLCTAELVDALQFVCGDRGFYFNKPTGYGSSSRRAPQTGIVDECCFRSCDLRRLEMYCAPLKPAKSA

SEQ ID NO: 252 - protein, 70 aa, synthetic construct
GPRTLCVAELVDALQFVCGDRGFYFNKPTGYGSSSRRAPQTGIVDECCFRSCDLRRLEMYCAPLKPAKSA

SEQ ID NO: 253 - protein, 70 aa, synthetic construct
GPRTLCWAELVDALQFVCGDRGFYFNKPTGYGSSSRRAPQTGIVDECCFRSCDLRRLEMYCAPLKPAKSA

SEQ ID NO: 254 - protein, 70 aa, synthetic construct
GPRTLCYAELVDALQFVCGDRGFYFNKPTGYGSSSRRAPQTGIVDECCFRSCDLRRLEMYCAPLKPAKSA

SEQ ID NO: 255 - protein, 67 aa, synthetic construct
TLCAAELVDALQFVCGDRGFYFNKPTGYGSSSRRAPQTGIVDECCFRSCDLRRLEMYCAPLKPAKSA

SEQ ID NO: 256 - protein, 67 aa, synthetic construct
TLCCAELVDALQFVCGDRGFYFNKPTGYGSSSRRAPQTGIVDECCFRSCDLRRLEMYCAPLKPAKSA

SEQ ID NO: 257 - protein, 67 aa, synthetic construct
TLCDAELVDALQFVCGDRGFYFNKPTGYGSSSRRAPQTGIVDECCFRSCDLRRLEMYCAPLKPAKSA

SEQ ID NO: 258 - protein, 67 aa, synthetic construct
TLCEAELVDALQFVCGDRGFYFNKPTGYGSSSRRAPQTGIVDECCFRSCDLRRLEMYCAPLKPAKSA

SEQ ID NO: 259 - protein, 67 aa, synthetic construct
TLCFAELVDALQFVCGDRGFYFNKPTGYGSSSRRAPQTGIVDECCFRSCDLRRLEMYCAPLKPAKSA

SEQ ID NO: 260 - protein, 67 aa, synthetic construct
TLCHAELVDALQFVCGDRGFYFNKPTGYGSSSRRAPQTGIVDECCFRSCDLRRLEMYCAPLKPAKSA

SEQ ID NO: 261 - protein, 67 aa, synthetic construct
TLCIAELVDALQFVCGDRGFYFNKPTGYGSSSRRAPQTGIVDECCFRSCDLRRLEMYCAPLKPAKSA

SEQ ID NO: 262 - protein, 67 aa, synthetic construct
TLCKAELVDALQFVCGDRGFYFNKPTGYGSSSRRAPQTGIVDECCFRSCDLRRLEMYCAPLKPAKSA

SEQ ID NO: 263 - protein, 67 aa, synthetic construct
TLCLAELVDALQFVCGDRGFYFNKPTGYGSSSRRAPQTGIVDECCFRSCDLRRLEMYCAPLKPAKSA

SEQ ID NO: 264 - protein, 67 aa, synthetic construct
TLCMAELVDALQFVCGDRGFYFNKPTGYGSSSRRAPQTGIVDECCFRSCDLRRLEMYCAPLKPAKSA

SEQ ID NO: 265 - protein, 67 aa, synthetic construct
TLCNAELVDALQFVCGDRGFYFNKPTGYGSSSRRAPQTGIVDECCFRSCDLRRLEMYCAPLKPAKSA

SEQ ID NO: 266 - protein, 67 aa, synthetic construct
TLCPAELVDALQFVCGDRGFYFNKPTGYGSSSRRAPQTGIVDECCFRSCDLRRLEMYCAPLKPAKSA

SEQ ID NO: 267 - protein, 67 aa, synthetic construct
TLCQAELVDALQFVCGDRGFYFNKPTGYGSSSRRAPQTGIVDECCFRSCDLRRLEMYCAPLKPAKSA

SEQ ID NO: 268 - protein, 67 aa, synthetic construct
TLCRAELVDALQFVCGDRGFYFNKPTGYGSSSRRAPQTGIVDECCFRSCDLRRLEMYCAPLKPAKSA

SEQ ID NO: 269 - protein, 67 aa, synthetic construct
TLCSAELVDALQFVCGDRGFYFNKPTGYGSSSRRAPQTGIVDECCFRSCDLRRLEMYCAPLKPAKSA

SEQ ID NO: 270 - protein, 67 aa, synthetic construct
TLCTAELVDALQFVCGDRGFYFNKPTGYGSSSRRAPQTGIVDECCFRSCDLRRLEMYCAPLKPAKSA

SEQ ID NO: 271 - protein, 67 aa, synthetic construct
TLCVAELVDALQFVCGDRGFYFNKPTGYGSSSRRAPQTGIVDECCFRSCDLRRLEMYCAPLKPAKSA

SEQ ID NO: 272 - protein, 67 aa, synthetic construct
TLCWAELVDALQFVCGDRGFYFNKPTGYGSSSRRAPQTGIVDECCFRSCDLRRLEMYCAPLKPAKSA

SEQ ID NO: 273 - protein, 67 aa, synthetic construct
TLCYAELVDALQFVCGDRGFYFNKPTGYGSSSRRAPQTGIVDECCFRSCDLRRLEMYCAPLKPAKSA

SEQ ID NO: 274 - protein, 70 aa, synthetic construct
GPRTLCGAEAVDALQFVCGDRGFYFNKPTGYGSSSRRAPQTGIVDECCFRSCDLRRLEMYCAPLKPAKSA

SEQ ID NO: 275 - protein, 70 aa, synthetic construct
GPRTLCGAECVDALQFVCGDRGFYFNKPTGYGSSSRRAPQTGIVDECCFRSCDLRRLEMYCAPLKPAKSA

SEQ ID NO: 276 - protein, 70 aa, synthetic construct
GPRTLCGAEDVDALQFVCGDRGFYFNKPTGYGSSSRRAPQTGIVDECCFRSCDLRRLEMYCAPLKPAKSA

SEQ ID NO: 277 - protein, 70 aa, synthetic construct
GPRTLCGAEEVDALQFVCGDRGFYFNKPTGYGSSSRRAPQTGIVDECCFRSCDLRRLEMYCAPLKPAKSA

SEQ ID NO: 278 - protein, 70 aa, synthetic construct
GPRTLCGAEFVDALQFVCGDRGFYFNKPTGYGSSSRRAPQTGIVDECCFRSCDLRRLEMYCAPLKPAKSA

SEQ ID NO: 279 - protein, 70 aa, synthetic construct
GPRTLCGAEGVDALQFVCGDRGFYFNKPTGYGSSSRRAPQTGIVDECCFRSCDLRRLEMYCAPLKPAKSA

SEQ ID NO: 280 - protein, 70 aa, synthetic construct
GPRTLCGAEHVDALQFVCGDRGFYFNKPTGYGSSSRRAPQTGIVDECCFRSCDLRRLEMYCAPLKPAKSA

SEQ ID NO: 281 - protein, 70 aa, synthetic construct
GPRTLCGAEIVDALQFVCGDRGFYFNKPTGYGSSSRRAPQTGIVDECCFRSCDLRRLEMYCAPLKPAKSA

SEQ ID NO: 282 - protein, 70 aa, synthetic construct
GPRTLCGAEKVDALQFVCGDRGFYFNKPTGYGSSSRRAPQTGIVDECCFRSCDLRRLEMYCAPLKPAKSA

SEQ ID NO: 283 - protein, 70 aa, synthetic construct
GPRTLCGAEMVDALQFVCGDRGFYFNKPTGYGSSSRRAPQTGIVDECCFRSCDLRRLEMYCAPLKPAKSA

SEQ ID NO: 284 - protein, 70 aa, synthetic construct
GPRTLCGAENVDALQFVCGDRGFYFNKPTGYGSSSRRAPQTGIVDECCFRSCDLRRLEMYCAPLKPAKSA

SEQ ID NO: 285 - protein, 70 aa, synthetic construct
GPRTLCGAEPVDALQFVCGDRGFYFNKPTGYGSSSRRAPQTGIVDECCFRSCDLRRLEMYCAPLKPAKSA

SEQ ID NO: 286 - protein, 70 aa, synthetic construct
GPRTLCGAEQVDALQFVCGDRGFYFNKPTGYGSSSRRAPQTGIVDECCFRSCDLRRLEMYCAPLKPAKSA

SEQ ID NO: 287 - protein, 70 aa, synthetic construct
GPRTLCGAERVDALQFVCGDRGFYFNKPTGYGSSSRRAPQTGIVDECCFRSCDLRRLEMYCAPLKPAKSA

SEQ ID NO: 288 - protein, 70 aa, synthetic construct
GPRTLCGAESVDALQFVCGDRGFYFNKPTGYGSSSRRAPQTGIVDECCFRSCDLRRLEMYCAPLKPAKSA

SEQ ID NO: 289 - protein, 70 aa, synthetic construct
GPRTLCGAETVDALQFVCGDRGFYFNKPTGYGSSSRRAPQTGIVDECCFRSCDLRRLEMYCAPLKPAKSA

SEQ ID NO: 290 - protein, 70 aa, synthetic construct
GPRTLCGAEVVDALQFVCGDRGFYFNKPTGYGSSSRRAPQTGIVDECCFRSCDLRRLEMYCAPLKPAKSA

SEQ ID NO: 291 - protein, 70 aa, synthetic construct
GPRTLCGAEWVDALQFVCGDRGFYFNKPTGYGSSSRRAPQTGIVDECCFRSCDLRRLEMYCAPLKPAKSA

SEQ ID NO: 292 - protein, 70 aa, synthetic construct
GPRTLCGAEYVDALQFVCGDRGFYFNKPTGYGSSSRRAPQTGIVDECCFRSCDLRRLEMYCAPLKPAKSA

SEQ ID NO: 293 - protein, 67 aa, synthetic construct
TLCGAEAVDALQFVCGDRGFYFNKPTGYGSSSRRAPQTGIVDECCFRSCDLRRLEMYCAPLKPAKSA

SEQ ID NO: 294 - protein, 67 aa, synthetic construct
TLCGAECVDALQFVCGDRGFYFNKPTGYGSSSRRAPQTGIVDECCFRSCDLRRLEMYCAPLKPAKSA

SEQ ID NO: 295 - protein, 67 aa, synthetic construct
TLCGAEDVDALQFVCGDRGFYFNKPTGYGSSSRRAPQTGIVDECCFRSCDLRRLEMYCAPLKPAKSA

SEQ ID NO: 296 - protein, 67 aa, synthetic construct
TLCGAEEVDALQFVCGDRGFYFNKPTGYGSSSRRAPQTGIVDECCFRSCDLRRLEMYCAPLKPAKSA

SEQ ID NO: 297 - protein, 67 aa, synthetic construct
TLCGAEFVDALQFVCGDRGFYFNKPTGYGSSSRRAPQTGIVDECCFRSCDLRRLEMYCAPLKPAKSA

SEQ ID NO: 298 - protein, 67 aa, synthetic construct
TLCGAEGVDALQFVCGDRGFYFNKPTGYGSSSRRAPQTGIVDECCFRSCDLRRLEMYCAPLKPAKSA

SEQ ID NO: 299 - protein, 67 aa, synthetic construct
TLCGAEHVDALQFVCGDRGFYFNKPTGYGSSSRRAPQTGIVDECCFRSCDLRRLEMYCAPLKPAKSA

SEQ ID NO: 300 - protein, 67 aa, synthetic construct
TLCGAEIVDALQFVCGDRGFYFNKPTGYGSSSRRAPQTGIVDECCFRSCDLRRLEMYCAPLKPAKSA

SEQ ID NO: 301 - protein, 67 aa, synthetic construct
TLCGAEKVDALQFVCGDRGFYFNKPTGYGSSSRRAPQTGIVDECCFRSCDLRRLEMYCAPLKPAKSA

SEQ ID NO: 302 - protein, 67 aa, synthetic construct
TLCGAEMVDALQFVCGDRGFYFNKPTGYGSSSRRAPQTGIVDECCFRSCDLRRLEMYCAPLKPAKSA

SEQ ID NO: 303 - protein, 67 aa, synthetic construct
TLCGAENVDALQFVCGDRGFYFNKPTGYGSSSRRAPQTGIVDECCFRSCDLRRLEMYCAPLKPAKSA

SEQ ID NO: 304 - protein, 67 aa, synthetic construct
TLCGAEPVDALQFVCGDRGFYFNKPTGYGSSSRRAPQTGIVDECCFRSCDLRRLEMYCAPLKPAKSA

SEQ ID NO: 305 - protein, 67 aa, synthetic construct
TLCGAEQVDALQFVCGDRGFYFNKPTGYGSSSRRAPQTGIVDECCFRSCDLRRLEMYCAPLKPAKSA

SEQ ID NO: 306 - protein, 67 aa, synthetic construct
TLCGAERVDALQFVCGDRGFYFNKPTGYGSSSRRAPQTGIVDECCFRSCDLRRLEMYCAPLKPAKSA

SEQ ID NO: 307 - protein, 67 aa, synthetic construct
TLCGAESVDALQFVCGDRGFYFNKPTGYGSSSRRAPQTGIVDECCFRSCDLRRLEMYCAPLKPAKSA

SEQ ID NO: 308 - protein, 67 aa, synthetic construct
TLCGAETVDALQFVCGDRGFYFNKPTGYGSSSRRAPQTGIVDECCFRSCDLRRLEMYCAPLKPAKSA

SEQ ID NO: 309 - protein, 67 aa, synthetic construct
TLCGAEVVDALQFVCGDRGFYFNKPTGYGSSSRRAPQTGIVDECCFRSCDLRRLEMYCAPLKPAKSA

SEQ ID NO: 310 - protein, 67 aa, synthetic construct
TLCGAEWVDALQFVCGDRGFYFNKPTGYGSSSRRAPQTGIVDECCFRSCDLRRLEMYCAPLKPAKSA

SEQ ID NO: 311 - protein, 67 aa, synthetic construct
TLCGAEYVDALQFVCGDRGFYFNKPTGYGSSSRRAPQTGIVDECCFRSCDLRRLEMYCAPLKPAKSA

SEQ ID NO: 312 - protein, 70 aa, synthetic construct
GPRTLCGAELVDALQFACGDRGFYFNKPTGYGSSSRRAPQTGIVDECCFRSCDLRRLEMYCAPLKPAKSA

SEQ ID NO: 313 - protein, 70 aa, synthetic construct
GPRTLCGAELVDALQFCCGDRGFYFNKPTGYGSSSRRAPQTGIVDECCFRSCDLRRLEMYCAPLKPAKSA

SEQ ID NO: 314 - protein, 70 aa, synthetic construct
GPRTLCGAELVDALQFDCGDRGFYFNKPTGYGSSSRRAPQTGIVDECCFRSCDLRRLEMYCAPLKPAKSA

SEQ ID NO: 315 - protein, 70 aa, synthetic construct
GPRTLCGAELVDALQFECGDRGFYFNKPTGYGSSSRRAPQTGIVDECCFRSCDLRRLEMYCAPLKPAKSA

SEQ ID NO: 316 - protein, 70 aa, synthetic construct
GPRTLCGAELVDALQFFCGDRGFYFNKPTGYGSSSRRAPQTGIVDECCFRSCDLRRLEMYCAPLKPAKSA

SEQ ID NO: 317 - protein, 70 aa, synthetic construct
GPRTLCGAELVDALQFGCGDRGFYFNKPTGYGSSSRRAPQTGIVDECCFRSCDLRRLEMYCAPLKPAKSA

SEQ ID NO: 318 - protein, 70 aa, synthetic construct
GPRTLCGAELVDALQFHCGDRGFYFNKPTGYGSSSRRAPQTGIVDECCFRSCDLRRLEMYCAPLKPAKSA

SEQ ID NO: 319 - protein, 70 aa, synthetic construct
GPRTLCGAELVDALQFICGDRGFYFNKPTGYGSSSRRAPQTGIVDECCFRSCDLRRLEMYCAPLKPAKSA

SEQ ID NO: 320 - protein, 70 aa, synthetic construct
GPRTLCGAELVDALQFKCGDRGFYFNKPTGYGSSSRRAPQTGIVDECCFRSCDLRRLEMYCAPLKPAKSA

SEQ ID NO: 321 - protein, 70 aa, synthetic construct
GPRTLCGAELVDALQFLCGDRGFYFNKPTGYGSSSRRAPQTGIVDECCFRSCDLRRLEMYCAPLKPAKSA

SEQ ID NO: 322 - protein, 70 aa, synthetic construct
GPRTLCGAELVDALQFMCGDRGFYFNKPTGYGSSSRRAPQTGIVDECCFRSCDLRRLEMYCAPLKPAKSA

SEQ ID NO: 323 - protein, 70 aa, synthetic construct
GPRTLCGAELVDALQFNCGDRGFYFNKPTGYGSSSRRAPQTGIVDECCFRSCDLRRLEMYCAPLKPAKSA

SEQ ID NO: 324 - protein, 70 aa, synthetic construct
GPRTLCGAELVDALQFPCGDRGFYFNKPTGYGSSSRRAPQTGIVDECCFRSCDLRRLEMYCAPLKPAKSA

SEQ ID NO: 325 - protein, 70 aa, synthetic construct
GPRTLCGAELVDALQFQCGDRGFYFNKPTGYGSSSRRAPQTGIVDECCFRSCDLRRLEMYCAPLKPAKSA

SEQ ID NO: 326 - protein, 70 aa, synthetic construct
GPRTLCGAELVDALQFRCGDRGFYFNKPTGYGSSSRRAPQTGIVDECCFRSCDLRRLEMYCAPLKPAKSA

SEQ ID NO: 327 - protein, 70 aa, synthetic construct
GPRTLCGAELVDALQFSCGDRGFYFNKPTGYGSSSRRAPQTGIVDECCFRSCDLRRLEMYCAPLKPAKSA

SEQ ID NO: 328 - protein, 70 aa, synthetic construct
GPRTLCGAELVDALQFTCGDRGFYFNKPTGYGSSSRRAPQTGIVDECCFRSCDLRRLEMYCAPLKPAKSA

SEQ ID NO: 329 - protein, 70 aa, synthetic construct
GPRTLCGAELVDALQFWCGDRGFYFNKPTGYGSSSRRAPQTGIVDECCFRSCDLRRLEMYCAPLKPAKSA

SEQ ID NO: 330 - protein, 70 aa, synthetic construct
GPRTLCGAELVDALQFYCGDRGFYFNKPTGYGSSSRRAPQTGIVDECCFRSCDLRRLEMYCAPLKPAKSA

SEQ ID NO: 331 - protein, 67 aa, synthetic construct
TLCGAELVDALQFACGDRGFYFNKPTGYGSSSRRAPQTGIVDECCFRSCDLRRLEMYCAPLKPAKSA

SEQ ID NO: 332 - protein, 67 aa, synthetic construct
TLCGAELVDALQFCCGDRGFYFNKPTGYGSSSRRAPQTGIVDECCFRSCDLRRLEMYCAPLKPAKSA

SEQ ID NO: 333 - protein, 67 aa, synthetic construct
TLCGAELVDALQFDCGDRGFYFNKPTGYGSSSRRAPQTGIVDECCFRSCDLRRLEMYCAPLKPAKSA

SEQ ID NO: 334 - protein, 67 aa, synthetic construct
TLCGAELVDALQFECGDRGFYFNKPTGYGSSSRRAPQTGIVDECCFRSCDLRRLEMYCAPLKPAKSA

SEQ ID NO: 335 - protein, 67 aa, synthetic construct
TLCGAELVDALQFFCGDRGFYFNKPTGYGSSSRRAPQTGIVDECCFRSCDLRRLEMYCAPLKPAKSA

SEQ ID NO: 336 - protein, 67 aa, synthetic construct
TLCGAELVDALQFGCGDRGFYFNKPTGYGSSSRRAPQTGIVDECCFRSCDLRRLEMYCAPLKPAKSA

SEQ ID NO: 337 - protein, 67 aa, synthetic construct
TLCGAELVDALQFHCGDRGFYFNKPTGYGSSSRRAPQTGIVDECCFRSCDLRRLEMYCAPLKPAKSA

SEQ ID NO: 338 - protein, 67 aa, synthetic construct
TLCGAELVDALQFICGDRGFYFNKPTGYGSSSRRAPQTGIVDECCFRSCDLRRLEMYCAPLKPAKSA

SEQ ID NO: 339 - protein, 67 aa, synthetic construct
TLCGAELVDALQFKCGDRGFYFNKPTGYGSSSRRAPQTGIVDECCFRSCDLRRLEMYCAPLKPAKSA

SEQ ID NO: 340 - protein, 67 aa, synthetic construct
TLCGAELVDALQFLCGDRGFYFNKPTGYGSSSRRAPQTGIVDECCFRSCDLRRLEMYCAPLKPAKSA

SEQ ID NO: 341 - protein, 67 aa, synthetic construct
TLCGAELVDALQFMCGDRGFYFNKPTGYGSSSRRAPQTGIVDECCFRSCDLRRLEMYCAPLKPAKSA

SEQ ID NO: 342 - protein, 67 aa, synthetic construct
TLCGAELVDALQFNCGDRGFYFNKPTGYGSSSRRAPQTGIVDECCFRSCDLRRLEMYCAPLKPAKSA

SEQ ID NO: 343 - protein, 67 aa, synthetic construct
TLCGAELVDALQFPCGDRGFYFNKPTGYGSSSRRAPQTGIVDECCFRSCDLRRLEMYCAPLKPAKSA

SEQ ID NO: 344 - protein, 67 aa, synthetic construct
TLCGAELVDALQFQCGDRGFYFNKPTGYGSSSRRAPQTGIVDECCFRSCDLRRLEMYCAPLKPAKSA

SEQ ID NO: 345 - protein, 67 aa, synthetic construct
TLCGAELVDALQFRCGDRGFYFNKPTGYGSSSRRAPQTGIVDECCFRSCDLRRLEMYCAPLKPAKSA

SEQ ID NO: 346 - protein, 67 aa, synthetic construct
TLCGAELVDALQFSCGDRGFYFNKPTGYGSSSRRAPQTGIVDECCFRSCDLRRLEMYCAPLKPAKSA

SEQ ID NO: 347 - protein, 67 aa, synthetic construct
TLCGAELVDALQFTCGDRGFYFNKPTGYGSSSRRAPQTGIVDECCFRSCDLRRLEMYCAPLKPAKSA

SEQ ID NO: 348 - protein, 67 aa, synthetic construct
TLCGAELVDALQFWCGDRGFYFNKPTGYGSSSRRAPQTGIVDECCFRSCDLRRLEMYCAPLKPAKSA

SEQ ID NO: 349 - protein, 67 aa, synthetic construct
TLCGAELVDALQFYCGDRGFYFNKPTGYGSSSRRAPQTGIVDECCFRSCDLRRLEMYCAPLKPAKSA

SEQ ID NO: 350 - protein, 70 aa, synthetic construct
GPRTLCGAELVDALQFVCGDRAFYFNKPTGYGSSSRRAPQTGIVDECCFRSCDLRRLEMYCAPLKPAKSA

SEQ ID NO: 351 - protein, 70 aa, synthetic construct
GPRTLCGAELVDALQFVCGDRCFYFNKPTGYGSSSRRAPQTGIVDECCFRSCDLRRLEMYCAPLKPAKSA

SEQ ID NO: 352 - protein, 70 aa, synthetic construct
GPRTLCGAELVDALQFVCGDRDFYFNKPTGYGSSSRRAPQTGIVDECCFRSCDLRRLEMYCAPLKPAKSA

SEQ ID NO: 353 - protein, 70 aa, synthetic construct
GPRTLCGAELVDALQFVCGDREFYFNKPTGYGSSSRRAPQTGIVDECCFRSCDLRRLEMYCAPLKPAKSA

SEQ ID NO: 354 - protein, 70 aa, synthetic construct
GPRTLCGAELVDALQFVCGDRFFYFNKPTGYGSSSRRAPQTGIVDECCFRSCDLRRLEMYCAPLKPAKSA

SEQ ID NO: 355 - protein, 70 aa, synthetic construct
GPRTLCGAELVDALQFVCGDRHFYFNKPTGYGSSSRRAPQTGIVDECCFRSCDLRRLEMYCAPLKPAKSA

SEQ ID NO: 356 - protein, 70 aa, synthetic construct
GPRTLCGAELVDALQFVCGDRIFYFNKPTGYGSSSRRAPQTGIVDECCFRSCDLRRLEMYCAPLKPAKSA

SEQ ID NO: 357 - protein, 70 aa, synthetic construct
GPRTLCGAELVDALQFVCGDRKFYFNKPTGYGSSSRRAPQTGIVDECCFRSCDLRRLEMYCAPLKPAKSA

SEQ ID NO: 358 - protein, 70 aa, synthetic construct
GPRTLCGAELVDALQFVCGDRLFYFNKPTGYGSSSRRAPQTGIVDECCFRSCDLRRLEMYCAPLKPAKSA

SEQ ID NO: 359 - protein, 70 aa, synthetic construct
GPRTLCGAELVDALQFVCGDRMFYFNKPTGYGSSSRRAPQTGIVDECCFRSCDLRRLEMYCAPLKPAKSA

SEQ ID NO: 360 - protein, 70 aa, synthetic construct
GPRTLCGAELVDALQFVCGDRNFYFNKPTGYGSSSRRAPQTGIVDECCFRSCDLRRLEMYCAPLKPAKSA

SEQ ID NO: 361 - protein, 70 aa, synthetic construct
GPRTLCGAELVDALQFVCGDRPFYFNKPTGYGSSSRRAPQTGIVDECCFRSCDLRRLEMYCAPLKPAKSA

SEQ ID NO: 362 - protein, 70 aa, synthetic construct
GPRTLCGAELVDALQFVCGDRQFYFNKPTGYGSSSRRAPQTGIVDECCFRSCDLRRLEMYCAPLKPAKSA

SEQ ID NO: 363 - protein, 70 aa, synthetic construct
GPRTLCGAELVDALQFVCGDRRFYFNKPTGYGSSSRRAPQTGIVDECCFRSCDLRRLEMYCAPLKPAKSA

SEQ ID NO: 364 - protein, 70 aa, synthetic construct
GPRTLCGAELVDALQFVCGDRSFYFNKPTGYGSSSRRAPQTGIVDECCFRSCDLRRLEMYCAPLKPAKSA

SEQ ID NO: 365 - protein, 70 aa, synthetic construct
GPRTLCGAELVDALQFVCGDRTFYFNKPTGYGSSSRRAPQTGIVDECCFRSCDLRRLEMYCAPLKPAKSA

SEQ ID NO: 366 - protein, 70 aa, synthetic construct
GPRTLCGAELVDALQFVCGDRVFYFNKPTGYGSSSRRAPQTGIVDECCFRSCDLRRLEMYCAPLKPAKSA

SEQ ID NO: 367 - protein, 70 aa, synthetic construct
GPRTLCGAELVDALQFVCGDRWFYFNKPTGYGSSSRRAPQTGIVDECCFRSCDLRRLEMYCAPLKPAKSA

SEQ ID NO: 368 - protein, 70 aa, synthetic construct
GPRTLCGAELVDALQFVCGDRYFYFNKPTGYGSSSRRAPQTGIVDECCFRSCDLRRLEMYCAPLKPAKSA

SEQ ID NO: 369 - protein, 67 aa, synthetic construct
TLCGAELVDALQFVCGDRAFYFNKPTGYGSSSRRAPQTGIVDECCFRSCDLRRLEMYCAPLKPAKSA

SEQ ID NO: 370 - protein, 67 aa, synthetic construct
TLCGAELVDALQFVCGDRCFYFNKPTGYGSSSRRAPQTGIVDECCFRSCDLRRLEMYCAPLKPAKSA

SEQ ID NO: 371 - protein, 67 aa, synthetic construct
TLCGAELVDALQFVCGDRDFYFNKPTGYGSSSRRAPQTGIVDECCFRSCDLRRLEMYCAPLKPAKSA

SEQ ID NO: 372 - protein, 67 aa, synthetic construct
TLCGAELVDALQFVCGDREFYFNKPTGYGSSSRRAPQTGIVDECCFRSCDLRRLEMYCAPLKPAKSA

SEQ ID NO: 373 - protein, 67 aa, synthetic construct
TLCGAELVDALQFVCGDRFFYFNKPTGYGSSSRRAPQTGIVDECCFRSCDLRRLEMYCAPLKPAKSA

SEQ ID NO: 374 - protein, 67 aa, synthetic construct
TLCGAELVDALQFVCGDRHFYFNKPTGYGSSSRRAPQTGIVDECCFRSCDLRRLEMYCAPLKPAKSA

SEQ ID NO: 375 - protein, 67 aa, synthetic construct
TLCGAELVDALQFVCGDRIFYFNKPTGYGSSSRRAPQTGIVDECCFRSCDLRRLEMYCAPLKPAKSA

SEQ ID NO: 376 - protein, 67 aa, synthetic construct
TLCGAELVDALQFVCGDRKFYFNKPTGYGSSSRRAPQTGIVDECCFRSCDLRRLEMYCAPLKPAKSA

SEQ ID NO: 377 - protein, 67 aa, synthetic construct
TLCGAELVDALQFVCGDRLFYFNKPTGYGSSSRRAPQTGIVDECCFRSCDLRRLEMYCAPLKPAKSA

SEQ ID NO: 378 - protein, 67 aa, synthetic construct
TLCGAELVDALQFVCGDRMFYFNKPTGYGSSSRRAPQTGIVDECCFRSCDLRRLEMYCAPLKPAKSA

SEQ ID NO: 379 - protein, 67 aa, synthetic construct
TLCGAELVDALQFVCGDRNFYFNKPTGYGSSSRRAPQTGIVDECCFRSCDLRRLEMYCAPLKPAKSA

SEQ ID NO: 380 - protein, 67 aa, synthetic construct
TLCGAELVDALQFVCGDRPFYFNKPTGYGSSSRRAPQTGIVDECCFRSCDLRRLEMYCAPLKPAKSA

SEQ ID NO: 381 - protein, 67 aa, synthetic construct
TLCGAELVDALQFVCGDRQFYFNKPTGYGSSSRRAPQTGIVDECCFRSCDLRRLEMYCAPLKPAKSA

SEQ ID NO: 382 - protein, 67 aa, synthetic construct
TLCGAELVDALQFVCGDRRFYFNKPTGYGSSSRRAPQTGIVDECCFRSCDLRRLEMYCAPLKPAKSA

SEQ ID NO: 383 - protein, 67 aa, synthetic construct
TLCGAELVDALQFVCGDRSFYFNKPTGYGSSSRRAPQTGIVDECCFRSCDLRRLEMYCAPLKPAKSA

SEQ ID NO: 384 - protein, 67 aa, synthetic construct
TLCGAELVDALQFVCGDRTFYFNKPTGYGSSSRRAPQTGIVDECCFRSCDLRRLEMYCAPLKPAKSA

SEQ ID NO: 385 - protein, 67 aa, synthetic construct
TLCGAELVDALQFVCGDRVFYFNKPTGYGSSSRRAPQTGIVDECCFRSCDLRRLEMYCAPLKPAKSA

SEQ ID NO: 386 - protein, 67 aa, synthetic construct
TLCGAELVDALQFVCGDRWFYFNKPTGYGSSSRRAPQTGIVDECCFRSCDLRRLEMYCAPLKPAKSA

SEQ ID NO: 387 - protein, 67 aa, synthetic construct
TLCGAELVDALQFVCGDRYFYFNKPTGYGSSSRRAPQTGIVDECCFRSCDLRRLEMYCAPLKPAKSA

SEQ ID NO: 388 - protein, 70 aa, synthetic construct
GPRTLCGAELVDALQFVCGDRGAYFNKPTGYGSSSRRAPQTGIVDECCFRSCDLRRLEMYCAPLKPAKSA

SEQ ID NO: 389 - protein, 70 aa, synthetic construct
GPRTLCGAELVDALQFVCGDRGCYFNKPTGYGSSSRRAPQTGIVDECCFRSCDLRRLEMYCAPLKPAKSA

SEQ ID NO: 390 - protein, 70 aa, synthetic construct
GPRTLCGAELVDALQFVCGDRGDYFNKPTGYGSSSRRAPQTGIVDECCFRSCDLRRLEMYCAPLKPAKSA

SEQ ID NO: 391 - protein, 70 aa, synthetic construct
GPRTLCGAELVDALQFVCGDRGEYFNKPTGYGSSSRRAPQTGIVDECCFRSCDLRRLEMYCAPLKPAKSA

SEQ ID NO: 392 - protein, 70 aa, synthetic construct
GPRTLCGAELVDALQFVCGDRGGYFNKPTGYGSSSRRAPQTGIVDECCFRSCDLRRLEMYCAPLKPAKSA

SEQ ID NO: 393 - protein, 70 aa, synthetic construct
GPRTLCGAELVDALQFVCGDRGHYFNKPTGYGSSSRRAPQTGIVDECCFRSCDLRRLEMYCAPLKPAKSA

SEQ ID NO: 394 - protein, 70 aa, synthetic construct
GPRTLCGAELVDALQFVCGDRGIYFNKPTGYGSSSRRAPQTGIVDECCFRSCDLRRLEMYCAPLKPAKSA

SEQ ID NO: 395 - protein, 70 aa, synthetic construct
GPRTLCGAELVDALQFVCGDRGKYFNKPTGYGSSSRRAPQTGIVDECCFRSCDLRRLEMYCAPLKPAKSA

SEQ ID NO: 396 - protein, 70 aa, synthetic construct
GPRTLCGAELVDALQFVCGDRGLYFNKPTGYGSSSRRAPQTGIVDECCFRSCDLRRLEMYCAPLKPAKSA

SEQ ID NO: 397 - protein, 70 aa, synthetic construct
GPRTLCGAELVDALQFVCGDRGMYFNKPTGYGSSSRRAPQTGIVDECCFRSCDLRRLEMYCAPLKPAKSA

SEQ ID NO: 398 - protein, 70 aa, synthetic construct
GPRTLCGAELVDALQFVCGDRGNYFNKPTGYGSSSRRAPQTGIVDECCFRSCDLRRLEMYCAPLKPAKSA

SEQ ID NO: 399 - protein, 70 aa, synthetic construct
GPRTLCGAELVDALQFVCGDRGPYFNKPTGYGSSSRRAPQTGIVDECCFRSCDLRRLEMYCAPLKPAKSA

SEQ ID NO: 400 - protein, 70 aa, synthetic construct
GPRTLCGAELVDALQFVCGDRGQYFNKPTGYGSSSRRAPQTGIVDECCFRSCDLRRLEMYCAPLKPAKSA

SEQ ID NO: 401 - protein, 70 aa, synthetic construct
GPRTLCGAELVDALQFVCGDRGRYFNKPTGYGSSSRRAPQTGIVDECCFRSCDLRRLEMYCAPLKPAKSA

SEQ ID NO: 402 - protein, 70 aa, synthetic construct
GPRTLCGAELVDALQFVCGDRGSYFNKPTGYGSSSRRAPQTGIVDECCFRSCDLRRLEMYCAPLKPAKSA

SEQ ID NO: 403 - protein, 70 aa, synthetic construct
GPRTLCGAELVDALQFVCGDRGTYFNKPTGYGSSSRRAPQTGIVDECCFRSCDLRRLEMYCAPLKPAKSA

SEQ ID NO: 404 - protein, 70 aa, synthetic construct
GPRTLCGAELVDALQFVCGDRGVYFNKPTGYGSSSRRAPQTGIVDECCFRSCDLRRLEMYCAPLKPAKSA

SEQ ID NO: 405 - protein, 70 aa, synthetic construct
GPRTLCGAELVDALQFVCGDRGWYFNKPTGYGSSSRRAPQTGIVDECCFRSCDLRRLEMYCAPLKPAKSA

SEQ ID NO: 406 - protein, 70 aa, synthetic construct
GPRTLCGAELVDALQFVCGDRGYYFNKPTGYGSSSRRAPQTGIVDECCFRSCDLRRLEMYCAPLKPAKSA

SEQ ID NO: 407 - protein, 67 aa, synthetic construct
TLCGAELVDALQFVCGDRGAYFNKPTGYGSSSRRAPQTGIVDECCFRSCDLRRLEMYCAPLKPAKSA

SEQ ID NO: 408 - protein, 67 aa, synthetic construct
TLCGAELVDALQFVCGDRGCYFNKPTGYGSSSRRAPQTGIVDECCFRSCDLRRLEMYCAPLKPAKSA

SEQ ID NO: 409 - protein, 67 aa, synthetic construct
TLCGAELVDALQFVCGDRGDYFNKPTGYGSSSRRAPQTGIVDECCFRSCDLRRLEMYCAPLKPAKSA

SEQ ID NO: 410 - protein, 67 aa, synthetic construct
TLCGAELVDALQFVCGDRGEYFNKPTGYGSSSRRAPQTGIVDECCFRSCDLRRLEMYCAPLKPAKSA

SEQ ID NO: 411 - protein, 67 aa, synthetic construct
TLCGAELVDALQFVCGDRGGYFNKPTGYGSSSRRAPQTGIVDECCFRSCDLRRLEMYCAPLKPAKSA

SEQ ID NO: 412 - protein, 67 aa, synthetic construct
TLCGAELVDALQFVCGDRGHYFNKPTGYGSSSRRAPQTGIVDECCFRSCDLRRLEMYCAPLKPAKSA

SEQ ID NO: 413 - protein, 67 aa, synthetic construct
TLCGAELVDALQFVCGDRGIYFNKPTGYGSSSRRAPQTGIVDECCFRSCDLRRLEMYCAPLKPAKSA

SEQ ID NO: 414 - protein, 67 aa, synthetic construct
TLCGAELVDALQFVCGDRGKYFNKPTGYGSSSRRAPQTGIVDECCFRSCDLRRLEMYCAPLKPAKSA

SEQ ID NO: 415 - protein, 67 aa, synthetic construct
TLCGAELVDALQFVCGDRGLYFNKPTGYGSSSRRAPQTGIVDECCFRSCDLRRLEMYCAPLKPAKSA

SEQ ID NO: 416 - protein, 67 aa, synthetic construct
TLCGAELVDALQFVCGDRGMYFNKPTGYGSSSRRAPQTGIVDECCFRSCDLRRLEMYCAPLKPAKSA

SEQ ID NO: 417 - protein, 67 aa, synthetic construct
TLCGAELVDALQFVCGDRGNYFNKPTGYGSSSRRAPQTGIVDECCFRSCDLRRLEMYCAPLKPAKSA

SEQ ID NO: 418 - protein, 67 aa, synthetic construct
TLCGAELVDALQFVCGDRGPYFNKPTGYGSSSRRAPQTGIVDECCFRSCDLRRLEMYCAPLKPAKSA

SEQ ID NO: 419 - protein, 67 aa, synthetic construct
TLCGAELVDALQFVCGDRGQYFNKPTGYGSSSRRAPQTGIVDECCFRSCDLRRLEMYCAPLKPAKSA

SEQ ID NO: 420 - protein, 67 aa, synthetic construct
TLCGAELVDALQFVCGDRGRYFNKPTGYGSSSRRAPQTGIVDECCFRSCDLRRLEMYCAPLKPAKSA

SEQ ID NO: 421 - protein, 67 aa, synthetic construct
TLCGAELVDALQFVCGDRGSYFNKPTGYGSSSRRAPQTGIVDECCFRSCDLRRLEMYCAPLKPAKSA

SEQ ID NO: 422 - protein, 67 aa, synthetic construct
TLCGAELVDALQFVCGDRGTYFNKPTGYGSSSRRAPQTGIVDECCFRSCDLRRLEMYCAPLKPAKSA

SEQ ID NO: 423 - protein, 67 aa, synthetic construct
TLCGAELVDALQFVCGDRGVYFNKPTGYGSSSRRAPQTGIVDECCFRSCDLRRLEMYCAPLKPAKSA

SEQ ID NO: 424 - protein, 67 aa, synthetic construct
TLCGAELVDALQFVCGDRGWYFNKPTGYGSSSRRAPQTGIVDECCFRSCDLRRLEMYCAPLKPAKSA

SEQ ID NO: 425 - protein, 67 aa, synthetic construct
TLCGAELVDALQFVCGDRGYYFNKPTGYGSSSRRAPQTGIVDECCFRSCDLRRLEMYCAPLKPAKSA

SEQ ID NO: 426 - protein, 70 aa, synthetic construct
GPRTLCGAELVDALQFVCGDRGFAFNKPTGYGSSSRRAPQTGIVDECCFRSCDLRRLEMYCAPLKPAKSA

SEQ ID NO: 427 - protein, 70 aa, synthetic construct
GPRTLCGAELVDALQFVCGDRGFCFNKPTGYGSSSRRAPQTGIVDECCFRSCDLRRLEMYCAPLKPAKSA

SEQ ID NO: 428 - protein, 70 aa, synthetic construct
GPRTLCGAELVDALQFVCGDRGFDFNKPTGYGSSSRRAPQTGIVDECCFRSCDLRRLEMYCAPLKPAKSA

SEQ ID NO: 429 - protein, 70 aa, synthetic construct
GPRTLCGAELVDALQFVCGDRGFEFNKPTGYGSSSRRAPQTGIVDECCFRSCDLRRLEMYCAPLKPAKSA

SEQ ID NO: 430 - protein, 70 aa, synthetic construct
GPRTLCGAELVDALQFVCGDRGFFFNKPTGYGSSSRRAPQTGIVDECCFRSCDLRRLEMYCAPLKPAKSA

SEQ ID NO: 431 - protein, 70 aa, synthetic construct
GPRTLCGAELVDALQFVCGDRGFGFNKPTGYGSSSRRAPQTGIVDECCFRSCDLRRLEMYCAPLKPAKSA

SEQ ID NO: 432 - protein, 70 aa, synthetic construct
GPRTLCGAELVDALQFVCGDRGFHFNKPTGYGSSSRRAPQTGIVDECCFRSCDLRRLEMYCAPLKPAKSA

SEQ ID NO: 433 - protein, 70 aa, synthetic construct
GPRTLCGAELVDALQFVCGDRGFIFNKPTGYGSSSRRAPQTGIVDECCFRSCDLRRLEMYCAPLKPAKSA

SEQ ID NO: 434 - protein, 70 aa, synthetic construct
GPRTLCGAELVDALQFVCGDRGFKFNKPTGYGSSSRRAPQTGIVDECCFRSCDLRRLEMYCAPLKPAKSA

SEQ ID NO: 435 - protein, 70 aa, synthetic construct
GPRTLCGAELVDALQFVCGDRGFLFNKPTGYGSSSRRAPQTGIVDECCFRSCDLRRLEMYCAPLKPAKSA

SEQ ID NO: 436 - protein, 70 aa, synthetic construct
GPRTLCGAELVDALQFVCGDRGFMFNKPTGYGSSSRRAPQTGIVDECCFRSCDLRRLEMYCAPLKPAKSA

SEQ ID NO: 437 - protein, 70 aa, synthetic construct
GPRTLCGAELVDALQFVCGDRGFNFNKPTGYGSSSRRAPQTGIVDECCFRSCDLRRLEMYCAPLKPAKSA

SEQ ID NO: 438 - protein, 70 aa, synthetic construct
GPRTLCGAELVDALQFVCGDRGFPFNKPTGYGSSSRRAPQTGIVDECCFRSCDLRRLEMYCAPLKPAKSA

SEQ ID NO: 439 - protein, 70 aa, synthetic construct
GPRTLCGAELVDALQFVCGDRGFQFNKPTGYGSSSRRAPQTGIVDECCFRSCDLRRLEMYCAPLKPAKSA

SEQ ID NO: 440 - protein, 70 aa, synthetic construct
GPRTLCGAELVDALQFVCGDRGFRFNKPTGYGSSSRRAPQTGIVDECCFRSCDLRRLEMYCAPLKPAKSA

SEQ ID NO: 441 - protein, 70 aa, synthetic construct
GPRTLCGAELVDALQFVCGDRGFSFNKPTGYGSSSRRAPQTGIVDECCFRSCDLRRLEMYCAPLKPAKSA

SEQ ID NO: 442 - protein, 70 aa, synthetic construct
GPRTLCGAELVDALQFVCGDRGFTFNKPTGYGSSSRRAPQTGIVDECCFRSCDLRRLEMYCAPLKPAKSA

SEQ ID NO: 443 - protein, 70 aa, synthetic construct
GPRTLCGAELVDALQFVCGDRGFVFNKPTGYGSSSRRAPQTGIVDECCFRSCDLRRLEMYCAPLKPAKSA

SEQ ID NO: 444 - protein, 70 aa, synthetic construct
GPRTLCGAELVDALQFVCGDRGFWFNKPTGYGSSSRRAPQTGIVDECCFRSCDLRRLEMYCAPLKPAKSA

SEQ ID NO: 445 - protein, 67 aa, synthetic construct
TLCGAELVDALQFVCGDRGFAFNKPTGYGSSSRRAPQTGIVDECCFRSCDLRRLEMYCAPLKPAKSA

SEQ ID NO: 446 - protein, 67 aa, synthetic construct
TLCGAELVDALQFVCGDRGFCFNKPTGYGSSSRRAPQTGIVDECCFRSCDLRRLEMYCAPLKPAKSA

SEQ ID NO: 447 - protein, 67 aa, synthetic construct
TLCGAELVDALQFVCGDRGFDFNKPTGYGSSSRRAPQTGIVDECCFRSCDLRRLEMYCAPLKPAKSA

SEQ ID NO: 448 - protein, 67 aa, synthetic construct
TLCGAELVDALQFVCGDRGFEFNKPTGYGSSSRRAPQTGIVDECCFRSCDLRRLEMYCAPLKPAKSA

SEQ ID NO: 449 - protein, 67 aa, synthetic construct
TLCGAELVDALQFVCGDRGFFFNKPTGYGSSSRRAPQTGIVDECCFRSCDLRRLEMYCAPLKPAKSA

SEQ ID NO: 450 - protein, 67 aa, synthetic construct
TLCGAELVDALQFVCGDRGFGFNKPTGYGSSSRRAPQTGIVDECCFRSCDLRRLEMYCAPLKPAKSA

SEQ ID NO: 451 - protein, 67 aa, synthetic construct
TLCGAELVDALQFVCGDRGFHFNKPTGYGSSSRRAPQTGIVDECCFRSCDLRRLEMYCAPLKPAKSA

SEQ ID NO: 452 - protein, 67 aa, synthetic construct
TLCGAELVDALQFVCGDRGFIFNKPTGYGSSSRRAPQTGIVDECCFRSCDLRRLEMYCAPLKPAKSA

SEQ ID NO: 453 - protein, 67 aa, synthetic construct
TLCGAELVDALQFVCGDRGFKFNKPTGYGSSSRRAPQTGIVDECCFRSCDLRRLEMYCAPLKPAKSA

SEQ ID NO: 454 - protein, 67 aa, synthetic construct
TLCGAELVDALQFVCGDRGFLFNKPTGYGSSSRRAPQTGIVDECCFRSCDLRRLEMYCAPLKPAKSA

SEQ ID NO: 455 - protein, 67 aa, synthetic construct
TLCGAELVDALQFVCGDRGFMFNKPTGYGSSSRRAPQTGIVDECCFRSCDLRRLEMYCAPLKPAKSA

SEQ ID NO: 456 - protein, 67 aa, synthetic construct
TLCGAELVDALQFVCGDRGFNFNKPTGYGSSSRRAPQTGIVDECCFRSCDLRRLEMYCAPLKPAKSA

SEQ ID NO: 457 - protein, 67 aa, synthetic construct
TLCGAELVDALQFVCGDRGFPFNKPTGYGSSSRRAPQTGIVDECCFRSCDLRRLEMYCAPLKPAKSA

SEQ ID NO: 458 - protein, 67 aa, synthetic construct
TLCGAELVDALQFVCGDRGFQFNKPTGYGSSSRRAPQTGIVDECCFRSCDLRRLEMYCAPLKPAKSA

SEQ ID NO: 459 - protein, 67 aa, synthetic construct
TLCGAELVDALQFVCGDRGFRFNKPTGYGSSSRRAPQTGIVDECCFRSCDLRRLEMYCAPLKPAKSA

SEQ ID NO: 460 - protein, 67 aa, synthetic construct
TLCGAELVDALQFVCGDRGFSFNKPTGYGSSSRRAPQTGIVDECCFRSCDLRRLEMYCAPLKPAKSA

SEQ ID NO: 461 - protein, 67 aa, synthetic construct
TLCGAELVDALQFVCGDRGFTFNKPTGYGSSSRRAPQTGIVDECCFRSCDLRRLEMYCAPLKPAKSA

SEQ ID NO: 462 - protein, 67 aa, synthetic construct
TLCGAELVDALQFVCGDRGFVFNKPTGYGSSSRRAPQTGIVDECCFRSCDLRRLEMYCAPLKPAKSA

SEQ ID NO: 463 - protein, 67 aa, synthetic construct
TLCGAELVDALQFVCGDRGFWFNKPTGYGSSSRRAPQTGIVDECCFRSCDLRRLEMYCAPLKPAKSA

SEQ ID NO: 464 - protein, 70 aa, synthetic construct
GPRTLCGAELVDALQFVCGDRGFYANKPTGYGSSSRRAPQTGIVDECCFRSCDLRRLEMYCAPLKPAKSA

SEQ ID NO: 465 - protein, 70 aa, synthetic construct
GPRTLCGAELVDALQFVCGDRGFYCNKPTGYGSSSRRAPQTGIVDECCFRSCDLRRLEMYCAPLKPAKSA

SEQ ID NO: 466 - protein, 70 aa, synthetic construct
GPRTLCGAELVDALQFVCGDRGFYDNKPTGYGSSSRRAPQTGIVDECCFRSCDLRRLEMYCAPLKPAKSA

SEQ ID NO: 467 - protein, 70 aa, synthetic construct
GPRTLCGAELVDALQFVCGDRGFYENKPTGYGSSSRRAPQTGIVDECCFRSCDLRRLEMYCAPLKPAKSA

SEQ ID NO: 468 - protein, 70 aa, synthetic construct
GPRTLCGAELVDALQFVCGDRGFYGNKPTGYGSSSRRAPQTGIVDECCFRSCDLRRLEMYCAPLKPAKSA

SEQ ID NO: 469 - protein, 70 aa, synthetic construct
GPRTLCGAELVDALQFVCGDRGFYHNKPTGYGSSSRRAPQTGIVDECCFRSCDLRRLEMYCAPLKPAKSA

SEQ ID NO: 470 - protein, 70 aa, synthetic construct
GPRTLCGAELVDALQFVCGDRGFYINKPTGYGSSSRRAPQTGIVDECCFRSCDLRRLEMYCAPLKPAKSA

SEQ ID NO: 471 - protein, 70 aa, synthetic construct
GPRTLCGAELVDALQFVCGDRGFYKNKPTGYGSSSRRAPQTGIVDECCFRSCDLRRLEMYCAPLKPAKSA

SEQ ID NO: 472 - protein, 70 aa, synthetic construct
GPRTLCGAELVDALQFVCGDRGFYLNKPTGYGSSSRRAPQTGIVDECCFRSCDLRRLEMYCAPLKPAKSA

SEQ ID NO: 473 - protein, 70 aa, synthetic construct
GPRTLCGAELVDALQFVCGDRGFYMNKPTGYGSSSRRAPQTGIVDECCFRSCDLRRLEMYCAPLKPAKSA

SEQ ID NO: 474 - protein, 70 aa, synthetic construct
GPRTLCGAELVDALQFVCGDRGFYNNKPTGYGSSSRRAPQTGIVDECCFRSCDLRRLEMYCAPLKPAKSA

SEQ ID NO: 475 - protein, 70 aa, synthetic construct
GPRTLCGAELVDALQFVCGDRGFYPNKPTGYGSSSRRAPQTGIVDECCFRSCDLRRLEMYCAPLKPAKSA

SEQ ID NO: 476 - protein, 70 aa, synthetic construct
GPRTLCGAELVDALQFVCGDRGFYQNKPTGYGSSSRRAPQTGIVDECCFRSCDLRRLEMYCAPLKPAKSA

SEQ ID NO: 477 - protein, 70 aa, synthetic construct
GPRTLCGAELVDALQFVCGDRGFYRNKPTGYGSSSRRAPQTGIVDECCFRSCDLRRLEMYCAPLKPAKSA

SEQ ID NO: 478 - protein, 70 aa, synthetic construct
GPRTLCGAELVDALQFVCGDRGFYSNKPTGYGSSSRRAPQTGIVDECCFRSCDLRRLEMYCAPLKPAKSA

SEQ ID NO: 479 - protein, 70 aa, synthetic construct
GPRTLCGAELVDALQFVCGDRGFYTNKPTGYGSSSRRAPQTGIVDECCFRSCDLRRLEMYCAPLKPAKSA

SEQ ID NO: 480 - protein, 70 aa, synthetic construct
GPRTLCGAELVDALQFVCGDRGFYVNKPTGYGSSSRRAPQTGIVDECCFRSCDLRRLEMYCAPLKPAKSA

SEQ ID NO: 481 - protein, 70 aa, synthetic construct
GPRTLCGAELVDALQFVCGDRGFYWNKPTGYGSSSRRAPQTGIVDECCFRSCDLRRLEMYCAPLKPAKSA

SEQ ID NO: 482 - protein, 70 aa, synthetic construct
GPRTLCGAELVDALQFVCGDRGFYYNKPTGYGSSSRRAPQTGIVDECCFRSCDLRRLEMYCAPLKPAKSA

SEQ ID NO: 483 - protein, 67 aa, synthetic construct
TLCGAELVDALQFVCGDRGFYANKPTGYGSSSRRAPQTGIVDECCFRSCDLRRLEMYCAPLKPAKSA

SEQ ID NO: 484 - protein, 67 aa, synthetic construct
TLCGAELVDALQFVCGDRGFYCNKPTGYGSSSRRAPQTGIVDECCFRSCDLRRLEMYCAPLKPAKSA

SEQ ID NO: 485 - protein, 67 aa, synthetic construct
TLCGAELVDALQFVCGDRGFYDNKPTGYGSSSRRAPQTGIVDECCFRSCDLRRLEMYCAPLKPAKSA

SEQ ID NO: 486 - protein, 67 aa, synthetic construct
TLCGAELVDALQFVCGDRGFYENKPTGYGSSSRRAPQTGIVDECCFRSCDLRRLEMYCAPLKPAKSA

SEQ ID NO: 487 - protein, 67 aa, synthetic construct
TLCGAELVDALQFVCGDRGFYGNKPTGYGSSSRRAPQTGIVDECCFRSCDLRRLEMYCAPLKPAKSA

SEQ ID NO: 488 - protein, 67 aa, synthetic construct
TLCGAELVDALQFVCGDRGFYHNKPTGYGSSSRRAPQTGIVDECCFRSCDLRRLEMYCAPLKPAKSA

SEQ ID NO: 489 - protein, 67 aa, synthetic construct
TLCGAELVDALQFVCGDRGFYINKPTGYGSSSRRAPQTGIVDECCFRSCDLRRLEMYCAPLKPAKSA

SEQ ID NO: 490 - protein, 67 aa, synthetic construct
TLCGAELVDALQFVCGDRGFYKNKPTGYGSSSRRAPQTGIVDECCFRSCDLRRLEMYCAPLKPAKSA

SEQ ID NO: 491 - protein, 67 aa, synthetic construct
TLCGAELVDALQFVCGDRGFYLNKPTGYGSSSRRAPQTGIVDECCFRSCDLRRLEMYCAPLKPAKSA

SEQ ID NO: 492 - protein, 67 aa, synthetic construct
TLCGAELVDALQFVCGDRGFYMNKPTGYGSSSRRAPQTGIVDECCFRSCDLRRLEMYCAPLKPAKSA

SEQ ID NO: 493 - protein, 67 aa, synthetic construct
TLCGAELVDALQFVCGDRGFYNNKPTGYGSSSRRAPQTGIVDECCFRSCDLRRLEMYCAPLKPAKSA

SEQ ID NO: 494 - protein, 67 aa, synthetic construct
TLCGAELVDALQFVCGDRGFYPNKPTGYGSSSRRAPQTGIVDECCFRSCDLRRLEMYCAPLKPAKSA

SEQ ID NO: 495 - protein, 67 aa, synthetic construct
TLCGAELVDALQFVCGDRGFYQNKPTGYGSSSRRAPQTGIVDECCFRSCDLRRLEMYCAPLKPAKSA

SEQ ID NO: 496 - protein, 67 aa, synthetic construct
TLCGAELVDALQFVCGDRGFYRNKPTGYGSSSRRAPQTGIVDECCFRSCDLRRLEMYCAPLKPAKSA

SEQ ID NO: 497 - protein, 67 aa, synthetic construct
TLCGAELVDALQFVCGDRGFYSNKPTGYGSSSRRAPQTGIVDECCFRSCDLRRLEMYCAPLKPAKSA

SEQ ID NO: 498 - protein, 67 aa, synthetic construct
TLCGAELVDALQFVCGDRGFYTNKPTGYGSSSRRAPQTGIVDECCFRSCDLRRLEMYCAPLKPAKSA

SEQ ID NO: 499 - protein, 67 aa, synthetic construct
TLCGAELVDALQFVCGDRGFYVNKPTGYGSSSRRAPQTGIVDECCFRSCDLRRLEMYCAPLKPAKSA

SEQ ID NO: 500 - protein, 67 aa, synthetic construct
TLCGAELVDALQFVCGDRGFYWNKPTGYGSSSRRAPQTGIVDECCFRSCDLRRLEMYCAPLKPAKSA

SEQ ID NO: 501 - protein, 67 aa, synthetic construct
TLCGAELVDALQFVCGDRGFYYNKPTGYGSSSRRAPQTGIVDECCFRSCDLRRLEMYCAPLKPAKSA

SEQ ID NO: 502 - protein, 70 aa, synthetic construct
GPRTLCGAELVDALQFVCGDRGFYFNKPTGYGSASRRAPQTGIVDECCFRSCDLRRLEMYCAPLKPAKSA

SEQ ID NO: 503 - protein, 70 aa, synthetic construct
GPRTLCGAELVDALQFVCGDRGFYFNKPTGYGSCSRRAPQTGIVDECCFRSCDLRRLEMYCAPLKPAKSA

SEQ ID NO: 504 - protein, 70 aa, synthetic construct
GPRTLCGAELVDALQFVCGDRGFYFNKPTGYGSDSRRAPQTGIVDECCFRSCDLRRLEMYCAPLKPAKSA

SEQ ID NO: 505 - protein, 70 aa, synthetic construct
GPRTLCGAELVDALQFVCGDRGFYFNKPTGYGSESRRAPQTGIVDECCFRSCDLRRLEMYCAPLKPAKSA

SEQ ID NO: 506 - protein, 70 aa, synthetic construct
GPRTLCGAELVDALQFVCGDRGFYFNKPTGYGSFSRRAPQTGIVDECCFRSCDLRRLEMYCAPLKPAKSA

SEQ ID NO: 507 - protein, 70 aa, synthetic construct
GPRTLCGAELVDALQFVCGDRGFYFNKPTGYGSGSRRAPQTGIVDECCFRSCDLRRLEMYCAPLKPAKSA

SEQ ID NO: 508 - protein, 70 aa, synthetic construct
GPRTLCGAELVDALQFVCGDRGFYFNKPTGYGSHSRRAPQTGIVDECCFRSCDLRRLEMYCAPLKPAKSA

SEQ ID NO: 509 - protein, 70 aa, synthetic construct
GPRTLCGAELVDALQFVCGDRGFYFNKPTGYGSISRRAPQTGIVDECCFRSCDLRRLEMYCAPLKPAKSA

SEQ ID NO: 510 - protein, 70 aa, synthetic construct
GPRTLCGAELVDALQFVCGDRGFYFNKPTGYGSKSRRAPQTGIVDECCFRSCDLRRLEMYCAPLKPAKSA

SEQ ID NO: 511 - protein, 70 aa, synthetic construct
GPRTLCGAELVDALQFVCGDRGFYFNKPTGYGSLSRRAPQTGIVDECCFRSCDLRRLEMYCAPLKPAKSA

SEQ ID NO: 512 - protein, 70 aa, synthetic construct
GPRTLCGAELVDALQFVCGDRGFYFNKPTGYGSMSRRAPQTGIVDECCFRSCDLRRLEMYCAPLKPAKSA

SEQ ID NO: 513 - protein, 70 aa, synthetic construct
GPRTLCGAELVDALQFVCGDRGFYFNKPTGYGSNSRRAPQTGIVDECCFRSCDLRRLEMYCAPLKPAKSA

SEQ ID NO: 514 - protein, 70 aa, synthetic construct
GPRTLCGAELVDALQFVCGDRGFYFNKPTGYGSPSRRAPQTGIVDECCFRSCDLRRLEMYCAPLKPAKSA

SEQ ID NO: 515 - protein, 70 aa, synthetic construct
GPRTLCGAELVDALQFVCGDRGFYFNKPTGYGSQSRRAPQTGIVDECCFRSCDLRRLEMYCAPLKPAKSA

SEQ ID NO: 516 - protein, 70 aa, synthetic construct; identical to SEQ ID NO: 4

SEQ ID NO: 517 - protein, 70 aa, synthetic construct
GPRTLCGAELVDALQFVCGDRGFYFNKPTGYGSTSRRAPQTGIVDECCFRSCDLRRLEMYCAPLKPAKSA

SEQ ID NO: 518 - protein, 70 aa, synthetic construct
GPRTLCGAELVDALQFVCGDRGFYFNKPTGYGSVSRRAPQTGIVDECCFRSCDLRRLEMYCAPLKPAKSA

SEQ ID NO: 519 - protein, 70 aa, synthetic construct
GPRTLCGAELVDALQFVCGDRGFYFNKPTGYGSWSRRAPQTGIVDECCFRSCDLRRLEMYCAPLKPAKSA

SEQ ID NO: 520 - protein, 70 aa, synthetic construct
GPRTLCGAELVDALQFVCGDRGFYFNKPTGYGSYSRRAPQTGIVDECCFRSCDLRRLEMYCAPLKPAKSA

SEQ ID NO: 521 - protein, 67 aa, synthetic construct
TLCGAELVDALQFVCGDRGFYFNKPTGYGSASRRAPQTGIVDECCFRSCDLRRLEMYCAPLKPAKSA

SEQ ID NO: 522 - protein, 67 aa, synthetic construct
TLCGAELVDALQFVCGDRGFYFNKPTGYGSCSRRAPQTGIVDECCFRSCDLRRLEMYCAPLKPAKSA

SEQ ID NO: 523 - protein, 67 aa, synthetic construct
TLCGAELVDALQFVCGDRGFYFNKPTGYGSDSRRAPQTGIVDECCFRSCDLRRLEMYCAPLKPAKSA

SEQ ID NO: 524 - protein, 67 aa, synthetic construct
TLCGAELVDALQFVCGDRGFYFNKPTGYGSESRRAPQTGIVDECCFRSCDLRRLEMYCAPLKPAKSA

SEQ ID NO: 525 - protein, 67 aa, synthetic construct
TLCGAELVDALQFVCGDRGFYFNKPTGYGSFSRRAPQTGIVDECCFRSCDLRRLEMYCAPLKPAKSA

SEQ ID NO: 526 - protein, 67 aa, synthetic construct
TLCGAELVDALQFVCGDRGFYFNKPTGYGSGSRRAPQTGIVDECCFRSCDLRRLEMYCAPLKPAKSA

SEQ ID NO: 527 - protein, 67 aa, synthetic construct
TLCGAELVDALQFVCGDRGFYFNKPTGYGSHSRRAPQTGIVDECCFRSCDLRRLEMYCAPLKPAKSA

SEQ ID NO: 528 - protein, 67 aa, synthetic construct
TLCGAELVDALQFVCGDRGFYFNKPTGYGSISRRAPQTGIVDECCFRSCDLRRLEMYCAPLKPAKSA

SEQ ID NO: 529 - protein, 67 aa, synthetic construct
TLCGAELVDALQFVCGDRGFYFNKPTGYGSKSRRAPQTGIVDECCFRSCDLRRLEMYCAPLKPAKSA

SEQ ID NO: 530 - protein, 67 aa, synthetic construct
TLCGAELVDALQFVCGDRGFYFNKPTGYGSLSRRAPQTGIVDECCFRSCDLRRLEMYCAPLKPAKSA

SEQ ID NO: 531 - protein, 67 aa, synthetic construct
TLCGAELVDALQFVCGDRGFYFNKPTGYGSMSRRAPQTGIVDECCFRSCDLRRLEMYCAPLKPAKSA

SEQ ID NO: 532 - protein, 67 aa, synthetic construct
TLCGAELVDALQFVCGDRGFYFNKPTGYGSNSRRAPQTGIVDECCFRSCDLRRLEMYCAPLKPAKSA

SEQ ID NO: 533 - protein, 67 aa, synthetic construct
TLCGAELVDALQFVCGDRGFYFNKPTGYGSPSRRAPQTGIVDECCFRSCDLRRLEMYCAPLKPAKSA

SEQ ID NO: 534 - protein, 67 aa, synthetic construct
TLCGAELVDALQFVCGDRGFYFNKPTGYGSQSRRAPQTGIVDECCFRSCDLRRLEMYCAPLKPAKSA

SEQ ID NO: 535 - protein, 67 aa, synthetic construct; identical to SEQ ID NO: 3

SEQ ID NO: 536 - protein, 67 aa, synthetic construct
TLCGAELVDALQFVCGDRGFYFNKPTGYGSTSRRAPQTGIVDECCFRSCDLRRLEMYCAPLKPAKSA

SEQ ID NO: 537 - protein, 67 aa, synthetic construct
TLCGAELVDALQFVCGDRGFYFNKPTGYGSVSRRAPQTGIVDECCFRSCDLRRLEMYCAPLKPAKSA

SEQ ID NO: 538 - protein, 67 aa, synthetic construct
TLCGAELVDALQFVCGDRGFYFNKPTGYGSWSRRAPQTGIVDECCFRSCDLRRLEMYCAPLKPAKSA

SEQ ID NO: 539 - protein, 67 aa, synthetic construct
TLCGAELVDALQFVCGDRGFYFNKPTGYGSYSRRAPQTGIVDECCFRSCDLRRLEMYCAPLKPAKSA

SEQ ID NO: 540 - protein, 70 aa, synthetic construct
GPRTLCGAELVDALQFVCGDRGFYFNKPTGYGSSSRRAPQTGIVDECCFRSCDLRRAEMYCAPLKPAKSA

SEQ ID NO: 541 - protein, 70 aa, synthetic construct
GPRTLCGAELVDALQFVCGDRGFYFNKPTGYGSSSRRAPQTGIVDECCFRSCDLRRCEMYCAPLKPAKSA

SEQ ID NO: 542 - protein, 70 aa, synthetic construct
GPRTLCGAELVDALQFVCGDRGFYFNKPTGYGSSSRRAPQTGIVDECCFRSCDLRRDEMYCAPLKPAKSA

SEQ ID NO: 543 - protein, 70 aa, synthetic construct
GPRTLCGAELVDALQFVCGDRGFYFNKPTGYGSSSRRAPQTGIVDECCFRSCDLRREEMYCAPLKPAKSA

SEQ ID NO: 544 - protein, 70 aa, synthetic construct
GPRTLCGAELVDALQFVCGDRGFYFNKPTGYGSSSRRAPQTGIVDECCFRSCDLRRFEMYCAPLKPAKSA

SEQ ID NO: 545 - protein, 70 aa, synthetic construct
GPRTLCGAELVDALQFVCGDRGFYFNKPTGYGSSSRRAPQTGIVDECCFRSCDLRRGEMYCAPLKPAKSA

SEQ ID NO: 546 - protein, 70 aa, synthetic construct
GPRTLCGAELVDALQFVCGDRGFYFNKPTGYGSSSRRAPQTGIVDECCFRSCDLRRHEMYCAPLKPAKSA

SEQ ID NO: 547 - protein, 70 aa, synthetic construct
GPRTLCGAELVDALQFVCGDRGFYFNKPTGYGSSSRRAPQTGIVDECCFRSCDLRRIEMYCAPLKPAKSA

SEQ ID NO: 548 - protein, 70 aa, synthetic construct
GPRTLCGAELVDALQFVCGDRGFYFNKPTGYGSSSRRAPQTGIVDECCFRSCDLRRKEMYCAPLKPAKSA

SEQ ID NO: 549 - protein, 70 aa, synthetic construct
GPRTLCGAELVDALQFVCGDRGFYFNKPTGYGSSSRRAPQTGIVDECCFRSCDLRRMEMYCAPLKPAKSA

SEQ ID NO: 550 - protein, 70 aa, synthetic construct
GPRTLCGAELVDALQFVCGDRGFYFNKPTGYGSSSRRAPQTGIVDECCFRSCDLRRNEMYCAPLKPAKSA

SEQ ID NO: 551 - protein, 70 aa, synthetic construct
GPRTLCGAELVDALQFVCGDRGFYFNKPTGYGSSSRRAPQTGIVDECCFRSCDLRRPEMYCAPLKPAKSA

SEQ ID NO: 552 - protein, 70 aa, synthetic construct
GPRTLCGAELVDALQFVCGDRGFYFNKPTGYGSSSRRAPQTGIVDECCFRSCDLRRQEMYCAPLKPAKSA

SEQ ID NO: 553 - protein, 70 aa, synthetic construct
GPRTLCGAELVDALQFVCGDRGFYFNKPTGYGSSSRRAPQTGIVDECCFRSCDLRRREMYCAPLKPAKSA

SEQ ID NO: 554 - protein, 70 aa, synthetic construct
GPRTLCGAELVDALQFVCGDRGFYFNKPTGYGSSSRRAPQTGIVDECCFRSCDLRRSEMYCAPLKPAKSA

SEQ ID NO: 555 - protein, 70 aa, synthetic construct
GPRTLCGAELVDALQFVCGDRGFYFNKPTGYGSSSRRAPQTGIVDECCFRSCDLRRTEMYCAPLKPAKSA

SEQ ID NO: 556 - protein, 70 aa, synthetic construct
GPRTLCGAELVDALQFVCGDRGFYFNKPTGYGSSSRRAPQTGIVDECCFRSCDLRRVEMYCAPLKPAKSA

SEQ ID NO: 557 - protein, 70 aa, synthetic construct
GPRTLCGAELVDALQFVCGDRGFYFNKPTGYGSSSRRAPQTGIVDECCFRSCDLRRWEMYCAPLKPAKSA

SEQ ID NO: 558 - protein, 70 aa, synthetic construct
GPRTLCGAELVDALQFVCGDRGFYFNKPTGYGSSSRRAPQTGIVDECCFRSCDLRRYEMYCAPLKPAKSA

SEQ ID NO: 559 - protein, 67 aa, synthetic construct
TLCGAELVDALQFVCGDRGFYFNKPTGYGSSSRRAPQTGIVDECCFRSCDLRRAEMYCAPLKPAKSA

SEQ ID NO: 560 - protein, 67 aa, synthetic construct
TLCGAELVDALQFVCGDRGFYFNKPTGYGSSSRRAPQTGIVDECCFRSCDLRRCEMYCAPLKPAKSA

SEQ ID NO: 561 - protein, 67 aa, synthetic construct
TLCGAELVDALQFVCGDRGFYFNKPTGYGSSSRRAPQTGIVDECCFRSCDLRRDEMYCAPLKPAKSA

SEQ ID NO: 562 - protein, 67 aa, synthetic construct
TLCGAELVDALQFVCGDRGFYFNKPTGYGSSSRRAPQTGIVDECCFRSCDLRREEMYCAPLKPAKSA

SEQ ID NO: 563 - protein, 67 aa, synthetic construct
TLCGAELVDALQFVCGDRGFYFNKPTGYGSSSRRAPQTGIVDECCFRSCDLRRFEMYCAPLKPAKSA

SEQ ID NO: 564 - protein, 67 aa, synthetic construct
TLCGAELVDALQFVCGDRGFYFNKPTGYGSSSRRAPQTGIVDECCFRSCDLRRGEMYCAPLKPAKSA

SEQ ID NO: 565 - protein, 67 aa, synthetic construct
TLCGAELVDALQFVCGDRGFYFNKPTGYGSSSRRAPQTGIVDECCFRSCDLRRHEMYCAPLKPAKSA

SEQ ID NO: 566 - protein, 67 aa, synthetic construct
TLCGAELVDALQFVCGDRGFYFNKPTGYGSSSRRAPQTGIVDECCFRSCDLRRIEMYCAPLKPAKSA

SEQ ID NO: 567 - protein, 67 aa, synthetic construct
TLCGAELVDALQFVCGDRGFYFNKPTGYGSSSRRAPQTGIVDECCFRSCDLRRKEMYCAPLKPAKSA

SEQ ID NO: 568 - protein, 67 aa, synthetic construct
TLCGAELVDALQFVCGDRGFYFNKPTGYGSSSRRAPQTGIVDECCFRSCDLRRMEMYCAPLKPAKSA

SEQ ID NO: 569 - protein, 67 aa, synthetic construct
TLCGAELVDALQFVCGDRGFYFNKPTGYGSSSRRAPQTGIVDECCFRSCDLRRNEMYCAPLKPAKSA

SEQ ID NO: 570 - protein, 67 aa, synthetic construct
TLCGAELVDALQFVCGDRGFYFNKPTGYGSSSRRAPQTGIVDECCFRSCDLRRPEMYCAPLKPAKSA

SEQ ID NO: 571 - protein, 67 aa, synthetic construct
TLCGAELVDALQFVCGDRGFYFNKPTGYGSSSRRAPQTGIVDECCFRSCDLRRQEMYCAPLKPAKSA

SEQ ID NO: 572 - protein, 67 aa, synthetic construct
TLCGAELVDALQFVCGDRGFYFNKPTGYGSSSRRAPQTGIVDECCFRSCDLRRREMYCAPLKPAKSA

SEQ ID NO: 573 - protein, 67 aa, synthetic construct
TLCGAELVDALQFVCGDRGFYFNKPTGYGSSSRRAPQTGIVDECCFRSCDLRRSEMYCAPLKPAKSA

SEQ ID NO: 574 - protein, 67 aa, synthetic construct
TLCGAELVDALQFVCGDRGFYFNKPTGYGSSSRRAPQTGIVDECCFRSCDLRRTEMYCAPLKPAKSA

SEQ ID NO: 575 - protein, 67 aa, synthetic construct
TLCGAELVDALQFVCGDRGFYFNKPTGYGSSSRRAPQTGIVDECCFRSCDLRRVEMYCAPLKPAKSA

SEQ ID NO: 576 - protein, 67 aa, synthetic construct
TLCGAELVDALQFVCGDRGFYFNKPTGYGSSSRRAPQTGIVDECCFRSCDLRRWEMYCAPLKPAKSA

SEQ ID NO: 577 - protein, 67 aa, synthetic construct
TLCGAELVDALQFVCGDRGFYFNKPTGYGSSSRRAPQTGIVDECCFRSCDLRRYEMYCAPLKPAKSA

SEQ ID NO: 578 - protein, 70 aa, synthetic construct
GPETACGAELVDALQFVCGDRGFYFNKPTGYGSSSRRAPQTGIVDECCFRSCDLRRLEMYCAPLKPAKSA

SEQ ID NO: 579 - protein, 70 aa, synthetic construct
GPETCCGAELVDALQFVCGDRGFYFNKPTGYGSSSRRAPQTGIVDECCFRSCDLRRLEMYCAPLKPAKSA

SEQ ID NO: 580 - protein, 70 aa, synthetic construct
GPETDCGAELVDALQFVCGDRGFYFNKPTGYGSSSRRAPQTGIVDECCFRSCDLRRLEMYCAPLKPAKSA

SEQ ID NO: 581 - protein, 70 aa, synthetic construct
GPETECGAELVDALQFVCGDRGFYFNKPTGYGSSSRRAPQTGIVDECCFRSCDLRRLEMYCAPLKPAKSA

SEQ ID NO: 582 - protein, 70 aa, synthetic construct
GPETFCGAELVDALQFVCGDRGFYFNKPTGYGSSSRRAPQTGIVDECCFRSCDLRRLEMYCAPLKPAKSA

SEQ ID NO: 583 - protein, 70 aa, synthetic construct
GPETGCGAELVDALQFVCGDRGFYFNKPTGYGSSSRRAPQTGIVDECCFRSCDLRRLEMYCAPLKPAKSA

SEQ ID NO: 584 - protein, 70 aa, synthetic construct
GPETHCGAELVDALQFVCGDRGFYFNKPTGYGSSSRRAPQTGIVDECCFRSCDLRRLEMYCAPLKPAKSA

SEQ ID NO: 585 - protein, 70 aa, synthetic construct
GPETICGAELVDALQFVCGDRGFYFNKPTGYGSSSRRAPQTGIVDECCFRSCDLRRLEMYCAPLKPAKSA

SEQ ID NO: 586 - protein, 70 aa, synthetic construct
GPETKCGAELVDALQFVCGDRGFYFNKPTGYGSSSRRAPQTGIVDECCFRSCDLRRLEMYCAPLKPAKSA

SEQ ID NO: 587 - protein, 70 aa, synthetic construct
GPETMCGAELVDALQFVCGDRGFYFNKPTGYGSSSRRAPQTGIVDECCFRSCDLRRLEMYCAPLKPAKSA

SEQ ID NO: 588 - protein, 70 aa, synthetic construct
GPETNCGAELVDALQFVCGDRGFYFNKPTGYGSSSRRAPQTGIVDECCFRSCDLRRLEMYCAPLKPAKSA

SEQ ID NO: 589 - protein, 70 aa, synthetic construct
GPETPCGAELVDALQFVCGDRGFYFNKPTGYGSSSRRAPQTGIVDECCFRSCDLRRLEMYCAPLKPAKSA

SEQ ID NO: 590 - protein, 70 aa, synthetic construct
GPETQCGAELVDALQFVCGDRGFYFNKPTGYGSSSRRAPQTGIVDECCFRSCDLRRLEMYCAPLKPAKSA

SEQ ID NO: 591 - protein, 70 aa, synthetic construct
GPETRCGAELVDALQFVCGDRGFYFNKPTGYGSSSRRAPQTGIVDECCFRSCDLRRLEMYCAPLKPAKSA

SEQ ID NO: 592 - protein, 70 aa, synthetic construct
GPETSCGAELVDALQFVCGDRGFYFNKPTGYGSSSRRAPQTGIVDECCFRSCDLRRLEMYCAPLKPAKSA

SEQ ID NO: 593 - protein, 70 aa, synthetic construct
GPETTCGAELVDALQFVCGDRGFYFNKPTGYGSSSRRAPQTGIVDECCFRSCDLRRLEMYCAPLKPAKSA

SEQ ID NO: 594 - protein, 70 aa, synthetic construct
GPETVCGAELVDALQFVCGDRGFYFNKPTGYGSSSRRAPQTGIVDECCFRSCDLRRLEMYCAPLKPAKSA

SEQ ID NO: 595 - protein, 70 aa, synthetic construct
GPETWCGAELVDALQFVCGDRGFYFNKPTGYGSSSRRAPQTGIVDECCFRSCDLRRLEMYCAPLKPAKSA

SEQ ID NO: 596 - protein, 70 aa, synthetic construct
GPETYCGAELVDALQFVCGDRGFYFNKPTGYGSSSRRAPQTGIVDECCFRSCDLRRLEMYCAPLKPAKSA

SEQ ID NO: 597 - protein, 70 aa, synthetic construct
GPETLCAAELVDALQFVCGDRGFYFNKPTGYGSSSRRAPQTGIVDECCFRSCDLRRLEMYCAPLKPAKSA

SEQ ID NO: 598 - protein, 70 aa, synthetic construct
GPETLCCAELVDALQFVCGDRGFYFNKPTGYGSSSRRAPQTGIVDECCFRSCDLRRLEMYCAPLKPAKSA

SEQ ID NO: 599 - protein, 70 aa, synthetic construct
GPETLCDAELVDALQFVCGDRGFYFNKPTGYGSSSRRAPQTGIVDECCFRSCDLRRLEMYCAPLKPAKSA

SEQ ID NO: 600 - protein, 70 aa, synthetic construct
GPETLCEAELVDALQFVCGDRGFYFNKPTGYGSSSRRAPQTGIVDECCFRSCDLRRLEMYCAPLKPAKSA

SEQ ID NO: 601 - protein, 70 aa, synthetic construct
GPETLCFAELVDALQFVCGDRGFYFNKPTGYGSSSRRAPQTGIVDECCFRSCDLRRLEMYCAPLKPAKSA

SEQ ID NO: 602 - protein, 70 aa, synthetic construct
GPETLCHAELVDALQFVCGDRGFYFNKPTGYGSSSRRAPQTGIVDECCFRSCDLRRLEMYCAPLKPAKSA

SEQ ID NO: 603 - protein, 70 aa, synthetic construct
GPETLCIAELVDALQFVCGDRGFYFNKPTGYGSSSRRAPQTGIVDECCFRSCDLRRLEMYCAPLKPAKSA

SEQ ID NO: 604 - protein, 70 aa, synthetic construct
GPETLCKAELVDALQFVCGDRGFYFNKPTGYGSSSRRAPQTGIVDECCFRSCDLRRLEMYCAPLKPAKSA

SEQ ID NO: 605 - protein, 70 aa, synthetic construct
GPETLCLAELVDALQFVCGDRGFYFNKPTGYGSSSRRAPQTGIVDECCFRSCDLRRLEMYCAPLKPAKSA

SEQ ID NO: 606 - protein, 70 aa, synthetic construct
GPETLCMAELVDALQFVCGDRGFYFNKPTGYGSSSRRAPQTGIVDECCFRSCDLRRLEMYCAPLKPAKSA

SEQ ID NO: 607 - protein, 70 aa, synthetic construct
GPETLCNAELVDALQFVCGDRGFYFNKPTGYGSSSRRAPQTGIVDECCFRSCDLRRLEMYCAPLKPAKSA

SEQ ID NO: 608 - protein, 70 aa, synthetic construct
GPETLCPAELVDALQFVCGDRGFYFNKPTGYGSSSRRAPQTGIVDECCFRSCDLRRLEMYCAPLKPAKSA

SEQ ID NO: 609 - protein, 70 aa, synthetic construct
GPETLCQAELVDALQFVCGDRGFYFNKPTGYGSSSRRAPQTGIVDECCFRSCDLRRLEMYCAPLKPAKSA

SEQ ID NO: 610 - protein, 70 aa, synthetic construct
GPETLCRAELVDALQFVCGDRGFYFNKPTGYGSSSRRAPQTGIVDECCFRSCDLRRLEMYCAPLKPAKSA

SEQ ID NO: 611 - protein, 70 aa, synthetic construct
GPETLCSAELVDALQFVCGDRGFYFNKPTGYGSSSRRAPQTGIVDECCFRSCDLRRLEMYCAPLKPAKSA

SEQ ID NO: 612 - protein, 70 aa, synthetic construct
GPETLCTAELVDALQFVCGDRGFYFNKPTGYGSSSRRAPQTGIVDECCFRSCDLRRLEMYCAPLKPAKSA

SEQ ID NO: 613 - protein, 70 aa, synthetic construct
GPETLCVAELVDALQFVCGDRGFYFNKPTGYGSSSRRAPQTGIVDECCFRSCDLRRLEMYCAPLKPAKSA

SEQ ID NO: 614 - protein, 70 aa, synthetic construct
GPETLCWAELVDALQFVCGDRGFYFNKPTGYGSSSRRAPQTGIVDECCFRSCDLRRLEMYCAPLKPAKSA

SEQ ID NO: 615 - protein, 70 aa, synthetic construct
GPETLCYAELVDALQFVCGDRGFYFNKPTGYGSSSRRAPQTGIVDECCFRSCDLRRLEMYCAPLKPAKSA

SEQ ID NO: 616 - protein, 70 aa, synthetic construct
GPETLCGAEAVDALQFVCGDRGFYFNKPTGYGSSSRRAPQTGIVDECCFRSCDLRRLEMYCAPLKPAKSA

SEQ ID NO: 617 - protein, 70 aa, synthetic construct
GPETLCGAECVDALQFVCGDRGFYFNKPTGYGSSSRRAPQTGIVDECCFRSCDLRRLEMYCAPLKPAKSA

SEQ ID NO: 618 - protein, 70 aa, synthetic construct
GPETLCGAEDVDALQFVCGDRGFYFNKPTGYGSSSRRAPQTGIVDECCFRSCDLRRLEMYCAPLKPAKSA

SEQ ID NO: 619 - protein, 70 aa, synthetic construct
GPETLCGAEEVDALQFVCGDRGFYFNKPTGYGSSSRRAPQTGIVDECCFRSCDLRRLEMYCAPLKPAKSA

SEQ ID NO: 620 - protein, 70 aa, synthetic construct
GPETLCGAEFVDALQFVCGDRGFYFNKPTGYGSSSRRAPQTGIVDECCFRSCDLRRLEMYCAPLKPAKSA

SEQ ID NO: 621 - protein, 70 aa, synthetic construct
GPETLCGAEGVDALQFVCGDRGFYFNKPTGYGSSSRRAPQTGIVDECCFRSCDLRRLEMYCAPLKPAKSA

SEQ ID NO: 622 - protein, 70 aa, synthetic construct
GPETLCGAEHVDALQFVCGDRGFYFNKPTGYGSSSRRAPQTGIVDECCFRSCDLRRLEMYCAPLKPAKSA

SEQ ID NO: 623 - protein, 70 aa, synthetic construct
GPETLCGAEIVDALQFVCGDRGFYFNKPTGYGSSSRRAPQTGIVDECCFRSCDLRRLEMYCAPLKPAKSA

SEQ ID NO: 624 - protein, 70 aa, synthetic construct
GPETLCGAEKVDALQFVCGDRGFYFNKPTGYGSSSRRAPQTGIVDECCFRSCDLRRLEMYCAPLKPAKSA

SEQ ID NO: 625 - protein, 70 aa, synthetic construct
GPETLCGAEMVDALQFVCGDRGFYFNKPTGYGSSSRRAPQTGIVDECCFRSCDLRRLEMYCAPLKPAKSA

SEQ ID NO: 626 - protein, 70 aa, synthetic construct
GPETLCGAENVDALQFVCGDRGFYFNKPTGYGSSSRRAPQTGIVDECCFRSCDLRRLEMYCAPLKPAKSA

SEQ ID NO: 627 - protein, 70 aa, synthetic construct
GPETLCGAEPVDALQFVCGDRGFYFNKPTGYGSSSRRAPQTGIVDECCFRSCDLRRLEMYCAPLKPAKSA

SEQ ID NO: 628 - protein, 70 aa, synthetic construct
GPETLCGAEQVDALQFVCGDRGFYFNKPTGYGSSSRRAPQTGIVDECCFRSCDLRRLEMYCAPLKPAKSA

SEQ ID NO: 629 - protein, 70 aa, synthetic construct
GPETLCGAERVDALQFVCGDRGFYFNKPTGYGSSSRRAPQTGIVDECCFRSCDLRRLEMYCAPLKPAKSA

SEQ ID NO: 630 - protein, 70 aa, synthetic construct
GPETLCGAESVDALQFVCGDRGFYFNKPTGYGSSSRRAPQTGIVDECCFRSCDLRRLEMYCAPLKPAKSA

SEQ ID NO: 631 - protein, 70 aa, synthetic construct
GPETLCGAETVDALQFVCGDRGFYFNKPTGYGSSSRRAPQTGIVDECCFRSCDLRRLEMYCAPLKPAKSA

SEQ ID NO: 632 - protein, 70 aa, synthetic construct
GPETLCGAEVVDALQFVCGDRGFYFNKPTGYGSSSRRAPQTGIVDECCFRSCDLRRLEMYCAPLKPAKSA

SEQ ID NO: 633 - protein, 70 aa, synthetic construct
GPETLCGAEWVDALQFVCGDRGFYFNKPTGYGSSSRRAPQTGIVDECCFRSCDLRRLEMYCAPLKPAKSA

SEQ ID NO: 634 - protein, 70 aa, synthetic construct
GPETLCGAEYVDALQFVCGDRGFYFNKPTGYGSSSRRAPQTGIVDECCFRSCDLRRLEMYCAPLKPAKSA

SEQ ID NO: 635 - protein, 70 aa, synthetic construct
GPETLCGAELVDALQFACGDRGFYFNKPTGYGSSSRRAPQTGIVDECCFRSCDLRRLEMYCAPLKPAKSA

SEQ ID NO: 636 - protein, 70 aa, synthetic construct
GPETLCGAELVDALQFCCGDRGFYFNKPTGYGSSSRRAPQTGIVDECCFRSCDLRRLEMYCAPLKPAKSA

SEQ ID NO: 637 - protein, 70 aa, synthetic construct
GPETLCGAELVDALQFDCGDRGFYFNKPTGYGSSSRRAPQTGIVDECCFRSCDLRRLEMYCAPLKPAKSA

SEQ ID NO: 638 - protein, 70 aa, synthetic construct
GPETLCGAELVDALQFECGDRGFYFNKPTGYGSSSRRAPQTGIVDECCFRSCDLRRLEMYCAPLKPAKSA

SEQ ID NO: 639 - protein, 70 aa, synthetic construct
GPETLCGAELVDALQFFCGDRGFYFNKPTGYGSSSRRAPQTGIVDECCFRSCDLRRLEMYCAPLKPAKSA

SEQ ID NO: 640 - protein, 70 aa, synthetic construct
GPETLCGAELVDALQFGCGDRGFYFNKPTGYGSSSRRAPQTGIVDECCFRSCDLRRLEMYCAPLKPAKSA

SEQ ID NO: 641 - protein, 70 aa, synthetic construct
GPETLCGAELVDALQFHCGDRGFYFNKPTGYGSSSRRAPQTGIVDECCFRSCDLRRLEMYCAPLKPAKSA

SEQ ID NO: 642 - protein, 70 aa, synthetic construct
GPETLCGAELVDALQFICGDRGFYFNKPTGYGSSSRRAPQTGIVDECCFRSCDLRRLEMYCAPLKPAKSA

SEQ ID NO: 643 - protein, 70 aa, synthetic construct
GPETLCGAELVDALQFKCGDRGFYFNKPTGYGSSSRRAPQTGIVDECCFRSCDLRRLEMYCAPLKPAKSA

SEQ ID NO: 644 - protein, 70 aa, synthetic construct
GPETLCGAELVDALQFLCGDRGFYFNKPTGYGSSSRRAPQTGIVDECCFRSCDLRRLEMYCAPLKPAKSA

SEQ ID NO: 645 - protein, 70 aa, synthetic construct
GPETLCGAELVDALQFMCGDRGFYFNKPTGYGSSSRRAPQTGIVDECCFRSCDLRRLEMYCAPLKPAKSA

SEQ ID NO: 646 - protein, 70 aa, synthetic construct
GPETLCGAELVDALQFNCGDRGFYFNKPTGYGSSSRRAPQTGIVDECCFRSCDLRRLEMYCAPLKPAKSA

SEQ ID NO: 647 - protein, 70 aa, synthetic construct
GPETLCGAELVDALQFPCGDRGFYFNKPTGYGSSSRRAPQTGIVDECCFRSCDLRRLEMYCAPLKPAKSA

SEQ ID NO: 648 - protein, 70 aa, synthetic construct
GPETLCGAELVDALQFQCGDRGFYFNKPTGYGSSSRRAPQTGIVDECCFRSCDLRRLEMYCAPLKPAKSA

SEQ ID NO: 649 - protein, 70 aa, synthetic construct
GPETLCGAELVDALQFRCGDRGFYFNKPTGYGSSSRRAPQTGIVDECCFRSCDLRRLEMYCAPLKPAKSA

SEQ ID NO: 650 - protein, 70 aa, synthetic construct
GPETLCGAELVDALQFSCGDRGFYFNKPTGYGSSSRRAPQTGIVDECCFRSCDLRRLEMYCAPLKPAKSA

SEQ ID NO: 651 - protein, 70 aa, synthetic construct
GPETLCGAELVDALQFTCGDRGFYFNKPTGYGSSSRRAPQTGIVDECCFRSCDLRRLEMYCAPLKPAKSA

SEQ ID NO: 652 - protein, 70 aa, synthetic construct
GPETLCGAELVDALQFWCGDRGFYFNKPTGYGSSSRRAPQTGIVDECCFRSCDLRRLEMYCAPLKPAKSA

SEQ ID NO: 653 - protein, 70 aa, synthetic construct
GPETLCGAELVDALQFYCGDRGFYFNKPTGYGSSSRRAPQTGIVDECCFRSCDLRRLEMYCAPLKPAKSA

SEQ ID NO: 654 - protein, 70 aa, synthetic construct
GPETLCGAELVDALQFVCGDRAFYFNKPTGYGSSSRRAPQTGIVDECCFRSCDLRRLEMYCAPLKPAKSA

SEQ ID NO: 655 - protein, 70 aa, synthetic construct
GPETLCGAELVDALQFVCGDRCFYFNKPTGYGSSSRRAPQTGIVDECCFRSCDLRRLEMYCAPLKPAKSA

SEQ ID NO: 656 - protein, 70 aa, synthetic construct
GPETLCGAELVDALQFVCGDRDFYFNKPTGYGSSSRRAPQTGIVDECCFRSCDLRRLEMYCAPLKPAKSA

SEQ ID NO: 657 - protein, 70 aa, synthetic construct
GPETLCGAELVDALQFVCGDREFYFNKPTGYGSSSRRAPQTGIVDECCFRSCDLRRLEMYCAPLKPAKSA

SEQ ID NO: 658 - protein, 70 aa, synthetic construct
GPETLCGAELVDALQFVCGDRFFYFNKPTGYGSSSRRAPQTGIVDECCFRSCDLRRLEMYCAPLKPAKSA

SEQ ID NO: 659 - protein, 70 aa, synthetic construct
GPETLCGAELVDALQFVCGDRHFYFNKPTGYGSSSRRAPQTGIVDECCFRSCDLRRLEMYCAPLKPAKSA

SEQ ID NO: 660 - protein, 70 aa, synthetic construct
GPETLCGAELVDALQFVCGDRIFYFNKPTGYGSSSRRAPQTGIVDECCFRSCDLRRLEMYCAPLKPAKSA

SEQ ID NO: 661 - protein, 70 aa, synthetic construct
GPETLCGAELVDALQFVCGDRKFYFNKPTGYGSSSRRAPQTGIVDECCFRSCDLRRLEMYCAPLKPAKSA

SEQ ID NO: 662 - protein, 70 aa, synthetic construct
GPETLCGAELVDALQFVCGDRLFYFNKPTGYGSSSRRAPQTGIVDECCFRSCDLRRLEMYCAPLKPAKSA

SEQ ID NO: 663 - protein, 70 aa, synthetic construct
GPETLCGAELVDALQFVCGDRMFYFNKPTGYGSSSRRAPQTGIVDECCFRSCDLRRLEMYCAPLKPAKSA

SEQ ID NO: 664 - protein, 70 aa, synthetic construct
GPETLCGAELVDALQFVCGDRNFYFNKPTGYGSSSRRAPQTGIVDECCFRSCDLRRLEMYCAPLKPAKSA

SEQ ID NO: 665 - protein, 70 aa, synthetic construct
GPETLCGAELVDALQFVCGDRPFYFNKPTGYGSSSRRAPQTGIVDECCFRSCDLRRLEMYCAPLKPAKSA

SEQ ID NO: 666 - protein, 70 aa, synthetic construct
GPETLCGAELVDALQFVCGDRQFYFNKPTGYGSSSRRAPQTGIVDECCFRSCDLRRLEMYCAPLKPAKSA

SEQ ID NO: 667 - protein, 70 aa, synthetic construct
GPETLCGAELVDALQFVCGDRRFYFNKPTGYGSSSRRAPQTGIVDECCFRSCDLRRLEMYCAPLKPAKSA

SEQ ID NO: 668 - protein, 70 aa, synthetic construct
GPETLCGAELVDALQFVCGDRSFYFNKPTGYGSSSRRAPQTGIVDECCFRSCDLRRLEMYCAPLKPAKSA

SEQ ID NO: 669 - protein, 70 aa, synthetic construct
GPETLCGAELVDALQFVCGDRTFYFNKPTGYGSSSRRAPQTGIVDECCFRSCDLRRLEMYCAPLKPAKSA

SEQ ID NO: 670 - protein, 70 aa, synthetic construct
GPETLCGAELVDALQFVCGDRVFYFNKPTGYGSSSRRAPQTGIVDECCFRSCDLRRLEMYCAPLKPAKSA

SEQ ID NO: 671 - protein, 70 aa, synthetic construct
GPETLCGAELVDALQFVCGDRWFYFNKPTGYGSSSRRAPQTGIVDECCFRSCDLRRLEMYCAPLKPAKSA

SEQ ID NO: 672 - protein, 70 aa, synthetic construct
GPETLCGAELVDALQFVCGDRYFYFNKPTGYGSSSRRAPQTGIVDECCFRSCDLRRLEMYCAPLKPAKSA

SEQ ID NO: 673 - protein, 70 aa, synthetic construct
GPETLCGAELVDALQFVCGDRGAYFNKPTGYGSSSRRAPQTGIVDECCFRSCDLRRLEMYCAPLKPAKSA

SEQ ID NO: 674 - protein, 70 aa, synthetic construct
GPETLCGAELVDALQFVCGDRGCYFNKPTGYGSSSRRAPQTGIVDECCFRSCDLRRLEMYCAPLKPAKSA

SEQ ID NO: 675 - protein, 70 aa, synthetic construct
GPETLCGAELVDALQFVCGDRGDYFNKPTGYGSSSRRAPQTGIVDECCFRSCDLRRLEMYCAPLKPAKSA

SEQ ID NO: 676 - protein, 70 aa, synthetic construct
GPETLCGAELVDALQFVCGDRGEYFNKPTGYGSSSRRAPQTGIVDECCFRSCDLRRLEMYCAPLKPAKSA

SEQ ID NO: 677 - protein, 70 aa, synthetic construct
GPETLCGAELVDALQFVCGDRGGYFNKPTGYGSSSRRAPQTGIVDECCFRSCDLRRLEMYCAPLKPAKSA

SEQ ID NO: 678 - protein, 70 aa, synthetic construct
GPETLCGAELVDALQFVCGDRGHYFNKPTGYGSSSRRAPQTGIVDECCFRSCDLRRLEMYCAPLKPAKSA

SEQ ID NO: 679 - protein, 70 aa, synthetic construct
GPETLCGAELVDALQFVCGDRGIYFNKPTGYGSSSRRAPQTGIVDECCFRSCDLRRLEMYCAPLKPAKSA

SEQ ID NO: 680 - protein, 70 aa, synthetic construct
GPETLCGAELVDALQFVCGDRGKYFNKPTGYGSSSRRAPQTGIVDECCFRSCDLRRLEMYCAPLKPAKSA

SEQ ID NO: 681 - protein, 70 aa, synthetic construct
GPETLCGAELVDALQFVCGDRGLYFNKPTGYGSSSRRAPQTGIVDECCFRSCDLRRLEMYCAPLKPAKSA

SEQ ID NO: 682 - protein, 70 aa, synthetic construct
GPETLCGAELVDALQFVCGDRGMYFNKPTGYGSSSRRAPQTGIVDECCFRSCDLRRLEMYCAPLKPAKSA

SEQ ID NO: 683 - protein, 70 aa, synthetic construct
GPETLCGAELVDALQFVCGDRGNYFNKPTGYGSSSRRAPQTGIVDECCFRSCDLRRLEMYCAPLKPAKSA

SEQ ID NO: 684 - protein, 70 aa, synthetic construct
GPETLCGAELVDALQFVCGDRGPYFNKPTGYGSSSRRAPQTGIVDECCFRSCDLRRLEMYCAPLKPAKSA

SEQ ID NO: 685 - protein, 70 aa, synthetic construct
GPETLCGAELVDALQFVCGDRGQYFNKPTGYGSSSRRAPQTGIVDECCFRSCDLRRLEMYCAPLKPAKSA

SEQ ID NO: 686 - protein, 70 aa, synthetic construct
GPETLCGAELVDALQFVCGDRGRYFNKPTGYGSSSRRAPQTGIVDECCFRSCDLRRLEMYCAPLKPAKSA

SEQ ID NO: 687 - protein, 70 aa, synthetic construct
GPETLCGAELVDALQFVCGDRGSYFNKPTGYGSSSRRAPQTGIVDECCFRSCDLRRLEMYCAPLKPAKSA

SEQ ID NO: 688 - protein, 70 aa, synthetic construct
GPETLCGAELVDALQFVCGDRGTYFNKPTGYGSSSRRAPQTGIVDECCFRSCDLRRLEMYCAPLKPAKSA

SEQ ID NO: 689 - protein, 70 aa, synthetic construct
GPETLCGAELVDALQFVCGDRGVYFNKPTGYGSSSRRAPQTGIVDECCFRSCDLRRLEMYCAPLKPAKSA

SEQ ID NO: 690 - protein, 70 aa, synthetic construct
GPETLCGAELVDALQFVCGDRGWYFNKPTGYGSSSRRAPQTGIVDECCFRSCDLRRLEMYCAPLKPAKSA

SEQ ID NO: 691 - protein, 70 aa, synthetic construct
GPETLCGAELVDALQFVCGDRGYYFNKPTGYGSSSRRAPQTGIVDECCFRSCDLRRLEMYCAPLKPAKSA

SEQ ID NO: 692 - protein, 70 aa, synthetic construct
GPETLCGAELVDALQFVCGDRGFAFNKPTGYGSSSRRAPQTGIVDECCFRSCDLRRLEMYCAPLKPAKSA

SEQ ID NO: 693 - protein, 70 aa, synthetic construct
GPETLCGAELVDALQFVCGDRGFCFNKPTGYGSSSRRAPQTGIVDECCFRSCDLRRLEMYCAPLKPAKSA

SEQ ID NO: 694 - protein, 70 aa, synthetic construct
GPETLCGAELVDALQFVCGDRGFDFNKPTGYGSSSRRAPQTGIVDECCFRSCDLRRLEMYCAPLKPAKSA

SEQ ID NO: 695 - protein, 70 aa, synthetic construct
GPETLCGAELVDALQFVCGDRGFEFNKPTGYGSSSRRAPQTGIVDECCFRSCDLRRLEMYCAPLKPAKSA

SEQ ID NO: 696 - protein, 70 aa, synthetic construct
GPETLCGAELVDALQFVCGDRGFFFNKPTGYGSSSRRAPQTGIVDECCFRSCDLRRLEMYCAPLKPAKSA

SEQ ID NO: 697 - protein, 70 aa, synthetic construct
GPETLCGAELVDALQFVCGDRGFGFNKPTGYGSSSRRAPQTGIVDECCFRSCDLRRLEMYCAPLKPAKSA

SEQ ID NO: 698 - protein, 70 aa, synthetic construct
GPETLCGAELVDALQFVCGDRGFHFNKPTGYGSSSRRAPQTGIVDECCFRSCDLRRLEMYCAPLKPAKSA

SEQ ID NO: 699 - protein, 70 aa, synthetic construct
GPETLCGAELVDALQFVCGDRGFIFNKPTGYGSSSRRAPQTGIVDECCFRSCDLRRLEMYCAPLKPAKSA

SEQ ID NO: 700 - protein, 70 aa, synthetic construct
GPETLCGAELVDALQFVCGDRGFKFNKPTGYGSSSRRAPQTGIVDECCFRSCDLRRLEMYCAPLKPAKSA

SEQ ID NO: 701 - protein, 70 aa, synthetic construct
GPETLCGAELVDALQFVCGDRGFLFNKPTGYGSSSRRAPQTGIVDECCFRSCDLRRLEMYCAPLKPAKSA

SEQ ID NO: 702 - protein, 70 aa, synthetic construct
GPETLCGAELVDALQFVCGDRGFMFNKPTGYGSSSRRAPQTGIVDECCFRSCDLRRLEMYCAPLKPAKSA

SEQ ID NO: 703 - protein, 70 aa, synthetic construct
GPETLCGAELVDALQFVCGDRGFNFNKPTGYGSSSRRAPQTGIVDECCFRSCDLRRLEMYCAPLKPAKSA

SEQ ID NO: 704 - protein, 70 aa, synthetic construct
GPETLCGAELVDALQFVCGDRGFPFNKPTGYGSSSRRAPQTGIVDECCFRSCDLRRLEMYCAPLKPAKSA

SEQ ID NO: 705 - protein, 70 aa, synthetic construct
GPETLCGAELVDALQFVCGDRGFQFNKPTGYGSSSRRAPQTGIVDECCFRSCDLRRLEMYCAPLKPAKSA

SEQ ID NO: 706 - protein, 70 aa, synthetic construct
GPETLCGAELVDALQFVCGDRGFRFNKPTGYGSSSRRAPQTGIVDECCFRSCDLRRLEMYCAPLKPAKSA

SEQ ID NO: 707 - protein, 70 aa, synthetic construct
GPETLCGAELVDALQFVCGDRGFSFNKPTGYGSSSRRAPQTGIVDECCFRSCDLRRLEMYCAPLKPAKSA

SEQ ID NO: 708 - protein, 70 aa, synthetic construct
GPETLCGAELVDALQFVCGDRGFTFNKPTGYGSSSRRAPQTGIVDECCFRSCDLRRLEMYCAPLKPAKSA

SEQ ID NO: 709 - protein, 70 aa, synthetic construct
GPETLCGAELVDALQFVCGDRGFVFNKPTGYGSSSRRAPQTGIVDECCFRSCDLRRLEMYCAPLKPAKSA

SEQ ID NO: 710 - protein, 70 aa, synthetic construct
GPETLCGAELVDALQFVCGDRGFWFNKPTGYGSSSRRAPQTGIVDECCFRSCDLRRLEMYCAPLKPAKSA

SEQ ID NO: 711 - protein, 70 aa, synthetic construct
GPETLCGAELVDALQFVCGDRGFYANKPTGYGSSSRRAPQTGIVDECCFRSCDLRRLEMYCAPLKPAKSA

SEQ ID NO: 712 - protein, 70 aa, synthetic construct
GPETLCGAELVDALQFVCGDRGFYCNKPTGYGSSSRRAPQTGIVDECCFRSCDLRRLEMYCAPLKPAKSA

SEQ ID NO: 713 - protein, 70 aa, synthetic construct
GPETLCGAELVDALQFVCGDRGFYDNKPTGYGSSSRRAPQTGIVDECCFRSCDLRRLEMYCAPLKPAKSA

SEQ ID NO: 714 - protein, 70 aa, synthetic construct
GPETLCGAELVDALQFVCGDRGFYENKPTGYGSSSRRAPQTGIVDECCFRSCDLRRLEMYCAPLKPAKSA

SEQ ID NO: 715 - protein, 70 aa, synthetic construct
GPETLCGAELVDALQFVCGDRGFYGNKPTGYGSSSRRAPQTGIVDECCFRSCDLRRLEMYCAPLKPAKSA

SEQ ID NO: 716 - protein, 70 aa, synthetic construct
GPETLCGAELVDALQFVCGDRGFYHNKPTGYGSSSRRAPQTGIVDECCFRSCDLRRLEMYCAPLKPAKSA

SEQ ID NO: 717 - protein, 70 aa, synthetic construct
GPETLCGAELVDALQFVCGDRGFYINKPTGYGSSSRRAPQTGIVDECCFRSCDLRRLEMYCAPLKPAKSA

SEQ ID NO: 718 - protein, 70 aa, synthetic construct
GPETLCGAELVDALQFVCGDRGFYKNKPTGYGSSSRRAPQTGIVDECCFRSCDLRRLEMYCAPLKPAKSA

SEQ ID NO: 719 - protein, 70 aa, synthetic construct
GPETLCGAELVDALQFVCGDRGFYLNKPTGYGSSSRRAPQTGIVDECCFRSCDLRRLEMYCAPLKPAKSA

SEQ ID NO: 720 - protein, 70 aa, synthetic construct
GPETLCGAELVDALQFVCGDRGFYMNKPTGYGSSSRRAPQTGIVDECCFRSCDLRRLEMYCAPLKPAKSA

SEQ ID NO: 721 - protein, 70 aa, synthetic construct
GPETLCGAELVDALQFVCGDRGFYNNKPTGYGSSSRRAPQTGIVDECCFRSCDLRRLEMYCAPLKPAKSA

SEQ ID NO: 722 - protein, 70 aa, synthetic construct
GPETLCGAELVDALQFVCGDRGFYPNKPTGYGSSSRRAPQTGIVDECCFRSCDLRRLEMYCAPLKPAKSA

SEQ ID NO: 723 - protein, 70 aa, synthetic construct
GPETLCGAELVDALQFVCGDRGFYQNKPTGYGSSSRRAPQTGIVDECCFRSCDLRRLEMYCAPLKPAKSA

SEQ ID NO: 724 - protein, 70 aa, synthetic construct
GPETLCGAELVDALQFVCGDRGFYRNKPTGYGSSSRRAPQTGIVDECCFRSCDLRRLEMYCAPLKPAKSA

SEQ ID NO: 725 - protein, 70 aa, synthetic construct
GPETLCGAELVDALQFVCGDRGFYSNKPTGYGSSSRRAPQTGIVDECCFRSCDLRRLEMYCAPLKPAKSA

SEQ ID NO: 726 - protein, 70 aa, synthetic construct
GPETLCGAELVDALQFVCGDRGFYTNKPTGYGSSSRRAPQTGIVDECCFRSCDLRRLEMYCAPLKPAKSA

SEQ ID NO: 727 - protein, 70 aa, synthetic construct
GPETLCGAELVDALQFVCGDRGFYVNKPTGYGSSSRRAPQTGIVDECCFRSCDLRRLEMYCAPLKPAKSA

SEQ ID NO: 728 - protein, 70 aa, synthetic construct
GPETLCGAELVDALQFVCGDRGFYWNKPTGYGSSSRRAPQTGIVDECCFRSCDLRRLEMYCAPLKPAKSA

SEQ ID NO: 729 - protein, 70 aa, synthetic construct
GPETLCGAELVDALQFVCGDRGFYYNKPTGYGSSSRRAPQTGIVDECCFRSCDLRRLEMYCAPLKPAKSA

SEQ ID NO: 730 - protein, 70 aa, synthetic construct
GPETLCGAELVDALQFVCGDRGFYFNKPTGYGSASRRAPQTGIVDECCFRSCDLRRLEMYCAPLKPAKSA

SEQ ID NO: 731 - protein, 70 aa, synthetic construct
GPETLCGAELVDALQFVCGDRGFYFNKPTGYGSCSRRAPQTGIVDECCFRSCDLRRLEMYCAPLKPAKSA

SEQ ID NO: 732 - protein, 70 aa, synthetic construct
GPETLCGAELVDALQFVCGDRGFYFNKPTGYGSDSRRAPQTGIVDECCFRSCDLRRLEMYCAPLKPAKSA

SEQ ID NO: 733 - protein, 70 aa, synthetic construct
GPETLCGAELVDALQFVCGDRGFYFNKPTGYGSESRRAPQTGIVDECCFRSCDLRRLEMYCAPLKPAKSA

SEQ ID NO: 734 - protein, 70 aa, synthetic construct
GPETLCGAELVDALQFVCGDRGFYFNKPTGYGSFSRRAPQTGIVDECCFRSCDLRRLEMYCAPLKPAKSA

SEQ ID NO: 735 - protein, 70 aa, synthetic construct
GPETLCGAELVDALQFVCGDRGFYFNKPTGYGSGSRRAPQTGIVDECCFRSCDLRRLEMYCAPLKPAKSA

SEQ ID NO: 736 - protein, 70 aa, synthetic construct
GPETLCGAELVDALQFVCGDRGFYFNKPTGYGSHSRRAPQTGIVDECCFRSCDLRRLEMYCAPLKPAKSA

SEQ ID NO: 737 - protein, 70 aa, synthetic construct
GPETLCGAELVDALQFVCGDRGFYFNKPTGYGSISRRAPQTGIVDECCFRSCDLRRLEMYCAPLKPAKSA

SEQ ID NO: 738 - protein, 70 aa, synthetic construct
GPETLCGAELVDALQFVCGDRGFYFNKPTGYGSKSRRAPQTGIVDECCFRSCDLRRLEMYCAPLKPAKSA

SEQ ID NO: 739 - protein, 70 aa, synthetic construct
GPETLCGAELVDALQFVCGDRGFYFNKPTGYGSLSRRAPQTGIVDECCFRSCDLRRLEMYCAPLKPAKSA

SEQ ID NO: 740 - protein, 70 aa, synthetic construct
GPETLCGAELVDALQFVCGDRGFYFNKPTGYGSMSRRAPQTGIVDECCFRSCDLRRLEMYCAPLKPAKSA

SEQ ID NO: 741 - protein, 70 aa, synthetic construct
GPETLCGAELVDALQFVCGDRGFYFNKPTGYGSNSRRAPQTGIVDECCFRSCDLRRLEMYCAPLKPAKSA

SEQ ID NO: 742 - protein, 70 aa, synthetic construct
GPETLCGAELVDALQFVCGDRGFYFNKPTGYGSPSRRAPQTGIVDECCFRSCDLRRLEMYCAPLKPAKSA

SEQ ID NO: 743 - protein, 70 aa, synthetic construct
GPETLCGAELVDALQFVCGDRGFYFNKPTGYGSQSRRAPQTGIVDECCFRSCDLRRLEMYCAPLKPAKSA

SEQ ID NO: 744 - protein, 70 aa, synthetic construct; identical to SEQ ID NO: 1

SEQ ID NO: 745 - protein, 70 aa, synthetic construct
GPETLCGAELVDALQFVCGDRGFYFNKPTGYGSTSRRAPQTGIVDECCFRSCDLRRLEMYCAPLKPAKSA

SEQ ID NO: 746 - protein, 70 aa, synthetic construct
GPETLCGAELVDALQFVCGDRGFYFNKPTGYGSVSRRAPQTGIVDECCFRSCDLRRLEMYCAPLKPAKSA

SEQ ID NO: 747 - protein, 70 aa, synthetic construct
GPETLCGAELVDALQFVCGDRGFYFNKPTGYGSWSRRAPQTGIVDECCFRSCDLRRLEMYCAPLKPAKSA

SEQ ID NO: 748 - protein, 70 aa, synthetic construct
GPETLCGAELVDALQFVCGDRGFYFNKPTGYGSYSRRAPQTGIVDECCFRSCDLRRLEMYCAPLKPAKSA

SEQ ID NO: 749 - protein, 70 aa, synthetic construct
GPETLCGAELVDALQFVCGDRGFYFNKPTGYGSSSRRAPQTGIVDECCFRSCDLRRAEMYCAPLKPAKSA

SEQ ID NO: 750 - protein, 70 aa, synthetic construct
GPETLCGAELVDALQFVCGDRGFYFNKPTGYGSSSRRAPQTGIVDECCFRSCDLRRCEMYCAPLKPAKSA

SEQ ID NO: 751 - protein, 70 aa, synthetic construct
GPETLCGAELVDALQFVCGDRGFYFNKPTGYGSSSRRAPQTGIVDECCFRSCDLRRDEMYCAPLKPAKSA

SEQ ID NO: 752 - protein, 70 aa, synthetic construct
GPETLCGAELVDALQFVCGDRGFYFNKPTGYGSSSRRAPQTGIVDECCFRSCDLRREEMYCAPLKPAKSA

SEQ ID NO: 753 - protein, 70 aa, synthetic construct
GPETLCGAELVDALQFVCGDRGFYFNKPTGYGSSSRRAPQTGIVDECCFRSCDLRRFEMYCAPLKPAKSA

SEQ ID NO: 754 - protein, 70 aa, synthetic construct
GPETLCGAELVDALQFVCGDRGFYFNKPTGYGSSSRRAPQTGIVDECCFRSCDLRRGEMYCAPLKPAKSA

SEQ ID NO: 755 - protein, 70 aa, synthetic construct
GPETLCGAELVDALQFVCGDRGFYFNKPTGYGSSSRRAPQTGIVDECCFRSCDLRRHEMYCAPLKPAKSA

SEQ ID NO: 756 - protein, 70 aa, synthetic construct
GPETLCGAELVDALQFVCGDRGFYFNKPTGYGSSSRRAPQTGIVDECCFRSCDLRRIEMYCAPLKPAKSA

SEQ ID NO: 757 - protein, 70 aa, synthetic construct
GPETLCGAELVDALQFVCGDRGFYFNKPTGYGSSSRRAPQTGIVDECCFRSCDLRRKEMYCAPLKPAKSA

SEQ ID NO: 758 - protein, 70 aa, synthetic construct
GPETLCGAELVDALQFVCGDRGFYFNKPTGYGSSSRRAPQTGIVDECCFRSCDLRRMEMYCAPLKPAKSA

SEQ ID NO: 759 - protein, 70 aa, synthetic construct
GPETLCGAELVDALQFVCGDRGFYFNKPTGYGSSSRRAPQTGIVDECCFRSCDLRRNEMYCAPLKPAKSA

SEQ ID NO: 760 - protein, 70 aa, synthetic construct
GPETLCGAELVDALQFVCGDRGFYFNKPTGYGSSSRRAPQTGIVDECCFRSCDLRRPEMYCAPLKPAKSA

SEQ ID NO: 761 - protein, 70 aa, synthetic construct
GPETLCGAELVDALQFVCGDRGFYFNKPTGYGSSSRRAPQTGIVDECCFRSCDLRRQEMYCAPLKPAKSA

SEQ ID NO: 762 - protein, 70 aa, synthetic construct
GPETLCGAELVDALQFVCGDRGFYFNKPTGYGSSSRRAPQTGIVDECCFRSCDLRRREMYCAPLKPAKSA

SEQ ID NO: 763 - protein, 70 aa, synthetic construct
GPETLCGAELVDALQFVCGDRGFYFNKPTGYGSSSRRAPQTGIVDECCFRSCDLRRSEMYCAPLKPAKSA

SEQ ID NO: 764 - protein, 70 aa, synthetic construct
GPETLCGAELVDALQFVCGDRGFYFNKPTGYGSSSRRAPQTGIVDECCFRSCDLRRTEMYCAPLKPAKSA

SEQ ID NO: 765 - protein, 70 aa, synthetic construct
GPETLCGAELVDALQFVCGDRGFYFNKPTGYGSSSRRAPQTGIVDECCFRSCDLRRVEMYCAPLKPAKSA

SEQ ID NO: 766 - protein, 70 aa, synthetic construct
GPETLCGAELVDALQFVCGDRGFYFNKPTGYGSSSRRAPQTGIVDECCFRSCDLRRWEMYCAPLKPAKSA

SEQ ID NO: 767 - protein, 70 aa, synthetic construct
GPETLCGAELVDALQFVCGDRGFYFNKPTGYGSSSRRAPQTGIVDECCFRSCDLRRYEMYCAPLKPAKSA

SEQ ID NO: 768 - protein, 70 aa, synthetic construct
GPKTLCGAELVDALQFVCGDRGFYFNKPTGYGSSSRRAPQTGIVDECCFRSCDLRRLEMYCAPLKPAKSA

SEQ ID NO: 769 - protein, 70 aa, synthetic construct
GPGTLCGAELVDALQFVCGDRGFYFNKPTGYGSSSRRAPQTGIVDECCFRSCDLRRLEMYCAPLKPAKSA

SEQ ID NO: 770 - protein, 70 aa, synthetic construct
GPKTLCGAELVDALQFVCGDRGFYFNKPTGYGSSSRRAPQTGIVDECCLRSCDLRRLEMYCAPLKPAKSA

SEQ ID NO: 771 - protein, 70 aa, synthetic construct
GPGTLCGAELVDALQFVCGDRGFYFNKPTGYGSSSRRAPQTGIVDECCLRSCDLRRLEMYCAPLKPAKSA

SEQ ID NO: 772 - protein, 13 aa, synthetic construct
MFPAMPLLSLFVN